SEQUENCE LISTING (94 sequences)

SEQ ID NO: 1 - DNA, 4138 bp, Triticum aestivum
  source 1..4138: mol_type = unassigned DNA
tttatgcatcacagtcgttgatggccagaagttggacacaagagagaccagcagccagcgctttctcggttttaaaaaaaagaggtcagggcctcttttttatatacaacaaaaataccacagttttagagatatcatggtttgttgaactgcagtattttttttatccaaacgactcaaagtattaaataccaaagtttttttcaaaaaccacagtattccagaaaaccacaaaaatacttcggcttccaaacgcaccctaagtgtaagaagaaatctactaagttcacaaaattcagaagaaactcgtttggaccatgagcggtacagaaaagaaaatggcgtgatctagaatagcagacattagggtctgtttgcagatattatggatcactgtatgtttgcaaaaaataataatttgtgcagctatcagatgcatgcattttctatgaaaattttgtagatccagtgttatacccaacaacaaaatttaatacttgggtcaccacaacccaagatggagctccaagcaaacttaagtaagaggataccgagatgtgatgatgatgtctgatcttgggggaatttttgtagtgagaaaatgccttaaacatcatcgcggaatccaaatggtcaatcaggactgacaccaccaatggcttgattcaccatctctatctgcccattttgacgtagcactatttggctttcgacgcgacggggtaatcaacgtgacaccatcctgtcatgagaaagtcatcatgccgagttcattttccaggcatcccgtgtggcatcttgccattctccccgtccatgagttgattacccaaaagcatctgatcccggagcacaacgaacgacatcagaagacatggttcgcatcacaaaacaccatttggtcatgttacgcatgttaggcacatctgaagaagggaaggcgttgagaacctcggccaccactagtaatagacttccaacttttagttgggacaacatgggcgggatttcacggcacaaaattcaccacctagatgctaaaaagatacccccccccccccatcccccacacatcctccttgaagacacgggcgccccctccgcctccctacctcgccgtcgccggaggaggccgccgggctaagcctgtgcggtggacgacggcggcggggcctctctgcagcgtccgggtctcatggtgagggaggcacgcctcctcgcagtgcggcgcggcggctacggcacgaagattctccgaagttgcgtctccagcgggtggcagtcgcggtggtgtcgtcggcaaactcctgggcttcggtccggcggctcttgctcggcgagtatgggcggggtggcggcccccctttccttcctttggtcattcgtctccgcgcaggtcgactgctctggccggcccttggtggttgttgtagtgctgctgcaagggggcgccagatccggcggttcttcattcggatccagctcgtctctcatcttactcagggtcggtcatcccggtgaccttgcctctggcagcaaggggtgtgtgtgctctatggtgggccgtgggccttgtgaggcgatggcgcggttgctgcgcaggggtgtgcatgccagcgattgcgcgggccagcttgtgtcggctgcgtgagggtgttggttgagtctgctccaagcgcaagcttgcccggctatggccggccggtgctggcggcgcctatgggcgtcgtttcccttgttgaaggcgttgttgtagagttcttcgacctactacaatctcgaatctggttcttcaggcgaaaacctcgcccatctaggtcgggcaacgatgtcgtccgtacggtgttttccccctttggggcgttgctttgaagaccggttatcctttcccgtgctacctttgggctggtcgtgcatggtgtcatggttggctggtgctcaaccggtgatacgagtggttggcgttgcgactcgtgcggtgacgaagatggtgatgcgcgaagctgggtcgcggcttgtccttctgatatcggcggttcggttcccctttgggtgcgtctgtgcttgtttctcactgtgacagagaagttgaagctacatgcggcgggggccctgcggcaatgatgactccatgagggtggtttcggcggacgatgctctccgcaggttgcactggactagcttggtgtgaggcttgcaactttctcctgtgatgctcatcaacaaaatcggagatgtcggtcctcgacgcctgcggccgatcatttggcttggctgtcgaggtcctcgggtgccgtttgttcggaggggtcttgatgggtcgtcagcgaggaaatcggagttgcctgctcgtggagaggcgatgacgatgacattgcagcctcgacggtgtcctctcgtcagcagtatgtgtgtgatcttgtcagtgccaggtcggctggagtgccctattcgatgggcgtgttttaattaaccgggcaactctcttcttcttaatcagtgaaaatggcaagtcttttacctcgtttcaaaaagagttaaatgcactggaggtcctaaaggtttcgtcggggtgtcactttagtcctatttcttccaaaatgcacctttaggtcctaattctttattaattggttcacccgaggtccttttccacgagcgctcgctgaccagcacacgtgccgcattgacccatgccacatcgatagagggtctaaccgccaggcgagaagcttcgcaaagagaccccccccctcttagtggcaccctctctcctctttcacggtactttggcgacattggaggcgagcgacagaggcaaccggaggtgatctggaggtaatttgtgtgggtgcccgtgaagatgccccttctatgacggccagtgagcaatcctcctccatgttaccgtaagaaccctaatcttgtcgatgatcttctcctgtagctcttctaatatgtcataactttgtcataatcaatagttgtagtccttgatttactttgatttgcataacggtgatacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggtttagggacgcgcgcgccgagagagagagcagatttggggaagaaataaagccagggactggggaatgagaagataatgtttataagggttttctgtaaaaagaaagagcaacctacgcagcccttctgtcgatttggcatgggtcaacgcaccacacgagcaggtcaacgagcgctcgtgagaaaaaggacctagggtaaaccacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacatactccctccgtccggaaatacttgtcgaagaatttgatgaaaatggatgcatctagaacaagaatacatctagatacatcaatctccctgacaagtatttccgagcggagggagtactagataatactccctccgttcctaaataattgtctttctagctatctcaaataaactacaacatacggatgtatgtagacatgttttagagtgtagattcactcattttgttccgtatgtagtcatttgttgaaatctctagagagacaattatttaggaacggagggagtaagataactaccctaaaaaaaaagataactgaaggttgccacctagcacattcacattggtacaacttggaaaagcacagccccgtcgtcctgctcccagttgagttcgcgacctacacaccggcc

SEQ ID NO: 2 - DNA, 22 bp, synthetic construct
  source 1..22: mol_type = other DNA
  misc_feature 1..22: note = primer
taatacgactcactatagggcg

SEQ ID NO: 3 - DNA, 17 bp, synthetic construct
  source 1..17: mol_type = other DNA
  misc_feature 1..17: note = primer
gttgaagtgaacttgcg

SEQ ID NO: 4 - protein, 753 aa, Triticum aestivum
MNNSDKKSVCSIPRRSSERRKQSSSELVSSAKRPTRHNTSSSYKPDHCSPPKRTVRTVRNVNLAKYIKNKYYRSPLSKRQSSNAATGKSATGPVRRRNRKRRRHNTDCDEATRMERRARYLLVKIKSEQNLIDAYSGDGWNGHSREKLKPEKELQRAKRQIIKCKIAIRDIIHQLDLYSSSGSMEDSVMPPDESVKSVNPDNTICSRCKSPESVPNNKFIFCEGACKMAYHEKCSEPPLDRSLPTGGPGWLCKFCLCKMKILEAVNAHLGTSLTVMCPSKDIFKEATKEVDSEDGLGEDWLSEYSGDEDYDPEENDFTSSSLDSGEESISDQSNCSGSPLYSPNDDIPGFISADFTDAEGFYHTNSNLGIDSGDDDVAEMLTYQRPKREVDYKRLNEEMFGKLTENEKQSEDEDWGTNNRRKKRRVHSAGVGVKSAEGLSDVKSNEKAQPQRRKLFRIPPEAVQVLRKSFAESELPAREVKESLSTELGISFEKIDKWFKNTRCAALRDRKAEGNSRVAGPSKRSRRSAGKSGSSVKIDSVDNSYLIPASEIISVPTHPQGIPERKPESTSRTVRRLVHDKGAPLCPPAEVKESTSPTTKPGLNPSHVADRIINTEHRAASREAPVAFSDDPFFDAILSYPTINTNDRAVSREDPVAFSDDPFLDVMLANRDIYTEERGVSHEDVKRPSGDQPFLDVIQNVCGLEHRLQRLKGDMLSSGAAPGGTGTAERDLQNQLVVLVPTAELKDKSQPSI

SEQ ID NO: 5 - DNA, 2262 bp, Triticum aestivum
  source 1..2262: mol_type = unassigned DNA
atgaataattcagacaagaagtcggtatgctccattcctagaagatcatcagaacggaggaaacagtcctcttctgagttggtttcttcagctaaaaggccaactcgccacaatacctccagcagttataagccagatcattgttctcctcccaagagaacagtaagaacagtcaggaatgttaatctggcaaaatatataaagaacaagtattatcgaagtcctctgagcaagcggcagtcttcaaatgctgctactggaaaaagtgcaacaggaccagtcaggaggaggaaccggaaacggagaaggcacaatactgattgtgatgaggcaactcgcatggaaagaagggcgagatatttgctggtcaaaataaaatcggagcagaatttaatagatgcatactctggagatggctggaatgggcacagccgagagaaattaaagccagagaaggagctacagcgtgctaagagacagatcattaaatgcaaaattgctatccgtgatattatccatcaacttgatttgtatagttctagtgggagcatggaagattccgtgatgcctccagatgagtctgttaagtctgttaaccctgataatactatatgttcaagatgcaagtctcctgaatcagttcccaacaataaatttatcttctgtgaaggggcctgcaaaatggcgtaccatgagaaatgttcagaacctcctctagacagaagtcttcctactggtggtcctggatggctttgtaaattctgcttgtgcaagatgaaaattttagaagctgtaaatgcacatttgggaactagccttacagtgatgtgtccctctaaagatatttttaaggaagcaactaaagaggtggactctgaggatggactaggagaggattggttatctgagtattcaggtgatgaggactatgaccctgaagaaaatgacttcacaagcagcagcctggatagtggtgaagaaagtatatctgatcagtccaattgttcaggaagtccgctttattctccaaacgatgatattccaggctttatatcagcagatttcactgatgcggaaggattctaccacaccaattcaaatttaggaattgattctggggatgatgatgttgccgagatgcttacctaccaaaggccaaagagagaggttgattataaaagacttaatgaggaaatgtttggaaagctgactgagaatgaaaagcagagtgaagatgaagattggggtactaataataggagaaagaaaagaagagtgcattcagctggtgttggagttaagtctgccgagggtttgtcagatgtaaaatcaaatgagaaagcccaaccgcagaggagaaaactctttagaattccacctgaggcagttcaggtactacgcaaatcttttgctgaaagtgagcttccagcccgggaggtgaaagagagtctctcaacagaattgggtatttcttttgaaaagattgacaagtggttcaaaaatacaaggtgtgctgctctcagagatcggaaggctgaaggaaatagtcgtgttgctggtcccagcaaaagatcgagacggagtgcagggaaatctggaagttcagtcaagattgattcagtggacaattcatatcttattcccgcgtctgaaatcatcagtgtacctacacatccacaagggattcctgagaggaagcccgaatcaactagtagaactgtgaggaggctggtacatgataaaggagctcccttgtgtccgcccgccgaagtcaaggagagtacatcacctacaaccaagcctggcttgaatcctagccatgtagcagatcgcatcatcaacaccgagcatagagctgcttctcgggaggctccagtggccttctcggatgaccctttcttcgatgcaatcctatcatatcccaccatcaacaccaatgacagagctgtttctcgggaggatccggtggccttctcggacgatcctttcctggatgtgatgctagcaaaccgcgacatctatactgaggagagaggtgtttctcatgaggacgtgaagcggccttctggtgaccagcctttcctggatgtgatccagaacgtgtgcgggctcgagcataggctgcagagactgaagggggacatgctatcatcaggagcagcaccaggcggcactggaaccgcggagcgtgacttgcagaaccaactggtggttctcgtaccaactgccgagctgaaggacaaatcacagcctagcatctga

SEQ ID NO: 6 - protein, 744 aa, Triticum aestivum
MNNSDKKSACSIPRRSSERRKQSSSELVSSAKKPTRHNTSSSYKPDHCSPPKRTVRTVRNVNLAKYIKNKYYRSPLSKRQASNAATGKTATGPVRRRNRKRRRHNTDCDEATRMERRARYLLVKIKSEQNLIDAYSGDGWNGHSREKLKPEKELQRAKKQIIKCKIAIRDIIHQLDLYSSSGSMEDSVMPPDESINPDNTICSRCKSPESVPNNKFIFCEGACKMAYHEKCSEPPLDKSLPTGGPGWLCKFCLCKMNILEAVNAHLGTSLTVMCPSKDIFKEATKQMDSEDGLGEDWLSEYSGDEDYDPEENDFTSSSLDSGEESISDQSNCSGSPLYSPNDDIPGFISADFTDAEGFYHTNSNLGIDSDDDDVAEMLTYQRPKREEMFGKLTENEKQSEDEDWGTNNRRKKRRVDSAGVGVKSAEGSLDVKSNEKAQPQRRKLFRIPPEAVQVLRKSFAESELPTREVKENLSTELGISFEKIDKWFKNTRCAALRDRKAEGNSRVAGPSKRSRQSAGKSGSSGKIDPVDNSYLLPVPEIISVPTHPQGIPERKPESTSRTARRRVHDKGAPLCPPAPTEVKESTSPTTKPGLNPSHVADRIINTEHRAASREDPVAFSDDPFFDAILSYPTINTNDRAVSREDPVAFSDDPFLDVMLANRDIYTEERAASHEDLRRPSSDQPFLDVIQNVCGLEHRLQRLKGDMLSSAAAPGGAGTAERDLQNQLVVLVPTAELKDKSQPSI

SEQ ID NO: 7 - DNA, 2235 bp, Triticum aestivum
  source 1..2235: mol_type = unassigned DNA
atgaataattcagacaagaagtcggcatgctccattcctagaagatcatcagaacggaggaaacagtcctcttctgagttggtttcttcagcaaaaaagccaactcgccacaatacctccagcagttataagccagatcattgttctcctcccaagagaacagtaagaacagtcaggaatgttaatctggcaaaatatataaagaacaagtattatcgtagtcctctgagcaagcggcaggcttcaaatgctgctactggaaaaactgcgacaggaccagtcaggaggaggaaccggaaacggagaaggcacaatactgattgtgatgaggcaactcgcatggaaagaagggcgagatatttgctggtcaaaataaaatcggagcagaatttaatagatgcatactctggagatggctggaatgggcacagccgagagaaattaaagccagagaaggagctacagcgtgctaagaaacagatcattaaatgcaaaattgctatccgtgatattatccatcaacttgatttgtatagttctagtgggagcatggaagattccgtgatgcctccagatgagtctattaaccctgataataccatatgttcaagatgcaagtctcctgaatcagttcccaacaataaatttatcttctgtgaaggggcctgcaaaatggcataccatgagaaatgttcagaacctcctctagacaaaagtcttcctactggtggtcctggatggctttgtaaattctgtttgtgcaagatgaacattttagaagctgtaaatgcacatttgggaacaagccttacagtgatgtgtccctctaaagatatttttaaggaagcaactaaacagatggactctgaggatggactaggagaggattggttatctgagtattcaggtgatgaggactatgaccctgaagaaaatgacttcacaagcagcagcctggatagtggtgaagaaagtatatctgatcagtccaattgttcaggaagtccgctttattctccaaacgatgatattccaggctttatatcagcagatttcactgatgcggaaggattctaccacaccaattcaaatttaggaattgattctgatgatgatgatgttgccgagatgcttacctaccaaaggccaaagagagaggaaatgtttggaaagctgactgagaatgaaaagcagagtgaagatgaagattggggtactaataataggagaaagaaaagaagagtggattcagctggtgttggagttaagtctgccgagggttccttagatgtaaaatcgaatgagaaagcccaaccgcagaggagaaaactcttcagaattccacctgaggcagttcaggtactacgcaaatcttttgctgaaagtgagcttccaacccgggaggtgaaagagaatctctcaacagaattgggtatttcttttgaaaagattgacaagtggttcaaaaatacaaggtgtgctgctctaagagatcggaaggctgaaggaaatagtcgtgttgctggtcccagcaaaagatcgagacagagtgcagggaaatctggaagttcaggcaagattgatccagtggacaattcatatcttcttcctgtgcctgaaatcatcagtgtacctacacatccacaagggattcctgagaggaagcccgaatcaactagtagaactgcaaggaggcgggtacacgataaaggagctcccttgtgtccgcccgcgcccaccgaagtcaaggagagtacatcacctacaaccaagcctggcttgaatcctagccatgtagcagatcgcatcatcaacaccgagcatagagctgcttctcgggaggatccagtggccttctcggacgacccattctttgatgcgatcctatcatatcccaccatcaacaccaacgacagagctgtttctcgggaggatccggtggccttctcagacgatcctttcctggatgtgatgctagcaaaccgtgacatctataccgaggagagagccgcttctcacgaggacctgaggcggccttccagcgaccagcctttcttggatgtgatccagaacgtgtgcggactcgagcataggctgcagagactcaagggggacatgctatcatcagcagcagcaccaggcggcgctggaaccgcggagcgtgacttgcagaaccaactggtggttctcgtgccgactgccgagctgaaggacaaatcacagcctagcatctga

SEQ ID NO: 8 - protein, 752 aa, Triticum aestivum
MNNSDKKSACSIPRRSSERRKQSSSELVSSAKRSTGHNTSSSYKPDHCSPPKRTVRTVRNVNLAKYIKNKYYRSPLSKRQASNAATGKTATGPVRRRNRKRRRHNTDCDEATRMERRARYLLVKIKSEQNLIDAYSGDGWNGHSREKLKPEKELQRAQKQIIKCKIAIRDIIHQLDLYSSSGSMEDSVMPPDESVNPDNTICSRCKSPESVPNNKFIFCEGACKMAYHEKCLEPPLDKSLPTGGPGWLCKFCLCKMKILEAVNAHLGTSLTVMCPSKDIFKEATKQMDSEDGLGEDWLSEYSGDEDYDPEENDFTSSSLDSGEESISDQSNCSGSPLYSPNDDIPGFISADFTDAEGFYHTNSNLGIDSDDDDVAEMLTYQRPKREVDYKRLNEEMFGKLTENEKQSEDEDWGTNNRRKKRRVDSAGVGVKSAEGSLDVKSNEKAQPQRRKLFRIPPEAVQVLRKSFAESELPSREVKENLSTELGISFEKIDKWFKNTRCAALRDRKAEGNSRVAGPSKRSKQSAGKSGSLGKIDSVDNSYLLPMPEIISVPTHPQGIPERKPESTSRTVRRRVHDKGAPLCPPAPAEVKESTSPTTKPGLNPSHVADRIINTEHRAASREDPVAFSDDPFFDAILSYPTINTNDRAVSREDPVAFSDDPFLDVMLANRDIYTEERAASHEDVRRPSSDQPFLDVIQNVCGLEHRLQRLKGDMLSSAAAPGGAGTAERDLQNQLVVLVPTAELKDKSQPSI

SEQ ID NO: 9 - DNA, 2259 bp, Triticum aestivum
  source 1..2259: mol_type = unassigned DNA
atgaataattcagacaagaaatcggcatgctccattcctagaagatcatcagaacggaggaaacagtcctcttctgagttggtttcttcagcaaaaaggtcaactggccacaatacctccagcagttataagccagatcattgctctcctcccaagagaacagtaagaacagtcaggaatgttaatctggcaaaatatataaagaacaagtattatcgtagtcctctgagcaagcggcaggcttcaaatgctgctactggaaaaactgcaacaggaccagtcaggaggaggaaccggaaacggagaaggcacaatactgattgtgatgaggcaactcgcatggaaagaagggcgagatatttgctggtcaaaataaaatcggagcagaatttaatagatgcatactctggagatggctggaatgggcacagccgagagaaattaaagccagagaaggagctacagcgtgctcagaaacagatcattaaatgcaaaattgctatccgtgatattatccatcaacttgatttgtatagttctagtgggagcatggaagattccgtgatgcctccagatgagtctgttaaccctgataataccatatgttcaagatgcaagtctcctgaatcagttcccaacaataaatttatcttctgtgaaggggcctgcaaaatggcataccatgagaaatgtttagaacctcctctagacaaaagtcttcctactggtggtcctggatggctttgtaaattctgtttgtgcaagatgaaaattttagaagctgtgaatgcacatttgggaacaagccttacagtgatgtgtccctctaaagacatttttaaggaagcaactaaacagatggactctgaggatggactaggagaggattggttatctgagtattcaggtgatgaggactatgaccctgaagaaaatgacttcacaagcagcagcctggatagtggtgaagaaagtatatctgatcagtccaattgttcaggaagtccgctttattctccaaacgatgatattccaggctttatatcagcagatttcactgatgcggaaggattctaccacaccaattcaaatttaggaattgattctgatgatgatgatgttgccgagatgcttacctaccaaaggccaaagagagaggttgattataaaagacttaatgaggaaatgtttggaaagctgactgagaatgaaaagcagagtgaagatgaagattggggtactaataataggagaaagaaaagaagagtggattcagctggtgttggagttaagtctgccgagggttccttagatgtaaaatcgaatgagaaagcccaaccgcagaggagaaaactcttcagaattccacctgaggcagttcaggtactgcgcaaatcttttgctgaaagtgagcttccatcccgggaggtgaaagagaatctctcaacagaattgggtatttcttttgaaaagattgacaagtggttcaaaaatacaaggtgtgctgctctcagagatcggaaggctgaaggaaatagtcgtgttgctggtcccagcaaaagatcgaaacagagtgcagggaaatctggaagtttaggcaagattgattcagtggacaattcatatcttcttcccatgcctgaaatcatcagtgtacctacacatccacaagggattcctgagaggaagcccgaatcaactagtagaactgtgaggaggcgggtacacgataaaggagctcccttgtgtccgcccgcgcccgccgaagtcaaggagagcacatcacctacaaccaagcctggcttgaatcctagccatgtagctgatcgcatcatcaacaccgagcatagagctgcttctcgggaggatccagtggccttctcggatgaccctttcttcgatgcgatcctatcatatcccaccatcaacaccaacgacagagctgtttctcgggaggatccggtggccttctcggacgatcctttcctggacgtgatgctagcaaaccgtgacatctataccgaggagagagccgcttctcacgaggacgtgaggcggccttccagcgaccagcctttcttggatgtgatccagaacgtgtgcgggctcgagcataggctgcagagactcaagggggacatgctatcatcagcagcagcaccaggcggcgctggaaccgcggagcgtgacttgcagaaccaactagtggttctcgtgccgactgccgagctgaaggacaaatcacagcctagcatctga

SEQ ID NO: 10 - DNA, 20 bp, Triticum aestivum
  source 1..20: mol_type = unassigned DNA
agtagtagtagtactacata

SEQ ID NO: 11 - DNA, 16 bp, Triticum aestivum
  source 1..16: mol_type = unassigned DNA
gtagtagtagtactac

SEQ ID NO: 12 - DNA, 15 bp, Triticum aestivum
  source 1..15: mol_type = unassigned DNA
gtagtagtactacta

SEQ ID NO: 13 - protein, 613 aa, Triticum aestivum
MMDNLAIVAKELGDVSDFEVDGIPNLSENDVSDEEIEAEELARRMWKDKVRLKRIKEKQQRLALEQAELEKSKPKKLSDLALRKKMARAQDGILKYMLKLMEVCNAQGFVYGIIPDKGKPVSGASDNIRAWWKEKVKFDKNGPAAIAKYEVENSLLVNAKSSGTMNQYSLMDLQDGTLGSLLSALMQHCSPQQRKYPLDKGIPPPWWPSGNEEWWIALGLPKGKTPPYRKPHDLKKVWKVGVLMGVIKHLSPNFDKIRYHVRKSKCLQDKMTAKESLIWLVVLQREEYVHSIGNGVSNTHLVDLGDKNESSYSSCDEYDVDCMEEPPQSTISKDDVGVHQPAVHIREENGSSSGNKKRHDKRSTQRLPSTKETKNPPKRRRHTGQCSVDGSEVEGTQRNDNMPEVLSNAIPDMKSNQMEVVCVANPLTSFNHAGTNGGSLQHQGDVQGNFVPPGVVVNNYSQAANIASSSIYMVDQPLASESNDYTNSWPGNTFQPDVGLGSIGFSSSSHDYQSSAAKDSVPLSMDNHVPAMGTGALNSSYSHMAGSGNSTSVAGGTQQIMSDAFYIDPDDKFIGSSFDGLPLDFIGINSPIPDLDELDELLDDNDLMQYLGT

SEQ ID NO: 14 - DNA, 1842 bp, Triticum aestivum
  source 1..1842: mol_type = unassigned DNA
atgatggataacttagctattgtcgcgaaggagctaggtgatgtgtcggattttgaggttgatggcatcccaaacctctctgagaatgatgtcagcgatgaggagattgaggctgaggagctggcccggcggatgtggaaagataaggtcaggctcaagaggatcaaggagaagcagcagaggcttgctttggagcaggccgaactggagaagtctaaaccaaagaagctgtctgatctagcccttcgcaagaagatggcaagagcccaggatgggattttgaagtacatgctcaagttgatggaagtatgcaatgcacagggttttgtttatgggatcattcccgataaagggaagcctgtcagtggagcatcagacaacattagagcttggtggaaggagaaggttaagtttgataagaacgggccagcagcaattgcaaaatatgaggttgagaactctctgttggttaatgctaagagcagtggaaccatgaatcaatatagcttgatggatctccaagatggtactctgggctcattgctttctgcattgatgcagcactgcagccctcagcagcgcaagtacccactggataagggtattccacccccatggtggccctcagggaacgaggagtggtggattgctttaggccttccaaagggtaaaacacctccatacagaaaacctcacgatcttaagaaagtttggaaggttggtgtgctgatgggtgtgatcaaacacttgtctccgaattttgataagataagatatcatgtacggaagtcaaagtgcttgcaggacaaaatgactgcaaaagagagcttgatttggctggttgttttgcaaagagaggagtatgttcacagtattggcaacggtgtatcaaatactcatcttgttgacctaggggacaaaaatgagagttcatacagcagctgtgatgagtatgatgtcgattgtatggaggagcctcctcagtctacaatatccaaagacgatgtgggagttcatcaaccagctgtgcacatcagagaagagaatggctcaagtagtgggaacaaaaaacgtcatgataaacgctcaactcaaaggctgcctagtactaaggaaactaaaaacccaccaaagcgaagaagacataccggacagtgttctgttgatgggtctgaagttgaaggaacacaaaggaatgataatatgccagaggttttgagcaacgcaattccggatatgaagagcaatcagatggaggtggtctgtgtcgctaacccgttgacaagcttcaatcatgccggtacaaatggaggatctttacaacatcaaggagatgttcaagggaactttgtaccccctggtgttgtggttaataattacagccaggctgcaaatattgcttcttcaagcatttatatggtcgaccagccattagcttctgaaagtaatgattacacaaactcctggcctggaaatacttttcaaccagacgttggtcttggatctattggctttagttcttcttcacatgattaccagtcttctgctgcaaaagactcagtaccactatctatggataaccatgtgcctgccatgggaacaggagctttgaacagttcttacagtcatatggcaggtagtgggaattcaacttctgttgctggtggcacgcaacagatcatgagtgatgctttttatattgaccctgatgataagtttataggcagttcttttgatggactgcctttagatttcatcggtattaatagtccaatccctgacctcgatgaactcgatgaactgctggatgacaatgacttgatgcaatatctgggaacgtaa

SEQ ID NO: 15 - protein, 614 aa, Triticum aestivum
MMDNLAIVTKELGDVSDFEVDGIPNLSENDVSDEEIEAEELARRMWKDKVRLKRIKEKQQRLALEQAELEKSKPKKLSDLALRKKMARAQDGILKYMLKLMEVCNAQGFVYGIIPDKGKPVSGASDNIRAWWKEKVKFDKNGPAAIAKYEVENSLLVNAKSSGTMNQYSLMDLQDGTLGSLLSALMQHCSPQQRKYPLDKGIPPPWWPSGNEEWWIALGLPKGKTPPYRKPHDLKKVWKVGVLMGVIKHLSPNFDKIRYHVRKSKCLQDKMTAKESLIWLVVLQREEYVHSIGNGVSDTHLVNLGDKHESSYSSCDEYDVDCMEEPPKSTISKDDVGVHQPAVHIREENGSSSGNKKRHDKRSTQTLPSTKETKKPPKRRRHTGQCSVDGSEVEGTQRNDNMPEVLSNAIPDMNSNQMEVVCVANPLTSFNHAGTNGGSLQHQGDVQGNFVPPGVVVNNYSQAANIAPSSIYMVDQPLASESNDYTNSWPGNTFQPDVGLGSIGFSSSSHDYQSSAAKHSVPLPTDNHVPAMGTGALNSSYSHHMAGSGNSTSVAGDTQQIMSDAFYIDPDDKFIGSSFDGLPLDFIGINSPIPDLDELDELLDDNDLMQYLGT

SEQ ID NO: 16 - DNA, 1845 bp, Triticum aestivum
  source 1..1845: mol_type = unassigned DNA
atgatggataatctagctattgtcacgaaggagctaggtgatgtgtcggattttgaggttgatggcatcccaaacctctctgagaatgatgtcagtgacgaggagatcgaggctgaggagctggcccggcggatgtggaaagataaggtcaggctcaagaggatcaaggagaagcagcagaggcttgctttggagcaggccgaactggagaagtctaaaccaaagaagctgtctgatctagcccttcgcaagaagatggcaagagcccaggatgggattttaaagtacatgctcaagttgatggaagtatgtaatgcacagggttttgtttatgggatcattcctgataaagggaagcctgtcagtggagcatcagacaacattagagcttggtggaaggagaaggttaagtttgataagaacgggccagcagcaattgcaaaatatgaggttgagaactctctgttggttaatgctaagagcagtggaaccatgaatcaatatagcttgatggatctccaagatggtactctgggctcattgctttctgcattgatgcagcactgcagccctcagcagcgcaagtacccactggataagggtattccacccccgtggtggccctcagggaatgaggagtggtggattgctttaggccttccaaagggtaaaacacctccatacagaaaacctcacgatcttaagaaggtttggaaggttggtgtgctgatgggtgtgatcaaacacttgtctccgaattttgataagataagatatcatgtacggaagtcaaagtgcttgcaggacaaaatgactgcaaaagagagcttgatttggctggttgttttgcaaagagaggagtatgttcacagtattggcaacggtgtatcagatactcatcttgttaacctaggggacaaacatgagagttcatacagcagctgtgatgagtatgatgtcgattgtatggaggagcctcctaagtctacaatatccaaagacgatgtgggagttcatcagccagctgtgcacatcagagaagagaatggctcaagtagtgggaacaaaaaacgtcatgataaacgctctactcaaacactgcctagtactaaggaaactaaaaaaccaccaaagcgaagaagacataccggacagtgttctgttgatgggtctgaggttgaaggaacacaaagaaatgataatatgccagaggttttgagcaacgcaattccggatatgaatagcaatcagatggaggtggtctgtgtcgctaacccgttgacaagcttcaatcatgccggtacaaatggaggatctttacaacatcaaggagatgttcaagggaactttgtaccccctggtgttgtggttaataattacagccaggctgcaaatattgctccttcaagcatttatatggtcgaccagccattagcttctgaaagtaatgattacacaaactcctggcctggaaatacttttcaaccagatgttggtcttggatctattggctttagttcttcttcacatgattaccagtcttctgctgcaaaacactcagtaccactacctacggataaccatgtccctgccatgggaacaggagctttgaacagttcttacagtcatcatatggcaggtagtgggaattcaacttctgttgctggtgacacgcaacaaatcatgagtgatgctttttatattgaccccgatgataagtttataggcagttcttttgatggactgcctttagatttcatcggtattaatagtccaatccctgacctcgatgaactcgatgaactgctggatgacaatgacttgatgcaatatctgggaacgtaa

SEQ ID NO: 17 - protein, 614 aa, Triticum aestivum
MMDNLAIVAKELGDVSDFEVDGIPNLSENDVSDEEIEAEELARRMWKDKVRLKRIKEKQQRLALEQAELEKSKPKKLSDLALRKKMARAQDGILKYMLKLMEVCNAQGFVYGIIPDKGKPVSGASDNIRAWWKEKVKFDKNGPAAIAKYEVENSLLVNAKSSGTMNQYSLMDLQDGTLGSLLSALMQHCSPQQRKYPLDKGIPPPWWPSGSEEWWIALGLPKGKTPPYRKPHDLKKVWKVGVLMGVIKHLAPNFDKIRYHVRKSKCLQDKMTAKESLIWLVVLQREEYVHSIGNGVSDTHLVDLGDKNECSYSSCDEYDVDCMEEPPQSTISKDDVGVHQPAVHIREENGSSSGNKKHHDKRSTQRLPSTKETKRPPKRRRHTGQCSVDGSEVEGTQRNDNNAEVLSNSIPDMKSNQMEVVCVANPLTSFNPAGTNGGSLQHQGDVQGNFVPPGVVVNNYSQAANIASSSIYMVDQPLASESNDYTNSWPGNTFQPDVGLGSIGFSSSSHDYQSSAAKHSVPLSVDNHVPAMGTGALNSSYSHHMAGSGNSTSVAGDTQPIMSDAFYIDPDDKFMGSSFDGLPFDFIGINSPIPDLDELDELLDDNDLMQYLGT

SEQ ID NO: 18 - DNA, 1845 bp, Triticum aestivum
  source 1..1845: mol_type = unassigned DNA
atgatggataatctagctattgtcgcgaaggagcttggtgatgtgtcggattttgaggttgatggcatcccaaacctctctgagaatgatgtcagtgacgaagagatcgaggctgaggagctggcccggcggatgtggaaagataaggtcaggctcaagaggatcaaggagaagcagcagaggcttgctttggagcaggccgaactggagaagtctaaaccaaagaagctgtctgatctagcccttcgcaagaagatggcaagagcccaggatgggattttgaagtacatgctcaagttgatggaagtatgcaatgcacagggttttgtttatgggatcattcctgataaagggaagcctgtcagtggagcatcagacaacattagagcttggtggaaggagaaggttaagtttgataagaatgggccagcagcaattgcaaaatatgaggttgagaactctctgttggttaatgctaagagcagtggaaccatgaatcaatatagcttgatggatctccaagatggtactctgggctcattgctttctgcattgatgcagcactgcagccctcagcagcgcaagtacccactggataagggtattccacccccgtggtggccctcagggagtgaggagtggtggattgctttaggccttccaaagggtaaaacacctccatacagaaaacctcacgatcttaagaaggtttggaaggttggtgtgctgatgggtgtgatcaaacacttggctcccaattttgataagataagatatcatgtacggaagtcaaagtgcttgcaggacaaaatgactgcaaaagagagcttgatttggctggttgttttgcaaagagaggagtatgttcacagtattggcaacggtgtatcagatactcatcttgttgacctaggggacaaaaatgagtgttcatacagcagctgtgatgagtatgatgtcgattgtatggaggagcctcctcagtctacaatatccaaagacgatgtgggagttcatcaaccagctgtgcacatcagagaagagaatggctcaagtagtgggaacaaaaaacatcatgataaacgctctactcaaaggcttcctagtactaaggaaactaaaagaccaccaaagcgaagaagacataccggacagtgttctgtggatgggtctgaggttgaaggaacacaaaggaatgataataatgcagaggttttgagcaactccattccggatatgaagagcaatcagatggaggtggtctgtgtcgctaacccgttgacaagcttcaatcctgccggtacaaatggaggatctttacaacatcaaggagatgttcaagggaactttgtaccccctggtgttgtggttaataattacagccaggctgcaaatattgcttcttcaagcatttatatggtcgaccagccattagcttctgaaagtaatgattacacaaactcctggcctggaaatacttttcaaccagacgttggtcttggatctatcggctttagctcttcttcacatgattaccagtcttctgctgcaaaacactcagtgccactatcagtggataaccatgtgcctgccatgggaacaggagctttgaacagttcttacagtcatcatatggcaggtagtgggaattcaacctctgttgctggtgacacgcaaccgatcatgagtgatgctttttatattgaccctgatgataagtttatgggcagttcttttgatggactgccttttgatttcatcggtattaatagtccaattcctgaccttgatgaactcgatgaactgctggatgacaatgatttgatgcaatatctgggaacgtaa

SEQ ID NO: 19 - DNA, 19 bp, Triticum aestivum
  source 1..19: mol_type = unassigned DNA
catctagatacatcaatct

SEQ ID NO: 20 - DNA, 9563 bp, synthetic construct
  source 1..9563: mol_type = other DNA
  misc_feature 1..9563: note = construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgggcccggccggccgcgatcgctacgtacctgcaggcccgggttaattaagcggccgcaatttatgcatcacagtcgttgatggccagaagttggacacaagagagaccagcagccagcgctttctcggttttaaaaaaaagaggtcagggcctcttttttatatacaacaaaaataccacagttttagagatatcatggtttgttgaactgcagtattttttttatccaaacgactcaaagtattaaataccaaagtttttttcaaaaaccacagtattccagaaaaccacaaaaatacttcggcttccaaacgcaccctaagtgtaagaagaaatctactaagttcacaaaattcagaagaaactcgtttggaccatgagcggtacagaaaagaaaatggcgtgatctagaatagcagacattagggtctgtttgcagatattatggatcactgtatgtttgcaaaaaataataatttgtgcagctatcagatgcatgcattttctatgaaaattttgtagatccagtgttatacccaacaacaaaatttaatacttgggtcaccacaacccaagatggagctccaagcaaacttaagtaagaggataccgagatgtgatgatgatgtctgatcttgggggaatttttgtagtgagaaaatgccttaaacatcatcgcggaatccaaatggtcaatcaggactgacaccaccaatggcttgattcaccatctctatctgcccattttgacgtagcactatttggctttcgacgcgacggggtaatcaacgtgacaccatcctgtcatgagaaagtcatcatgccgagttcattttccaggcatcccgtgtggcatcttgccattctccccgtccatgagttgattacccaaaagcatctgatcccggagcacaacgaacgacatcagaagacatggttcgcatcacaaaacaccatttggtcatgttacgcatgttaggcacatctgaagaagggaaggcgttgagaacctcggccaccactagtaatagacttccaacttttagttgggacaacatgggcgggatttcacggcacaaaattcaccacctagatgctaaaaagatacccccccccccccatcccccacacatcctccttgaagacacgggcgccccctccgcctccctacctcgccgtcgccggaggaggccgccgggctaagcctgtgcggtggacgacggcggcggggcctctctgcagcgtccgggtctcatggtgagggaggcacgcctcctcgcagtgcggcgcggcggctacggcacgaagattctccgaagttgcgtctccagcgggtggcagtcgcggtggtgtcgtcggcaaactcctgggcttcggtccggcggctcttgctcggcgagtatgggcggggtggcggcccccctttccttcctttggtcattcgtctccgcgcaggtcgactgctctggccggcccttggtggttgttgtagtgctgctgcaagggggcgccagatccggcggttcttcattcggatccagctcgtctctcatcttactcagggtcggtcatcccggtgaccttgcctctggcagcaaggggtgtgtgtgctctatggtgggccgtgggccttgtgaggcgatggcgcggttgctgcgcaggggtgtgcatgccagcgattgcgcgggccagcttgtgtcggctgcgtgagggtgttggttgagtctgctccaagcgcaagcttgcccggctatggccggccggtgctggcggcgcctatgggcgtcgtttcccttgttgaaggcgttgttgtagagttcttcgacctactacaatctcgaatctggttcttcaggcgaaaacctcgcccatctaggtcgggcaacgatgtcgtccgtacggtgttttccccctttggggcgttgctttgaagaccggttatcctttcccgtgctacctttgggctggtcgtgcatggtgtcatggttggctggtgctcaaccggtgatacgagtggttggcgttgcgactcgtgcggtgacgaagatggtgatgcgcgaagctgggtcgcggcttgtccttctgatatcggcggttcggttcccctttgggtgcgtctgtgcttgtttctcactgtgacagagaagttgaagctacatgcggcgggggccctgcggcaatgatgactccatgagggtggtttcggcggacgatgctctccgcaggttgcactggactagcttggtgtgaggcttgcaactttctcctgtgatgctcatcaacaaaatcggagatgtcggtcctcgacgcctgcggccgatcatttggcttggctgtcgaggtcctcgggtgccgtttgttcggaggggtcttgatgggtcgtcagcgaggaaatcggagttgcctgctcgtggagaggcgatgacgatgacattgcagcctcgacggtgtcctctcgtcagcagtatgtgtgtgatcttgtcagtgccaggtcggctggagtgccctattcgatgggcgtgttttaattaaccgggcaactctcttcttcttaatcagtgaaaatggcaagtcttttacctcgtttcaaaaagagttaaatgcactggaggtcctaaaggtttcgtcggggtgtcactttagtcctatttcttccaaaatgcacctttaggtcctaattctttattaattggttcacccgaggtccttttccacgagcgctcgctgaccagcacacgtgccgcattgacccatgccacatcgatagagggtctaaccgccaggcgagaagcttcgcaaagagaccccccccctcttagtggcaccctctctcctctttcacggtactttggcgacattggaggcgagcgacagaggcaaccggaggtgatctggaggtaatttgtgtgggtgcccgtgaagatgccccttctatgacggccagtgagcaatcctcctccatgttaccgtaagaaccctaatcttgtcgatgatcttctcctgtagctcttctaatatgtcataactttgtcataatcaatagttgtagtccttgatttactttgatttgcataacggtgatacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggtttagggacgcgcgcgccgagagagagagcagatttggggaagaaataaagccagggactggggaatgagaagataatgtttataagggttttctgtaaaaagaaagagcaacctacgcagcccttctgtcgatttggcatgggtcaacgcaccacacgagcaggtcaacgagcgctcgtgagaaaaaggacctagggtaaaccacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacatactccctccgtccggaaatacttgtcgaagaatttgatgaaaatggatgcatctagaacaagaatacatctagatacatcaatctccctgacaagtatttccgagcggagggagtactagataatactccctccgttcctaaataattgtctttctagctatctcaaataaactacaacatacggatgtatgtagacatgttttagagtgtagattcactcattttgttccgtatgtagtcatttgttgaaatctctagagagacaattatttaggaacggagggagtaagataactaccctaaaaaaaaagataactgaaggttgccacctagcacattcacattggtacaacttggaaaagcacagccccgtcgtcctgctcccagttgagttcgcgacctacacaccggccaggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaccatggtccgtcctgtagaaaccccaacccgtgaaatcaaaaaactcgacggcctgtgggcattcagtctggatcgcgaaaactgtggaattgatcagcgttggtgggaaagcgcgttacaagaaagccgggcaattgctgtgccaggcagttttaacgatcagttcgccgatgcagatattcgtaattatgcgggcaacgtctggtatcagcgcgaagtctttataccgaaaggttgggcaggccagcgtatcgtgctgcgtttcgatgcggtcactcattacggcaaagtgtgggtcaataatcaggaagtgatggagcatcagggcggctatacgccatttgaagccgatgtcacgccgtatgttattgccgggaaaagtgtacgtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcaggtatcaccgtttgtgtgaacaacgaactgaactggcagactatcccgccgggaatggtgattaccgacgaaaacggcaagaaaaagcagtcttacttccatgatttctttaactatgccggaatccatcgcagcgtaatgctctacaccacgccgaacacctgggtggacgatatcaccgtggtgacgcatgtcgcgcaagactgtaaccacgcgtctgttgactggcaggtggtggccaatggtgatgtcagcgttgaactgcgtgatgcggatcaacaggtggttgcaactggacaaggcactagcgggactttgcaagtggtgaatccgcacctctggcaaccgggtgaaggttatctctatgaactgtgcgtcacagccaaaagccagacagagtgtgatatctacccgcttcgcgtcggcatccggtcagtggcagtgaagggcgaacagttcctgattaaccacaaaccgttctactttactggctttggtcgtcatgaagatgcggacttgcgtggcaaaggattcgataacgtgctgatggtgcacgaccacgcattaatggactggattggggccaactcctaccgtacctcgcattacccttacgctgaagagatgctcgactgggcagatgaacatggcatcgtggtgattgatgaaactgctgctgtcggctttaacctctctttaggcattggtttcgaagcgggcaacaagccgaaagaactgtacagcgaagaggcagtcaacggggaaactcagcaagcgcacttacaggcgattaaagagctgatagcgcgtgacaaaaaccacccaagcgtggtgatgtggagtattgccaacgaaccggatacccgtccgcaaggtgcacgggaatatttcgcgccactggcggaagcaacgcgtaaactcgacccgacgcgtccgatcacctgcgtcaatgtaatgttctgcgacgctcacaccgataccatcagcgatctctttgatgtgctgtgcctgaaccgttattacggatggtatgtccaaagcggcgatttggaaacggcagagaaggtactggaaaaagaacttctggcctggcaggagaaactgcatcagccgattatcatcaccgaatacggcgtggatacgttagccgggctgcactcaatgtacaccgacatgtggagtgaagagtatcagtgtgcatggctggatatgtatcaccgcgtctttgatcgcgtcagcgccgtcgtcggtgaacaggtatggaatttcgccgattttgcgacctcgcaaggcatattgcgcgttggcggtaacaagaaagggatcttcactcgcgaccgcaaaccgaagtcggcggcttttctgctgcaaaaacgctggactggcatgaacttcggtgaaaaaccgcagcagggaggcaaacaatgattgctagcacgcgtccctagacttgtacatcttctggattggccaacttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccggcgcgccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 21 - DNA, 2000 bp, Triticum aestivum
  source 1..2000: mol_type = unassigned DNA
tggtttcggcggacgatgctctccgcaggttgcactggactagcttggtgtgaggcttgcaactttctcctgtgatgctcatcaacaaaatcggagatgtcggtcctcgacgcctgcggccgatcatttggcttggctgtcgaggtcctcgggtgccgtttgttcggaggggtcttgatgggtcgtcagcgaggaaatcggagttgcctgctcgtggagaggcgatgacgatgacattgcagcctcgacggtgtcctctcgtcagcagtatgtgtgtgatcttgtcagtgccaggtcggctggagtgccctattcgatgggcgtgttttaattaaccgggcaactctcttcttcttaatcagtgaaaatggcaagtcttttacctcgtttcaaaaagagttaaatgcactggaggtcctaaaggtttcgtcggggtgtcactttagtcctatttcttccaaaatgcacctttaggtcctaattctttattaattggttcacccgaggtccttttccacgagcgctcgctgaccagcacacgtgccgcattgacccatgccacatcgatagagggtctaaccgccaggcgagaagcttcgcaaagagaccccccccctcttagtggcaccctctctcctctttcacggtactttggcgacattggaggcgagcgacagaggcaaccggaggtgatctggaggtaatttgtgtgggtgcccgtgaagatgccccttctatgacggccagtgagcaatcctcctccatgttaccgtaagaaccctaatcttgtcgatgatcttctcctgtagctcttctaatatgtcataactttgtcataatcaatagttgtagtccttgatttactttgatttgcataacggtgatacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggtttagggacgcgcgcgccgagagagagagcagatttggggaagaaataaagccagggactggggaatgagaagataatgtttataagggttttctgtaaaaagaaagagcaacctacgcagcccttctgtcgatttggcatgggtcaacgcaccacacgagcaggtcaacgagcgctcgtgagaaaaaggacctagggtaaaccacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacatactccctccgtccggaaatacttgtcgaagaatttgatgaaaatggatgcatctagaacaagaatacatctagatacatcaatctccctgacaagtatttccgagcggagggagtactagataatactccctccgttcctaaataattgtctttctagctatctcaaataaactacaacatacggatgtatgtagacatgttttagagtgtagattcactcattttgttccgtatgtagtcatttgttgaaatctctagagagacaattatttaggaacggagggagtaagataactaccctaaaaaaaaagataactgaaggttgccacctagcacattcacattggtacaacttggaaaagcacagccccgtcgtcctgctcccagttgagttcgcgacctacacaccggcc

SEQ ID NO: 22 - DNA, 1423 bp, Triticum aestivum
  source 1..1423: mol_type = unassigned DNA
aaccgccaggcgagaagcttcgcaaagagaccccccccctcttagtggcaccctctctcctctttcacggtactttggcgacattggaggcgagcgacagaggcaaccggaggtgatctggaggtaatttgtgtgggtgcccgtgaagatgccccttctatgacggccagtgagcaatcctcctccatgttaccgtaagaaccctaatcttgtcgatgatcttctcctgtagctcttctaatatgtcataactttgtcataatcaatagttgtagtccttgatttactttgatttgcataacggtgatacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggtttagggacgcgcgcgccgagagagagagcagatttggggaagaaataaagccagggactggggaatgagaagataatgtttataagggttttctgtaaaaagaaagagcaacctacgcagcccttctgtcgatttggcatgggtcaacgcaccacacgagcaggtcaacgagcgctcgtgagaaaaaggacctagggtaaaccacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacatactccctccgtccggaaatacttgtcgaagaatttgatgaaaatggatgcatctagaacaagaatacatctagatacatcaatctccctgacaagtatttccgagcggagggagtactagataatactccctccgttcctaaataattgtctttctagctatctcaaataaactacaacatacggatgtatgtagacatgttttagagtgtagattcactcattttgttccgtatgtagtcatttgttgaaatctctagagagacaattatttaggaacggagggagtaagataactaccctaaaaaaaaagataactgaaggttgccacctagcacattcacattggtacaacttggaaaagcacagccccgtcgtcctgctcccagttgagttcgcgacctacacaccggcc

SEQ ID NO: 23 - DNA, 1260 bp, Triticum aestivum
  source 1..1260: mol_type = unassigned DNA
aaccgccaggcgagaagcttcgcaaagagaccccccccctcttagtggcaccctctctcctctttcacggtactttggcgacattggaggcgagcgacagaggcaaccggaggtgatctggaggtaatttgtgtgggtgcccgtgaagatgccccttctatgacggccagtgagcaatcctcctccatgttaccgtaagaaccctaatcttgtcgatgatcttctcctgtagctcttctaatatgtcataactttgtcataatcaatagttgtagtccttgatttactttgatttgcataacggtgatacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggtttagggacgcgcgcgccgagagagagagcagatttggggaagaaataaagccagggactggggaatgagaagataatgtttataagggttttctgtaaaaagaaagagcaacctacgcagcccttctgtcgatttggcatgggtcaacgcaccacacgagcaggtcaacgagcgctcgtgagaaaaaggacctagggtaaaccacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacatactccctccgtccggaaatacttgtcgaagaatttgatgaaaatggatgcatctagaacaagaatacatctagatacatcaatctccctgacaagtatttccgagcggagggagtactagataataagataactaccctaaaaaaaaagataactgaaggttgccacctagcacattcacattggtacaacttggaaaagcacagccccgtcgtcctgctcccagttgagttcgcgacctacacaccggcc

SEQ ID NO: 24 - DNA, 1296 bp, synthetic construct
  source 1..1296: mol_type = other DNA
  misc_feature 1..1296: note = duplicated region in promoter
aaccgccaggcgagaagcttcgcaaagagaccccccccctcttagtggcaccctctctcctctttcacggtactttggcgacattggaggcgagcgacagaggcaaccggaggtgatctggaggtaatttgtgtgggtgcccgtgaagatgccccttctatgacggccagtgagcaatcctcctccatgttaccgtaagaaccctaatcttgtcgatgatcttctcctgtagctcttctaatatgtcataactttgtcataatcaatagttgtagtccttgatttactttgatttgcataacggtgatacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggtttagggacgcgcgcgccgagagagagagcagatttggggaagaaataaagccagggactggggaatgagaagataatgtttataagggttttctgtaaaaagaaagagcaacctacgcagcccttctgtcgatttggcatgggtcaacgcaccacacgagcaggtcaacgagcgctcgtgagaaaaaggacctagggtaaaccacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacatactccctccgtccggaaatacttgtcgaagaatttgatgaaaatggatgcatctagaacaagaatacatctagatacatcaatctcatctagaacaagaatacatctagatacatcaatctccctgacaagtatttccgagcggagggagtactagataataagataactaccctaaaaaaaaagataactgaaggttgccacctagcacattcacattggtacaacttggaaaagcacagccccgtcgtcctgctcccagttgagttcgcgacctacacaccggcc

SEQ ID NO: 25 - DNA, 1260 bp, synthetic construct
  source 1..1260: mol_type = other DNA
  misc_feature 1..1260: note = duplicated sequence
aaccgccaggcgagaagcttcgcaaagagaccccccccctcttagtggcaccctctctcctctttcacggtactttggcgacattggaggcgagcgacagaggcaaccggaggtgatctggaggtaatttgtgtgggtgcccgtgaagatgccccttctatgacggccagtgagcaatcctcctccatgttaccgtaagaaccctaatcttgtcgatgatcttctcctgtagctcttctaatatgtcataactttgtcataatcaatagttgtagtccttgatttactttgatttgcataacggtgatacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggtttagggacgcgcgcgccgagagagagagcagatttggggaagaaataaagccagggactggggaatgagaagataatgtttataagggttttctgtaaaaagaaagagcaacctacgcagcccttctgtcgatttggcatgggtcaacgcaccacacgagcaggtcaacgagcgctcgtgagaaaaaggacctagggtaaaccacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacatactccctccgtccggaaatacttgtcgaagaatttgatgaaaatggatgcatctagaacaagaatacatctagacaaattactctccctgacaagtatttccgagcggagggagtactagataataagataactaccctaaaaaaaaagataactgaaggttgccacctagcacattcacattggtacaacttggaaaagcacagccccgtcgtcctgctcccagttgagttcgcgacctacacaccggcc

SEQ ID NO: 26 - DNA, 1394 bp, synthetic construct
  source 1..1394: mol_type = other DNA
  misc_feature 1..1394: note = duplicated sequence in promoter
aaccgccaggcgagaagcttcgcaaagagaccccccccctcttagtggcaccctctctcctctttcacggtactttggcgacattggaggcgagcgacagaggcaaccggaggtgatctggaggtaatttgtgtgggtgcccgtgaagatgccccttctatgacggccagtgagcaatcctcctccatgttaccgtaagaaccctaatcttgtcgatgatcttctcctgtagctcttctaatatgtcataactttgtcataatcaatagttgtagtccttgatttactttgatttgcataacggtgatacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggtttagggacgcgcgcgccgagagagagagcagatttggggaagaaataaagccagggactggggaatgagaagataatgtttataagggttttctgtaaaaagaaagagcaacctacgcagcccttctgtcgatttggcatgggtcaacgcaccacacgagcaggtcaacgagcgctcgtgagaaaaaggacctagggtaaaccacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacatactccctccgtccggaaatacttgtcgaagaatttgatgaaaatggatgcatctagaacaagaatacatctagatacatcaatctccctgacaagtatttccaaaagtaagtaatagtagtagtagtactacatactccctccgtccggaaatacttgtcgaagaatttgatgaaaatggatgcatctagaacaagaatacatctagatacatcaatctccctgacaagtatttccgagcggagggagtactagataataagataactaccctaaaaaaaaagataactgaaggttgccacctagcacattcacattggtacaacttggaaaagcacagccccgtcgtcctgctcccagttgagttcgcgacctacacaccggcc

SEQ ID NO: 27 - DNA, 4749 bp, synthetic construct
  source 1..4749: mol_type = other DNA
  misc_feature 1..4749: note = gene construct
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgggcccggccggccgcgatcgctacgtacctgcaggcccgggttaattaagcggccgcaacatggagtcaaaaattcagatcgaggatctaacagaactcgccgtgaagactggcgaacagttcatacagagtcttttacgactcaatgacaagaagaaaatcttcgtcaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcccctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggactcgagctcatttctctattacttcagccataacaaaagaactcttttctcttcttattaaaccaggtaaccaccccgcccctctcctctttctttctccgttttttttttccgtctcggtctcgatctttggccttggtagtttgggtgggcgagaggcggcttcgtgcgcgcccagatcggtgcgcgggaggggcgggatctcgcggctggggctctcgccggcgtggatccggcccggatctcgcggggaatggggctctcggatgtagatctgcgatccgccgttgttgggggagatgatggggggtttaaaatttccgccatgctaaacaagatcaggaagaggggaaaagggcactatggtttatatttttatatatttctgctgcttcgtcaggcttagatgtgctagatctttctttcttctttttgtgggtagaatttgaatccctcagcattgttcatcggtagtttttcttttcatgatttgtgacaaatgcagcctcgtgcggagcttttttgtaggtagaccatggaattcacgcgtgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcggcgcgccagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 28 - DNA, 6439 bp, synthetic construct
  source 1..6439: mol_type = other DNA
  misc_feature 1..6439: note = gene construct
ctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcttagggcccggtagttctacttctgtccatgtttgtgttagatccgtgtttgtgttagatccgtgctactagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggtcgaccgccggggatcaccaaaaccatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtaattctagcaagcttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcatgcatggtacagcggccgcgttaacgcgtatactctagagcgatcgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaaagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatacctgcaggcaattggtacctacgtatgcatggcgcgccataagcttgcatgc

SEQ ID NO: 29 - DNA, 134 bp, Triticum aestivum
  source 1..134: mol_type = unassigned DNA
aaaagtaagtaatagtagtagtagtactacatactccctccgtccggaaatacttgtcgaagaatttgatgaaaatggatgcatctagaacaagaatacatctagatacatcaatctccctgacaagtatttcc

SEQ ID NO: 30 - DNA, 14 bp, Triticum aestivum
  source 1..14: mol_type = unassigned DNA
agtagtagtactac

SEQ ID NO: 31 - DNA, 22 bp, Triticum aestivum
  source 1..22: mol_type = unassigned DNA
agtagtagtactactagataag

SEQ ID NO: 32 - DNA, 22 bp, Triticum aestivum
  source 1..22: mol_type = unassigned DNA
agtagtagtagtactacatact

SEQ ID NO: 33 - DNA, 290 bp, Triticum aestivum
  source 1..290: mol_type = unassigned DNA
atttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacatactccctccgtccggaaatacttgtcgaagaatttgatgaaaatggatgcatctagaacaagaatacatctagatacatcaatctccctgacaagtatttccgagcggagggagtactagataata

SEQ ID NO: 34 - DNA, 290 bp, Triticum aestivum
  source 1..290: mol_type = unassigned DNA
atttgatggcttcctagtatatttctagcgcttacaactaaatttatattcttggtctaaataaaattataatctgataaattatgtgatatgtatatagccatatgagaccataatttaattttacctgcaaaaagtaagtaatagtaatagtagtactacatactccctccgtccgaaaatacttgtcgaagaatttgatgaaaatggatgcatctagaacaagaatacatctagatacatcaatctccccgacaagtatttccgaacggagggagtactagataata

SEQ ID NO: 35 - DNA, 234 bp, Triticum aestivum
  source 1..234: mol_type = unassigned DNA
gcttcctagtatatttctagggcttacaactaaatttatattcttggtctaaataaaattataatctgataaattatgtgatatgaatatgtcatataggcatatgagaccataatttaattttacctgcaaaaaataagtaatagtagtagtactactagataagataacttggtaaagcactagccccgtcgtctcccagttcaggatctacctacactgcacaccaaggcc

SEQ ID NO: 36 - DNA, 2000 bp, Triticum aestivum
  source 1..2000: mol_type = unassigned DNA
cgaagatggtgatgcgcggagctgggtcgcggcttgtccttctgatatcggcggttcggttcccctttgggtgcgtctgtgcttgtttctcactgtgacagagaagttgaagctacatgcggcgggggccctgcggcaatgatgactccatgagggtggcttcggcggacgatgctctccgcaggttgcactggactagcttggtgtgaggcttgcaactttctcctgtgatgctcatcaacaaaatcggagatgtcggtcctcgacgcctgcggccgatcgtttggcttggctgtcgaggtcctcgggtgccgtttgttcggaggggtcttgatgggtcgtcagcgaggaaatcggagttgcctgctcgtggagaggcgatgacgatgacattgcttgcagcctcgacggtgtcctctcgtcagcagtatgtgtgtgatcttgtcagtgccaggtcggccggagtgctctattcgatgggcgtgttttaattaaccgggcaactctcttcttcttaatcaatgaaaatggcaagtcttttacctcgtttcaaaaagagttaaatgcactggaggtcctaaaggtttcgtcggggtgtcactttagtcctatttcttccaaaatgcacctttaggtcctaattctttattaattggttcacccgaggtccttttccacgagcgctagctgaccagcacacgtgccgcattgacccacgccacatcgatagagggtctaaccgccaggcgagaagcttcgcaaagagaccccccccctcttagtggcaccctctctcctctttcacggtactttggcgacattggaggcgagcgacagaggcaaccggaggtgatctggaggtaatttgtgtgggtgcccgtgaagatgccccttctacgacggccagtgagcaatcctcctccatgttaccgtaagaaccctaatcttgtcgacgatcttctcctgtagctcttctaatatgtcataactttgccataatcaatagttgtagtccttgatttactttgatttgcataacggtgctacggacatggcaaaaaggacacaccaccagcggcaacatacccgccgaagttggagcttgcttcgccgcaccggcggcggtgctgaggatgttgccatgctcctcatcttagttgtttccttgccgaggagcttgaagttccggccatggataggtttagggaacgcgcgcgccgagagagagagcagatttggggaagaaataaagccagggactggggaatgagaagataatgtttataaggggttttctgtaaaaagaaagagcaacctacgcggcccttctgtcgatttggcatgggtcaacgcaccacacgagcaggtcaacgagcgctcgtgagaaaaaggacctagggtaaaccacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagcgcttacaactaaatttatattcttggtctaaataaaattataatctgataaattatgtgatatgtatatagccatatgagaccataatttaattttacctgcaaaaagtaagtaatagtaatagtagtactacatactccctccgtccgaaaatacttgtcgaagaatttgatgaaaatggatgcatctagaacaagaatacatctagatacatcaatctccccgacaagtatttccgaacggagggagtactagataataagataactatccaaaaaaaaaaagataactgaaggttgccacctagcacattcacattggtacaacttggaaaagcacagccccgtcgtcctgctcccagttgagttcgcgacctacacaccggcc

SEQ ID NO: 37 - DNA, 1054 bp, Triticum aestivum
  source 1..1054: mol_type = unassigned DNA
cggaggtgatctggaggcaatctgagtgggtgcccgtgaagatgccccttctacgacggccagtgagcgatcctcctccatgttaccgtgagaaccctaatcttgtcgatgctcttctcctgtagctcttctaatatgccataactctgccataatcaatagttgtagtccttgatttactttgatttgcataacggtgctacggacacggcaaaaaggacacaccaccggcgacaacatacccgccgaagttggagcttgcttcgccgcgccggtggcggtgctgacgatgctgccatgctcctcatcttcagttgtttccttgccgaggagcttgaagttccggccatggataggtttagggcacacgagagagcagatttggggaagaaataaagtcagggactggggaatgagaaggtaatgttcataaggggttttctgtaaaaagaaagagcaacctaagcgacccctttgtcgacttggcgtgggtcaatgcgccacacaggcaggtcaatgagcgcttgtgagaaaaaggtcctcgggtaaaccacttgcttaaagaattactccctccgtcccatattataagaacgtttttgacactagtgtagtgtcaaaaacgttcttatattatgggacggagggagtagtacctaaaggtgcattttggactaaataagactaaagtgacacccagacgaaacttttaggacctcgagtgcatctaactcttaaaaaaacaaagatgctaaaaaactaaatgcttaaaaatatatttttctatgggaatacttaaaaatactattataattttatggcttcctagtatatttctagggcttacaactaaatttatattcttggtctaaataaaattataatctgataaattatgtgatatgaatatgtcatataggcatatgagaccataatttaattttacctgcaaaaaataagtaatagtagtagtactactagataagataacttggtaaagcactagccccgtcgtctcccagttcaggatctacctacactgcacaccaaggcc

SEQ ID NO: 38 - DNA, 16 bp, Triticum aestivum
  source 1..16: mol_type = unassigned DNA
gtagtagtactactag

SEQ ID NO: 39 - DNA, 14 bp, Triticum aestivum
  source 1..14: mol_type = unassigned DNA
agatacatcaatct

SEQ ID NO: 40 - DNA, 16 bp, Triticum aestivum
  source 1..16: mol_type = unassigned DNA
  misc_feature 1..16: note = binding site present the RFL29a promoter (16 bp)
gtaatagtagtactac

SEQ ID NO: 41 - DNA, 15 bp, Triticum aestivum
  source 1..15: mol_type = unassigned DNA
  misc_feature 1..15: note = PHD binding site, shorter version of SEQ ID NO: 40 (15 bp)
gtaatagtagtacta

SEQ ID NO: 42 - DNA, 15 bp, Triticum aestivum
  source 1..15: mol_type = unassigned DNA
  misc_feature 1..15: note = PHD binding site, shorter version of SEQ ID NO: 11 (15 bp)
gtagtagtagtacta

SEQ ID NO: 43 - DNA, 2373 bp, Triticum timopheevi
  source 1..2373: mol_type = unassigned DNA
atgccccgcttctcctccaccacgccaatgtcgccaccccgcctccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcaccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttattgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattcgccttcatgggtatgccaccgaaggatgcctagttgacatgatcaatctcttcaattccatggcaacacactgcattctacctaactgtcatatattcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatag

SEQ ID NO: 44 - protein, 790 aa, Triticum timopheevi
MPRFSSTTPMSPPRLRLRLRLCARHSSSTSHPSRIWDPHAAFAAAAQRASSGTLTTEDAHHLFDELLRRGNPVQERPLNKFLAALARAPASASCCDGPALAVTLFGRLSRDVGRRVAQPNVFTYGVLMDCCCRACRTDLVLAFFGRLLKTGLEANQVVFNTLLKGLCHTKRADEALDVLLHRMPELGCTPNVVAYNTVIHGFFKEGHVSKACNLFHEMAQQGVKPNVVTYNSVIDALCKARAMDKAEVVLRQMIDDGVGPDNVTYSSLIHGYSSSGHWKEAVRVFKEMTSRRVTADVHTYNMFMTFLCKHGRSKEAAGIFDTMAIKGLKPDNVSYAIRLHGYATEGCLVDMINLFNSMATHCILPNCHIFNILINAYAKSGKLDKAMLIFNEMQKQGVSPNAVTYSTVIHAFCKKGRLDDAVIKFNQMIDTGVRPDASVYRPLIQGFCTHGDLVKAKEYVTEMMKKGMPPPDIMFFSSIMQNLCTEGRVTEARDILDLIVHIGMRPNVIIFNLLIGGYCLVRKMADALKVFDDMVSYGLEPCNFTYGILINGYCKNRRIDDGLILFKEMLHKGLKPTTFNYNVILDGLFLAGQTVAAKEKFDEMVESGVSVCIDTYSIILGGLCRNSCSSEAITLFRKLSAMNVKFDITIVNIIIGALYRVERNQEAKDLFAAMPANGLVPNAVTYTVMMTNLIKEGSVEEADNLFLSMEKSGCTANSCLLNHIIRRLLEKGEIVKAGNYMSKVDAKSYSLEAKTVSLLISLFSRKGKYREHIKLLPTKYQFLEEAATVE

SEQ ID NO: 45 - RNA, 19 nt, Triticum timopheevi
  source 1..19: mol_type = unassigned RNA
gggtaggttggatgatgct

SEQ ID NO: 46 - DNA, 19 bp, Triticum timopheevi
  source 1..19: mol_type = unassigned DNA
gggtaggttggatgatgct

SEQ ID NO: 47 - RNA, 21 nt, Triticum timopheevi
  source 1..21: mol_type = unassigned RNA
tagcatcatccatcctaccca

SEQ ID NO: 48 - RNA, 21 nt, Triticum timopheevi
  source 1..21: mol_type = unassigned RNA
agggtaggttggatgatgctg

SEQ ID NO: 49 - DNA, 321 bp, synthetic construct
  source 1..321: mol_type = other DNA
  misc_feature 1..321: note = sequence encoding PPR units 8 to 10 of the wheat Rf3-58 protein, optimized for expression in wheat
atggcctgccacatcttcaacatcctgataaacgcctacgccaagtcaggcaagctcgacaaggcgatgctcatcttcaacgagatgcagaagcaaggcgtctcaccgaatgccgttacctacagcacggtgatccacgccttctgcaagaaaggacgcctagacgacgcggtcatcaagttcaaccagatgatcgacaccggcgttaggcctgatgcttcggtgtatcgcccactaattcagggcttctgcactcatggggacctcgttaaggcgaaggagtacgtcaccgagatgatgaagaagggcatgccgccaccg

SEQ ID NO: 50 - RNA, 19 nt, synthetic construct
  source 1..19: mol_type = other RNA
  misc_feature 1..19: note = mutated miRNA binding site (pBas04648)
aggacgcctagacgacgcg

SEQ ID NO: 51 - RNA, 19 nt, Triticum timopheevi
  source 1..19: mol_type = unassigned RNA
gggtaggttggatgatgct

SEQ ID NO: 52 - RNA, 19 nt, synthetic construct
  source 1..19: mol_type = other RNA
  misc_feature 1..19: note = modified miRNA binding site
aggacgcctagacgacgcg

SEQ ID NO: 53 - RNA, 19 nt, synthetic construct
  source 1..19: mol_type = other RNA
  misc_feature 1..19: note = modified miRNA binding site
gggtcgcttagacgacgct

SEQ ID NO: 54 - RNA, 19 nt, synthetic construct
  source 1..19: mol_type = other RNA
  misc_feature 1..19: note = modified miRNA binding site
gggtcgcttagatgatgct

SEQ ID NO: 55 - RNA, 19 nt, synthetic construct
  source 1..19: mol_type = other RNA
  misc_feature 1..19: note = modified miRNA binding site
gggtcgcttggatgatgct

SEQ ID NO: 56 - RNA, 19 nt, synthetic construct
  source 1..19: mol_type = other RNA
  misc_feature 1..19: note = modified miRNA binding site
gggtaggttagacgatgct

SEQ ID NO: 57 - RNA, 19 nt, synthetic construct
  source 1..19: mol_type = other RNA
  misc_feature 1..19: note = modified miRNA binding site
gggtaggttggacgacgct

SEQ ID NO: 58 - RNA, 19 nt, synthetic construct
  source 1..19: mol_type = other RNA
  misc_feature 1..19: note = modified miRNA binding site
gggtaggttagatgatgct

SEQ ID NO: 59 - RNA, 19 nt, synthetic construct
  source 1..19: mol_type = other RNA
  misc_feature 1..19: note = modified miRNA binding site
gggtaagttggatgatgct

SEQ ID NO: 60 - RNA, 19 nt, synthetic construct
  source 1..19: mol_type = other RNA
  misc_feature 1..19: note = modified miRNA binding site
gggtagattggatgatgct

SEQ ID NO: 61 - RNA, 19 nt, synthetic construct
  source 1..19: mol_type = other RNA
  misc_feature 1..19: note = modified miRNA binding site
gggtaggttggatgatgtt

SEQ ID NO: 62 - DNA, 2367 bp, Triticum timopheevi
  source 1..2367: mol_type = unassigned DNA
atgccccgcttctcctccaccacgccaatgtcgccaccccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatag

SEQ ID NO: 63 - protein, 788 aa, Triticum timopheevi
MPRFSSTTPMSPPRLRLRLCARHSSSTSHPSRIWDPHAAFAAAAQRASSGTLTTEDAHHLFDELLRRGNPVQERPLNKFLAALARAPASASCCDGPALAVALFGRLSRDVGRRVAQPNVFTYGVLMDCCCRACRTDLVLAFFGRLLKTGLEANQVVFNTLLKGLCHTKRADEALDVLLHRMPELGCTPNVVAYNTVIHGFFKEGHVSKACNLFHEMAQQGVKPNVVTYNSVIDALCKARAMDKAEVVLRQMIDDGVGPDNVTYSSLIHGYSSSGHWKEAVRVFKEMTSRRVTADVHTYNMFMTFLCKHGRSKEAAGIFDTMAIKGLKPDNVSYAILLHGYAAEGCLVDMINLFNSMERDCILPDCRIFNILINAYAKSGKLDKAMLIFNEMQKQGVSPNAVTYSTVIHAFCKKGRLDDAVIKFNQMIDTGVRPDASVYRPLIQGFCTHGDLVKAKEYVTEMMKKGMPPPDIMFFSSIMQNLCTEGRVTEARDILDLIVHIGMRPNVIIFNLLIGGYCLVRKMADALKVFDDMVSYGLEPCNFTYGILINGYCKNRRIDDGLILFKEMLHKGLKPTTFNYNVILDGLFLAGQTVAAKEKFDEMVESGVSVCIDTYSIILGGLCRNSCSSEAITLFRKLSAMNVKFDITIVNIIIGALYRVERNQEAKDLFAAMPANGLVPNAVTYTVMMTNLIKEGSVEEADNLFLSMEKSGCTANSCLLNHIIRRLLEKGEIVKAGNYMSKVDAKSYSLEAKTVSLLISLFSRKGKYREHIKLLPTKYQFLEEAATVE

SEQ ID NO: 64 - DNA, 2376 bp, Triticum timopheevi
  source 1..2376: mol_type = unassigned DNA
atgcctcgcttctcctccaccacgccaatgtcgccaccccgcctcctcctccggctcggcgcccgccactcctcctccacctctcatccctcacgcatctgggatccccacgccgccttcgccgctgcgacgcagcgggcgcgctctggcacgctcaccacggaggacgcacaccacctgtttgatgaattgctgcggcagggcaatcctgtccaggagcgtcccttgactaactttctggctgccctcgcccgcgcgcccgcgtccgcattctgcagcgatggccctgccctggccgtcgccctcttcggccgtttgtcccgaggcgccggacgacgggtggcgcagccaaatgtcttcacctatggcgtcctcatggactgctgctgccgtgcgcgccgcctggatctagcgatcgccttcttcgcccgtctcctcaagacgggactggaggcaaaccaagtcatcttctgcaccctcctcaagggactctgccacgcaaagcgctcagatgaggctttggacgtggtgcttcacaggatgcctgagctaggctgcacccccaacgtggtggcctataccacggtcatccacggcttcttgaaggaaggccaagtaggcaaggcatgcaatctattccatggaatggcgcagcagggcgttgcgcctgatgtggtgacatataactcggttatcgatgcgttgtgcaaggccagagcaatggacaaggcagagtatttccttcgtgaaatggttgataatggtgtcgtacctaataatgtgacatataatagcctcatccatggatattcctctttgggccatcagaaggaggctgttagggtgctgaaagaaatgacaagacagggtatcataccagatgtcattacctgcacctcactcatgaccttcctttgcaagaatggaaaaagcaaggaagctgcagaaatttttgattcaatggccacgaagggcctgaaacatgacgccgtttcatatgctattctccttcatgggtatgccactgaaggatgcttggttgatatgattaatctcttcaattcgatggacagagactgtattctacctaactgtcatatcttcaacatactgatttatgcatatgctaaatctgggaagcttgataaggctatgcttatatttagagatatgcagaaacaaggagtgagcccagatgcattcacatattcaaccttaatacatgcattttgtaaaaagggtcggttggacgatgctatgataaagtttaatcagatggttgatacaggagtacgacagggcacagctgtttatggttctctaatccagggtttttgtacacacggcgatttggtgaaaggaaaggaattggttactgaaatgatgaacaaaggtatacctcctcctgacattatgttcttccattcaatcatgcagaacctatgcacagaaggaagggtagtagaagcacgggatatccttggcttgatagcacacataggtatgaggcctaatgtttgcacatttaatatactgattggtggatactgcctagtccgcaagatggaggatgcctcaaaaatatttcatgatatgatgtcatatggtttagaaccttctaatgttacgtatggtattcttattaatggctattgcaaaaacagaaggattgatgacgggctgattctgttcaaagaaatgttgcgcaagggacttaaacctacaacttttaattacaacatcatactggatggattatttctggctggacgaactgttgctgcaaaggaaaagtttgatgagatggttgaatctggagtaagtatgtgcatcagtacttactctatagttcttcgtggactttgtagaaataattgtagcggcgaagccatcacgctattccagacattaagcgcaatggatgtgaaattcaatattagaattgtcaatatcatgattgatgccttcttcagggttcagcgaaagcaagaagctaaggatttgtttgctgcaataacagccaatgggttggttgctaatgtttttacctacagcctaatgatgacaaatcttataaaagaagggtcagtggaagaggctgacacactctttttatcgatggagatgagcggctgtacttcgaactcgtggatgttaaatcttattatcagagggttgctggaaaaaggagagatagtcaaggctggatgttatatgtctaaagttgatgcgaagagctactcacttgaagctaaaactgtttcgttgctgatctatctcttttcagggaaagggaaatacagagaacacataagattgctacctacaaagtatcagtttctcgaagaagcagccacagttgaatggtttgctata

SEQ ID NO: 65 - protein, 792 aa, Triticum timopheevi
MPRFSSTTPMSPPRLLLRLGARHSSSTSHPSRIWDPHAAFAAATQRARSGTLTTEDAHHLFDELLRQGNPVQERPLTNFLAALARAPASAFCSDGPALAVALFGRLSRGAGRRVAQPNVFTYGVLMDCCCRARRLDLAIAFFARLLKTGLEANQVIFCTLLKGLCHAKRSDEALDVVLHRMPELGCTPNVVAYTTVIHGFLKEGQVGKACNLFHGMAQQGVAPDVVTYNSVIDALCKARAMDKAEYFLREMVDNGVVPNNVTYNSLIHGYSSLGHQKEAVRVLKEMTRQGIIPDVITCTSLMTFLCKNGKSKEAAEIFDSMATKGLKHDAVSYAILLHGYATEGCLVDMINLFNSMDRDCILPNCHIFNILIYAYAKSGKLDKAMLIFRDMQKQGVSPDAFTYSTLIHAFCKKGRLDDAMIKFNQMVDTGVRQGTAVYGSLIQGFCTHGDLVKGKELVTEMMNKGIPPPDIMFFHSIMQNLCTEGRVVEARDILGLIAHIGMRPNVCTFNILIGGYCLVRKMEDASKIFHDMMSYGLEPSNVTYGILINGYCKNRRIDDGLILFKEMLRKGLKPTTFNYNIILDGLFLAGRTVAAKEKFDEMVESGVSMCISTYSIVLRGLCRNNCSGEAITLFQTLSAMDVKFNIRIVNIMIDAFFRVQRKQEAKDLFAAITANGLVANVFTYSLMMTNLIKEGSVEEADTLFLSMEMSGCTSNSWMLNLIIRGLLEKGEIVKAGCYMSKVDAKSYSLEAKTVSLLIYLFSGKGKYREHIRLLPTKYQFLEEAATVEWFAI

SEQ ID NO: 66 - DNA, 19 bp, Triticum timopheevi
  source 1..19: mol_type = unassigned DNA
gggtcggttggacgatgct

SEQ ID NO: 67 - RNA, 19 nt, Triticum timopheevi
  source 1..19: mol_type = unassigned RNA
gggtcggttggacgatgct

SEQ ID NO: 68 - RNA, 21 nt, Triticum timopheevi
  source 1..21: mol_type = unassigned RNA
agggtcggttggacgatgcta

SEQ ID NO: 69 - RNA, 19 nt, synthetic construct
  source 1..19: mol_type = other RNA
  misc_feature 1..19: note = miRNA binding site which is 100 percent complementary to miRNA3619
gggtaggatggatgatgct

SEQ ID NO: 70 - DNA, 144 bp, Triticum sp.
  source 1..144: mol_type = unassigned DNA
  misc_feature 1..144: note = Wheat enhancer sequence
tagacacgttgtgagctgccttgacttgggtaggcagatccagacctgaaacgtcaggctcatggataaggatcccggccatctgtcactgacgtgtcgtcagtttgcccctagccttgtgggattgccatctccatctgccta

SEQ ID NO: 71 - DNA, 4138 bp, Triticum sp.; identical to SEQ ID NO: 1
  source 1..4138: mol_type = unassigned DNA
  misc_feature 1..4138: note = Wheat promoter fragment

SEQ ID NO: 72 - DNA, 2000 bp, Triticum sp.; identical to SEQ ID NO: 21
  source 1..2000: mol_type = unassigned DNA
  misc_feature 1..2000: note = Wheat promoter fragment

SEQ ID NO: 73 - DNA, 1423 bp, Triticum sp.; identical to SEQ ID NO: 22
  source 1..1423: mol_type = unassigned DNA
  misc_feature 1..1423: note = Wheat promoter fragment

SEQ ID NO: 74 - DNA, 1260 bp, synthetic construct; identical to SEQ ID NO: 23
  source 1..1260: mol_type = other DNA
  misc_feature 1..1260: note = Wheat promoter fragment variant

SEQ ID NO: 75 - DNA, 9155 bp, synthetic construct
  source 1..9155: mol_type = other DNA
  misc_feature 1..9155: note = sequence from vector pBay02430
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatcctgcaggcccgggttaattaagcggccgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgatcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctccccccccccccctctctaccttctctagatcggcgttccggtccatgcttagggcccggtagttctacttctgtccatgtttgtgttagatccgtgtttgtgttagatccgtgctactagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttctgcaggaccatggcacccaagaagaagaggaaggtgggcatccacggcgtcccagcagcaatggacaagaagtactccatcggcctcgacatcggcaccaacagcgtcggctgggccgtgatcaccgacgagtacaaggtgccaagcaagaagttcaaggtgctgggcaacaccgaccgtcactccatcaagaagaacctcatcggcgccctcctcttcgactcaggtgagacggccgaggcgacaaggttgaagaggacggcaagaagaaggtacacaagaaggaagaacaggatctgctacctccaggagatcttcagcaacgagatggccaaggtggatgacagcttcttccaccggctagaggagagcttcttggtggaggaggacaagaagcatgagaggcatcccatcttcggcaacatcgtcgacgaggtggcctaccatgagaagtaccccaccatctaccatctccgcaagaagctggtggactcaacggacaaggccgacctccgcctcatctacctcgccttggcccacatgatcaagttcagaggccacttcctcatcgagggcgacctcaaccctgacaacagcgatgtggacaagctcttcatccagctggtgcagacctacaaccagctctttgaggagaaccccatcaacgcctccggcgtcgacgccaaggccatcctctcggcaaggttgagcaagtcaagaaggctggagaacctcatcgctcagctccccggcgagaagaaaaatggcctcttcggcaacctcatcgccttgagcctcggcctcacccccaacttcaagtcaaactttgacctcgccgaggatgccaagctccagctctccaaggacacctacgacgacgacctcgacaacctcctcgcccagatcggcgaccagtacgccgacctcttcctcgccgccaagaacctctcagatgccatcctcctgagcgacatcctccgcgtcaacaccgagatcaccaaggcgccgctctccgcctccatgatcaagaggtatgatgagcaccatcaagatctcaccctcctcaaggccttggtgaggcagcagctacctgagaagtacaaggagatcttcttcgaccagagcaagaatggctacgccggctacatcgacggcggcgcctcccaagaagagttctacaagttcatcaagcccatcttggagaagatggatggcaccgaggagctactggtgaagctcaacagagaagatctcctcaggaagcagaggaccttcgacaacggcagcatccctcaccagatccaccttggtgagctccatgccatcttgagaaggcaagaagacttctaccccttcctcaaggacaacagggagaagatcgagaagatattgacattcaggatcccctactacgtcggacccttggcaagaggcaactcaaggttcgcctggatgacaaggaagagcgaggaaaccatcaccccctggaactttgaggaggtggtggacaagggcgcctcagctcagagcttcatcgagaggatgaccaacttcgacaagaacctccccaacgagaaggtgctccccaagcacagcctcctctacgagtacttcaccgtctacaatgagctcaccaaggtcaagtacgtcaccgagggcatgaggaagccggccttcctctcaggagagcagaagaaggccatcgtcgacctcctcttcaagaccaacaggaaggtcaccgtcaagcagctcaaggaggactacttcaagaagatcgagtgcttcgactctgtggagatctccggcgtggaggacaggttcaacgcctccctcggcacctaccatgatctcctgaagatcatcaaggacaaggacttcctcgacaacgaggagaacgaggacatcttagaagacatcgtcctcaccctcaccctcttcgaggacagggagatgatcgaggagaggctcaagacctatgctcacctctttgatgacaaggtgatgaagcagctcaagagaagaaggtacaccggctggggccgcctctcaaggaagctcatcaacggcatcagggacaagcagagcggcaagaccatcctcgacttcctcaagagcgatggcttcgccaacaggaacttcatgcagctcatccatgatgacagcttgaccttcaaggaggacatccagaaggctcaagtctccggccaaggagattccctccatgagcacatcgccaacctcgccggctcaccggccatcaagaagggcatcctccagaccgtcaaggtggtggatgagctggtgaaggtgatgggccgtcacaagccggagaacatcgtcatcgagatggcaagggagaaccagaccacccagaagggccagaagaactcaagggagaggatgaagaggattgaagaaggcatcaaggagctcggcagccagatcctcaaggagcaccctgtggagaacacccagctacagaacgagaagctctacctctactacctccagaatggccgtgacatgtacgtcgaccaagagctggacatcaacaggttgagcgactacgacgtcgaccacatcgtcccccagagcttcctcaaggatgacagcatcgacaacaaggtgctgacaagatcagacaagaaccgtggcaagagcgacaatgtcccctccgaggaggtggtgaagaagatgaagaactactggaggcagctcctcaacgccaagctcatcacccagaggaagttcgacaacctcaccaaggctgaaagaggaggcctctcagagctggacaaggccggcttcatcaagaggcagctggtggagacaaggcagatcaccaagcatgttgctcagatccttgattcaaggatgaacaccaagtacgacgagaacgacaagctcatcagggaggtcaaggtcatcaccctcaagagcaagctggtgtccgacttcaggaaggacttccagttctacaaggtgagggagatcaacaactaccaccatgctcatgatgcctacctcaacgccgtggtgggcaccgccctcatcaagaagtaccccaagctggagtcagagttcgtctacggcgactacaaggtgtatgatgtgaggaagatgatcgccaagagcgagcaggagatcggcaaggccaccgccaagtacttcttctactcaaacatcatgaacttcttcaagaccgagatcaccctcgccaacggcgagatcaggaagaggccgctcattgagacaaatggtgagactggtgagatcgtctgggacaagggccgagacttcgccaccgtgaggaaggtgctctccatgcctcaagtcaacatcgtcaagaagaccgaggtgcagaccggaggcttctcaaaggagagcatcctccccaagaggaactctgacaagctgatcgcaaggaagaaggactgggaccccaagaagtacggcggctttgattcaccaacggtggcctactccgtgctggtggtggccaaggtggagaagggcaagagcaagaagctcaagagcgtcaaggagctcctcggcatcaccatcatggagagatcatccttcgagaagaaccccatcgacttccttgaggccaagggctacaaggaggtgaagaaggacctcatcatcaagctccccaagtacagcctcttcgagctggagaatggccgcaagaggatgctggcatcagctggagagctccagaagggcaacgagctggccctcccaagcaagtacgtgaacttcctctacctcgcctcccactacgagaagctcaagggctcaccagaagacaatgagcagaagcagctcttcgtggagcagcacaagcactacctcgacgagatcatcgagcagatctccgagttctccaagagggtgatcctcgccgacgccaacctcgacaaggtgctctcagcctacaacaagcaccgtgacaagcccatcagggagcaagctgagaacatcatccacctcttcaccctcaccaacctcggcgccccggcggccttcaagtacttcgacaccaccatcgaccgcaagaggtacacctcaaccaaggaggtgcttgatgccaccttgatccaccagagcatcaccggcctctacgagacaaggatcgacctctcccagctcggcggcgacaagaggccggcggccaccaagaaggccggccaagccaagaagaagaagtgattgctagcacgcgttggacacgctgaaatcaccagtctctctctacaaatctatctctctctattttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcggcgcgccatcccaatggcgcgccgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 76 - DNA, 6635 bp, synthetic construct
  source 1..6635: mol_type = other DNA
  misc_feature 1..6635: note = sequence from vector pBay02032
gtgcaccatacctgcaggcaattggccgctgtaccatgcatgatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaaatagagagagatagatttgtagagagagactggtgatttcagcgtgtccaagcttgctagagtcgcggccgcttcagatctcggtgacgggcaggaccggacgtggcggtaccggcaggctgaagtccagctgccagaaacccacgtcatgccagttcccgtgcttgaagccggccgcccgcagcatgccgcggggggcatatccgagcgcctcgtgcatgcgcacgctcgggtcgttgggcagcccgatgacagcgaccacgctcttgaagccctgtgcctccagggacttcagcaggtgggtgtagagcgtggagcccagtcccgtccgctggtggcggggggagacgtacacggtcgactcggccgtccagtcgtaggcgttgcgtgccttccaggggcccgcgtaggcgatgccggcgacctcgccgtccacctcggcgacgagccagggatagcgctcccgcagacggacgaggtcgtccgtccactcctgcggttcctgcggctcggtacggaagttgaccgtgcttgtctcgatgtagtggttgacgatggtgcagaccgccggcatgtccgcctcggtggcacggcggatgtcggccgggcgtcgttctgggtccatcttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggttttggtttaataagaagagaaaagagttcttttgttatggctgaagtaatagagaaatgagctcgagtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcaggggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttgacgaagattttcttcttgtcattgagtcgtaaaagactctgtatgaactgttcgccagtcttcacggcgagttctgttagatcctcgatctgaatttttgactccatgtatggtgcatatggcgcgccatagatccgtcaattctcactcattaggcaccccaggctttacactttatgcttccggctcgtataatgtgtggaattgtgagcggataacaatttcacacaggaaacaggatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccagatccggcccatgatcatgtggattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccctgtacagcggccgcgttaacgcgtatactctagagcgatcgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgaga

SEQ ID NO: 77 - DNA, 3120 bp, synthetic construct
  source 1..3120: mol_type = other DNA
  misc_feature 1..3120: note = sequence from vector pBas03477
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaaccctgcagggaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgcagatgattgatgatggtgtgttttagagctagaaatagcaagttaaaataaggctagtccgttatcaacttgaaaaagtggcaccgagtcggtgctttttttttttaattaagttccatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 78 - DNA, 3454 bp, synthetic construct
  source 1..3454: mol_type = other DNA
  misc_feature 1..3454: note = sequence from vector pBas03482
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgacgcgtattgggatggaacggcgcgccctgcatttcattgaagataagcatagccttatcaagcttcccagatttagcatatgcattaatcagtatgttgaagatacgacagtcaggtagaatacaatctctttccattgaattgaagagattaatcatatcaactaagcatccttcggcggcatacccatgaaggagaatagcatatgaaacgttgtcaggtttcaggcccttgatagccatggtatcaaaaattcctgcagcttctttgcttcttccatgtttgcaaagaaaggtcataaacatgttgtaagtatgcacatctgctgtaaccctccgacttgtcatctctttgaataccctaactgcctccttccagtggcctgaagaggaatatccatggatgaggctactatacgtcacattatcaggcccaactccgtcatcaatcatctgacgaaggaccacctctgccttgtccatggctctggccttgcacagcgcatcgataactgagttatatgtcaccacattaggcttaacgccctgctgcgccatttcatggaacagattgcaggccttgcttacatggccttccttaaagaagccatggataacggtgttatacgccaccacatcaggagtgcagcccagctcaggcatcctgtgaagcagcacgtccagagcctcatccgcccgctttgtgtggcaaaggcccttgaggagggtgttgaagacgacttggtttgcctccaggcccgtcttgaggagacggccaaagaaggcgagcgccagatctgtgcggcaagcgcggcagcagcagtccatgttaattaagttccatcccaatggcgcgccgagcttggctcgagcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccagggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctagagcaagacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 79 - DNA, 2968 bp, synthetic construct
  source 1..2968: mol_type = other DNA
  misc_feature 1..2968: note = sequence from vector pBas03682
ggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagaaacccaattgggtaccgagctccctgcaggaagcttaattaagatgagaccgaggtcgtgatgtctgaagtacgccaatacatctatggcatcggtacgcagtgaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgaaaagaaagagcaacctacggttttagagctagaaatagcaagttaaaataaggctagtccgttatcaacttgaaaaagtggcaccgagtcggtgctttttttttggcgcgccacctgctagcgctcgcttggatccgaattcaaacctgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatccgcgcacatttccccgaaaagtgccacctgacgtctaagaaaccattattatcatgacattaacctataaaaataggcgtatcacga

SEQ ID NO: 80 - DNA, 2968 bp, synthetic construct
  source 1..2968: mol_type = other DNA
  misc_feature 1..2968: note = sequence from vector pBas03683
ggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagaaacccaattgggtaccgagctccctgcaggaagcttaattaagatgagaccgaggtcgtgatgtctgaagtacgccaatacatctatggcatcggtacgcagtgaccaagcccgttattctgacagttctggtgctcaacacatttatatttatcaaggagcacattgttactcactgctaggagggaatcgaactaggaatattgatcagaggaactacgagagagctgaagataactgccctctagctctcactgatctgggtcgcatagtgagatgcagcccacgtgagttcagcaacggtctagcgctgggcttttaggcccgcatgatcgggcttttgtcgggtggtcgacgtgttcacgattggggagagcaacgcagcagttcctcttagtttagtcccacctcgcctgtccagcagagttctgaccggtttataaactcgcttgctgcatcagacttgacgtatagtagcctcatccagttttagagctagaaatagcaagttaaaataaggctagtccgttatcaacttgaaaaagtggcaccgagtcggtgctttttttttggcgcgccacctgctagcgctcgcttggatccgaattcaaacctgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgatccgcgcacatttccccgaaaagtgccacctgacgtctaagaaaccattattatcatgacattaacctataaaaataggcgtatcacga

SEQ ID NO: 81 - DNA, 5030 bp, synthetic construct
  source 1..5030: mol_type = other DNA
  misc_feature 1..5030: note = sequence from vector pBas03913
gggtgttggcgggtgtcggggctggcttaactatgcggcatcagagcagattgtactgagagtttcccaattggtcgacctcgagttaatttaattaaggcttttggcggaatgatcaattgccctcgtttattgcaccgctgatttccggtgggcatattaacctacgacggccagtgagcaatcctcctccatgttaccgtaagaaccctaatcttgtcgatgatcttctcctgtagctcttctaatatgtcataactttgccataatcaatagttgtagtccttgatttactttgatttgcataacggtgctacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggcttaggggacgcgcgcgccgagagagagagcagatttggggaagaaataaagccagggactggggaatgagaagataatgtttataaggggttttctgtaaaaagaaagagcaacctacgcagcccctctgtcgatttggcatgggtcaacgcaccacacgagcaggtcaacgagcgctcgtgagaaaaaggacctagggtaaatcacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacgtactccctccgtccggaaatacttgtcgaagaatttgatgaaaatggatgcatctagaacaagaatacatctagatacatcaatctccccgacaagtatttccgaacggagggagtactagataatagacacgttgtgagctgccttgacttgggtaggcagatccagacctgaaacgtcaggctcatggataaggatcccggccatctgtcactgacgtgtcgtcagtttgcccctagccttgtgggattgccatctccatctgcctataagataactaccctaaaaaaaaaagataactgaaggttgccacctagcacattcacattggtacaacttggaaaagcacagccccgtcgtcctgctcccagttgagttcgcgacctacacaccggccatgccccgcttctcctccaccacgccaatgtcgccaccccgcctccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcaccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttattgatgcgctgtgcaaggccagagccatggacaaggcagaggtggtccttcgtcagatgattgatgacggagttgggcctgataatgtgacgtatagtagcctgattcatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattcgccttcatgggtatgccaccgaaggatgcctagttgacatgatcaatctcttcaattccatggcaacacactgcattctacctaactgtcatatattcaacatactgattaatgcatatgctaaatctgggaagcttgataaggctatgcttatcttcaatgaaatgcagaaacaaggagtgagtccaaatgcagtcacatattcaaccggcgcgcccgaattctttcctgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaaaatattccggaattgccagctggggcgccctctggtaaggttgggaagccctgcaaagtaaactggatggctttcttgccgccaaggatctgatggcgcaggggatcaagatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatcccaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaaccggtaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacgatgaaaacctctgacacatgcagctcccggagacggtcacagcttgtctgtaagcggatgccgggagcagacaagcccgtcagggcgcgtcagc

SEQ ID NO: 82 - DNA, 6905 bp, synthetic construct
  source 1..6905: mol_type = other DNA
  misc_feature 1..6905: note = gene construct
atgtcacatctcaacaacgtttttccaaaaccatggtattccaaaacctaagtatttcaatacatggtcaataaatacttcagtttctaaaaaccatagtttttctcagttttgataaaactgcgaggtgtttggcaagtggcagtttttctcagttttctttggtttgcatcgactgttacgtgcatgcatattcagctacactcagggatcagcttgtactaatagccatcgtatgcattcagccttgcatgagcatgttctcctagatgctaagatctacagtagagttatctcttgtaatcaatcaaagaccgaggtagttaacacatgcactgaaggtcgacgtttgcagctcgtgtacgagtacatccgcctccagccggacatatcgatgggctacctatcgatttatgcatcacagtcgttgatggccagaagttggatacaagagagaccagcaaccagcgctttctcggttttaaaaaaaagaggtcagggcctcttttttatatacaacaaaaataccacagttttagagataccatggtctgttgaactgcagtattttttttatccaaacgactcaaagtattaaataccaaagtttttttcaaaaaccacagtattccagaaaaccacaaaaatacttcggcttccaaacgcaccctaagtgtaagaagaaatctactaagttcacaaaattcagaagaaactcgtttggaccatgagcggtacagaaaagaaaatggcgtgatctagaatagcagacattagggtctgtttgcaggtattatggatcactgtatgtttgcaaaaaataataatttgtgcagctatcagatgcatgcattttctatgaaaattttgtagatccagtgttatacccaacaacaaaatttaatacttgggtcaccacaacccaagatggagctccaagcaaacttaagtaagaggataccgagatgtgatgatgatgtctgatcttgggggaatttttgtagtgagaaaatgccttaaacatcatcgcggaatccaaatggtcaatcaggactgacaccaccaatggcttgattcaccatctctatctgcccattttgacgtaacactatttggctttcgacgcgacgggataatcaacgtgacaatcctgtcatgagaaagtcatcatgccgagttcattttccaggcatcccgtgtggcatcttgccattctctccgtccatgagttgattacccaaaagcatctgatcccggagcacaacgaacgacatcagaagacatggttcgcatcacaaaacaccatttgatcatgttacgcatgttaggcacatctgaagaagggaaggcgttgagaacctcggccaccactagtagtagacttccaacttttagttgggacaacatgggcgggatttcacggcacaaaattcaccacctagatgctaaaaagataccccccccccccccccatcccccgcacatcctccttgaagacacgggcgccccctccgcctccctacctcgccgtcgccggaggaggccgccgggctaagcctgtgcggcggacgacggcggcagggcctctctgcaacgtccgggtctcatggtgagggaggcacgcctccttgcagtgcggcacggcggctacggcacgaagattctcggaagttgcgtctccagcgggtggcagtcgcggtggtgtcgtcggcaaactcctgggcttcggtccggcggctcttgctcggcgagtatgggcggggtggcggcccccctttcctgcctttggtcattcgtctccgcgcaggtcgactgctctggccggcccttggtggttgttgtagtgctgctgcaagggggcgccagatccggcggttcttcattcggatccagctcgtctctcgtcttactcagggtcggtcatcccggtgaccttgcctctggcagcgagggggtgtgcgtgctctatggtgggccttgtgaggcgatggcgcggttgctgcgcaggggtgtgcatgccagcgattgcgcgggccagcttgtgtcggctgcgtgagggtgttggttgagtctgctccaagcgcaagcttgcccggctatggccggccggtgctggcggcgcctatgggcgtcgtttcccttgttgaaggcgttgttgtggagttcttcgacctactacaatctcgaatctggttcttcgggcgaaaacctcgccccatctaggtcgggcaacgacgtcgtccgtacggtgttttccccctttggggcgttgctttgaagaccggttatcctttcccgtgctacctttgggctggtcgtgcatggtgtcatggttggctggtgctcaatcggtgatgcgagtggttggcgttgcgacttgtgcggtgacgaagatggtgatgcgcggagctgggtcgcggcttgtccttctgatatcggcggttcggttcccctttgggtgcgtctgtgcttgtttctcactgtgacagagaagttgaagctacatgcggcgggggccctgcggcaatgatgactccatgagggtggtttcggcggacgatgctctccgcaggttgcactggactagcttggtgtgaggcttgcaactttctcctgtgatgctcatcaacaaaatcagagatgtcggtcctcgacgcctgcggccgatcgtttggcttggctgtcgaggtcctcgggtgccgtttgttcggaggggtcttgatgggtcgtcagcgaggaaatcggagttgcctgctcgtggagaggcgatgacgatgacattgcttgcagcctcgatggtgtcctctcgtcagcagtatgtgtgtgatcttgtcagtgccaggtcggccggagtgccctattcgatgggcgtgttttaattaaccgggcaactctcttcttcttaatcaatgaaaatggcaagtcttttacctcgtttcaaaaagagttaaatgcactggaggtcctaaaggtttcgtcggggtgtcactttagtcctatttcttccaaaatgcacctttaggtcctaattctttattaattggttcacccgaggtccttttccacgagcgctcgctgaccagcacacgtgccgcattgacccacgccacatcgatagagggtctaaccgtcaggcgagaagcttcgcaaagagaccccccccctcttagtggcaccctctctcctctttcacggtactttggcgacattggaggcgagcgacagaggcaaccggaggtgatctggaggtaatttgtgtgggtgcccgtgaagatgccccttctacgacggccagtgagcaatcctcctccatgttaccgtaagaaccctaatcttgtcgatgatcttctcctgtagctcttctaatatgtcataactttgccataatcaatagttgtagtccttgatttactttgatttgcataacggtgctacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggcttaggggacgcgcgcgccgagagagagagcagatttggggaagaaataaagccagggactggggaatgagaagataatgtttataaggggttttctgtaaaaagaaagagcaacctacgcagcccctctgtcgatttggcatgggtcaacgcaccacacgagcaggtcaacgagcgctcgtgagaaaaaggacctagggtaaatcacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacgtactccctccgtccggaaatacttgtcgaagaatttgatgaaaatggatgcatctagaacaagaatacatctagatacatcaatctccccgacaagtatttccgaacggagggagtactagataatagacacgttgtgagctgccttgacttgggtaggcagatccagacctgaaacgtcaggctcatggataaggatcccggccatctgtcactgacgtgtcgtcagtttgcccctagccttgtgggattgccatctccatctgcctataagataactaccctaaaaaaaaaagataactgaaggttgccacctagcacattcacattggtacaacttggaaaagcacagccccgtcgtcctgctcccagttgagttcgcgacctacacaccggccatgccccgcttctcctccaccacgccaatgtcgccaccccgcctccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcaccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttattgatgcgctgtgcaaggccagagccatggacaaggcagaggtggtccttcgtcagatgattgatgacggagttgggcctgataatgtgacgtatagtagcctgattcatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattcgccttcatgggtatgccaccgaaggatgcctagttgacatgatcaatctcttcaattccatggcaacacactgcattctacctaactgtcatatattcaacatactgattaatgcatatgctaaatctgggaagcttgataaggctatgcttatcttcaatgaaatgcagaaacaaggagtgagtccaaatgcagtcacatattcaaccgtaatacatgcattttgcaagaaaggacgcctagacgacgccgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatag

SEQ ID NO: 83 - DNA, 3170 bp, synthetic construct
  source 1..3170: mol_type = other DNA
  misc_feature 1..3170: note = gene construct
tacgacggccagtgagcaatcctcctccatgttaccgtaagaaccctaatcttgtcgatgatcttctcctgtagctcttctaatatgtcataactttgccataatcaatagttgtagtccttgatttactttgatttgcataacggtgctacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggcttaggggacgcgcgcgccgagagagagagcagatttggggaagaaataaagccagggactggggaatgagaagataatgtttataaggggttttctgtaaaaagaaagagcaacctacgcggcccctctgtcgatttggcatgggtcaacgcaccacacgagcaggtcaacgagcgctcgtgagaaaaaggacctagggtaaatcacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacgtactccctccgtccggaaatacttgtcgaagaatttgatgaaaatggatgcatctagaacaagaatacatctagatacatcaatctccccgacaagtatttccgaaagtaagtaatagtagtagtagtactacatactccctccgtccggaaatacttgtcgaagaatttgatgaaaatggatgcatctagaacaagaatacatctagatacatcaatctccctgacaagtatttccgagcggagggagtactagataatagacacgttgtgagctgccttgacttgggtaggcagatccagacctgaaacgtcaggctcatggataaggatcccggccatctgtcactgacgtgtcgtcagtttgcccctagccttgtgggattgccatctccatctgcctataagataactaccctaaaaaaaaaagataactgaaggttgccacctagcacattcacattggtacaacttggaaaagcacagccccgtcgtcctgctcccagttgagttcgcgacctacacagcggccatgccccgcttctcctccaccacgccaatgtcgccaccccgcctccgcctccgactctgcgcccgccactcctcctgcacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccaagacgtcagacgacgggtggcgcagccaaatgtcttcacctatggcgtcctcatggactgctgctgccgcgcttgccgcacagatctggcgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctgatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaaaggacgcctagacgacgccgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaa

SEQ ID NO: 84 - DNA, 7033 bp, synthetic construct
  source 1..7033: mol_type = other DNA
  misc_feature 1..7033: note = gene construct
atgtcacatctcaacaacgtttttccaaaaccatggtattccaaaacctaagtatttcaatacatggtcaataaatacttcagtttctaaaaaccatagtttttctcagttttgataaaactgcgaggtgtttggcaagtggcagtttttctcagttttctttggtttgcatcgactgttacgtgcatgcatattcagctacactcagggatcagcttgtactaatagccatcgtatgcattcagccttgcatgagcatgttctcctagatgctaagatctacagtagagttatctcttgtaatcaatcaaagaccgaggtagttaacacatgcactgaaggtcgacgtttgcagctcgtgtacgagtacatccgcctccagccggacatatcgatgggctacctatcgatttatgcatcacagtcgttgatggccagaagttggatacaagagagaccagcaaccagcgctttctcggttttaaaaaaaagaggtcagggcctcttttttatatacaacaaaaataccacagttttagagataccatggtctgttgaactgcagtattttttttatccaaacgactcaaagtattaaataccaaagtttttttcaaaaaccacagtattccagaaaaccacaaaaatacttcggcttccaaacgcaccctaagtgtaagaagaaatctactaagttcacaaaattcagaagaaactcgtttggaccatgagcggtacagaaaagaaaatggcgtgatctagaatagcagacattagggtctgtttgcaggtattatggatcactgtatgtttgcaaaaaataataatttgtgcagctatcagatgcatgcattttctatgaaaattttgtagatccagtgttatacccaacaacaaaatttaatacttgggtcaccacaacccaagatggagctccaagcaaacttaagtaagaggataccgagatgtgatgatgatgtctgatcttgggggaatttttgtagtgagaaaatgccttaaacatcatcgcggaatccaaatggtcaatcaggactgacaccaccaatggcttgattcaccatctctatctgcccattttgacgtaacactatttggctttcgacgcgacgggataatcaacgtgacaatcctgtcatgagaaagtcatcatgccgagttcattttccaggcatcccgtgtggcatcttgccattctctccgtccatgagttgattacccaaaagcatctgatcccggagcacaacgaacgacatcagaagacatggttcgcatcacaaaacaccatttgatcatgttacgcatgttaggcacatctgaagaagggaaggcgttgagaacctcggccaccactagtagtagacttccaacttttagttgggacaacatgggcgggatttcacggcacaaaattcaccacctagatgctaaaaagataccccccccccccccccatcccccgcacatcctccttgaagacacgggcgccccctccgcctccctacctcgccgtcgccggaggaggccgccgggctaagcctgtgcggcggacgacggcggcagggcctctctgcaacgtccgggtctcatggtgagggaggcacgcctccttgcagtgcggcacggcggctacggcacgaagattctcggaagttgcgtctccagcgggtggcagtcgcggtggtgtcgtcggcaaactcctgggcttcggtccggcggctcttgctcggcgagtatgggcggggtggcggcccccctttcctgcctttggtcattcgtctccgcgcaggtcgactgctctggccggcccttggtggttgttgtagtgctgctgcaagggggcgccagatccggcggttcttcattcggatccagctcgtctctcgtcttactcagggtcggtcatcccggtgaccttgcctctggcagcgagggggtgtgcgtgctctatggtgggccttgtgaggcgatggcgcggttgctgcgcaggggtgtgcatgccagcgattgcgcgggccagcttgtgtcggctgcgtgagggtgttggttgagtctgctccaagcgcaagcttgcccggctatggccggccggtgctggcggcgcctatgggcgtcgtttcccttgttgaaggcgttgttgtggagttcttcgacctactacaatctcgaatctggttcttcgggcgaaaacctcgccccatctaggtcgggcaacgacgtcgtccgtacggtgttttccccctttggggcgttgctttgaagaccggttatcctttcccgtgctacctttgggctggtcgtgcatggtgtcatggttggctggtgctcaatcggtgatgcgagtggttggcgttgcgacttgtgcggtgacgaagatggtgatgcgcggagctgggtcgcggcttgtccttctgatatcggcggttcggttcccctttgggtgcgtctgtgcttgtttctcactgtgacagagaagttgaagctacatgcggcgggggccctgcggcaatgatgactccatgagggtggtttcggcggacgatgctctccgcaggttgcactggactagcttggtgtgaggcttgcaactttctcctgtgatgctcatcaacaaaatcagagatgtcggtcctcgacgcctgcggccgatcgtttggcttggctgtcgaggtcctcgggtgccgtttgttcggaggggtcttgatgggtcgtcagcgaggaaatcggagttgcctgctcgtggagaggcgatgacgatgacattgcttgcagcctcgatggtgtcctctcgtcagcagtatgtgtgtgatcttgtcagtgccaggtcggccggagtgccctattcgatgggcgtgttttaattaaccgggcaactctcttcttcttaatcaatgaaaatggcaagtcttttacctcgtttcaaaaagagttaaatgcactggaggtcctaaaggtttcgtcggggtgtcactttagtcctatttcttccaaaatgcacctttaggtcctaattctttattaattggttcacccgaggtccttttccacgagcgctcgctgaccagcacacgtgccgcattgacccacgccacatcgatagagggtctaaccgtcaggcgagaagcttcgcaaagagaccccccccctcttagtggcaccctctctcctctttcacggtactttggcgacattggaggcgagcgacagaggcaaccggaggtgatctggaggtaatttgtgtgggtgcccgtgaagatgccccttctacgacggccagtgagcaatcctcctccatgttaccgtaagaaccctaatcttgtcgatgatcttctcctgtagctcttctaatatgtcataactttgccataatcaatagttgtagtccttgatttactttgatttgcataacggtgctacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggcttaggggacgcgcgcgccgagagagagagcagatttggggaagaaataaagccagggactggggaatgagaagataatgtttataaggggttttctgtaaaaagaaagagcaacctacgcggcccctctgtcgatttggcatgggtcaacgcaccacacgagcaggtcaacgagcgctcgtgagaaaaaggacctagggtaaatcacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacgtactccctccgtccggaaatacttgtcgaagaatttgatgaaaatggatgcatctagaacaagaatacatctagatacatcaatctccccgacaagtatttccgaaagtaagtaatagtagtagtagtactacatactccctccgtccggaaatacttgtcgaagaatttgatgaaaatggatgcatctagaacaagaatacatctagatacatcaatctccctgacaagtatttccgagcggagggagtactagataatagacacgttgtgagctgccttgacttgggtaggcagatccagacctgaaacgtcaggctcatggataaggatcccggccatctgtcactgacgtgtcgtcagtttgcccctagccttgtgggattgccatctccatctgcctataagataactaccctaaaaaaaaaagataactgaaggttgccacctagcacattcacattggtacaacttggaaaagcacagccccgtcgtcctgctcccagttgagttcgcgacctacacagcggccatgccccgcttctcctccaccacgccaatgtcgccaccccgcctccgcctccgactctgcgcccgccactcctcctgcacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccaagacgtcagacgacgggtggcgcagccaaatgtcttcacctatggcgtcctcatggactgctgctgccgcgcttgccgcacagatctggcgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctgatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggacctgataatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaaaggacgcctagacgacgccgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatag

SEQ ID NO: 85 - DNA, 144 bp, Triticum sp.; identical to SEQ ID NO: 70
  source 1..144: mol_type = unassigned DNA
  misc_feature 1..144: note = Wheat enhancer sequence EN1390

SEQ ID NO: 86 - DNA, 144 bp, Triticum sp.
  source 1..144: mol_type = unassigned DNA
  misc_feature 1..144: note = Wheat enhancer sequence EN5458
cagtacgatcaatgcagtgactaagtgccgtgctgtcaagtgcgtactggtaatcgggatagaaccagctcgtcatcgccagcacctagaaagagtagatagatagatgccccggtcgacacgataggataaccatcggcgcgg

SEQ ID NO: 87 - DNA, 144 bp, Triticum sp.
  source 1..144: mol_type = unassigned DNA
  misc_feature 1..144: note = Wheat enhancer sequence EN2393
gtctccgtccggaacgtccatcgccgccgcttcctcgtcgtggccgccgcgtgcgggctggaaagggacggccagcttttccgacgcctccctgacgtgtcagcccagccccgaaccaccacacgcgtccatccccatccgctg

SEQ ID NO: 88 - DNA, 144 bp, Triticum sp.
  source 1..144: mol_type = unassigned DNA
  misc_feature 1..144: note = Wheat enhancer sequence EN2968
ttcccccgcaaaagaaaaagtacaaggaggcatgcagcatggcgtccatgcatgctgctagatgacgtgtacccttgagcggttcagcctgttcatcctctccaaagggaaataaaaaaaaatgtccacgtgcatcgaaccgga

SEQ ID NO: 89 - DNA, 144 bp, Triticum sp.
  source 1..144: mol_type = unassigned DNA
  misc_feature 1..144: note = Wheat enhancer sequence EN1391
ttgccatctccatctgcctaaccctcacccgatgcgtgtcctgtttcttgggggttcggtgattggtgtcaccaatttgtttcttctttgattgggacctgtacagtggaagatttactcttggaattgtctgccgggatagga

SEQ ID NO: 90 - DNA, 144 bp, Triticum sp.
  source 1..144: mol_type = unassigned DNA
  misc_feature 1..144: note = Wheat enhancer sequence EN4730
atgcggctagatgacgtgtaccctttgagcggttcatcctgtccaacgtgaaaaaaaatgtccacgtgcatcgaaccggacaataggccagcccacactgcccatgccatcacgtttcatgcagcgggagaatattttacgggg

SEQ ID NO: 91 - DNA, 144 bp, Triticum sp.
  source 1..144: mol_type = unassigned DNA
  misc_feature 1..144: note = Wheat enhancer sequence EN3681
gcccgtggccgccgagcgctgggcttgtttattcggagggaggcggccgagatgcgcaggtgcgacgggtggattgaccccggttcgtgtgggtacgttgaacctggacgtctttttcggtgagaaaagcacgattcgattccc

SEQ ID NO: 92 - DNA, 6905 bp, synthetic construct
  source 1..6905: mol_type = other DNA
  misc_feature 1..6905: note = Sequence from Fig. 22
atgtcacatctcaacaacgtttttccaaaaccatggtattccaaaacctaagtatttcaatacatggtcaataaatacttcagtttctaaaaaccatagtttttctcagttttgataaaactgcgaggtgtttggcaagtggcagtttttctcagttttctttggtttgcatcgactgttacgtgcatgcatattcagctacactcagggatcagcttgtactaatagccatcgtatgcattcagccttgcatgagcatgttctcctagatgctaagatctacagtagagttatctcttgtaatcaatcaaagaccgaggtagttaacacatgcactgaaggtcgacgtttgcagctcgtgtacgagtacatccgcctccagccggacatatcgatgggctacctatcgatttatgcatcacagtcgttgatggccagaagttggatacaagagagaccagcaaccagcgctttctcggttttaaaaaaaagaggtcagggcctcttttttatatacaacaaaaataccacagttttagagataccatggtctgttgaactgcagtattttttttatccaaacgactcaaagtattaaataccaaagtttttttcaaaaaccacagtattccagaaaaccacaaaaatacttcggcttccaaacgcaccctaagtgtaagaagaaatctactaagttcacaaaattcagaagaaactcgtttggaccatgagcggtacagaaaagaaaatggcgtgatctagaatagcagacattagggtctgtttgcaggtattatggatcactgtatgtttgcaaaaaataataatttgtgcagctatcagatgcatgcattttctatgaaaattttgtagatccagtgttatacccaacaacaaaatttaatacttgggtcaccacaacccaagatggagctccaagcaaacttaagtaagaggataccgagatgtgatgatgatgtctgatcttgggggaatttttgtagtgagaaaatgccttaaacatcatcgcggaatccaaatggtcaatcaggactgacaccaccaatggcttgattcaccatctctatctgcccattttgacgtaacactatttggctttcgacgcgacgggataatcaacgtgacaatcctgtcatgagaaagtcatcatgccgagttcattttccaggcatcccgtgtggcatcttgccattctctccgtccatgagttgattacccaaaagcatctgatcccggagcacaacgaacgacatcagaagacatggttcgcatcacaaaacaccatttgatcatgttacgcatgttaggcacatctgaagaagggaaggcgttgagaacctcggccaccactagtagtagacttccaacttttagttgggacaacatgggcgggatttcacggcacaaaattcaccacctagatgctaaaaagataccccccccccccccccatcccccgcacatcctccttgaagacacgggcgccccctccgcctccctacctcgccgtcgccggaggaggccgccgggctaagcctgtgcggcggacgacggcggcagggcctctctgcaacgtccgggtctcatggtgagggaggcacgcctccttgcagtgcggcacggcggctacggcacgaagattctcggaagttgcgtctccagcgggtggcagtcgcggtggtgtcgtcggcaaactcctgggcttcggtccggcggctcttgctcggcgagtatgggcggggtggcggcccccctttcctgcctttggtcattcgtctccgcgcaggtcgactgctctggccggcccttggtggttgttgtagtgctgctgcaagggggcgccagatccggcggttcttcattcggatccagctcgtctctcgtcttactcagggtcggtcatcccggtgaccttgcctctggcagcgagggggtgtgcgtgctctatggtgggccttgtgaggcgatggcgcggttgctgcgcaggggtgtgcatgccagcgattgcgcgggccagcttgtgtcggctgcgtgagggtgttggttgagtctgctccaagcgcaagcttgcccggctatggccggccggtgctggcggcgcctatgggcgtcgtttcccttgttgaaggcgttgttgtggagttcttcgacctactacaatctcgaatctggttcttcgggcgaaaacctcgccccatctaggtcgggcaacgacgtcgtccgtacggtgttttccccctttggggcgttgctttgaagaccggttatcctttcccgtgctacctttgggctggtcgtgcatggtgtcatggttggctggtgctcaatcggtgatgcgagtggttggcgttgcgacttgtgcggtgacgaagatggtgatgcgcggagctgggtcgcggcttgtccttctgatatcggcggttcggttcccctttgggtgcgtctgtgcttgtttctcactgtgacagagaagttgaagctacatgcggcgggggccctgcggcaatgatgactccatgagggtggtttcggcggacgatgctctccgcaggttgcactggactagcttggtgtgaggcttgcaactttctcctgtgatgctcatcaacaaaatcagagatgtcggtcctcgacgcctgcggccgatcgtttggcttggctgtcgaggtcctcgggtgccgtttgttcggaggggtcttgatgggtcgtcagcgaggaaatcggagttgcctgctcgtggagaggcgatgacgatgacattgcttgcagcctcgatggtgtcctctcgtcagcagtatgtgtgtgatcttgtcagtgccaggtcggccggagtgccctattcgatgggcgtgttttaattaaccgggcaactctcttcttcttaatcaatgaaaatggcaagtcttttacctcgtttcaaaaagagttaaatgcactggaggtcctaaaggtttcgtcggggtgtcactttagtcctatttcttccaaaatgcacctttaggtcctaattctttattaattggttcacccgaggtccttttccacgagcgctcgctgaccagcacacgtgccgcattgacccacgccacatcgatagagggtctaaccgtcaggcgagaagcttcgcaaagagaccccccccctcttagtggcaccctctctcctctttcacggtactttggcgacattggaggcgagcgacagaggcaaccggaggtgatctggaggtaatttgtgtgggtgcccgtgaagatgccccttctacgacggccagtgagcaatcctcctccatgttaccgtaagaaccctaatcttgtcgatgatcttctcctgtagctcttctaatatgtcataactttgccataatcaatagttgtagtccttgatttactttgatttgcataacggtgctacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggcttaggggacgcgcgcgccgagagagagagcagatttggggaagaaataaagccagggactggggaatgagaagataatgtttataaggggttttctgtaaaaagaaagagcaacctacgcagcccctctgtcgatttggcatgggtcaacgcaccacacgagcaggtcaacgagcgctcgtgagaaaaaggacctagggtaaatcacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacgtactccctccgtccggaaatacttgtcgaagaatttgatgaaaatggatgcatctagaacaagaatacatctagatacatcaatctccccgacaagtatttccgaacggagggagtactagataatagacacgttgtgagctgccttgacttgggtaggcagatccagacctgaaacgtcaggctcatggataaggatcccggccatctgtcactgacgtgtcgtcagtttgcccctagccttgtgggattgccatctccatctgcctataagataactaccctaaaaaaaaaagataactgaaggttgccacctagcacattcacattggtacaacttggaaaagcacagccccgtcgtcctgctcccagttgagttcgcgacctacacaccggccatgccccgcttctcctccaccacgccaatgtcgccaccccgcctccgcctccgcctccgactctgcgcccgccactcctcctccacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcaccctcttcggccgtttgtcccgagacgtcggacgacgggtggcgcagccaaatgtcttcacctatggcgtcctcatggactgctgctgccgcgcttgccgcacagatctggtgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctaatgtggtggcgtataacaccgttatccatggcttctttaaggaaggccatgtaagcaaggcctgcaatctgttccatgaaatggcgcagcagggcgttaagcctaatgtggtgacatataactcagttattgatgcgctgtgcaaggccagagccatggacaaggcagaggtggtccttcgtcagatgattgatgacggagttgggcctgataatgtgacgtatagtagcctgattcatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattcgccttcatgggtatgccaccgaaggatgcctagttgacatgatcaatctcttcaattccatggcaacacactgcattctacctaactgtcatatattcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatag

SEQ ID NO: 93 - DNA, 7033 bp, synthetic construct; identical to SEQ ID NO: 84
  source 1..7033: mol_type = other DNA
  misc_feature 1..7033: note = Sequence from Fig. 29

SEQ ID NO: 94 - DNA, 6753 bp, Triticum aestivum
  source 1..6753: mol_type = genomic DNA; note = Rf3-58 variant
atgtcacatctcaacaacgtttttccaaaaccatggtattccaaaacctaagtatttcaatacatggtcaataaatacttcagtttctaaaaaccatagtttttctcagttttgataaaactgcgaggtgtttggcaagtggcagtttttctcagttttctttggtttgcatcgactgttacgtgcatgcatattcagctacactcagggatcagcttgtactaatagccatcgtatgcattcagccttgcatgagcatgttctcctagatgctaagatctacagtagagttatctcttgtaatcaatcaaagaccgaggtagttaacacatgcactgaaggtcgacgtttgcagctcgtgtacgagtacatccgcctccagccggacatatcgatgggctacctatcgatttatgcatcacagtcgttgatggccagaagttggatacaagagagaccagcaaccagcgctttctcggttttaaaaaaaagaggtcagggcctcttttttatatacaacaaaaataccacagttttagagataccatggtctgttgaactgcagtattttttttatccaaacgactcaaagtattaaataccaaagtttttttcaaaaaccacagtattccagaaaaccacaaaaatacttcggcttccaaacgcaccctaagtgtaagaagaaatctactaagttcacaaaattcagaagaaactcgtttggaccatgagcggtacagaaaagaaaatggcgtgatctagaatagcagacattagggtctgtttgcaggtattatggatcactgtatgtttgcaaaaaataataatttgtgcagctatcagatgcatgcattttctatgaaaattttgtagatccagtgttatacccaacaacaaaatttaatacttgggtcaccacaacccaagatggagctccaagcaaacttaagtaagaggataccgagatgtgatgatgatgtctgatcttgggggaatttttgtagtgagaaaatgccttaaacatcatcgcggaatccaaatggtcaatcaggactgacaccaccaatggcttgattcaccatctctatctgcccattttgacgtaacactatttggctttcgacgcgacgggataatcaacgtgacaatcctgtcatgagaaagtcatcatgccgagttcattttccaggcatcccgtgtggcatcttgccattctctccgtccatgagttgattacccaaaagcatctgatcccggagcacaacgaacgacatcagaagacatggttcgcatcacaaaacaccatttgatcatgttacgcatgttaggcacatctgaagaagggaaggcgttgagaacctcggccaccactagtagtagacttccaacttttagttgggacaacatgggcgggatttcacggcacaaaattcaccacctagatgctaaaaagataccccccccccccccccatcccccgcacatcctccttgaagacacgggcgccccctccgcctccctacctcgccgtcgccggaggaggccgccgggctaagcctgtgcggcggacgacggcggcagggcctctctgcaacgtccgggtctcatggtgagggaggcacgcctccttgcagtgcggcacggcggctacggcacgaagattctcggaagttgcgtctccagcgggtggcagtcgcggtggtgtcgtcggcaaactcctgggcttcggtccggcggctcttgctcggcgagtatgggcggggtggcggcccccctttcctgcctttggtcattcgtctccgcgcaggtcgactgctctggccggcccttggtggttgttgtagtgctgctgcaagggggcgccagatccggcggttcttcattcggatccagctcgtctctcgtcttactcagggtcggtcatcccggtgaccttgcctctggcagcgagggggtgtgcgtgctctatggtgggccttgtgaggcgatggcgcggttgctgcgcaggggtgtgcatgccagcgattgcgcgggccagcttgtgtcggctgcgtgagggtgttggttgagtctgctccaagcgcaagcttgcccggctatggccggccggtgctggcggcgcctatgggcgtcgtttcccttgttgaaggcgttgttgtggagttcttcgacctactacaatctcgaatctggttcttcgggcgaaaacctcgccccatctaggtcgggcaacgacgtcgtccgtacggtgttttccccctttggggcgttgctttgaagaccggttatcctttcccgtgctacctttgggctggtcgtgcatggtgtcatggttggctggtgctcaatcggtgatgcgagtggttggcgttgcgacttgtgcggtgacgaagatggtgatgcgcggagctgggtcgcggcttgtccttctgatatcggcggttcggttcccctttgggtgcgtctgtgcttgtttctcactgtgacagagaagttgaagctacatgcggcgggggccctgcggcaatgatgactccatgagggtggtttcggcggacgatgctctccgcaggttgcactggactagcttggtgtgaggcttgcaactttctcctgtgatgctcatcaacaaaatcagagatgtcggtcctcgacgcctgcggccgatcgtttggcttggctgtcgaggtcctcgggtgccgtttgttcggaggggtcttgatgggtcgtcagcgaggaaatcggagttgcctgctcgtggagaggcgatgacgatgacattgcttgcagcctcgatggtgtcctctcgtcagcagtatgtgtgtgatcttgtcagtgccaggtcggccggagtgccctattcgatgggcgtgttttaattaaccgggcaactctcttcttcttaatcaatgaaaatggcaagtcttttacctcgtttcaaaaagagttaaatgcactggaggtcctaaaggtttcgtcggggtgtcactttagtcctatttcttccaaaatgcacctttaggtcctaattctttattaattggttcacccgaggtccttttccacgagcgctcgctgaccagcacacgtgccgcattgacccacgccacatcgatagagggtctaaccgtcaggcgagaagcttcgcaaagagaccccccccctcttagtggcaccctctctcctctttcacggtactttggcgacattggaggcgagcgacagaggcaaccggaggtgatctggaggtaatttgtgtgggtgcccgtgaagatgccccttctacgacggccagtgagcaatcctcctccatgttaccgtaagaaccctaatcttgtcgatgatcttctcctgtagctcttctaatatgtcataactttgccataatcaatagttgtagtccttgatttactttgatttgcataacggtgctacggacacggcaaaaaggacacaccaccagcggcaacatacccgccgaagttggagcttgcttcgccgcgccggcggcggtgctgaggatgttgccatgctcctcatcttagttgtttccttgccgaggagcttgaagttccggccatggataggcttaggggacgcgcgcgccgagagagagagcagatttggggaagaaataaagccagggactggggaatgagaagataatgtttataaggggttttctgtaaaaagaaagagcaacctacgcggcccctctgtcgatttggcatgggtcaacgcaccacacgagcaggtcaacgagcgctcgtgagaaaaaggacctagggtaaatcacttactaaagaattaggacctaaaggtgcattttggacgaaataagactaaagtgacaccctgacgaattttttaggaccttgagtgcatttaactctttcaaaaaataaagatgctaaaaaaactaaatgcttaaaaatacatttttctatgggaatacttaaaaatactagtataatttgatggcttcctagtatatttctagggcttacaactaaatttatattcttggtctaaataaaattataatctgataaattatgtgatatgaatatagccatatgagaccataatttaattttacctgcaaaaagtaagtaatagtagtagtagtactacgtactccctccgtccggaaatacttgtcgaagaatttgatgaaaatggatgcatctagaacaagaatacatctagatacatcaatctccccgacaagtatttccgaacggagggagtactagataataagataactaccctaaaaaaaaaagataactgaaggttgccacctagcacattcacattggtacaacttggaaaagcacagccccgtcgtcctgctcccagttgagttcgcgacctacacagcggccatgccccgcttctcctccaccacgccaatgtcgccaccccgcctccgcctccgactctgcgcccgccactcctcctgcacctctcatccctcacgcatctgggatccccacgccgccttcgccgccgcggcacagcgggcgagctctggcacgctcactacggaggacgcacaccacctgtttgacgaattgctgcggcggggcaatcctgtccaggagcgtcccttgaataaatttctggctgccctcgcccgcgcgcccgcgtccgcatcctgctgcgatggccccgccctggcagtcgccctcttcggccgtttgtcccaagacgtcagacgacgggtggcgcagccaaatgtcttcacctatggcgtcctcatggactgctgctgccgcgcttgccgcacagatctggcgctcgccttctttggccgtctcctcaagacgggcctggaggcaaaccaagtcgtcttcaacaccctcctcaagggcctttgccacacaaagcgggcggatgaggctctggacgtgctgcttcacaggatgcctgagctgggctgcactcctgatgtggtggcgtataacaccgttatccatggcttctttaaggaaggccatgtaagcaaggcctgcaatctgttccatgaaatggcgcagcagggcgttaagcctaatgtggtgacatataactcagttatcgatgcgctgtgcaaggccagagccatggacaaggcagaggtggtccttcgtcagatgattgatgatggtgttggaccttaatgtgacgtatagtagcctcatccatggatattcctcttcaggccactggaaggaggcagttagggtattcaaagagatgacaagtcggagggttacagcagatgtgcatacttacaacatgtttatgacctttctttgcaaacatggaagaagcaaagaagctgcaggaatttttgataccatggctatcaagggcctgaaacctgacaacgtttcatatgctattctccttcatgggtatgccgccgaaggatgcttagttgatatgattaatctcttcaattcaatggaaagagattgtattctacctgactgtcgtatcttcaacatactgattaatgcatatgctaaatctgggaagcttgataaggctatgcttatcttcaatgaaatgcagaaacaaggagtgagtccaaatgcagtcacatattcaaccgtaatacatgcattttgcaagaagggtaggttggatgatgctgtgataaagtttaatcagatgattgatacaggagtacgaccggacgcatctgtttatcgtcccctaatccagggtttttgtacacatggcgatttggtgaaagcaaaggaatatgttactgaaatgatgaagaaaggtatgcctcctcctgatattatgttcttcagttcaatcatgcagaacctatgcacagaaggaagggtaacagaagcacgggatatccttgacttgatagtgcacattggtatgaggcctaatgttatcatatttaatttgctgatcggtggatactgcctagtccgcaagatggcagatgcattgaaagtatttgatgatatggtgtcatatggtttagaaccttgtaactttacgtatggtatacttattaatggctattgcaaaaatagaaggattgatgacgggcttattctgttcaaagagatgctgcacaagggacttaaacctacaacttttaattataacgtcatactggatggattatttctggctggacaaactgttgctgcaaaagagaagtttgatgagatggttgaatctggagtaagtgtgtgcattgatacatactctataattcttggtggactttgtagaaatagctgcagtagcgaagcgatcacccttttccggaaattaagcgcaatgaatgtgaaatttgatattacaattgtcaatatcattattggtgccttatacagggtcgagagaaaccaagaggctaaggatttgtttgctgctatgccagccaatggcttggttcctaatgctgttacctacaccgtaatgatgacaaatcttataaaagaaggttcagtggaagaagctgacaatcttttcttatccatggagaagagcggctgtactgccaactcttgcctgttaaatcatatcatcagaaggttactggaaaaaggagagatagtcaaggctggaaattatatgtctaaagttgatgcaaagagctactcacttgaagctaaaactgtttcgctgctgatctctctgttttcaaggaaagggaaatatagagaacacatcaaattgcttcctacaaagtatcagtttctggaagaagcagccacagttgaatag

(19) United States
(12) Patent Application Publication
(10) Pub. No.: US 2025/0059556 A1
(43) Pub. Date: Feb. 20, 2025

(54) REGULATORY NUCLEIC ACID MOLECULES FOR MODIFYING GENE EXPRESSION IN CEREAL PLANTS

(71) Applicant: BASF SE, Ludwigshafen Am Rhein (DE)

(72) Inventors: Mark DAVEY, Gent (BE); Frank MEULEWAETER, Gent (BE); Jonny JACOBS, Gent (BE); Andrea ARIANI, Gent (BE); Petrus OUWERKERK, Gent (BE); Michel VAN THOURNOUT, Gent (BE); Katelijn D'HALLUIN, Gent (BE)

(21) Appl. No.: 18/722,537

(22) PCT Filed: Dec. 22, 2022
(86) PCT No.: PCT/EP2022/087662

§ 371 Date: Jun. 20, 2024

(30) Foreign Application Priority Data
Dec. 22, 2021 (EP) 21217191.2
Jun. 17, 2022 (EP) 22179610.5

(51) Int. Cl. C12N 15/82; C07K 14/415

(52) U.S. Cl. CPC C12N 15/8289; C07K 14/415

(57) ABSTRACT

The present invention relates to the field of plant molecular biology and provides materials and methods for modulating expression of a gene of interest in plants. In particular, the invention provides modified plant promoters or modified coding sequences having increased expression, for example, in developing spikes as well as methods for producing promoters or coding sequences having increased expression. The modified promoters comprise i) at least one binding site for an EIL3 transcription factor and/or at least one binding site for a PHD transcription factor and/or ii) one or more enhancer elements. Moreover, the present invention concerns a nucleic acid molecule encoding a functional restorer polypeptide for wheat G-type cytoplasmic male sterility comprising in the coding sequence a mutated microRNA (“miRNA”) binding site. In some embodiments, said nucleic acid molecule is operably linked to the modified promoter of the present invention.

### FIELD OF THE INVENTION

The present invention relates to the field of plant molecular biology and provides materials and methods for modulating expression of a gene of interest in plants. In particular, the invention provides modified plant promoters or modified coding sequences having increased expression, for example, in developing spikes as well as methods for producing promoters or coding sequences having increased expression. The modified promoters comprise i) at least one binding site for an EIL3 transcription factor and/or at least one binding site for a PHD transcription factor and/or ii) one or more enhancer elements. Moreover, the present invention concerns a nucleic acid molecule encoding a functional restorer polypeptide for wheat G-type cytoplasmic male sterility comprising in the coding sequence a mutated microRNA (“miRNA”) binding site. In some embodiments, said nucleic acid molecule is operably linked to the modified promoter of the present invention.

### BACKGROUND

Cytoplasmic male sterility (CMS) is a major trait of interest in cereals such as wheat in the context of commercial hybrid seed production. The cytoplasms of Triticum timopheevi (G-type) and Aegilops kotschyi (K-type) are widely studied as inducers of male sterility in common, hexaploid wheat (Triticum aestivum), due to few deleterious effects.

In a hybrid seed production system using the G-type cytoplasm, fertility restoration is critical. Most hexaploid wheat varieties do not naturally contain fertility restoration (Rf) genes. In the complicated restoration system of T. timopheevi, up to nine different Rf loci are reported to restore the fertility against T. timopheevii cytoplasm, and their chromosome locations have been determined, namely, Rf1 (Chr1A), Rf2 (Chr7D), Rf3 (Chr1B), Rf4 (Chr6B), Rf5 (Chr6D), Rf6 (Chr5D), Rf7 (Chr7B), Rf8 (Chr2D) and Rf9 (Chr6A) (Shahinnia et al.).

The majority of fertility restoration (Rf) genes come from a clade of genes encoding pentatricopeptide repeat (PPR) proteins (Fuji et al. 2011). PPR genes functioning as fertility restoration (Rf) genes are referred to in Fuji et al. 2011 as Rf-PPR genes. These Rf-PPR genes are usually P-type PPR genes (Barkan and Small 2014; Dahan and Mireau 2013) and are often present in clusters of similar Rf-PPR-like genes, which show a number of common characteristic features compared with other PPR genes. They are typically comprised primarily of tandem arrays of 15-20 PPR motifs, each composed of 35 amino acids, together with an N-terminal mitochondrial targeting peptide sequence.

PPR proteins are classified based on their domain architecture. P-class PPR proteins possess the canonical 35 amino acid motif and normally lack additional domains. Members of this class have functions in most aspects of organelle gene expression. PLS-class PPR proteins have three different types of PPR motifs, which vary in length; P (35 amino acids), L (long, 35-36 amino acids) and S (short, ˜31 amino acids), and members of this class are thought to mainly function in RNA editing. Subtypes of the PLS class are categorized based on the additional C-terminal domains they possess (reviewed by Manna, 2015).

Most of the Rf PPR genes identified and cloned to date belong to the P-class PPR subfamily, although PLS-class PPR Rf genes have also been identified, and both classes are characterized by the presence of tandem arrays of 15 to 20 PPR motifs. High substitution rates are observed for particular amino acids within otherwise well-conserved PPR motif sequences, indicating diversifying selection and prompting the conclusion that these residues might be directly involved in binding to RNA targets. This has led to the development of a “PPR code” which allows the prediction of RNA targets of naturally occurring PPR proteins as well as the design of synthetic PPR proteins that can bind RNA molecules of interest. Here, the mRNA sequence binding specificity is ensured by distinct patterns of hydrogen bonding between each RNA base and the amino acid side chains at positions 5 and 35 in the aligned PPR motif (Barkan et al. 2012).

WO 2018/015403 reports the identification of a functional restorer (Rf3) gene for wheat G-type cytoplasmic male sterility (i.e., T. timopheevi cytoplasm) located on chromosome 1B (short arm 1 BS), as well as markers associated therewith. The functional restorer gene was shown to encode a P-type pentatricopeptide repeat (PPR) protein. According to WO 2018/015403, restoration capacity for wheat G-type cytoplasmic male sterility could be increased by increasing expression of Rf3. The document describes, inter alia, that the plant genome could be modified to increase expression of the Rf3 polypeptide by modifying the native promoter to include regulatory elements that increase transcription, such as certain enhancer elements, but also by inactivating or removing certain negative regulatory elements, such as repressor elements or target sites for miRNAs or IncRNAs. WO 2018/015403 also describes that the Rf3 gene does have multiple putative miRNA binding sites in the region 160-270 bp 5′ to the ATG start. However, these miRNA binding sites were not confirmed.

WO 2018/015403 also reports that expression can be increased by providing the plant with the (recombinant) chromosome fragment or the (isolated) nucleic acid molecule or the chimeric gene as described herein, whereby the nucleic acid encoding the functional restorer gene allele is under the control of appropriate regulatory elements such as a promoter driving expression in the desired tissues/cells. Further, the document discloses that transcription factors may be provided to plant that e.g. (specifically) recognise the promoter region and promote transcription, such as TALeffectors, dCas, dCpf1 etc. coupled to transcriptional enhancers.

WO 2019/086510 describes that sequence comparison shows that the 5′UTR sequence of the RFL29a (Rf3 variant) gene contains a 163 bp-long deletion identified in the 5′UTR of RFL29b (Rf3 variant) corresponding sequence. WO 2019/086510 further describes that sequence comparison between the different accessions listed in Table 12 shows that all “Rf3 weak” accessions harbor the 163 bp insertion and that all the “Rf3” accessions harbor the 163 bp deletion, and because of the 163 bp deletion in the 5′UTR sequence of RFL29a gene, it is expected that the 163 bp region impairs the expression of RFL29b gene such that the fertility level is weak in lines harboring the RFL29b allele compared to lines harboring the RFL29a allele. Example 15 in WO2019/086510 describes the deletion of (part of) this 163 bp region in the promoter of the (“Rf3 weak”) RFL29b gene by genome editing, so as to increase RFL29b expression. However, no results are shown.

Also, EP 3 718 397 A1 describes in the context of Rf genes for wheat G-type cytoplasmic male sterility located on chromosome 1A or 1B, that the term “genome editing” refers to strategies and techniques for the targeted, specific modification of any genetic information or genome of a plant cell by means of or involving a double-stranded DNA break—inducing enzyme or single-stranded DNA or RNA break—inducing enzyme, and as such, the terms comprise gene editing, but also the editing of regions other than gene encoding regions of a genome, such as intronic sequences, non-coding RNAs, miRNAs, sequences of regulatory elements like promoter, terminator, transcription activator binding sites, cis- or trans-acting elements. Additionally, the terms may comprise base editing for targeted replacement of single nucleobases. It can further comprise the editing of the nuclear genome as well as of other genetic information of a plant cell, i.e. mitochondrial genome or chloroplast genome as well as miRNA, pre-mRNA or mRNA.

Li et al. (2019) investigated a K-type CMS restoration system based on Aegilops kotschyi cytoplasm. The tae-miR9674b has been reported to regulate PPR (pentatricopeptide repeat) genes in wheat. Specifically, the miRNA was reported to target 33 PPR genes, of which the expression of 22 genes were negatively correlated with the expression of tae miR39674b (expression repressed by tae_miR39674b). None of these genes were located on Chr1B.

Further, general enhancers have been identified that are important cis-regulatory DNA elements that regulate transcription by recruiting transcription factors and directing them to the promoters of target genes in a cell-type/tissue-specific manner. The expression of a gene can be regulated by one or multiple enhancers (Marand et al., 2017).

WO2021/048316A1 describes methods for enhancing expression conferred by plant promoter. The method comprises the step of functionally linking one or more wheat enhancers to said promoter. In WO2021/048316A1 the enhancers are referred to as “nucleic acid expression enhancing nucleic acid (NEENA) molecules”.

Espley et al. (2009) reported that rearrangement in the upstream regulatory region of the gene encoding an apple transcription factor led to a phenotype that includes red foliage and red fruit flesh.

Mao et al. (2021) reported that an insertion in the promoter of wheat transcription factor alters its expression level and contributes to drought tolerance in wheat.

Previous studies have indicated that combinations of two or three major Rf genes and restorer genes with small effect or low penetrance (modifier loci) can modify the degree of fertility restoration (Ahmed et al., 2001; Zhou et al., 2005; Stojalowski et al., 2013). Consequently, attempts are made to pyramid multiple dominant or partially dominant alleles of the most favorable genes or quantitative trait loci (QTL), including those involved in epistatic interactions to achieve complete fertility restoration in hybrid wheat (Gupta et al., 2019).

Currently, restoration of fertility of wheat G-type CMS, thus, requires multiple restorer loci for optimal fertility of hybrids. To make the wheat hybrid breeding process more efficient improved restorer genes would be needed. Thus, there remains a need for improving Rf genes in breeding, which are particularly useful for hybrid seed production, and for improved methods for fertility restoration in hexaploid wheat possessing T. timopheevi cytoplasm.

### FIGURE LEGENDS

FIG. 1: Activity of Rf3-58 promoter in wheat protoplasts. A: Activity of Rf3 promoter fragments: a, pRf3-4; b, pRf3-2; c, pRf3-1.4; d, pRf3-1.2. B: Effect of EIL3 or PHD overexpression on Rf3 promoter activity: pRf3-1.2>GUS was co-expressed with a, p35S>GFP; b, p35S>EIL; c, p35S>PHD. The vertical axis shows the GUS activities from the tested promoter fragments corrected for variation in protoplast transfection efficiency using the luciferase activities of a co-introduced pKA63 plasmid.

FIG. 2: Duplication of EIL3 and PHD binding sites increases Rf3 promoter activity in wheat mesophyll protoplasts only when the corresponding transcription factor is overexpressed. A: Impact of the EIL3 binding site duplication and mutation: pRf3-1.2>GUS (a, d), pRf3-1.2-EIL>GUS (b, e), and pRf3-1.2-EIL*>GUS (c, f) were co-expressed with p35S>EIL (a, b, c) or p35S>PHD (d, e, f). B: Impact of the EIL3 and PHD binding site duplication when EIL3 is overexpressed: pRf3-1.2>GUS (a), pRf3-1.2-EIL>GUS (b) and pRf3-1.2-PHD-EIL>GUS (c) were coexpressed with p35S>EIL. C: Impact of the EIL3 and PHD binding site duplication when PHD is overexpressed: pRf3-1.2>GUS (a, c) and pRf3-1.2-PHD-EIL>GUS (b, d) were expressed with (a, b) and without (c, d) p35S>PHD. The vertical axis shows the GUS activities from the tested promoter fragments corrected for variation in protoplast transfection efficiency using the luciferase activities of a co-introduced pKA63 plasmid.

FIG. 3: Rf3-58 promoter fragment sequence (SEQ ID NO: 33). The identified transcription factor binding sites are highlighted in bold and italics. The PHD binding site (SEQ ID NO: 11) is underlined once, the EIL3 binding site (SEQ ID NO: 19) is underlined twice. The sequence that was duplicated in the examples (SEQ ID NO: 29) is highlighted in grey.

FIG. 4: RFL29a promoter fragment sequence (SEQ ID NO: 34). The transcription factor binding sites are highlighted in grey. The PHD binding site (gtaatagtagtactac, SEQ ID NO: 40)) is underlined once, the EIL3 binding site (SEQ ID NO: 19) is underlined twice. The PHD binding site in this promoter differs at one position (highlighted in bold) from the binding site present in the Rf3-58 promoter.

FIG. 5 Rf1-09 promoter fragment sequence (SEQ ID NO: 35). The PHD transcription factor binding site (gtagtagtactactag, SEQ ID NO: 38) is underlined and highlighted in grey. The PHD binding site in this promoter differs at two positions (highlighted in bold) from the PHD binding site present in the Rf3-58 promoter.

FIG. 6 Interaction between miRNA3619 (lower sequence in each alignment, SEQ ID NO: 47) and its putative binding site (in capital letters) in A) the Rf3-58 mRNA coding sequence (upper sequence (“Target”) in A, SEQ ID NO: 48), in B) the Rf1-09 mRNA coding sequence (upper sequence (“Target”) in B, SEQ ID NO: 68), and in C) the Rf3-29a mRNA coding sequence (upper sequence (“Target”) in C, SEQ ID NO: 48) The numbers on top indicate the nucleotide numbers of the binding site as used for the mutant descriptions. RF3-29a is also referred to as RFL29a herein.

FIG. 7 Impact of mutations in the putative miRNA3619 binding site of Rf3 (here the Rf3-58 allele with sequence of SEQ ID NO: 43 (this is the coding sequence of Rf3-58 (PPR58 is an alternative name for Rf3-58)) on expression of a Rf3-GUS fusion protein in transiently transformed wheat protoplasts. The horizontal axis legend shows whether the Rf3 sequence (outside the mutated miRNA3619 binding site) was the original wheat sequence (“WT”) or optimized for expression in wheat (“opt”) and whether the putative miRNA binding site was left intact (“intact”) or mutated (“mutant”). The following plasmids were used: pBas04646, pBas04648, pBas04649 and pBas04647 (see Table 1). GUS activities were corrected for variation in protoplast transfection efficiency using the luciferase activities of a co-introduced pKA63 plasmid. Expression of the construct with the WT Rf3 sequence with intact (unmodified) miRNA3619 binding site sequence was set at 1. The y-axis shows mean relative GUS/LUC activity.

FIG. 8: Impact of mutations in the putative miRNA3619 binding site of Rf3-58 on expression of a Rf3-GUS fusion protein in transiently transformed wheat protoplasts. The horizontal axis legend shows the nt positions of the mutations in the miRNA3619 binding site (using the numbering in FIG. 6) and the name of the introduced plasmid. The Rf3 sequence outside the miRNA3619 binding site was optimized for expression in wheat. GUS activities were corrected for variation in protoplast transfection efficiency using the luciferase activities of a co-introduced pKA63 plasmid. Expression of the construct with the intact miRNA3619 binding site sequence (pBas04649) was set at 1. The y-axis shows mean relative GUS/LUC activity.

FIG. 9 Impact of mutations in the putative miRNA3619 binding site of Rf3 on expression of a Rf3-GUS fusion protein in transiently transformed wheat protoplasts. The horizontal axis legend shows the nt position(s) of the mutation(s) in the miRNA3619 binding site (using the numbering in FIG. 6) and the name of the introduced plasmid. The Rf3 sequence outside the miRNA3619 binding site was optimized for expression in wheat. GUS activities were corrected for variation in protoplast transfection efficiency using the luciferase activities of a co-introduced pKA63 plasmid. Expression of the construct with the intact miRNA3619 binding site sequence was set at 1.

FIG. 10 Rf3-58 coding sequence (SEQ ID NO: 43). The identified miRNA binding site is highlighted in bold and italics. The portion of the sequence that corresponds to the Rf3 sequence used in the Examples section is underlined.

FIG. 11 Rf3-58 coding sequence (SEQ ID NO: 43) and protein sequence (SEQ ID NO: 44). The amino acids encoded by the miRNA binding site are highlighted in bold and italics.

FIG. 12 Seed production of 1-copy plants with intact miRNA3619 binding site (miR-BS, pBAS04254 (n=31)) compared to 1-copy plants with disrupted miRNA3619 binding site (pBAS04255 (n=44)). The disrupted miRNA binding site comprises a sequence as show in SEQ ID NO: 50 (same as in pBas04648).

FIG. 13 Rf3-58 expression in 1-copy-plants with intact miRNA3619 binding site (miR-BS, pBAS04254 (n=31)) and 1-copy plants with disrupted miRNA3619 binding site (pBAS04255 (n=44)). The disrupted miRNA binding site comprises a sequence as show in SEQ ID NO: 50 (same as in pBas04648).

FIG. 14 Rf1-09 coding sequence (SEQ ID NO: 64). The identified miRNA binding site is highlighted in bold and italics.

FIG. 15 Rf1-09 coding sequence (SEQ ID NO: 64) and protein sequence (SEQ ID NO: 65). The amino acids encoded by the miRNA binding site are highlighted in bold and italics.

FIG. 16 Activity of different fragments of the wheat Rf3-58 promoter in transiently transformed wheat protoplasts. The horizontal axis legend shows the size of promoter sequence (upstream of the translation start codon) tested. The tested plasmids contain the promoter fragments upstream of the rice actin-1 intron and the GUS coding sequence. GUS activities were corrected for variation in protoplast transfection efficiency using the luciferase activities of a co-introduced pKA63 plasmid.

FIG. 17 Impact of wheat enhancers on activity of the Rf3-58 promoter in transiently transformed wheat protoplasts. The enhancer fragments were inserted at position −127 relative to the translation start site. GUS activities were corrected for variation in protoplast transfection efficiency using the luciferase activities of a co-introduced pKA63 plasmid. Activity of the promoter without enhancer was set at 1.

FIG. 18. Impact of wheat enhancers on activity of the Rf3-58 promoter in transiently transformed wheat protoplasts. The horizontal axis legend shows the enhancer fragment name and the position in the promoter (relative to the translation start site) where the enhancer was inserted. GUS activities were corrected for variation in protoplast transfection efficiency using the luciferase activities of a co-introduced pKA63 plasmid. Activity of the promoter without enhancer was set at 1.

FIG. 19 Impact of the EN1390 enhancer on activity of the Rf3-58 promoter in transiently transformed wheat protoplasts. The horizontal axis legend shows the position in the promoter (relative to the translation start site) where the enhancer was inserted. GUS activities were corrected for variation in protoplast transfection efficiency using the luciferase activities of a co-introduced pKA63 plasmid. Activity of the promoter without enhancer was set at 1.

FIG. 20 Impact of the EN1390 enhancer on activity of the Rf3-58 promoter in transiently transformed wheat protoplasts. The horizontal axis legend shows the position in the promoter (relative to the translation start site) where the enhancer was inserted and the copy number and orientation of the insert. GUS activities were corrected for variation in protoplast transfection efficiency using the luciferase activities of a co-introduced pKA63 plasmid. Activity of the promoter without enhancer was set at 1.

FIG. 21 Seed set upon selfing of CMS-containing G1 plants containing 1 Rf3 allele from Naxos and either a precisely edited Rf3 Fielder allele with a repaired coding sequence (PE), a wild-type Rf3 Fielder allele (WT), or a Rf3 indel allele that has either an insertion of 1 G (+G) or a modification that prevents amplification of the allele by PCR (?). The numbers at the x axes indicate the G0 event name and the Naxos plant number on which the G1 seed was produced (eg 22-09: event TMTA0423-0022-B01 crossed with Naxos plant 9).

FIG. 22 Sequence of an edited Fielder Rf3 gene with EN1390 enhancer insertion and repaired coding sequence frameshift (SEQ ID NO: 92). The EN1390 sequence is underlined, the translation start codon is indicated in bold with a grey background, the 2-nt insertion in the CDS is underlined and indicated in bold and italic.

FIG. 23 Relative Rf3 RNA expression levels in leaves of G0 plants (indicated as PE/IN) compared to unedited Fielder plants (WT). G0 plants contain 1 precisely edited RF3-58 allele with a repaired coding sequence and the EN1390 insertion (PE) and 1 Rf3 indel allele (IN).

FIG. 24 Relative Rf3 RNA expression levels in developing spikes of G1 plants compared to unedited Fielder plants (WT). G1 plants contain 1 Rf3 allele from Naxos (“N”) and either a precisely edited Rf3 allele with a repaired coding sequence and the EN1390 insertion (“PE”) or a Rf3 indel allele (“IN”).

FIG. 25 Relative Rf3 RNA expression levels in developing spikes of G1 plants compared to unedited Fielder plants (WT/WT). G1 plants contain 1 Rf3 allele from Naxos (N) and a precisely edited Rf3 allele with a repaired coding sequence (PE) but with no enhancer insertion.

FIG. 26 Seed set of CMS-containing G1 plants compared to Fielder plants lacking CMS (F). G1 plants contain 1 non-functional Rf3 allele from Naxos (N) and either a precisely edited Rf3 allele with a repaired coding sequence and the EN1390 insertion (PE) or an Rf3 indel allele (IN).

FIG. 27 Seed set of CMS-containing G1S1 plants compared to Fielder plants lacking CMS and transgenic plants expressing an optimized Rf3 CDS under control of the maize ubiquitin promoter (pUbi58). G1 plants have segregating Rf3 alleles, one from Naxos (N) and either an allele with a repaired coding sequence and the EN1390 insertion (EN-RES) or an allele with only a repaired coding sequence (RES).

FIG. 28. Relative Rf3 RNA expression levels in leaves and developing spikes of G1S1 plants. Plants contain 1 Rf3 allele from Naxos (N) and either a precisely edited Rf3 allele with a repaired coding sequence and the EN1390 insertion (EN-RES) or an allele with only a repaired coding sequence (RES).

FIG. 29 Sequence of an edited Fielder Rf3 gene with EN1390 enhancer insertion, transcription factor binding site region, miRNA binding site inactivation and repaired coding sequence frameshift (SEQ ID NO: 93). The EN1390 sequence is underlined, the duplicated transcription factor binding site region is double underlined, the translation start codon is indicated in bold with a grey background, the 2-nt insertion in the CDS is underlined and indicated in bold and italic, whereas the mutated miRNA binding site is underlined with the mutated nucleotides indicated in small bold letters.

### OVERVIEW

The present invention concerns means and methods for increasing expression of functional restorer genes for wheat cytoplasmic male sterility. The means and methods are based on modified restorer genes for wheat cytoplasmic male sterility, such as G-type wheat cytoplasmic male sterility.

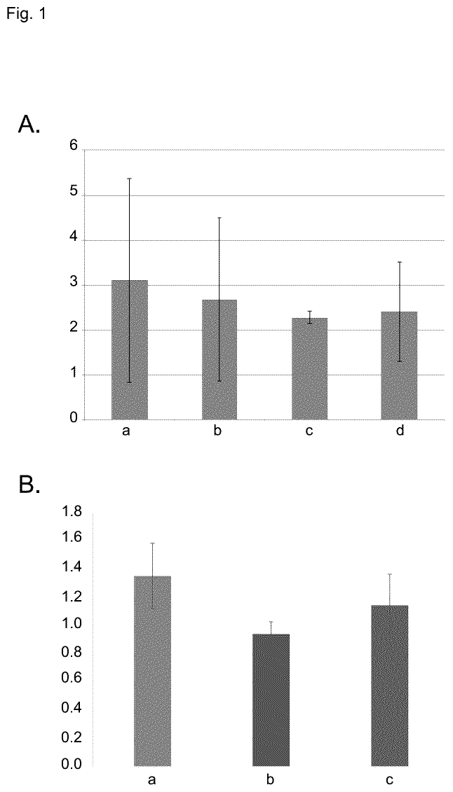

In a first aspect, the present invention relates to a modified promoter comprising at least one heterologous binding site for an EIL3 transcription factor and/or at least one heterologous binding site for a PHD transcription factor, as well as to use of said modified promoter. In some embodiments, the promoter is a modified promoter of a functional restorer gene for wheat cytoplasmic male sterility, such as G-type wheat cytoplasmic male sterility. This first aspect is described in Section A. The results for this aspect are, e.g., shown in Examples 1 to 6 and in FIGS. 1 to 5.

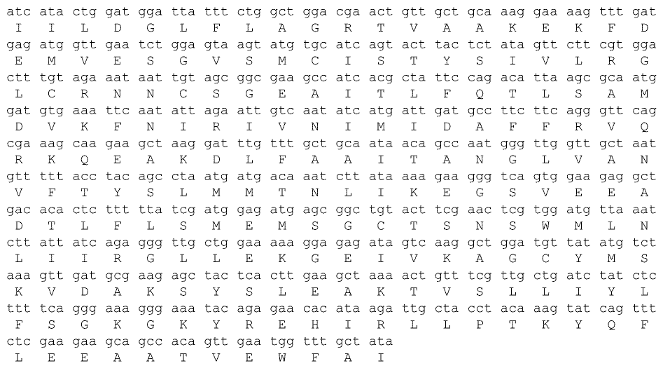

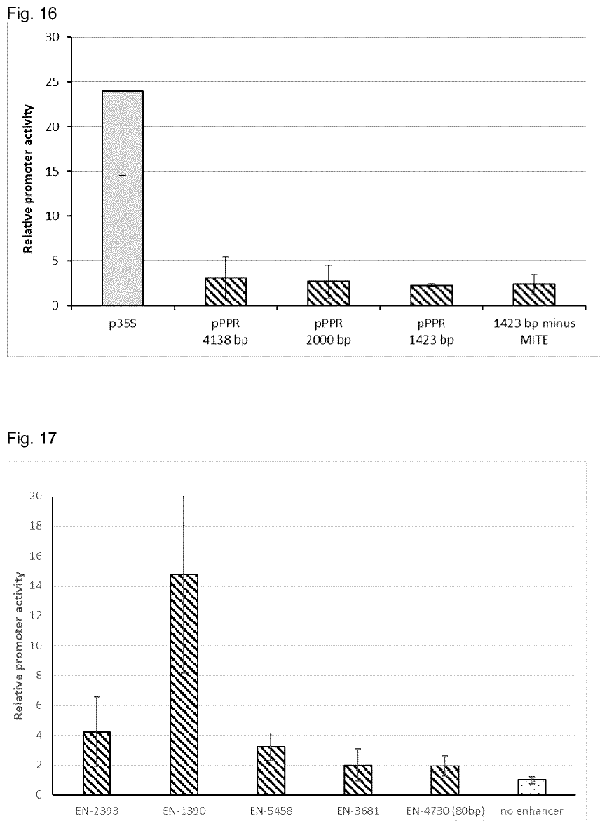

In a second aspect, the present invention relates to a modified promoter of a functional restorer gene for wheat cytoplasmic male sterility (such as G-type wheat cytoplasmic male sterility) comprising one or more enhancers (herein also referred to as “nucleic acid expression enhancing nucleic acid” (NEENA) molecules) as well as to the use of said modified promoter. This second aspect is described in Section B. The results for this aspect are, e.g., shown in Examples 11 and 12 and in FIGS. 16 to 28.

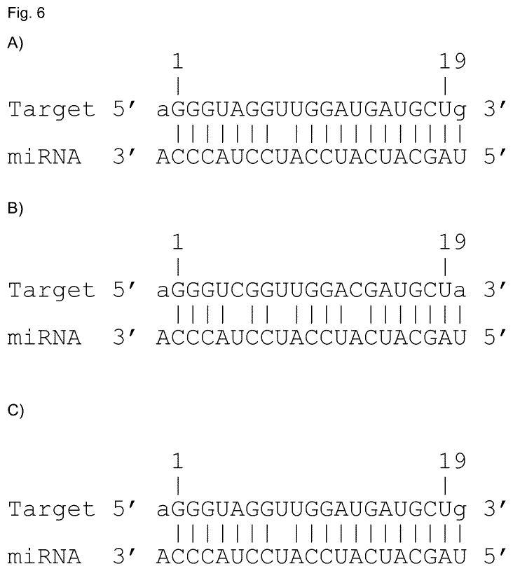

In a third aspect, the present invention relates to a nucleic acid molecule encoding a functional restorer polypeptide for wheat cytoplasmic male sterility, such as G-type wheat cytoplasmic male sterility. Said nucleic acid molecule comprises, in the coding sequence, a mutated microRNA (“miRNA”) binding site. This third aspect is described in Section C. The results for this aspect are, e.g., shown in Examples 7 to 10 and in FIGS. 6 to 15.

The three aspects can also combined.

In preferred embodiment, the modified promoter comprising at least one heterologous binding site for an EIL3 transcription factor and/or at least one heterologous binding site for a PHD transcription factor as defined in Section A (such as in any one of the listed embodiments 1 to 39 in Section A) is used for expressing the nucleic acid molecule encoding a functional restorer polypeptide for wheat cytoplasmic male sterility as defined in Section C (such as in any one of the listed embodiments 1 to 26 in Section C). Thus, it is operably linked to said nucleic acid molecule.

In another preferred embodiment, the modified promoter of a functional restorer gene for wheat cytoplasmic male sterility (such as G-type wheat cytoplasmic male sterility) comprising one or more enhancers as defined in Section B (such as in any one of the listed embodiments 1 to 33 in Section B) is used for expressing the nucleic acid molecule encoding a functional restorer polypeptide for wheat cytoplasmic male sterility as defined in Section C (such as in any one of the listed embodiments 1 to 26 in Section C). Thus, it is operably linked to said nucleic acid molecule.

Moreover, the present invention relates to a promoter of a functional restorer gene for wheat cytoplasmic male sterility comprising the promoter modifications as described in Section A (such as in any one of the listed embodiments 1 to 39 in Section A) and in Section B (such as in any one of the listed embodiments 1 to 33 in Section B). Thus, the present invention also relates to a modified promoter of a functional restorer gene for wheat cytoplasmic male sterility (such as G-type wheat cytoplasmic male sterility), said promoter comprising i) at least one heterologous binding site for an EIL3 transcription factor and/or at least one heterologous binding site for a PHD transcription factor as defined in Section A (such as in any one of the listed embodiments 1 to 39 in Section A), and ii) one or more enhancers as described in Section B (such as in any one of the listed embodiments 1 to 33 in Section B).

In a preferred embodiment of the present invention, said promoter of a functional restorer gene for wheat cytoplasmic male sterility comprising the promoter modifications as described in Section A (such as in any one of the listed embodiments 1 to 39 in Section A) and in Section B (such as in any one of the listed embodiments 1 to 33 in Section B) is used for expressing the nucleic acid molecule encoding a functional restorer polypeptide for wheat cytoplasmic male sterility as defined in Section C (such as in any one of the listed embodiments 1 to 26 in Section C). Thus, it is operably linked to said nucleic acid molecule.

Typically, the definitions provided herein in the individual sections, i.e. in Section A, B and C apply mutatis mutandis to the other sections.

### Section A: Modified Promoters with Heterologous EIL3 and/or PHD Transcription Factor Binding Site(s)

### Brief Summary of the First Aspect of the Present Invention (SECTION A)

In a first aspect, the present invention provides a method for producing a plant promoter having increased activity in the presence of an EIL3 (Ethylene insensitive 3-like) transcription factor and/or a PHD (Plant homeodomain) transcription factor, comprising the steps of
a) providing a plant promoter, and
b1) introducing at least one binding site for the EIL3 transcription factor and/or at least one binding site for the PHD transcription factor into the plant promoter, and/or
b2) modifying at least one existing binding site for the EIL3 transcription factor and/or at least one existing binding site for the PHD transcription factor in the promoter, such that binding of the EIL3 or PHD transcription factor to said binding site is improved.

The first aspect of the present invention is also directed to a plant promoter obtained or obtainable by the method of the present invention.

In particular, the first aspect of the present invention is directed to a plant promoter comprising at least one heterologous binding site for an EIL3 transcription factor and/or at least one heterologous binding site for a PHD transcription factor.

Also, the first aspect of the present invention is directed to a plant promoter comprising at least one modified binding site for an EIL3 transcription factor and/or at least one modified binding site for a PHD transcription factor.

In a preferred embodiment of the first aspect of the present invention, the plant promoter of the present invention is a promoter of a functional restorer gene for wheat G-type cytoplasmic male sterility, e.g. for an Rf1 or Rf3 gene. Thus, the plant promoter of the first aspect of the present invention is operably linked to nucleic acid molecule that encodes a functional restorer polypeptide for wheat cytoplasmic male sterility, such as G-type or K-type cytoplasmic male sterility (preferably wheat G-type cytoplasmic male sterility).

Furthermore, the first aspect of the invention relates to a chimeric nucleic acid molecule comprising the following operably linked elements
a) the plant promoter of the present invention;
b) a nucleic acid molecule of interest; and optionally
c) a transcription termination and polyadenylation region functional in plant cells.

In a preferred embodiment, the nucleic acid molecule of interest under b) encodes a functional restorer polypeptide for wheat cytoplasmic male sterility, such as for wheat G-type or K-type cytoplasmic male sterility.

The first aspect of the present invention is further directed to a plant cell, plant or seed, such as a cereal plant cell, plant or seed, comprising the plant promoter of the present invention or the chimeric nucleic acid molecule of the present invention. In an embodiment, the cereal plant cell, plant or seed is a wheat plant cell, plant or seed.

The first aspect of the present invention further pertains to a method for producing a plant cell or plant or seed thereof, such as a cereal plant cell or plant or seed thereof, comprising the step of providing said plant cell or plant with the plant promoter or the chimeric nucleic acid molecule of the invention.

The first aspect of the present invention also relates to a method for increasing expression of a functional restorer polypeptide for wheat G-type cytoplasmic male sterility, or for increasing restoration capacity for wheat G-type cytoplasmic male sterility (“CMS”) in a cereal plant, such as a wheat plant, comprising the step of providing said plant cell or plant with the plant promoter or the chimeric nucleic acid molecule of the invention.

Moreover, the first aspect of the present invention relates to a method for identifying and/or selecting a cereal plant having increased expression of a functional restorer polypeptide for wheat G-type cytoplasmic male sterility and/or increased restoration capacity for wheat G-type cytoplasmic male sterility, said method comprising the steps of:
a) identifying or detecting in said plant the presence of plant promoter or the chimeric nucleic acid molecule of the present invention, and
b) selecting said plant comprising said plant promoter or chimeric nucleic acid molecule.

The first aspect of the present invention further relates to a method for producing hybrid seed, comprising the steps of:
a) providing a i) male cereal parent plant, such as a wheat plant, produced according to the method of the present invention and/or ii) a male cereal parent plant, such as a wheat plant, comprising the plant promoter or the chimeric nucleic acid molecule of the present invention, wherein said promoter or chimeric nucleic acid molecule is preferably present in homozygous form,
b) providing a female cereal parent plant that is a G-type cytoplasmic male sterile cereal plant,
c) crossing said female cereal parent plant with a said male cereal parent plant; and optionally,
d) harvesting hybrid seeds from said female parent plant.

The first aspect of the present invention further relates to the use of the plant promoter or the chimeric nucleic acid molecule of the present invention for the identification of a plant comprising said functional restorer gene allele for wheat G-type cytoplasmic male sterility.

The first aspect of the present invention further relates to the use of a plant of the present invention or a plant obtained or obtainable by the method of the present invention for restoring fertility in a progeny of a cytoplasmic male sterile cereal plant, such as a G-type or K-type cytoplasmic male sterile wheat plant.

The first aspect of the present invention further relates to the use of a plant of the present invention or a plant obtained or obtainable by the method of the present invention for producing hybrid seed or a population of hybrid cereal plants, such as wheat seed or plants.

The first aspect of the present invention further relates to the use of at least one heterologous binding site for an EIL3 transcription factor and/or at least one heterologous binding site for a PHD transcription factor for increasing the activity of a plant promoter in developing spikes.

The first aspect of the present invention further relates to the use of the plant promoter of the present invention for increasing expression of a nucleic acid molecule of interest in a plant, wherein the plant promoter is operably linked to the nucleic acid molecule of interest. Preferably, expression is increased in developing spikes.

### Detailed Description of the First Aspect of the Present Invention (Section A)

The Rf3-58 gene is a functional restorer gene for wheat G-type cytoplasmic male sterility used in wheat hybrid breeding. Increased expression levels of Rf3-58 gene leads to better restoration of the fertility in the progeny of a cross with a G-type cytoplasmic male sterility (“CMS”) line.

In the studies underlying the present invention, the inventors have identified two wheat transcription factors that are capable of binding to the promoter of the Rf3-58 gene: a PHD transcription factor and an EIL3 transcription factor (see Example 1). Moreover, the transcription factor binding sites for the PHD transcription factor and the EIL3 transcription factor were identified (see Example 3 and 4). In silico expression analysis carried out for three wheat homeologs of the identified transcription factors showed that the homeologs are expressed in developing spikes, i.e. in a stage in which the Rf3-58 gene is naturally expressed. In leaves, the expression is lower than in the early stages of developing spikes.

Moreover, it was shown that a Rf3-58 promoter containing a duplication of a region comprising the EIL3 and PHD transcription factor binding sites had increased activity in wheat protoplasts derived from leaves only when either one of the 2 transcription factors are overexpressed (see Example 6). This indicates that the promoter duplication will lead to an increased expression when the EIL3 and/or PHD transcription factor is present, e.g. in developing spikes. Since the Rf3-58 gene is expressed in developing spikes, the introduction of one or more additional EIL3 and/or PHD transcription factor binding sites into its promoter would be, thus, a way to increase its expression in the developing spike and to improve restoration.

Alternatively, the increased expression could be achieved by modifying binding sites for the EIL3 and/or PHD transcription factor which already exist in a plant promoter. Preferably, the binding sites are modified such that binding of the EIL3 and/or PHD transcription factor to said binding sites is improved.

Interestingly, the promoter of the Rf3-29a gene, an allelic variant of the Rf3-58 gene, comprises binding sites for the EIL3 and PHD transcription factors as well. Whereas the binding site for EIL3 is the same as in the Rf3-58 promoter, the binding site for PHD deviates in one nucleotide from the binding site in the Rf3-58 promoter (see FIG. 4). Moreover, the promoter of an Rf1 gene, the Rf1-09 gene, comprises a binding site for the PHD transcription factor, but does not comprise an EIL3 transcription factor binding site (see FIG. 5). The PHD binding site in the Rf1-promoter differs in two nucleotides from the PHD binding site in the Rf3-58 promoter

In summary, the results described in the Examples section show that the EIL3 and PHD transcription factor binding sites could be used for engineering plant promoters having increased activity in the presence of the EIL3 and PHD transcription factors. Engineered plant promoters according to the present invention would thus have increased activity in plant tissues and/or at developmental stages in which the EIL3 transcription factor and/or the PHD transcription factor is (are) abundant, such as in developing spikes.

Accordingly, the present invention relates to a method for producing a plant promoter having increased activity in the presence of an EIL3 (Ethylene insensitive 3-like) transcription factor and/or a PHD (Plant homeodomain) transcription factor, comprising the steps of
a) providing a plant promoter, and
b1) introducing at least one binding site for the EIL3 transcription factor and/or at least one binding site for the PHD transcription factor into the plant promoter, and/or
b2) modifying at least one existing binding site for the EIL3 transcription factor and/or at least one existing binding site for the PHD transcription factor in the promoter, such that binding of the EIL3 or PHD transcription factor to said binding site is improved.

In accordance with the above method of the present invention, a promoter is produced having increased promoter activity. Preferably, the activity of the promoter is increased as compared to the activity of a control promoter. Typically, the control promoter does not comprise the modification(s) described herein. Preferably, the control promoter is the plant promoter provided in step a) of the present invention.

Preferably, the activity of a promoter produced by the method of the present invention is increased, by at least 20%, more preferably, by at least 40% and, even more preferably, by at least 60%, and most preferably by at least 100% as compared to the control promoter.

Whether the activity of a promoter is increased, or not, can be assessed by the skilled person without further ado. For example, the promoter can be operably linked to a reporter gene and the activity of the promoter can be quantified by determining the amount of the reporter gene product. This amount can be compared to the amount of reporter gene product generated by the control promoter. To check the relevance of the presence of the relevant transcription factor for a promoter having a transcription factor binding site, the amount of the reporter gene product measured in the presence of a relevant transcription factor can also be compared to the amount of reporter gene product produced by the same promoter, but in the absence of the relevant transcription factor. Reporter genes are well known in the art. For example, the reporter gene can be, but is not limited to, a GUS gene, a luciferase gene, or a GFP gene. These genes were used in the studies underlying the present invention (see Examples 1, 5 and 6).

It is to be understood that the activity of the produced promoter is only increased in the presence of an EIL3 (Ethylene insensitive 3-like) transcription factor and/or a PHD (Plant homeodomain) transcription factor. The transcription factors are described elsewhere herein in more detail. Thus, promoter activity is increased in plant cells, plant tissues and/or at developmental stages in which the EIL3 transcription factor and/or the PHD transcription factor is (are) expressed. In particular, promoter activity is increased in plant cells, plant tissues and/or at developmental stages in which the transcription factors are abundant, such as in developing spikes. Accordingly, the produced promoter, preferably, has increased activity in developing spikes (e.g. of cereal plants, preferably wheat plants). More preferably, the produced promoter has increased activity in early spike development. Most preferably, the produced promoter has increased activity in developing spikes at Zadok stages Z39-Z41 (tetrad phase), Z45-Z48 (uninucleate phase), Z50-Z59 (binucleate phase), and/or Z60-Z69 (trinucleate phase). Accordingly, the present invention also relates to a method for producing a plant promoter having increased activity at the aforementioned stages. The Zadok stages are well known in the art, and are, e.g. described by Zadoks et al. (J. C. Zadoks, T. T. Chang, C. F. Konzak, “A Decimal Code for the Growth Stages of Cereals”, Weed Research 1974 14:415-421))

In an embodiment, the promoter has increased activity in spikes at Zadok stages Z39-Z41.

In an embodiment, the promoter has increased activity in spikes at Zadok stages Z45-Z48.

In an embodiment, the promoter has increased activity in spikes at Zadok stages Z50-Z59

In an embodiment, the promoter has increased activity in spikes at Zadok stages Z60-Z69 (trinucleate phase).

Moreover, it is envisaged that the produced promoter has increased activity in tissues involved in (early) pollen development and meiosis, such as in the anther or, more specifically, in the tapetum, or in developing microspores.

In step a) of the present invention, a plant promoter is provided.

The term “promoter” refers to a regulatory nucleic acid sequence capable of effecting expression of the sequences to which they are ligated. The term “promoter” as used herein refers to a nucleic acid control sequence located upstream from the translational start of a gene and which is involved in recognizing and binding of RNA polymerase and other proteins, thereby directing transcription of an operably linked nucleic acid.

In accordance with the present invention, a “plant promoter” typically comprises regulatory elements, which mediate the expression of a coding sequence segment in a plant and/or in plant cells. Preferably, the plant promoter is of plant origin and, thus, is a promoter which is naturally present in plants. For example, the plant promoter provided in step a) of the above method may be a promoter from a cereal plant, such as a wheat plant. However, the promoter provided in step a) of the present invention is not limited to promoters which are naturally present in plants. For example, the promoter provided in step a) may comprise already one or more modification(s), e.g. one or more nucleotide substitution(s), insertion(s) and/or deletion(s), provided that the promoter is still active in plants. Moreover, the plant promoter may originate from viruses, for example from viruses which attack plant cells.

In an embodiment, the plant promoter provided in step a) of the method of the present invention, i.e. the promoter to be modified, is a plant promoter which has at least some basal activity in the plant cells, plant tissues and/or at developmental stages in which the EIL3 transcription factor and/or the PHD transcription factor is (are) expressed, for example in developing spikes of a cereal plant. Thus, the provided plant promoter shall be active during spike development, in particular during early spike development. For example, the promoter provided in step a) shall be capable of directing expression of the operably linked nucleic acid at least during (early) pollen development and meiosis, such as in anther or, more specifically, tapetum, or developing microspores. This can for example be a constitutive promoter, an inducible promoter, but also a pollen-, anther- or, more specifically a tapetum- or microspore-specific/preferential promoter. Pollen/microspore-active promoters include, e.g., a maize pollen specific promoter (see, e.g., Guerrero (1990) Mol. Gen. Genet. 224:161 168), PTA29, PTA26 and PTAI 3 (e.g., see U.S. Pat. No. 5,792,929) and as described in, e.g., Baerson et al. (1994 Plant Mol. Biol. 26: 1947-1959), the NMT19 microspore-specific promoter as, e.g., described in WO97/30166. Further anther/pollen-specific or anther/pollen-active promoters are described in, e.g., Khurana et al., 2012 (Critical Reviews in Plant Sciences, 31: 359-390), WO2005100575, WO 2008037436. Other suitable promoters are e.g the barley vrn1 promoter, such as described in Alonso-Peral et al. (2001, PLoS One. 2011; 6(12):e29456). Atapetum specific promoter is, preferably, pOsg6B (T Tsuchiya et al 1994 doi: 10.1007/BF00019488), pE1 (WO1992/13956A1) or pCA55 (U.S. Pat. No. 5,589,610A). A pollen-specific promoter is preferably pZM13 (Hamilton et al. 1989. Sex Plant Reprod 2: 208-212).

In a preferred embodiment, the plant promoter provided in step a) of the method of the present invention is a promoter derived from a plant, i.e. a promoter which is naturally present in a plant.

The term “plant” as used herein preferably relates to a cereal plant. Cereal plants are members of the monocotyledonous family Poaceae which are cultivated for the edible components of their grain. These grains are composed of endosperm, germ and bran. Maize, wheat and rice together account for more than 80% of the worldwide grain production. Other members of the cereal family comprise rye, oats, barley, triticale, sorghum, wild rice, spelt, einkorn, emmer, and durum wheat. Accordingly, the plant is typically a cereal plant selected from the group consisting of wheat, rice, maize, rye, oats, barley, triticale, sorghum, spelt, einkorn and emmer.

In one embodiment, a cereal plant as set forth herein is a cereal plant that comprises at least a B genome or related genome, such as wheat (Triticum aestivum; ABD), spelt (Triticum spelta; ABD) durum (T. turgidum; AB), barley (Hordeum vulgare; H) and rye (Secale cereale; R).

In a specific embodiment, the cereal plant according to the invention is wheat (Triticum aestivum; ABD). Accordingly, the promoter provided in step a) is preferably a wheat promoter.

In a preferred embodiment, the plant promoter to be provided in step a) of the above method is a promoter of a functional restorer gene for cytoplasmic male sterility. In particular, the promoter is a promoter of a functional restorer gene for wheat G-type or K-type cytoplasmic male sterility.

The term “male sterility” in connection with the present invention refers to the failure or partial failure of plants to produce functional pollen or male gametes. This can be due to natural or artificially introduced genetic predispositions or to human intervention on the plant in the field. Male fertility on the other hand relates to plants capable of producing normal functional pollen and male gametes. Male sterility/fertility can be reflected in seed set upon selfing, e.g., by bagging heads to induce self-fertilization. Likewise, fertility restoration can also be described in terms of seed set upon crossing a male sterile plant with a plant carrying a functional restorer gene, when compared to seed set resulting from crossing (or selfing) fully fertile plants. A male parent (or pollen parent), is a parent plant that provides the male gametes (pollen) for fertilization, while a female parent or seed parent is the plant that provides the female gametes for fertilization, said female plant being the one bearing the (hybrid) seeds.

A functional restorer gene for wheat G-type cytoplasmic male sterility encodes a polypeptide which allows for restoring cytoplasmic male sterility (abbreviated “CMS”). “CMS” refers to cytoplasmic male sterility. CMS is total or partial male sterility in plants (e.g., as the result of specific nuclear and/or mitochondrial interactions) and is maternally inherited via the cytoplasm. Male sterility is the failure of plants to produce functional anthers, pollen, or male gametes although CMS plants still produce viable female gametes. Cytoplasmic male sterility is used in agriculture to facilitate the production of hybrid seed.

A functional restorer polypeptide for wheat G-type cytoplasmic male sterility has the capacity to restore fertility in the progeny of a cross with a G-type cytoplasmic male sterile cereal plant (when expressed in a (sexually compatible) cereal plant). Thus, it is capable of restoring the fertility in the progeny of a cross with a G-type cytoplasmic male sterility (“CMS”) line, i.e., a line carrying common wheat nuclear genes but cytoplasm from Triticum timopheevii.

Restoration against G-type cytoplasm has been described in the art. The restorer genes encoding such polypeptides are also referred to as Rf (restorer of fertility) genes. Most fertility restoration polypeptides come from a clade of genes encoding pentatricopeptide repeat (PPR) proteins (Fuji et al., 2011, PNAS 108(4), 1723-1728—herein incorporated by reference). So far, up to nine different Rf loci have been reported to restore the fertility against T. timopheevii cytoplasm (Shahinnia et al.), and their chromosome locations have been determined, namely, Rf1 (Chr1A), Rf2 (Chr7D), Rf3 (Chr1B), Rf4 (Chr6B), Rf5 (Chr6D), Rf6 (Chr5D), Rf7 (Chr7B) Rf8 (Chr2D) and Rf9 (Chr6A).

Accordingly, the promoter provided in step a) of the above method is preferably a promoter of a functional restorer gene for wheat G-type cytoplasmic male sterility selected from the group consisting of an Rf1 gene, an Rf2 gene, an Rf3 gene, an Rf4 gene, an Rf5 gene, an Rf6 gene, an Rf7 gene, an Rf8 gene and an Rf9 gene.

In a preferred embodiment of the present invention, the promoter provided in step a) of the method of the present invention is the promoter of an Rf3 gene, such as the promoter of the Rf3-58 gene or the promoter of the Rf3-29a gene.

In another preferred embodiment of the present invention, the promoter provided in step a) of the method of the present invention is the promoter of an Rf1 gene, such as the promoter of the Rf1-09 gene.

The promoters of the Rf3-58 gene and the promoter of the Rf3-29a gene already comprise EIL3 and PHD binding sites. Further, the promoter of the Rf1 gene comprises a PHD binding site. In an embodiment, the promoter to be provided in step a) of the above method, thus, already comprises at least one EIL3 binding site and/or at least one PHD binding site (preferably both). Thus, at least one additional EIL3 binding site and/or at least one additional PHD binding site is introduced in step b1). Preferably, the introduction of the at least one additional binding site does not disrupt the existing binding sites.

The promoter of a gene, typically, comprises the region upstream (5′) to translation start site (herein also referred to as “start codon”) of a gene (typically ATG). The transcription factor binding site(s) as referred to herein shall be introduced into said region. Preferably, said region shall allow for the expression of a gene that is operably linked to the promoter region. Typically, said region has a length of at least 200 bp, at least 250 bp, at least 300 bp, at least 400 bp, at least 500 bp, at least 750 bp, at least 1000 bp, at least 1500 bp, or at least 2000 bp. Whether a region allows for the expression of a gene being operably linked to it, can be determined by the skilled person without further ado. Suitable experiments are described for the Rf3-58 promoter in the Examples section. Here, regions/fragments having a length of about 4 kb (SEQ ID NO: 1), about 2 kb (SEQ ID NO: 21), about 1.4 kb (SEQ ID NO: 22), or about 1.2 kb (SEQ ID NO: 23) were tested. As shown in FIG. 1A, the promoter activity of all fragments tested is comparable in wheat protoplasts.

Accordingly, the promoter of the Rf3-58 gene, preferably, comprises the following sequence:
a) a promoter comprising a nucleic acid sequence as shown in SEQ ID NO: 23,
b) a fragment of the nucleic acid sequence shown in SEQ ID NO: 23,
c) a variant of the promoter of a) or the fragment of b), said fragment or variant having a sequence being at least 80%, 85%; 86%; 87%; 88%; 89%; 90%; 91%; 92%; 93%; 94%; 95%; 96%; 97%; 98%; or 99% identical to the sequence of a) or b).

Preferably, the promoter of the Rf3-29a gene comprises the following sequence:
a) a promoter comprising a nucleic acid sequence as shown in SEQ ID NO: 36,
b) a fragment of the nucleic acid sequence shown in SEQ ID NO: 36,
c) a variant of the promoter of a) or the fragment of b), said fragment or variant having a sequence being at least 80%, 85%; 86%; 87%; 88%; 89%; 90%; 91%; 92%; 93%; 94%; 95%; 96%; 97%; 98%; or 99% identical to the sequence of a) or b).

Accordingly, the promoter of the Rf1-09 gene comprises the following sequence:
a) a promoter comprising a nucleic acid sequence as shown in SEQ ID NO: 37,
b) a fragment of the nucleic acid sequence shown in SEQ ID NO: 37,
c) a variant of the promoter of a) or the fragment of b), said fragment or variant having a sequence being at least 80%, 85%; 86%; 87%; 88%; 89%; 90%; 91%; 92%; 93%; 94%; 95%; 96%; 97%; 98%; or 99% identical to the sequence of a) or b).

Preferably, the fragment under b) or the variant under c) has essentially the same promoter activity of the promoter under a). A promoter activity of at least 80%, at least 90%, or at least 95% or at least 98% is considered to be essentially the same promoter activity.

Preferably, the fragment under b) has a length of at least 200 bp, at least 250 bp, at least 300 bp, at least 400 bp, at least 500 bp, at least 750 bp, at least 1000 bp, at least 1500 bp, or at least 2000 bp.

The term “variant” with respect to a parent sequence (e.g., a polypeptide or nucleic acid sequence) is intended to mean substantially similar sequences.

Polypeptide or nucleic acid variants may be defined by their sequence identity when compared to a parent polypeptide or nucleic acid. Sequences of variants are considered as substantially similar, if they are, in increasing order of preference, at least 50%, 60%, 70%, 75%, 80%, 85%; 86%; 87%; 88%; 89%; 90%; 91%; 92%; 93%; 94%; 95%; 96%; 97%; 98%; or 99% identical to the parent sequence. Sequence identity usually is provided as “% sequence identity” or “% identity” (or % identical). To optimally determine the percent-identity between two amino acid/nucleic acid sequences in a first step a pairwise sequence alignment is generated between those two sequences, wherein the two sequences are aligned over their complete length (i.e., a pairwise global alignment, also called an optimal alignment herein). The optimal alignment is generated with a program implementing the Needleman and Wunsch algorithm (J. Mol. Biol. (1970) 48, p. 443-453), preferably by using the program “NEEDLE” (The European Molecular Biology Open Software Suite (EMBOSS), see, e.g., https://www.ebi.ac.uk/Tools/psa/emboss_needle/) with the programs default settings/parameters (gapopen=10.0, gapextend=0.5 and matrix=EBLOSUM62 for proteins, and matrix EDNAFULL for DNA).

The following example is meant to illustrate two nucleotide sequences, but the same calculations apply to protein sequences:
Seq A: AAGATACTG length: 9 bases
Seq B: GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “|” symbol in the alignment indicates identical residues (which means bases for DNA or amino acids for proteins). The number of identical residues is 6.

The “-” symbol in the alignment indicates gaps. The number of gaps introduced by alignment within the Seq B is 1. The number of gaps introduced by alignment at borders of Seq B is 2, and at borders of Seq A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

The alignment length showing Seq A over its complete length would be 9 (meaning Seq A is the sequence of the invention).

Accordingly, the alignment length showing (shorter) Seq B over its complete length would be 8 (meaning Seq B is the sequence of the invention).

After aligning two sequences, in a second step, an identity value is determined from the alignment produced. For purposes of this description, percent identity is calculated by %−identity=(identical residues/length of the alignment region which is showing the two aligned sequences over their complete length)*100. Thus, sequence identity in relation to comparison of two amino acid or nucleic acid sequences according to this embodiment is calculated by dividing the number of identical residues by the length of the alignment region which is showing the two aligned sequences over their complete length. This value is multiplied with 100 to give “% identity”. According to the example provided above, the % identity here, using an optimal alignment, is: (6/10)*100=60%.

In an embodiment, step b) of the above method of the present invention comprises step b1) of introducing at least one binding site for the EIL3 transcription factor and/or at least one binding site for the PHD transcription factor into the plant promoter, i.e. into the plant promoter provided in step a) of the above method.

The introducing of the at least one binding site for the EIL3 transcription factor and/or the at least one binding site for the PHD transcription factor can be done by any method deemed appropriate.

In a preferred embodiment of the method of the present invention, the at least one binding site is introduced into the plant promoter by genome editing. Thus, the introduction is carried out in a plant cell.

The term “genome editing”, as used herein, refers to the targeted modification of genomic DNA using sequence-specific enzymes (such as endonuclease, nickases, base conversion enzymes/base editors) and/or donor nucleic acids (e.g., dsDNA, oligos) to introduce desired changes in the DNA. Sequence-specific nucleases that can be programmed to recognize specific DNA sequences include meganucleases (MGNs), zinc-finger nucleases (ZFNs), TAL-effector nucleases (TALENs) and RNA-guided or DNA-guided nucleases such as Cas9, Cpf1, CasX, CasY, C2c1, C2c3, certain argonout systems (see e.g. Osakabe and Osakabe, Plant Cell Physiol. 2015 March; 56(3):389-400; Ma et al., Mol Plant. 2016 Jul. 6; 9(7):961-74; Bortesie et al., Plant Biotech J, 2016, 14; Murovec et al., Plant Biotechnol J. 2017 Apr. 1; Nakade et al., Bioengineered 8-3, 2017; Burstein et al., Nature 542, 37-241; Komor et al., Nature 533, 420-424, 2016; all incorporated herein by reference). Donor nucleic acids can be used as a template for repair of the DNA break induced by a sequence specific nuclease, but can also be used as such for gene targeting (without DNA break induction) to introduce a desired change into the genomic DNA. Genome editing also includes technologies like prime editing (can mediate targeted insertions, deletions, and base-to-base conversions without the need for double strand breaks or donor DNA templates), see, e.g., Anzalone et al. 2019). In accordance with the present invention, plants that have been generated by genome editing are not considered as transgenic plants.

By using the above technologies, plant promoters can be converted to plant promoters having at least one (additional) binding site for the EIL3 transcription factor and/or at least one (additional) binding site for the PHD transcription factor, thereby increasing the expressing of the gene that is operably linked to the promoter, preferably in developing spikes. If the modified promoter is the promoter of an Rf gene, such as of an Rf3 or Rf1 gene, restoration capacity for wheat G-type cytoplasmic male sterility (“CMS”) in a cereal plant can be improved.

Example 6 describes the duplication of an Rf3-58 promoter fragment that contains an EIL3 and a PHD binding site (SEQ ID NO 29) by genome editing. The fragment is flanked by Cas9 target sites so that it could be duplicated in the wheat genome using a Cas9 nuclease or nickase and sgRNAs targeting these sites.

The introduction step b1) is, however, not limited to genome editing. Rather, the step could be carried out by conventional cloning methods or by gene synthesis methods. A promoter generated by such methods could be introduced into a plant by transformation.

In step b1) of the method of the present invention, the following element or elements shall be introduced into the plant promoter:
i) at least one binding site for the EIL3 transcription factor,
ii) at least one binding site for the PHD transcription factor, or
iii) at least one binding site for the EIL3 transcription factor and at least one binding site for the PHD transcription factor.

The term “at least one” as used herein, preferably, means one or more than one. Thus, at least two, three, four etc. binding sites can be introduced.

Advantageously, the use of two, three or four 22 bp fragments containing a PHD binding site into the promoter of Rf1-09 (SEQ ID NO: 31) or of Rf3-58 (SEQ ID NO: 32) leads to much better growth of yeast cells, when tested in Yeast-One-Hybrid assays (see Example 3).

Preferably, at least one binding site for the EIL3 transcription factor and at least one binding site for said PHD transcription factor are introduced into the plant promoter. Advantageously, it was shown that the introduction of both the EIL3 and PHD binding site into the Rf3 promoter resulted—in presence of the EIL3 transcription factor—in an even further increase of promoter activity as compared to duplicating the EIL3 binding site alone (see Example 6).

The introduction of both binding sites into a plant promoter can be achieved, for example, by introducing a fragment having a sequence as shown in SEQ ID NO: 29 into the Rf3-58 promoter. The, thus produced promoter comprises a sequence as shown in SEQ ID NO: 26.

A “binding site” of a transcription factor, herein also referred to as “transcription factor binding site” refers to a short nucleic acid sequence which can be specifically bound by a transcription factor in a plant cell or in vitro under conditions approximating intracellular physical conditions.

The binding site is typically present in the promoter of a gene. In accordance with the present invention, binding of a transcription factor, such as EIL3 and PHD, to its binding site results in increased transcription of the gene that is operably linked to the promoter.

Preferably, the EIL3 and PHD transcription factors as referred to herein are cereal transcription factors, in particular wheat transcription factors.

### PHD Transcription Factor

The PHD transcription factor that was identified in the studies underlying the present invention as being capable of binding to the Rf3-58 promoter comprises an amino acid sequence as shown in SEQ ID NO: 4. The transcription factor is encoded by a polynucleotide comprising a nucleic acid sequence as shown in SEQ ID NO: 5. The term “PHD transcription factor”, as used herein, is not limited to the identified transcription factor. Rather, the term also encompasses variants of the transcription factor.

Accordingly, the PHD transcription factor, preferably comprises the following sequence
a) an amino acid sequence as shown in SEQ ID NO: 4; or
b) an amino acid sequence being at least 50%, 60%, 70%, 75%, 80%, 85%; 86%; 87%; 88%; 89%; 90%; 91%; 92%; 93%; 94%; 95%; 96%; 97%; 98%; or 99% identical to SEQ ID NO: 4.

Three wheat homeologs of the PHD transcription factor identified in Example 1 are present in the wheat genome:
one in the B subgenome (SEQ ID NOs: 4 and 5, TraesCS6B02G145900),
one in the D subgenome (amino acid sequence: SEQ ID NO: 6, coding nucleic acid sequence SEQ ID NO: 7, TraesCS6D02G107700), and
one in the A subgenome (amino acid sequence: SEQ ID NO: 8, coding nucleic acid sequence SEQ ID NO: 9, TraesCS6A02G117600).

The identified PHD transcription factor is thus present in the B subgenome. However, the PHD transcription in the sense of the present invention may be also the PHD transcription factor present in the D or A subgenome, or a variant thereof.

Accordingly, the PHD transcription factor may comprise:
a) an amino acid sequence as shown in SEQ ID NO: 6 or 8; or
b) an amino acid sequence being at least 50%, 60%, 70%, 75%, 80%, 85%; 86%; 87%; 88%; 89%; 90%; 91%; 92%; 93%; 94%; 95%; 96%; 97%; 98%; or 99% identical to SEQ ID NO: 6 or 8.

The closest ortholog of the PHD transcription factor of SEQ ID NO: 4 is the rice transcription factor Os02g0147800 (also known as LOC_0s02g05450). Accordingly, the term “PHD transcription factor” as referred to herein, typically, relates to the sequence of a PHD transcription factor that clusters with the sequence of this rice transcription factor, when used in the construction of a phylogenetic tree.

### PHD Transcription Factor Binding Site

The PHD transcription factor as set forth herein is capable of binding to the PHD transcription factor binding site (when present in a promoter), e.g. in a plant cell, such as in wheat cell. Typically, binding of the PHD transcription factor to its binding site (which is present in a promoter) causes increased expression of the gene operably linked to the promoter.

A PHD binding site was identified in the RF3-58 promoter (SEQ ID NO: 23), the RFL29a promoter (SEQ ID NO: 36), and the Rf1-09 promoter (SEQ ID NO: 37). The binding sites are as follows:

For RF3-58:
(SEQ ID NO: 11)
gtagtagtagtactac
For RFL29a:
(SEQ ID NO: 40)
gtaatagtagtactac
For Rf1-09:
(SEQ ID NO: 38)
gtagtagtactactag

In an embodiment, the PHD transcription factor binding site comprises or consists of a sequence as shown in SEQ ID NO 11.

In an alternative embodiment, the PHD transcription factor binding site comprises or consists of a sequence as shown in SEQ ID NO: 40.

In an alternative embodiment, the PHD transcription factor binding site comprises or consists of a sequence as shown in SEQ ID NO: 38.

The nucleic acid sequences shown in SEQ ID NO: 11, 40 and 38 have a length of 16 bp. The PHD binding sites may be also shorter. For example, the PHD binding site may comprise or consist of a nucleic acid sequence as shown in SEQ ID NO: 42, SEQ ID NO: 41 or SEQ ID NO: 12.

For RF3-58:
(SEQ ID NO: 42)
gtagtagtagtacta
For RFL29a:
(SEQ ID NO: 41)
gtaatagtagtacta
For Rf1-09:
(SEQ ID NO: 12)
gtagtagtactacta

In an embodiment, the PHD transcription factor binding site comprises or consists of a sequence as shown in SEQ ID NO 42.

In an alternative embodiment, the PHD transcription factor binding site comprises or consists of a sequence as shown in SEQ ID NO: 41.

In an alternative embodiment, the PHD transcription factor binding site comprises or consists of a sequence as shown in SEQ ID NO: 12.

In the Examples section, the following PHD transcription factor binding sites were tested

agtagtagtagtactacata
(SEQ ID NO: 10): present in Rf3-58,
longer version of SEQ ID NO: 11,
AGTAGTAGTAGTACTACATACT
(SEQ ID NO: 32) present in Rf3-58,
longer version of SEQ ID NO: 11)
AGTAGTAGTACTACTAGATAAG
((SEQ ID NO: 31) present in Rf1-09,
longer version of SEQ ID NO: 38)

Accordingly, the PHD transcription factor binding site to be introduced, preferably, has a nucleic acid sequence as shown in SEQ ID NO: 10, SEQ ID NO 11, SEQ ID NO: 40, SEQ ID NO: 31, SEQ ID NO: 32, SEQ ID NO: 38, SEQ ID NO: 42, SEQ ID NO: 41 or SEQ ID NO: 12, or is a variant thereof.

### EIL3 Transcription Factor

The EIL3 transcription factor that was identified in the studies underlying the present invention as being capable of binding to the Rf3-58 promoter comprises an amino acid sequence as shown in SEQ ID NO: 13. The transcription factor is encoded by a polynucleotide comprising a nucleic acid sequence as shown in SEQ ID NO: 14. The term “EIL3 transcription factor”, as used herein, is not limited to the identified transcription factor. Rather, the term also encompasses variants of the transcription factor.

Accordingly, the EIL3 transcription factor may comprise:
a) an amino acid sequence as shown in SEQ ID NO: 13; or
b) an amino acid sequence being at least 50%, 60%, 70%, 75%, 80%, 85%; 86%; 87%; 88%; 89%; 90%; 91%; 92%; 93%; 94%; 95%; 96%; 97%; 98%; or 99% identical to SEQ ID NO: 13.

Three homeologs of the EIL3 transcription factor are present in the wheat genome:
one in the B subgenome (SEQ ID NOs: 13 and 14),
one in the D subgenome (SEQ ID NOs: 15 and 16), and
one on the A subgenome (SEQ ID NOs: 17 and 18).

The identified EIL3 transcription factor is thus present in the B subgenome. However, the EIL3 transcription in the sense of the present invention may be also the EIL3 transcription factor present in the D or A subgenome, or a variant thereof.

Accordingly, the EIL3 transcription factor may comprise:
a) an amino acid sequence as shown in SEQ ID NO: 15 or 17; or
b) an amino acid sequence being at least 50%, 60%, 70%, 75%, 80%, 85%; 86%; 87%; 88%; 89%; 90%; 91%; 92%; 93%; 94%; 95%; 96%; 97%; 98%; or 99% identical to SEQ ID NO: 15 or 17.

### EIL3 Transcription Factor Binding Site

The EIL3 transcription factor as set forth herein is capable of binding to the EIL3 transcription factor binding site, e.g. in a plant cell, such as in wheat cell (present in a promoter). Typically, binding of the EIL3 transcription factor to its binding site (which is present in a promoter) causes increased expression of the gene operably linked to the promoter.

An EIL3 binding site was identified in the RF3-58 promoter and the RFL29a promoter. The identified binding site is as follows: CATCTAGATACATCAATCT (SEQ ID NO: 19). Accordingly, the EIL3 transcription factor binding site may comprise or consist of a sequence as shown in SEQ ID NO: 19.

The binding site may be also shorter than SEQ ID NO: 19. For example, the binding site may be: AGATACATCAATCT (SEQ ID NO: 39). Accordingly, the EIL3 transcription factor binding site may comprise or consist of a sequence as shown in SEQ ID NO: 39.

Accordingly, the EIL3 transcription factor binding site to be introduced, preferably, has a sequence as shown in SEQ ID NO: 19 or 39, or is a variant thereof.

The EIL3 transcription factor identified in Examples 1 was assigned as EIL3 ortholog by a tool which incorporates across-species evolutionary relationships into the clustering (such as PLAZA). The EIL3 transcription factor as referred to herein is, thus, related to the Arabidopsis Ethylene-insensitive3-like3 (abbreviated as “At-EIL3”) gene, and can cluster with Os-EIL4 from rice based on sequence. Accordingly, the term “EIL3 transcription factor” as referred to herein, typically, relates to the sequence of an EIL3 transcription factor that clusters with the Oryza sativa EIL4 transcription factor sequence, when used in the construction of a phylogenetic tree.

The sequence variants of a transcription factor as referred to herein are preferably capable of binding the transcription factor binding site of the parent transcription factor (i.e., the EIL3 transcription factor of SEQ ID NO: 13 or the PHD transcription factor of SEQ ID NO: 4), thereby activating or increasing transcription of the gene that is operably linked to the promoter. The binding sites are defined elsewhere herein.

The term “transcription factor binding site” also includes variants of the transcription factor binding sites as referred to herein, i.e. of the PHD transcription factor binding site having a nucleic acid sequence as shown in SEQ ID NO: 10, SEQ ID NO 11, SEQ ID NO: 40, SEQ ID NO: 31, SEQ ID NO: 32, SEQ ID NO: 38, SEQ ID NO: 42, SEQ ID NO: 41 or SEQ ID NO: 12, or of the EIL3 transcription factor binding site having a nucleic acid sequence as shown in SEQ ID NO: 19 or SEQ ID NO: 39. These sequences are herein referred to as “reference binding sites”.

In an embodiment, the variant is a fragment of the reference binding site, such as a fragment having a length of at least 10, at least 11, at least 12, or at least 13 bp. Moreover, the fragment may have a length of at 14, at least 15, at least 16, or at least 17 bp.

In an alternative embodiment, a variant of a reference binding site is a binding site that has not more than three substitutions (i.e., nucleotide substitutions) as compared to the reference binding site (i.e. the variant has 1, 2 or 3 nucleotide substitutions). In an embodiment, the variant has not more than two nucleotide substitutions as compared to the reference binding site, i.e. the variant has 1 or 2 nucleotide substitutions. In an embodiment, the variant has not more than one substitution as compared to the reference binding site, i.e. the variant has only 1 substitution.

A variant of a transcription factor binding site, typically, is a binding site, which is capable of being bound by the respective transcription factor, i.e. by PHD or EIL3 (preferably, when present in a promoter in a cell, such as a wheat cell).

According to step b1) of the method of the present invention, the binding site(s) should be introduced into the promoter to be modified. Preferably, the binding site(s) are introduced at one or more positions within 1000 bp, such as within 500 bp or within 300 bp upstream (5′) to the translation start site of the gene that is operably linked to said promoter.

As described above, native Rf1 or Rf3 promoters comprise a binding site for the EIL3 transcription factor and/or a binding site for the PHD transcription factor. A mutated PHD and/or EIL3 binding site can also lead to an increased activity of the promoter (as compared to the nonmutated promoter), if the mutated binding site has increased binding of the relevant transcription factor. Therefore, the present invention also concerns the modification/optimization of existing transcription factor binding sites in a promoter.

Specifically, step b2) of the above method of the present invention comprises the modification of at least one existing binding site for the EIL3 transcription factor and/or at least one existing binding site for the PHD transcription factor in the plant promoter provided in step a). Thus, the promoter provided in step a) shall comprise at least one binding site for the EIL3 transcription factor and/or at least one binding site for the PHD transcription factor.

The modification in step b2) of the present invention, or the changing of an existing plant (such as wheat) promoter sequence (such as an Rf promoter sequence) to become a transcription factor binding site as described herein, is preferably a mutation. The term “mutation” as used in the first aspect of the present invention refers to any type of nucleic acid alterations such as the insertion of one or more nucleotides into the transcription factor binding site, the deletion of one or more nucleotides of the transcription factor binding site, and a substitution (i.e., change) of one or more nucleotides in an transcription factor binding site, or combinations thereof.

In an embodiment, the binding site is mutated by chemical mutagenesis, such as by EMS (ethyl methanesulfonate) mutagenesis, NaN3 (sodium azide) mutagenesis, or ENU (N-ethyl-N-nitrosourea) mutagenesis. Thus, the mutation(s) in the binding site as referred to herein has (have) been introduced by EMS (Ethyl methanesulfonate) mutagenesis, NaN3 (sodium azide) mutagenesis, or ENU (N-ethyl-N-nitrosourea) mutagenesis. EMS is a mutagenic compound that produces mutations at random positions in genetic material by nucleotide substitution; particularly through G:C to A:T transitions induced by guanine alkylation. Similarly, NaN3 is a mutagenic compound that produces mutations at random positions in genetic material by nucleotide substitution; particularly through A:T to GC transitions and G:C to A:T transitions and G:C to T:A transversions and A:T to T:A transversions. Similarly, ENU is a mutagenic compound that produces mutations at random positions in genetic material by nucleotide substitution; particularly through A:T to T:A transversions and G:C to A:T transitions and A:T to G:C transitions.

In another embodiment, the mutation(s) in the binding site as referred to herein has (have) been introduced by radiation induced mutagenesis.

Moreover, the mutation(s) as referred to herein can be introduced during somatic embryogenesis.

The modification of the existing binding site, preferably, leads to an improved (i.e. increased) binding of the EIL3 or PHD transcription factor to the modified binding site. Binding should be improved as compared to the binding of the transcription factor to the unmodified binding site. Typically, the improved binding will lead to an increased activity of the generated promoter, i.e. increased expression. This can be e.g. assessed in reporter gene assays (e.g. in protoplasts) or Yeast-One-Hybrid assays. Whether binding is improved can be also assessed by carrying out electrophoretic mobility shift assays (frequently also referred to as “gel shift assay”).

The definitions and explanations provided herein above, preferably, apply mutatis mutandis to the plant promoter, chimeric nucleic acid molecule, cereal plant cell, cereal plant, seed, method or use of the present invention.

The present invention also concerns a plant promoter obtained or obtainable by the above method of the present invention.

The present invention is further directed to a plant promoter comprising at least one heterologous binding site for an EIL3 transcription factor and/or at least one heterologous binding site for a PHD transcription factor.

In an embodiment, the term “heterologous” in connection with a transcription factor binding site, preferably, means that the binding site is not naturally present at the position at which the binding site is present. In another embodiment, the term means that the transcription factor binding site is not naturally present in the promoter. Thus, a heterologous binding site is a) a binding site which is not naturally present in the promoter or b) a binding site that is naturally present in the promoter, but at a different position as compared to its position in the promoter of the present invention.

Also, the present invention is directed to a plant promoter comprising at least one modified binding site for an EIL3 transcription factor and/or at least one modified binding site for a PHD transcription factor. Preferably, the promoter has an increased activity as compared to the unmodified promoter.

Preferably, the plant promoter of the present invention is operably linked to a nucleic acid of interest. More preferably, the plant promoter of the present invention is operably linked to a nucleic acid molecule that encodes a functional restorer polypeptide for wheat G-type cytoplasmic male sterility. Said nucleic acid molecule of interest may be a naturally occurring nucleic acid molecule or a modified nucleic acid molecule. In an embodiment, the nucleic acid molecule of interest is the nucleic acid molecule as defined in Section C herein below, i.e. the nucleic acid molecule encoding a functional restorer polypeptide for wheat G-type cytoplasmic male sterility, wherein said nucleic acid molecule comprises a mutated miRNA binding site in the coding sequence. For example, the nucleic acid molecule may be the nucleic acid molecule of any one embodiments 1 to 11 in Section C. The embodiments can be found at the end of Section C.

The term “operably linked” as used herein refers to a functional linkage between the promoter sequence and the gene of interest, such that the promoter sequence is able to initiate transcription of the gene of interest.

Furthermore, the invention relates to a chimeric nucleic acid molecule comprising the following operably linked elements
a) the plant promoter of the present invention;
b) a nucleic acid molecule of interest; and optionally
c) a transcription termination and polyadenylation region functional in plant cells.

In a preferred embodiment, the nucleic acid molecule under b) encodes a functional restorer polypeptide for wheat cytoplasmic male sterility, such as for G-type or K-type cytoplasmic male sterility. Said nucleic acid molecule may be a naturally occurring nucleic acid molecule or a modified nucleic acid molecule.

As used herein a “chimeric gene” refers to a nucleic acid construct which is not normally found in a plant species. “Chimeric DNA construct” and “chimeric gene” are used interchangeably to denote a gene in which the promoter or one or more other regulatory regions, such as the transcription termination and polyadenylation region of the gene are not associated in nature with part or all of the transcribed DNA region, or a gene which is present in a locus in the plant genome in which it does not occur naturally or present in a plant in which it does not naturally occur. In other words, the gene and the operably-linked regulatory region or the gene and the genomic locus or the gene and the plant are heterologous with respect to each other, i.e. they do not naturally occur together (such as when either the coding sequence or the regulatory elements operably-linked to such coding sequence (such as the promoter) have been modified by nucleotide substitution (e.g., via transformation, genome editing or mutagenesis).

The transcription termination and polyadenylation region is a terminator. The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene.

The plant promoter of the present invention or the chimeric gene of the present invention may be present in a plant.

Thus, the invention is further directed to a plant cell, plant or seed thereof, such as a cereal plant cell or plant or seed thereof, comprising the plant promoter of the present invention or the chimeric nucleic acid molecule of the present invention. Preferably, the cereal plant cell, plant or seed thereof is a wheat plant cell, plant or seed thereof.

In an embodiment of the plant cell, the plant or seed of the present invention, the plant cell, plant or seed is a hybrid plant cell, plant or seed.

Preferably, the plant cell, plant or seed of the present invention expresses an EIL3 transcription factor and/or a PHD transcription factor. Definitions for the EIL3 and PHD transcription factors are provided above.

Preferred cereal plants are disclosed above. Thus, cereal plants, plant parts, plant cells, or seeds thereof, especially wheat, comprising the plant promoter or chimeric gene of the present invention are provided. If the promoter is operably linked to a gene encoding a functional restorer polypeptide as set forth herein, said plant has an improved capacity to restore fertility against wheat G-type cytoplasmic male sterility. In one embodiment, the promoter or chimeric gene is heterologous to the plant, such as a transgenic, mutated or genome edited cereal plant (e.g. a wheat plant). This also includes plant cells or cell cultures comprising such plant promoter or chimeric gene of the present invention, independent whether introduced by transgenic methods or by breeding methods. The cells are, e.g., in vitro and are regenerable into plants comprising the plant promoter or chimeric gene of the present invention of the invention. Said plants, plant parts, plant cells and seeds may also be hybrid plants, plant parts, plant cells or seeds.

Whenever reference to a “plant” or “plants” according to the invention is made, it is understood that also plant parts (cells, tissues or organs, seed pods, seeds, severed parts such as roots, leaves, flowers, pollen, etc.), progeny of the plants which retain the distinguishing characteristics of the parents (especially the restoring capacity), such as seed obtained by selfing or crossing, e.g. hybrid seed (obtained by crossing two inbred parental lines), hybrid plants and plant parts derived there from are encompassed herein, unless otherwise indicated.

The plant promoter or chimeric gene of the present invention may be introduced into a plant by any method deemed appropriate.

As used herein, the term “introduction” encompasses any method for introducing a gene or transcription factor binding site of the invention into a plant. In an embodiment, the plant promoter, chimeric gene or transcription factor binding site is introduced into a plant by crossing two plants. For example, the plant promoter, chimeric gene or transcription factor binding site is introduced into a plant by crossing two plants, whereas one plant comprises the plant promoter or chimeric gene or transcription factor binding site of the present invention. The second plant may lack said nucleic acid molecule or chimeric gene or transcription factor binding site. In an alternative embodiment, the gene or transcription factor binding site is introduced by modifying an existing promoter by mutation or genome editing. In a third embodiment, the gene or transcription factor binding site is introduced by transformation. The term “transformation” as referred to herein encompasses the transfer of an exogenous polynucleotide into a host cell. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation, as used herein, means introducing a nucleotide sequence into a plant in a manner to cause stable or transient expression of the sequence. Transformation and regeneration of both monocotyledonous and dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium-mediated transformation. Transgenic plants are preferably produced via Agrobacterium-mediated transformation. The genetically modified plant cells can be regenerated via all methods with which the skilled worker is familiar. After introduction, the plant may be selected for the presence of plant promoter or chimeric gene of the present invention.

In an embodiment, the plant has been generated by genome editing (as described above).

In another embodiment, the plant of the present invention has been generated by chemical mutagenesis (as described above, such as by EMS (ethyl methanesulfonate) mutagenesis, NaN₃ (sodium azide) mutagenesis, or ENU (N-ethyl-N-nitrosourea) mutagenesis (as described above). In an embodiment, the chemical mutagenesis is EMS (ethyl methanesulfonate) mutagenesis.

In another embodiment, the plant of the present invention has been generated by irradiation induced mutagenesis, in particular gamma irradiation or fast-neutron irradiation, or X-ray irradiation. Thus, the mutation(s) in the existing transcription factor binding site as referred to herein has (have) been introduced by radiation induced mutagenesis.

In one aspect, the plant promoter or chimeric gene of the present invention is stably integrated into the cereal (e.g., wheat) genome.

In an embodiment, the plant, or plant cell of the present invention has not been obtained exclusively by an essentially biological process for the production of plants.

The obtained plants according to the invention can be used in a conventional breeding scheme to produce more plants with the same characteristics or to introduce the characteristic of the presence of the restorer gene according to the invention in other varieties of the same or related plant species, or in hybrid plants. The obtained plants can further be used for creating propagating material. Plants according to the invention can further be used to produce gametes, seeds, flour, embryos, either zygotic or somatic, progeny or hybrids of plants obtained by methods of the invention. Seeds obtained from the plants according to the invention are also encompassed by the invention. The term “plant” also encompasses the offspring/progeny of the plant of the present invention, provided that the offspring/progneny comprises the promoter obtained or obtainable by the method of the present invention.

The present invention further pertains to a method for producing a cereal plant cell or plant or seed thereof, such as a wheat plant cell or plant or seed thereof, comprising the step of providing said plant cell or plant with the plant promoter or the chimeric nucleic acid molecule or the transcription factor binding site of the invention. The plant promoter or chimeric gene or transcription factor binding site of the present invention may be provided as described elsewhere herein, such as by transformation, crossing, backcrossing, genome editing or mutagenesis.

As set forth elsewhere herein, the plant promoter is preferably operably linked to a functional restorer gene for wheat G-type cytoplasmic male sterility, such as an Rf1 or Rf3 gene. This allows for increasing expression of the said restorer gene during spike development, thereby increasing restoration capacity for wheat G-type cytoplasmic male sterility in a cereal plant.

The present invention therefore relates to a method for increasing expression of a functional restorer polypeptide for wheat G-type cytoplasmic male sterility, or for increasing restoration capacity for wheat G-type cytoplasmic male sterility (“CMS”) in a cereal plant, such as a wheat plant, comprising the step of providing said plant cell or plant with the plant promoter or the chimeric nucleic acid molecule of the invention, wherein the plant promoter is operably linked to functional restorer gene for wheat G-type cytoplasmic male sterility.

The plant, plant part, or plant cell of the present invention or produced by the method of the present invention has at least an increased expression of the gene that is operably linked to the modified promoter. Specifically, expression of the gene shall be increased as compared to the expression of the gene under control of the unmodified promoter.

If the plant promoter is preferably operably linked to a functional restorer gene for wheat G-type cytoplasmic male sterility, the plant of the present invention or the plant produced by the method of the present invention has at least one, preferably both of the following characteristics:
it has an increased restoration capacity for wheat G-type cytoplasmic male sterility (CMS) as compared to a control plant, and/or
it has an increased expression of the functional restorer polypeptide for wheat G-type cytoplasmic male sterility as compared to a control plant.

The choice of suitable control plants is a routine part of an experimental setup and may include a corresponding wild type plant or a corresponding plant comprising the nucleic acid molecule encoding a functional restorer polypeptide for wheat G-type cytoplasmic male under control of the corresponding unmodified promoter. The control plant is typically of the same plant species or even of the same variety as the plant to be assessed. Further, a control plant has been grown under equal growing conditions to the growing conditions of the plants of the invention. Typically, the control plant is grown under equal growing conditions and hence in the vicinity of the plants of the invention and at the same time. A “control plant” as used herein refers not only to whole plants, but also to plant parts, including the anther and pollen.

Whether the expression of the functional restorer polypeptide is increased as compared to the expression in a control plant, or not, can be determined by well-known methods. The terms “increase”, “improve” or “enhance” are interchangeable and mean an increase of expression of at least 20%, more preferably at least 40%, and most preferably at least 60% in comparison to a control plant as defined herein. Preferably, said increase in expression is during spike development as set forth elsewhere herein. Moreover, said increase may be at least during (the early phases of) pollen development and meiosis, such as in anther or, more specifically, tapetum, or developing microspores.

Restoration capacity, as used herein, means the capacity of a plant to restore fertility in the progeny of a cross with a G-type cytoplasmic male sterility (“CMS”) line. Whether a plant has an increased restoration capacity for wheat G-type cytoplasmic male sterility (“CMS”) compared to a control can be assessed by well-known methods. For example, the plant promoter or chimeric gene of the present invention of the invention might be introduced into a cereal (wheat) plant having G-type CMS, or in a (wheat) plant lacking G-type CMS which is then crossed with a G-type cytoplasmic male sterile (wheat) plant and evaluating seed set in the progeny. The number of set seed is indicative for the restoration capacity of the plant. A seed set which is at least 10%, at least 20% or at least 30% higher than the seed set in the control plant is considered to be indicative for an increased restoration capacity.

Moreover, pollen accumulation and pollen viability can be quantified in order to assess the restoration capacity. The promoter modifications described herein lead to higher numbers of viable pollen (in plants with G-type CMS).

Moreover, the present invention relates to a method for identifying and/or selecting a cereal plant having increased expression of a functional restorer polypeptide for wheat G-type cytoplasmic male sterility and/or increased restoration capacity for wheat G-type cytoplasmic male sterility, said method comprising the steps of:
a) identifying or detecting in said plant the presence of the plant promoter or the chimeric nucleic acid molecule of the present invention, and
b) selecting said plant comprising said plant promoter or chimeric nucleic acid molecule.

The present invention further relates to a method for producing hybrid seed, comprising the steps of:
a) providing a male cereal parent plant, such as a wheat plant, produced according to the method of the present invention and/or comprising the plant promoter or the chimeric nucleic acid molecule of the present invention, wherein the promoter or chimeric nucleic acid molecule is preferably present in homozygous form, and wherein the promoter is operably linked to a functional restorer gene for wheat G-type cytoplasmic male sterility
b) providing a female cereal parent plant that is a G-type cytoplasmic male sterile cereal plant,
c) crossing said female cereal parent plant with a said male cereal parent plant; and optionally,
d) harvesting seeds.

As used herein, the term “homozygous” means a genetic condition existing when two identical alleles reside at a specific locus, but are positioned individually on corresponding pairs of homologous chromosomes in the cell. Conversely, the term “heterozygous” means a genetic condition existing when two different alleles reside at a specific locus, but are positioned individually on corresponding pairs of homologous chromosomes in the cell.

Also provided herein is a G-type CMS restorer gene promoter for use in wheat (such as a Rf1 or Rf3 gene promoter used in wheat), comprising a heterologous or a duplicated EIL3 and/or PHD transcription factor binding site as described herein (e.g., the PHD transcription factor binding site having a nucleotide sequence as shown in SEQ ID NO: 10, SEQ ID NO 11, SEQ ID NO: 40, SEQ ID NO: 31, SEQ ID NO: 32, SEQ ID NO: 38, SEQ ID NO: 42, SEQ ID NO: 41 or SEQ ID NO: 12, or the EIL3 transcription factor binding site having a nucleotide sequence as shown in SEQ ID NO: 19 or SEQ ID NO: 39, or said sequences wherein 1, 2, or 3 nucleotides have been deleted or substituted), and a wheat cell or plant or seed containing it. In one embodiment, this promoter (and cell, plant or seed) comprises 2, 3 or 4 of said EIL3 and/or PHD transcription factor binding sites. In one embodiment, this promoter (and wheat cell, plant or seed) comprises 2, 3 or 4 of said EIL3 and PHD transcription factor binding sites.

The present invention further relates to the use of the plant promoter or the chimeric nucleic acid molecule of the present invention for the identification of a plant comprising said functional restorer gene allele for wheat G-type cytoplasmic male sterility.

The present invention further relates to the use of a plant of the present invention or a plant obtained or obtainable by the method of the present invention for restoring fertility in a progeny of a G-type cytoplasmic male sterile cereal plant, such as a wheat plant.

The present invention further relates to the use of a plant of the present invention or a plant obtained or obtainable by the method of the present invention for producing hybrid seed or a population of hybrid cereal plants, such as wheat seed or plants.

The present invention further relates to the use of at least one heterologous binding site for an EIL3 transcription factor and/or at least one heterologous binding site for a PHD transcription factor for increasing the activity of a plant promoter in developing spikes.

The present invention further relates to the use of the plant promoter of the present invention for increasing expression of a nucleic acid molecule of interest in a plant, wherein the plant promoter is operably linked to the nucleic acid molecule of interest. Preferably, expression is increased in developing spikes.

As used herein (in any one of the aspects of this invention, or in any combinations as described), the term “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a nucleic acid which is functionally or structurally defined, may comprise additional DNA regions etc.

### Section A: Embodiments of the First Aspect of the Present Invention (Promoter with EIL3 and/or PHD Transcription Factor Binding Site(s))

The methods, promoters, plants, constructs, uses etc. as described in section A are further illustrated by the following embodiments and combinations of embodiments as indicated by the respective dependencies and back-references. The definitions and explanations given herein above apply mutatis mutandis to the following embodiments.
1. A method for producing a plant promoter having increased activity in the presence of an EIL3 (Ethylene insensitive 3-like) transcription factor and/or a PHD (Plant Homeodomain) transcription factor, comprising the steps of
   a) providing a plant promoter, and
   b1) introducing at least one binding site for the EIL3 transcription factor and/or at least one binding site for the PHD transcription factor into the plant promoter, and/or
   b2) modifying at least one existing binding site for the EIL3 transcription factor and/or at least one existing binding site for the PHD transcription factor in the promoter such that binding of the EIL3 or PHD transcription factor to said binding site is improved.
2. The method of embodiment 1, wherein, in step b1), at least one binding site for the EIL3 transcription factor and at least one binding site for said PHD transcription factor are introduced into the plant promoter.
3. The method of embodiment 1 or 2, wherein the plant promoter has increased activity in developing spikes, such as in Zadok stages Z39-Z41, Z45-Z48, Z50-Z59, and/or Z60-Z69.
4. The method of any one of embodiments 1 to 3, wherein, in step b1), the at least one binding site is introduced into the plant promoter by genome editing.
5. The method of any one of embodiments 1 to 4, wherein, in step b2), the at least one binding site is modified by chemical mutagenesis, by irradiation induced mutagenesis, or by somatic embryogenesis/mutagenesis.
6. The method of embodiment 1 or 5, wherein the promoter provided in step a) is a wheat promoter.
7. The method of any one of embodiments 1 to 6, wherein the promoter provided in step a) is a promoter of a functional restorer gene for wheat cytoplasmic male sterility, such as for wheat K-type or G-type cytoplasmic male sterility
8. The method of embodiment 7, wherein the promoter is the promoter of an Rf1 or Rf3 gene.
9. The method of embodiment 8, wherein the promoter comprises a sequence as shown in SEQ ID NO: 23, SEQ ID NO:36 or SEQ ID NO: 37, or a variant thereof being at least 90% identical thereto.
10. The method of any of the preceding embodiments, wherein the binding site for the PHD transcription factor has a sequence as shown in SEQ ID NO: 10, SEQ ID NO 11, SEQ ID NO: 40, SEQ ID NO: 31, SEQ ID NO: 32, SEQ ID NO: 38, SEQ ID NO: 42, SEQ ID NO: 41 or SEQ ID NO: 12, or is a variant thereof.
11. The method of any of the preceding embodiments, wherein the binding site for the EIL3 transcription factor has a sequence as shown in SEQ ID NO: 19 or SEQ ID NO: 39, or is a variant thereof.
12. The method of any one of the preceding embodiments, wherein at least two EIL3 and/or PHD transcription factor binding sites are introduced into the promoter, such as at least three or four binding sites.
13. The method of any one of the preceding embodiments, wherein a fragment having a sequence as shown in SEQ ID NO: 29 is introduced into the promoter provided in step a).
14. The method of any of the preceding embodiments, wherein the EIL3 transcription factor, when used in the construction of a phylogenetic tree, clusters with the Oryza sativa EIL4 transcription factor.
15. The method of any one of the preceding embodiments, wherein the EIL3 transcription factor comprises:
   a) an amino acid sequence as shown in SEQ ID NO: 13; or
   b) an amino acid sequence being at least 50%, 60%, 70%, 75%, 80%, 85%; 86%; 87%; 88%; 89%; 90%; 91%; 92%; 93%; 94%; 95%; 96%; 97%; 98%; or 99% identical to SEQ ID NO: 13.
16. The method of any one of the preceding embodiments, wherein the sequence of the PHD transcription factor, when used in the construction of a phylogenetic tree, clusters with the sequence of the rice transcription factor Os02g0147800.
17. The method of any one of the preceding embodiments, wherein the PHD transcription factor comprises:
   a) an amino acid sequence as shown in SEQ ID NO: 4; or
   b) an amino acid sequence being at least 50%, 60%, 70%, 75%, 80%, 85%; 86%; 87%; 88%; 89%; 90%; 91%; 92%; 93%; 94%; 95%; 96%; 97%; 98%; or 99% identical to SEQ ID NO: 4.
18. A plant promoter obtained or obtainable by the method of any one of the preceding embodiments.
19. A plant promoter comprising at least one heterologous binding site for an EIL3 transcription factor and/or at least one heterologous binding site for a PHD transcription factor.
20. The plant promoter of embodiment 18 or 19, wherein the plant promoter is operably linked to a nucleic acid of interest.
21. A chimeric nucleic acid molecule comprising the following operably linked elements
   a) the plant promoter of embodiment 18 or 19;
   b) a nucleic acid molecule of interest; and optionally
   c) a transcription termination and polyadenylation region functional in plant cells.
22. The plant promoter of embodiment 20 or the chimeric molecule of embodiment 21, wherein the nucleic acid molecule of interest encodes a functional restorer polypeptide for wheat cytoplasmic male sterility.
23. A cereal plant cell, a cereal plant or seed thereof, such as a wheat plant cell, wheat plant or seed thereof, comprising the plant promoter of any one of embodiments 18 to 20 or 22, or the chimeric nucleic acid molecule of embodiment 21 or 22.
24. The cereal plant cell, plant or seed of embodiment 22, which is a hybrid plant cell, plant or seed.
25. A method for producing a cereal plant cell or plant or seed thereof, such as a wheat plant cell or plant or seed thereof, comprising the step of providing said plant cell or plant with the plant promoter of any one of embodiments 18 to 20 or 22, or the chimeric nucleic acid molecule of embodiment 21 or 22.
26. A method for increasing expression of a functional restorer polypeptide for wheat G-type cytoplasmic male sterility, or for increasing restoration capacity for wheat G-type cytoplasmic male sterility (“CMS”) in a cereal plant, such as a wheat plant, comprising the step of providing said plant cell or plant with the plant promoter of any one of embodiment 18 to or 22 or the chimeric nucleic acid molecule of embodiment 21 or 22.
27. A method for identifying and/or selecting a cereal plant having increased expression of a functional restorer polypeptide for wheat G-type cytoplasmic male sterility and/or in creased restoration capacity for wheat G-type cytoplasmic male sterility, said method comprising the steps of:
   a) identifying or detecting in said plant the presence of the plant promoter or the chimeric nucleic acid molecule of embodiment 22, and
   b) selecting said plant comprising said plant promoter or chimeric nucleic acid molecule.
28. A method for producing hybrid seed, comprising the steps of:
   a) providing a male cereal parent plant, such as a wheat plant, according to embodiment 23 and/or providing a male cereal parent plant, such as a wheat plant, comprising the plant promoter or the chimeric nucleic acid molecule of embodiment 22, wherein said nucleic acid molecule or chimeric gene is preferably present in homozygous form,
   b) providing a female cereal parent plant that is a G-type cytoplasmic male sterile cereal plant,
   c) crossing said female cereal parent plant with a said male cereal parent plant; and optionally,
   d) harvesting hybrid seeds on said female cereal parent plant.
29. Use of the plant promoter or the chimeric nucleic acid molecule of embodiment 22 for the identification of a plant comprising said functional restorer gene allele for wheat G-type cytoplasmic male sterility.
30. Use of a plant according to embodiment 23 or 24, or a plant obtained or obtainable by the method of embodiment 25 for restoring fertility in a progeny of a cytoplasmic male sterile cereal plant, such as a K-type or G-type cytoplasmic male sterile cereal plant wheat plant.
31. Use of a plant according to embodiment 23 or 24, or a plant obtained or obtainable by the method of embodiment 25 for producing hybrid seed or a population of hybrid cereal plants, such as wheat seed or plants.
32. Use of at least one heterologous binding site for an EIL3 transcription factor and/or at least one heterologous binding site for a PHD transcription factor for increasing the activity of a plant promoter in developing spikes.
33. Use of the plant promoter of any one of embodiments 18 to 20 or 22 for increasing expression of a nucleic acid molecule of interest in a plant, wherein the plant promoter is operably linked to the nucleic acid molecule of interest.
34. The use of embodiment 33, wherein expression is increased in developing spikes, such as in Zadok stages Z39-Z41, Z45-Z48, Z50-Z59, and/or Z60-Z69.
35. A wheat G-type CMS fertility restorer gene promoter, such as a Rf1 or Rf3 gene promoter expressing the Rf1 or Rf3 fertility restorer protein in wheat, comprising a heterologous or a duplicated EIL3 and/or PHD transcription factor binding site.
36. The promoter of embodiment 35, wherein said PHD transcription factor binding site comprises the nucleotide sequence of SEQ ID NO: 10, SEQ ID NO 11, SEQ ID NO: 40, SEQ ID NO: 31, SEQ ID NO: 32, SEQ ID NO: 38, SEQ ID NO: 42, SEQ ID NO: 41 or SEQ ID NO: 12, or such a sequence wherein 1, 2, or 3 nucleotides have been deleted or substituted; and said EIL3 transcription factor binding site comprises the sequence of SEQ ID NO: 19 or SEQ ID NO: 39, or such a sequence wherein 1, 2, or 3 nucleotides have been deleted or substituted.
37. A wheat cell or plant or seed containing the promoter of embodiment 35 or 36.
38. The promoter of embodiment 35 or 36 or the cell, plant or seed of embodiment 37, comprising 2, 3 or 4 of said EIL3 and/or PHD transcription factor binding sites.
39. The promoter of embodiment 35 or 36 or the cell, plant or seed of embodiment 37, comprising 2, 3 or 4 of said EIL3 and PHD transcription factor binding sites, such as SEQ ID NO: 29.
40. The method, use, plant, cell or promoter of any of the above embodiments, where said promoter also comprises a cereal, such as a wheat, enhancer sequence, capable of increasing expression of said promoter, such as an enhancer sequence which does not comprise a PHD or EIL3 transcription factor binding site.

### SECTION B (Promoters with Heterologous Enhancer Element(s))

### Brief Summary of the First Aspect of the Second Aspect of the Present Invention (Section B)

In a second aspect, the present invention provides a method for increasing expression conferred by a plant promoter of a functional restorer gene for wheat cytoplasmic male sterility, comprising introducing at least one nucleic acid expression enhancing nucleic acid (NEENA) molecule into said promoter.

In a preferred embodiment of the second aspect of the present invention, said at least one NEENA molecule
i) comprises (or consists of) a nucleic acid sequence as shown in SEQ ID NO: 70, 86, 87, 90 or 91, in particular as shown in SEQ ID NO: 70
ii) comprises (or consists of) a nucleic acid sequence with an identity of at least 90%, at least 91%, at least 92%, at least 93%, at least 94%, at least 95%, at least 96%, at least 97%, at least 98%, or at least 99% to SEQ ID NO: 70, 86, 87, 90 or 91, in particular to SEQ ID NO: 70,
iii) comprises (or consists of) a fragment of at least 30, at least 40, in particular at least 50, at least 80, at least 100 or at least 120 consecutive bases of a nucleic acid molecule of i) or ii), or
iv) is the complement or reverse complement of any of the previously mentioned nucleic acid molecules under i) to iii),
wherein the nucleic acid molecule of ii), iii) and iv) is capable of increasing expression conferred by the plant promoter of the functional restorer gene for wheat cytoplasmic male sterility.

The at least one NEENA molecule is preferably introduced into a promoter of a functional restorer gene for wheat cytoplasmic male sterility, in particular for wheat G-type cytoplasmic male sterility.

The second aspect of the present invention is also directed to a plant promoter obtained or obtainable by the above method of the present invention. In a preferred embodiment, said promoter wherein such at least one NEENA/enhancer above is introduced is selected from
a) a promoter comprising a nucleic acid sequence as shown in SEQ ID NO: 23, 36, 37, 73 or 74, in particular SEQ ID NO: 23
b) a fragment of the nucleic acid sequence shown in SEQ ID NO: 23, 36, 37, 73 or 74, in particular SEQ ID NO: 23
c) a variant of the promoter of a) or the fragment of b), said fragment or variant having a sequence being at least 80%, 85%; 86%; 87%; 88%; 89%; 90%; 91%; 92%; 93%; 94%; 95%; 96%; 97%; 98%; or 99% identical to the sequence of a) or b).

In particular, the second aspect of the present invention is directed to a promoter comprising a promotor of a functional restorer gene for wheat cytoplasmic male sterility functionally linked to at least one nucleic acid expression enhancing nucleic acid (NEENA) molecule as set forth above.

Preferably, the wheat cytoplasmic male sterility when referred to in this invention is G-type or K-type cytoplasmic male sterility (in particular wheat G-type cytoplasmic male sterility).

The promoter according to the second aspect may further comprise at least one modified binding site for an EIL3 transcription factor and/or at least one modified binding site for a PHD transcription factor as defined in Section A. In particular, the promoter according the second aspect may further comprise at least one heterologous binding site for an EIL3 transcription factor and/or at least one heterologous binding site for a PHD transcription factor as defined in the previous section, i.e. in Section A. All definitions and explanations apply accordingly.

The plant promoter according to the second aspect is a modified promoter of a functional restorer gene for wheat G-type cytoplasmic male sterility, e.g., for an Rf1 or Rf3 gene. Specifically, the promoter has been modified by introducing the at least one nucleic acid expression enhancing nucleic acid (NEENA) molecule as set forth above into said promoter (and optionally at least one heterologous binding site for a EIL3 transcription factor and/or at least one heterologous binding site for a PHD transcription factor into said promoter).

Preferably, the promoter according to the second aspect of the present invention is operably linked to a nucleic acid molecule encoding a functional restorer polypeptide for wheat cytoplasmic male sterility.

Preferably, the at least one NEENA molecule, and optionally the transcription factor binding site(s) as set forth above is (are) introduced into the promoter by genome editing.

Furthermore, the second aspect of the invention relates to a chimeric nucleic acid molecule comprising the following operably linked elements
a) the plant promoter according to the second aspect of the present invention;
b) a nucleic acid molecule of interest; and optionally
c) a transcription termination and polyadenylation region functional in plant cells.

In a preferred embodiment, the nucleic acid molecule of interest under b) encodes a functional restorer polypeptide for wheat cytoplasmic male sterility, such as for wheat G-type or K-type cytoplasmic male sterility. In an embodiment, the nucleic acid molecule of interest is the nucleic acid molecule as defined in Section C herein, i.e. the nucleic acid molecule encoding a functional restorer polypeptide for wheat G-type cytoplasmic male sterility, wherein said nucleic acid molecule comprises a mutated miRNA binding site in the coding sequence. For example, the nucleic acid molecule may be the nucleic acid molecule of any one embodiments 1 to 11 in Section C. The embodiments can be found at the end of Section C. This nucleic acid of interest can then be operably linked to the above promoter comprising the enhancer of the section B, with or without at least one added or modified binding site for an EIL3 transcription factor and/or at least one added or modified binding site for a PHD transcription factor as defined in the previous section, i.e. in Section A.

The second aspect of the present invention is further directed to a plant cell, plant or seed, such as a cereal plant cell, plant or seed, comprising the plant promoter of the second aspect of the present invention or the chimeric nucleic acid molecule of this aspect. In an embodiment, the cereal plant cell, plant or seed is a wheat plant cell, plant or seed.

The second aspect of the present invention further pertains to a method for producing a plant cell or plant or seed thereof, such as a cereal plant cell or plant or seed thereof, comprising the step of providing said plant cell or plant with the plant promoter or the chimeric nucleic acid molecule of the second aspect of the invention.

The second aspect of the present invention also relates to a method for increasing expression of a functional restorer polypeptide for wheat G-type cytoplasmic male sterility, or for increasing restoration capacity for wheat G-type cytoplasmic male sterility (“CMS”) in a cereal plant, such as a wheat plant, comprising the step of providing said plant cell or plant with the plant promoter or the chimeric nucleic acid molecule of the second aspect of the invention.

Moreover, the second aspect of the present invention relates to a method for identifying and/or selecting a cereal plant having increased expression of a functional restorer polypeptide for wheat G-type cytoplasmic male sterility and/or increased restoration capacity for wheat G-type cytoplasmic male sterility, said method comprising the steps of:
a) identifying or detecting in said plant the presence of the plant promoter or the chimeric nucleic acid molecule of the second aspect of the present invention, and
b) selecting for a plant comprising said plant promoter or chimeric nucleic acid molecule.

The second aspect of the present invention further relates to a method for producing hybrid seed, comprising the steps of:
a) providing a i) male cereal parent plant, such as a wheat plant, produced according to the method of the second aspect of the present invention and/or ii) a male cereal parent plant, such as a wheat plant, comprising the plant promoter or the chimeric nucleic acid molecule of the present invention, wherein said promoter or chimeric nucleic acid molecule is preferably present in homozygous form,
b) providing a female cereal parent plant, such as a wheat plant, that is a G-type cytoplasmic male sterile cereal plant,
c) crossing said female cereal parent plant with a said male cereal parent plant; and optionally,
d) harvesting hybrid seeds from said female parent plant.

The second aspect of the present invention further relates to the use of the plant promoter or the chimeric nucleic acid molecule of the second aspect of the present invention for the identification of a plant comprising said functional restorer gene allele for wheat G-type cytoplasmic male sterility.

The second aspect of the present invention further relates to the use of a plant of the second aspect of the present invention or a plant obtained or obtainable by the method of the second aspect of the present invention for restoring fertility in a progeny of a cytoplasmic male sterile cereal plant, such as a G-type or K-type cytoplasmic male sterile wheat plant.

The second aspect of the present invention further relates to the use of a plant of the second aspect of the present invention or a plant obtained or obtainable by the method of the second aspect of the present invention for producing hybrid seed or a population of hybrid cereal plants, such as wheat seed or plants.

The second aspect of the present invention further relates to the use of at least one nucleic acid expression enhancing nucleic acid (NEENA) molecule as defined above, and optionally of at least one heterologous binding site for an EIL3 transcription factor and/or at least one heterologous binding site for a PHD transcription factor for increasing the activity conferred by a plant promoter of a functional restorer gene for wheat cytoplasmic male sterility, for example in developing spikes.

The second aspect of the present invention further relates to the use of the plant promoter of the second aspect of the present invention for increasing expression conferred by a plant promoter of a functional restorer gene for wheat cytoplasmic male sterility. In some embodiment, expression is increased in developing spikes.

### Detailed Description of the Second Aspect of the Present Invention (Section B)

As set forth in Section A, the Rf3-58 gene is a functional restorer gene for wheat G-type cytoplasmic male sterility used in wheat hybrid breeding. Increased expression levels of Rf3-58 gene leads to better restoration of the fertility in the progeny of a cross with a G-type cytoplasmic male sterility (“CMS”) line.

In the studies underlying the present invention, the inventors have found that the introduction of certain enhancer elements into the promoter allows for increasing expression of the restorer gene (see Example 11). The strongest effect was observed for the enhancer designated “EN1390” (SEQ ID NO: 70). Notably, the effect was seen at various positions with the tested promoter. Further, the effect was largely independent of the orientation of the enhancer (see Example 11).

Advantageously, the introduction of the EN1390 enhancer in the Rf3-58 promoter improved restoration capacity of Rf3 (see Example 12).

In summary, the results described in the Examples section show that enhancer sequences such as the EN1390 enhancer sequence could be used for engineering plant promoters having increased activity.

Engineered plant promoters according to the present invention would thus have increased activity in plant tissues and/or at developmental stages in which the EIL3 transcription factor and/or the PHD transcription factor is (are) abundant, such as in developing spikes.

Accordingly, a second aspect of the present invention relates to a method for increasing expression conferred by a plant promoter of a functional restorer gene for wheat cytoplasmic male sterility, comprising introducing at least one nucleic acid expression enhancing nucleic acid (NEENA) molecule into said promoter, wherein said at least one NEENA molecule
i) comprises (or consists of) a nucleic acid sequence as shown in SEQ ID NO: 70, 86, 87, 90 or 91,
ii) comprises (or consists of) a nucleic acid sequence with an identity of at least 90%, at least 91%, at least 92%, at least 93%, at least 94%, at least 95%, at least 96%, at least 97%, at least 98%, or at least 99% to SEQ ID NO: 70, 86, 87, 90 or 91,
iii) comprises (or consists of) a fragment of at least 30, at least 40, in particular at least 50 consecutive bases of a nucleic acid molecule of i) or ii), or
iv) is the complement or reverse complement of any of the previously mentioned nucleic acid molecules under i) to iii),
wherein the nucleic acid molecule of ii), iii) and iv) is capable of increasing expression conferred by the plant promoter of the functional restorer gene for wheat cytoplasmic male sterility.

The NEENA molecule is herein also referred to as an enhancer.

In accordance with the above method of the present invention, a promoter is produced having increased promoter activity. Preferably, the activity of the promoter is increased as compared to the activity of a control promoter. Typically, the control promoter does not comprise the modification(s) described herein, i.e. the enhancer.

Preferably, the activity of the promoter of the second aspect of the present invention is increased, by at least 50%, by at least 100%, by at least 200% or by at least 300% as compared to the control promoter. In one embodiment, the activity of the promoter of the first or second aspect of the present invention is increased between 50% and 300%, between 50% and 200%, or between 50% to 100%, such as between 100% and 300%, or between 100% and 200%, as compared to the control promoter (as measured using standard methods, such as those exemplified below to measure expression). In one embodiment, the activity of the promoter of the first or second aspect of the present invention is increased in such a way that 1 Rf gene with such promoter provides for full restoration for wheat G-type CMS, such as Rf1 or Rf3 with such improved promoter.

Whether the activity of a promoter is increased, or not, can be assessed by the skilled person without further ado and as described in Section A above. Preferably, the resulting promoter, preferably, has increased activity in developing spikes (e.g., of cereal plants, preferably wheat plants). More preferably, the produced promoter has increased activity in early spike development. Most preferably, the resulting promoter has increased activity in developing spikes at Zadok stages Z39-Z41 (tetrad phase), Z45-Z48 (uninucleate phase), Z50-Z59 (binucleate phase), and/or Z60-Z69 (trinucleate phase). Accordingly, the second aspect of present invention also relates to a method for producing a plant promoter having increased activity at the aforementioned stages. Further preferred stages are described in Section A.

The promoter to be modified according to the above method, shall be the promoter of a functional restorer gene for cytoplasmic male sterility. In particular, the promoter is a promoter of a functional restorer gene for wheat G-type or K-type cytoplasmic male sterility (or a variant thereof). Such promoters are described in detail in Section A. The definitions apply accordingly.

In an embodiment of the above method, the promoter is preferably a promoter of a functional restorer gene for wheat G-type cytoplasmic male sterility selected from the group consisting of an Rf1 gene, an Rf2 gene, an Rf3 gene, an Rf4 gene, an Rf5 gene, an Rf6 gene, an Rf7 gene, an Rf8 gene and an Rf9 gene.

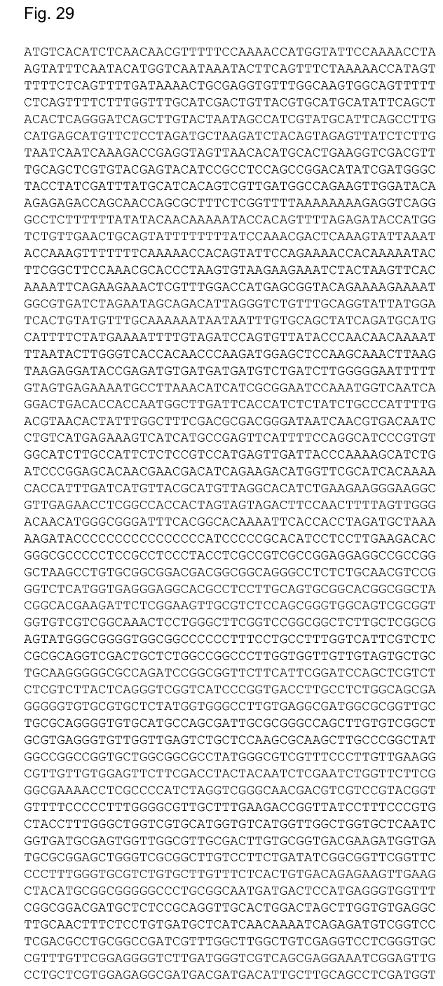

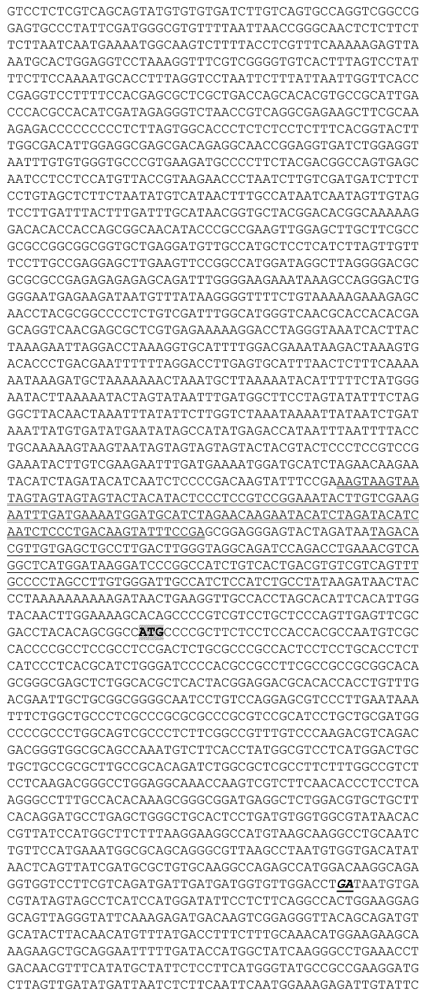

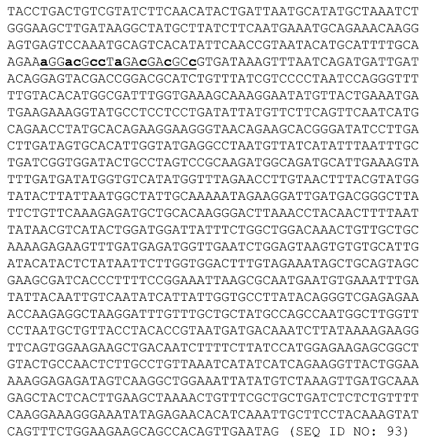

In particular, the promoter is the promoter of an Rf3 gene, such as the promoter of the Rf3-58 gene (or the promoter of the Rf3 allele in cultivar Fielder, as shown in SEQ ID NO: 94) or the promoter of the Rf3-29a gene (or a variant thereof). SEQ ID NO: 94 is the native Fielder sequence which was used for the modifications described in FIG. 29 (the 2 nt as indicated in FIG. 29 (in bold, underlining and italics) are to be introduced in this sequence to repair a frameshift and get a functioning Rf3 coding sequence)—such repaired Fielder coding sequence and gene sequence is included in this invention.

Alternative, the promoter is the promoter of an Rf1 gene, such as the promoter of the Rf1-09 gene (or a variant thereof).

Accordingly, the promoter of the Rf3-58 gene, preferably, comprises the following sequence:
a) a promoter comprising a nucleic acid sequence as shown in SEQ ID NO: 23, 73, or 74
b) a fragment of the nucleic acid sequence shown in SEQ ID NO: 23, 73 or 74
c) a variant of the promoter of a) or the fragment of b), said fragment or variant having a sequence being at least 80%, 85%; 86%; 87%; 88%; 89%; 90%; 91%; 92%; 93%; 94%; 95%; 96%; 97%; 98%; or 99% identical to the sequence of a) or b).

Preferably, the promoter of the Rf3-29a gene comprises the following sequence:
a) a promoter comprising a nucleic acid sequence as shown in SEQ ID NO: 36,
b) a fragment of the nucleic acid sequence shown in SEQ ID NO: 36, such as SEQ ID NO: 34,
c) a variant of the promoter of a) or the fragment of b), said fragment or variant having a sequence being at least 80%, 85%; 86%; 87%; 88%; 89%; 90%; 91%; 92%; 93%; 94%; 95%; 96%; 97%; 98%; or 99% identical to the sequence of a) or b).

Accordingly, the promoter of the Rf1-09 gene comprises the following sequence:
a) a promoter comprising a nucleic acid sequence as shown in SEQ ID NO: 37,
b) a fragment of the nucleic acid sequence shown in SEQ ID NO: 37, such as SEQ ID NO: 35,
c) a variant of the promoter of a) or the fragment of b), said fragment or variant having a sequence being at least 80%, 85%; 86%; 87%; 88%; 89%; 90%; 91%; 92%; 93%; 94%; 95%; 96%; 97%; 98%; or 99% identical to the sequence of a) or b).

As described in section A, the fragment under b) or the variant under c) has essentially the same promoter activity of the promoter under a). A promoter activity of at least 80%, at least 90%, or at least 95% or at least 98% is considered to be essentially the same promoter activity.

Preferably, the fragment under b) has a length of at least 200 bp, at least 250 bp, at least 300 bp, at least 400 bp, at least 500 bp, at least 750 bp, at least 1000 bp, at least 1500 bp, or at least 2000 bp.

How to determine the degree of sequence identity is described in Section A.

Typically, in one embodiment of section B, the promoter to be modified also comprises at least one EIL3 and/or at least one PHD binding site(s), such as a heterologous or added (such as a duplicated or triplicated) EIL3 or PHD binding site, or a modified EIL3 or PHD site with improved binding by its' transcription factor. Preferably, the EIL3 and PHD binding sites are not disrupted by the introduction of the at least one nucleic acid expression enhancing nucleic acid (NEENA) molecule (herein also referred to as “enhancer element”) into said binding sites.

The term “functional linkage” or “functionally linked” is to be understood as meaning, for example, the sequential arrangement of a regulatory element (e.g., a promoter) or (a) further regulatory elements (such as e.g., NEENA and/or the transcription factor binding site(s)) in such a way that each of the regulatory elements can fulfill its intended function to allow, modify, facilitate or otherwise influence expression conferred by the promoter, preferably increase expression of the promoter, particularly in spike tissue.

Preferably, the at least one NEENA molecule is introduced at one or more positions within 1000 bp, such as within 500 bp or within 300 bp upstream (i.e., 5′) to the translation start codon of the gene that is operably linked to said promoter. More preferably, the at least one NEENA molecule is introduced at a position within 250 to 80 bp upstream (5′) to the translation start codon of the gene that is operably linked to said promoter. Most preferably, the at least one NEENA molecule is introduced at a position within 200 to 100 bp, 110 to 150, 120 to 140, or within 125 to 135 bp, or within 125 to 130 bp, upstream (5′) of the translation start site of the gene that is operably linked to said promoter, such as at a position 126, 127, 128 or 129 nt upstream of the translation start site. Thus, the modified promoter comprises one or more NEENA molecule at one of more of these positions.

Also, the one or more NEENA molecules are introduced (i.e., are present) at position −127 (minus 127), −128, −190, −83, −76, −70, −64 relative to the translation start codon, e.g. to the start codon of the Rf3-58 promoter. In particular, the one or more NEENA molecules are introduced (i.e., are present) at position −127 or −128 relative to the translation start codon, e.g. to the start codon of the Rf3 promoter, such as the Rf3-58 or Rf3-29a promoter, or the promoter of the Fielder Rf3 allele (that promoter is the sequence upstream of the ATG translation start site in SEQ ID NO: 94). For example, the start codon of the Rf3 variant in Fielder is shown in the sequence (of the repaired and edited Rf3 sequence) in FIG. 29, and the start codon in Rf3-58 is nt 1-3 of SEQ ID NO: 43, and the start codon in Rf3-29a is nt 1-3 of SEQ ID NO: 62, and the start codon in Rf1-09 is nt 1-3 of SEQ ID NO: 64. The nucleotide preceding the A in the start codon has position “minus 1” (−1).

The introducing of the at least one NEENA molecule, and optionally of the at least one binding site for the EIL3 transcription factor and/or the at least one binding site for the PHD transcription factor can be done by any method deemed appropriate, in particular by the methods as described in section A. Further, Example 12 describes the insertion of the EN1390 enhancer in the Rf3-58 promoter by genome editing. The fragment is flanked by Cas9 target sites so that it could be duplicated in the wheat genome using a Cas9 nuclease or nickase and sgRNAs targeting these sites.

In a preferred embodiment of the above method of the present invention, the at least one binding site is introduced into the plant promoter by genome editing. Thus, the introduction is carried out in a plant cell.

By using such technologies, plant promoters can be converted to plant promoters having at least one heterologous enhancer element, thereby increasing the expressing of the gene that is operably linked to the promoter, preferably in developing spikes. If the modified promoter is the promoter of an Rf gene, such as of an Rf3 or Rf1 gene, restoration capacity for wheat G-type cytoplasmic male sterility (“CMS”) in a cereal plant can be improved.

The introduction is, however, not limited to genome editing. Rather, the step could be carried out by conventional cloning methods or by gene synthesis methods. A promoter generated by such methods could be introduced into a plant by transformation.

According to above method of the present invention, the following element or elements are introduced into the plant promoter:
i) at least one nucleic acid expression enhancing nucleic acid (NEENA) molecule as defined above,
ii) and optionally at least one heterologous binding site for the EIL3 transcription factor and at least one heterologous binding site for the PHD transcription factor (as defined in Section A in more detail).

The term “at least one” as used herein, preferably, means one or more than one. For example, two or three (NEENA) molecules are introduced.

The definitions and explanations provided herein above, preferably, apply mutatis mutandis to the plant promoter, chimeric nucleic acid molecule, cereal plant cell, cereal plant, seed, method or use of the second aspect of the present invention.

The second aspect of present invention also concerns a plant promoter obtained or obtainable by the above method of the present invention.

The second aspect present invention is further directed to a promoter comprising a promotor of a functional restorer gene for wheat cytoplasmic male sterility functionally linked to at least one nucleic acid expression enhancing nucleic acid (NEENA) molecule having a sequence as shown above. In a preferred embodiment, the promoter further comprises at least one heterologous binding site for an EIL3 transcription factor and/or at least one heterologous binding site for a PHD transcription factor.

In an embodiment, the term “heterologous” in connection with NEENA molecule, preferably, means that the molecule is not naturally present at the position at which the molecule is present.

Preferably, the plant promoter of the second aspect of the present invention is operably linked to a nucleic acid of interest. More preferably, the plant promoter of the second aspect of the present invention is operably linked to a nucleic acid molecule that encodes a functional restorer polypeptide for wheat G-type cytoplasmic male sterility, such as a naturally occurring nucleic acid molecule or a modified nucleic acid molecule. Most preferably, the promotor is operably linked to of a functional restorer gene for wheat cytoplasmic male sterility according to the third aspect of the present invention (with a modified miRNA binding site, as defined in Section C in more detail), with or without the heterologous or added (such as a duplicated or triplicated) EIL3 and/or PHD binding site, or a modified EIL3 and/or PHD site with improved binding by its' transcription factor, according to section A of this invention.

Furthermore, the invention relates to a chimeric nucleic acid molecule comprising the following operably linked elements
a) the plant promoter of the second aspect of the present invention;
b) a nucleic acid molecule of interest; and optionally
c) a transcription termination and polyadenylation region functional in plant cells.

In a preferred embodiment, the nucleic acid molecule under b) encodes a functional restorer polypeptide for wheat cytoplasmic male sterility, such as for G-type or K-type cytoplasmic male sterility. Said nucleic acid molecule may be a naturally occurring nucleic acid molecule or a modified nucleic acid molecule. Most preferably, the nucleic acid molecule of interest is the nucleic acid molecule encoding the functional restorer gene for wheat cytoplasmic male sterility according to the third aspect of the present invention (with a modified miRNA binding site, as defined in Section C).

The plant promoter of the second aspect of the present invention or the chimeric gene of the second aspect of the present invention may be present in a plant.

Thus, the invention is further directed to a plant cell, plant or seed thereof, such as a cereal plant cell or plant or seed thereof, comprising the plant promoter of the second aspect of the present invention or the chimeric nucleic acid molecule of the second aspect of the present invention. Preferably, the cereal plant cell, plant or seed thereof is a wheat plant cell, plant or seed thereof.

In one embodiment the plant cell, the plant or seed of the present invention, is a hybrid plant cell, plant or seed.

Preferred cereal plants are disclosed in Section A.

The plant promoter or chimeric gene of the second aspect of the present invention may be introduced into a plant by any method deemed appropriate. Preferred methods are described in Section A.

In an embodiment, the plant has been generated by genome editing (as described above).

In one aspect, the plant promoter or chimeric gene of the second aspect of the present invention is stably integrated into the cereal (e.g., wheat) genome.

In an embodiment, the plant, or plant cell of the second aspect of the present invention has not been obtained exclusively by an essentially biological process for the production of plants.

As described in Section A, the plants according to the second aspect of the invention can be used in a conventional breeding scheme to produce more plants with the same characteristics or to introduce the characteristic of the presence of the restorer gene according to the invention in other varieties of the same or related plant species, or in hybrid plants. The obtained plants can further be used for creating propagating material. Plants according to the invention can further be used to produce gametes, seeds, flour, embryos, either zygotic or somatic, progeny or hybrids of plants obtained by methods of the invention. Seeds obtained from the plants according to the invention are also encompassed by the invention. The term “plant” also encompasses the offspring/progeny of the plant of the present invention, provided that the offspring/progeny comprises the promoter obtained or obtainable by the method of the second aspect of the present invention.

The second aspect of the present invention further pertains to a method for producing a cereal plant cell or plant or seed thereof, such as a wheat plant cell or plant or seed thereof, comprising the step of providing said plant cell or plant with the plant promoter or the chimeric nucleic acid molecule of the second aspect of the present invention or introducing at least one nucleic acid expression enhancing nucleic acid (NEENA) molecule of the invention. The plant promoter or chimeric gene or transcription factor binding site of the second aspect of the present invention may be provided as described above.

As set forth elsewhere herein, the plant promoter is preferably operably linked to a functional restorer gene for wheat G-type cytoplasmic male sterility, such as an Rf1 or Rf3 gene. This allows for increasing expression of the said restorer gene during spike development, thereby increasing restoration capacity for wheat G-type cytoplasmic male sterility in a cereal plant.

The second aspect of the present invention therefore relates to a method for increasing expression of a functional restorer polypeptide for wheat G-type cytoplasmic male sterility, or for increasing restoration capacity for wheat G-type cytoplasmic male sterility (“CMS”) in a cereal plant, such as a wheat plant, comprising the step of providing said plant cell or plant with the plant promoter or the chimeric nucleic acid molecule of the invention, wherein the plant promoter is operably linked to functional restorer gene for wheat G-type cytoplasmic male sterility.

The plant, plant part, or plant cell of the second aspect of the present invention or produced by the method of the second aspect of the present invention has at least an increased expression of the gene that is operably linked to the modified promoter. Specifically, expression of the gene shall be increased as compared to the expression of the gene under control of the unmodified promoter.

The plant of the second aspect of the present invention, preferably, has an increased restoration capacity for wheat G-type cytoplasmic male sterility (CMS) as compared to a control plant. Alternatively or additionally, it has an increased expression of the functional restorer polypeptide for wheat G-type cytoplasmic male sterility as compared to a control plant.

The choice of suitable control plants is a routine part of an experimental setup and is described, e.g., in section A.

Whether the expression of the functional restorer polypeptide is increased as compared to the expression in a control plant, or not, can be determined by the methods described in section A. Preferable, said increase in expression is during spike development as set forth elsewhere herein. Moreover, said increase may be at least during (the early phases of) pollen development and meiosis, such as in anther or, more specifically, tapetum, or developing microspores.

Moreover, the present invention relates to a method for identifying and/or selecting a cereal plant having increased expression of a functional restorer polypeptide for wheat G-type cytoplasmic male sterility and/or increased restoration capacity for wheat G-type cytoplasmic male sterility, said method comprising the steps of:
a) identifying or detecting in said plant the presence of the plant promoter or the chimeric nucleic acid molecule of the second aspect present invention, and
b) selecting said plant comprising said plant promoter or chimeric nucleic acid molecule.

The present invention further relates to a method for producing hybrid seed, comprising the steps of:
a) providing a male cereal parent plant, such as a wheat plant, produced according to the method of the second aspect of the present invention and/or comprising the plant promoter or the chimeric nucleic acid molecule of the second aspect of present invention, wherein the promoter or chimeric nucleic acid molecule is preferably present in homozygous form, and wherein the promoter is operably linked to a functional restorer gene for wheat G-type cytoplasmic male sterility
b) providing a female cereal parent plant that is a G-type cytoplasmic male sterile cereal plant,
c) crossing said female cereal parent plant with a said male cereal parent plant; and optionally,
d) harvesting seeds.

The present invention further relates to the use of a plant of the second aspect of the present invention or a plant obtained or obtainable by the method of the second aspect of the present invention for restoring fertility in a progeny of a G-type cytoplasmic male sterile cereal plant, such as a wheat plant.

The present invention further relates to the use of a plant of the second aspect of the present invention or a plant obtained or obtainable by the method of the second aspect of the present invention for producing hybrid seed or a population of hybrid cereal plants, such as wheat seed or plants.

The present invention further relates to the use of at least one nucleic acid expression enhancing nucleic acid (NEENA) molecule for increasing the activity of a plant promoter in developing spikes.

The present invention further relates to the use of the plant promoter of the second aspect of the present invention for increasing expression of a nucleic acid molecule of interest in a plant, wherein the plant promoter is operably linked to the nucleic acid molecule of interest. Preferably, expression is increased in developing spikes. As set forth elsewhere herein, the nucleic acid molecule of interest preferably encodes a functional restorer polypeptide for wheat cytoplasmic male sterility. More preferably, it encodes the functional restorer polypeptide which is naturally linked to the (unmodified) promoter. However, the nucleic acid molecule of interest can be modified as well (in particular as described in Section C).

### Embodiments for the Second Aspect of the Present Invention (SECTION B, Promoter with EIL3 and/or PHD Transcription Factor Binding Site(s))

The methods, promoters, plants, constructs, uses etc. as described in SECTION B are further illustrated by the following embodiments and combinations of embodiments as indicated by the respective dependencies and back-references. The definitions and explanations given herein above apply mutatis mutandis to the following embodiments.
1. A method for increasing expression conferred by a plant promoter of a functional restorer gene for wheat cytoplasmic male sterility, comprising introducing at least one nucleic acid expression enhancing nucleic acid (NEENA) molecule into said promoter, wherein said at least one NEENA molecule
   i) comprises (or consists of) a nucleic acid sequence as shown in SEQ ID NO: 70, 86, 87, 90 or 91,
   ii) comprises (or consists of) a nucleic acid sequence with an identity of at least 90%, at least 91%, at least 92%, at least 93%, at least 94%, at least 95%, at least 96%, at least 97%, at least 98%, or at least 99% to SEQ ID NO: 70, 86, 87, 90 or 91,
   iii) comprises (or consists of) a fragment of at least 30, at least 40, in particular at least 50, at least 80, at least 100 or at least 120 consecutive bases of a nucleic acid molecule of i) or ii), or
   iv) is the complement or reverse complement of any of the previously mentioned nucleic acid molecules under i) to iii),
   wherein the nucleic acid molecule of ii), iii) and iv) is capable of increasing expression conferred by the plant promoter of the functional restorer gene for wheat cytoplasmic male sterility.
2. The method of embodiment 1, wherein the at least one NEENA molecule
   i) comprises (or consists of) a nucleic acid sequence as shown in SEQ ID NO 70,
   ii) comprises (or consists of) a nucleic acid sequence with an identity of at least 90%, at least 91%, at least 92%, at least 93%, at least 94%, at least 95%, at least 96%, at least 97%, at least 98%, or at least 99% to SEQ ID NO: 70,
   iii) comprises (or consists of) a fragment of at least 30, at least 40, in particular at least 50, at least 80, at least 100 or at least 120 consecutive bases of a nucleic acid molecule of i) or ii), or
   iv) is the complement or reverse complement of any of the previously mentioned nucleic acid molecules under i) to iii),
   wherein the nucleic acid molecule of ii), iii) and iv) is capable of increasing expression conferred by the plant promoter of the functional restorer gene for wheat cytoplasmic male sterility
3. The method of embodiment 1 or 2, wherein the promoter is a promoter of a functional restorer gene for wheat K-type cytoplasmic male sterility, in particular for wheat G-type cytoplasmic male sterility.
4. The method of any one of embodiments 1 to 3, wherein the promoter is the promoter of an Rf3 or Rf1 gene.
5. The method of embodiment 4, wherein the promoter is
   a) a promoter comprising a nucleic acid sequence as shown in SEQ ID NO: 23, 73 or 74, in particular 74
   b) a fragment of the nucleic acid sequence shown in SEQ ID NO: 23, 73 or 74
   c) a variant of the promoter of a) or the fragment of b), said fragment or variant having a sequence being at least 80%, 85%; 86%; 87%; 88%; 89%; 90%; 91%; 92%; 93%; 94%; 95%; 96%; 97%; 98%; or 99% identical to the sequence of a) or b), such as the sequence upstream of the ATG translation start site in SEQ ID NO: 94.
6. The method of embodiment 4, wherein the promoter is
   a) a promoter comprising a nucleic acid sequence as shown in SEQ ID NO: 36,
   b) a fragment of the nucleic acid sequence shown in SEQ ID NO: 36, such as SEQ ID NO: 34,
   c) a variant of the promoter of a) or the fragment of b), said fragment or variant having a sequence being at least 80%, 85%; 86%; 87%; 88%; 89%; 90%; 91%; 92%; 93%; 94%; 95%; 96%; 97%; 98%; or 99% identical to the sequence of a) or b).
7. The method of embodiment 4, wherein the promoter is
   a) a promoter comprising a nucleic acid sequence as shown in SEQ ID NO: 37,
   b) a fragment of the nucleic acid sequence shown in SEQ ID NO: 36, such as SEQ ID NO: 35,
   c) a variant of the promoter of a) or the fragment of b), said fragment or variant having a sequence being at least 80%, 85%; 86%; 87%; 88%; 89%; 90%; 91%; 92%; 93%; 94%; 95%; 96%; 97%; 98%; or 99% identical to the sequence of a) or b).
8. The method of any of the preceding embodiments, wherein the at least one NEENA molecule is introduced into the plant promoter by genome editing.
9. The method of any one of the preceding embodiments, wherein the at least one NEENA molecule is introduced at one or more positions within 1000 bp, such as within 500 bp or within 300 bp upstream (5′) to the translation start codon of the gene that is operably linked to said promoter.
10. The method embodiment 9, wherein the at least one NEENA molecule is introduced at a position within 250 to 80 bp, 200 to 100 bp, 110 to 150 bp, 120 to 140 bp, 125 to 135 bp, or within 125 to 130 bp, upstream (5′) to the translation start codon of the gene that is operably linked to said promoter, such as at the position −126 (minus 126), −127, −128, −129, 60−190, −83, −76, −70, −64 relative to the translation start codon, e.g. to the start codon of the Rf3 gene (as shown in FIG. 29).
11. The method of embodiment 9, wherein the at least one NEENA molecule is introduced at a position within 200 to 100 bp upstream (5′) to the translation start site of the gene that is operably linked to said promoter.
12. The method of any one of the preceding embodiments, wherein the resulting plant promoter has increased activity in developing spikes, such as in Zadok stages Z39-Z41, Z45-Z48, Z50-Z59, and/or Z60-Z69.
13. The method of any one of the preceding embodiments, wherein the method further comprises introducing into the plant promoter at least one heterologous binding site for an EIL3 transcription factor and/or at least one heterologous binding site for a PHD transcription factor, such as introducing at least one binding site for the EIL3 transcription factor and/or at least one binding site for said PHD transcription factor into the plant promoter, or modifying an existing EIL3 and/or PHD transcription factor binding site so that it has improved binding for its' transcription factor.
14. The method of embodiment 13, wherein the binding site for the PHD transcription factor has a sequence as shown in SEQ ID NO: 10, SEQ ID NO 11, SEQ ID NO: 40, SEQ ID NO: 31, SEQ ID NO: 32, SEQ ID NO: 38, SEQ ID NO: 42, SEQ ID NO: 41 or SEQ ID NO: 12, or is a variant thereof.
15. The method of embodiment 14, wherein the binding site for the EIL3 transcription factor has a sequence as shown in SEQ ID NO: 19 or SEQ ID NO: 39, or is a variant thereof.
16. The method of any one of embodiments 13 to 15, wherein a fragment having a sequence as shown in SEQ ID NO: 29 is introduced into the promoter, or a sequence differing in 1-5, such as in 1, 2, 3, 4 or 5 nucleotides from the sequence of SEQ ID NO: 29, such as the sequence double underlined in FIG. 29, or a sequence differing in 1-5, such as in 1, 2, 3, 4 or 5 nucleotides from the sequence of SEQ ID NO: 29, such as the sequence double underlined in FIG. 29.
17. A promoter obtained or obtainable by the method of any one of the preceding embodiments.
18. A promoter comprising a promotor of a functional restorer gene for wheat cytoplasmic male sterility functionally linked to at least one nucleic acid expression enhancing nucleic acid (NEENA) molecule as defined in embodiment 1 or 2.
19. The promoter of embodiment 18, wherein the promotor of a functional restorer gene for wheat cytoplasmic male sterility is a promoter as defined in any one of embodiments 2 to 7.
20. The promoter of any one of embodiments 17 to 19, further comprising at least one heterologous binding site for an EIL3 transcription factor and/or at least one heterologous binding site for a PHD transcription factor.
21. The promoter of any one of embodiments 18 to 20, wherein the plant promoter is operably linked to a nucleic acid of interest, for example wherein the nucleic acid molecule of interest encodes a functional restorer polypeptide for wheat cytoplasmic male sterility, such as a nucleic acid of interest with a mutated miRNA binding site as described in section C, e.g., a nucleic acid of interest that comprises a mutated miRNA binding site in the coding sequence as described in any one of embodiments 1-26 in the third aspect of the invention.
22. A chimeric nucleic acid molecule comprising the following operably linked elements
   a) the plant promoter of any one of embodiments 17 to 20;
   b) a nucleic acid molecule of interest; and optionally
   c) a transcription termination and polyadenylation region functional in plant cells.
23. The chimeric molecule of embodiment 22, wherein the nucleic acid molecule of interest encodes a functional restorer polypeptide for wheat cytoplasmic male sterility, such as a nucleic acid of interest with a mutated miRNA binding site as described in section C.
24. A cereal plant cell, a cereal plant or seed thereof, such as a wheat plant cell, wheat plant or seed thereof, comprising the plant promoter of any one of embodiments 17 to 21, or the chimeric nucleic acid molecule of embodiment 22 or 23.
25. The cereal plant cell, plant or seed of embodiment 24, which is a hybrid plant cell, plant or seed.
26. A method for producing a cereal plant cell or plant or seed thereof, such as a wheat plant cell or plant or seed thereof, comprising the step of providing said plant cell or plant with the plant promoter of any one of embodiments 17 to 21, or the chimeric nucleic acid molecule of embodiment 22 or 23.
27. A method for increasing expression of a functional restorer polypeptide for wheat G-type cytoplasmic male sterility, or for increasing restoration capacity for wheat G-type cytoplasmic male sterility (“CMS”) in a cereal plant, such as a wheat plant, comprising the step of providing said plant cell or plant with the plant promoter of embodiment 17 to 21 or the chimeric nucleic acid molecule of embodiment 22 or 23.
28. A method for identifying and/or selecting a cereal plant having increased expression of a functional restorer polypeptide for wheat G-type cytoplasmic male sterility and/or increased restoration capacity for wheat G-type cytoplasmic male sterility, said method comprising the steps of:
   a) identifying or detecting in said plant the presence of the plant promoter of embodiment 17 to 21, or the chimeric nucleic acid molecule of embodiment 22 or 23, and
   b) selecting said plant comprising said plant promoter or chimeric nucleic acid molecule.
29. A method for producing hybrid seed, comprising the steps of:
   a) providing a male cereal parent plant, such as a wheat plant, according to embodiment 24 and/or providing a male cereal parent plant, such as a wheat plant, comprising the plant promoter of embodiment 17 to 21 or the chimeric nucleic acid molecule of embodiment 22 or 23, wherein said nucleic acid molecule or chimeric gene is preferably present in homozygous form,
   b) providing a female cereal parent plant that is a G-type cytoplasmic male sterile cereal plant,
   c) crossing said female cereal parent plant with a said male cereal parent plant; and optionally,
   d) harvesting hybrid seeds on said female cereal parent plant.
30. Use of the plant promoter of embodiment 17 to 21 or the chimeric nucleic acid molecule of embodiment 22 or 23 for the identification of a plant comprising said functional restorer gene allele for wheat G-type cytoplasmic male sterility.
31. Use of a plant according to embodiment 24 or 25, or a plant obtained or obtainable by the method of embodiment 26 for restoring fertility in a progeny of a cytoplasmic male sterile cereal plant, such as a K-type or G-type cytoplasmic male sterile cereal plant wheat plant.
32. Use of a plant according to embodiment 24 or 25 or a plant obtained or obtainable by the method of embodiment 26 for producing hybrid seed or a population of hybrid cereal plants, such as wheat seed or plants.
33. Use of at least one nucleic acid expression enhancing nucleic acid (NEENA) molecule as defined embodiment 1 or 2 for increasing expression conferred by a plant promoter of a functional restorer gene for wheat cytoplasmic male sterility.

### Section C (Modified MIRNA Binding Site)

### Brief Summary of the First Aspect of the Present Invention (SECTION C)

In the third aspect, the present invention relates to a plant (such as a cereal plant, e.g., wheat) nucleic acid molecule comprising a miRNA binding site in the coding sequence, in particular a miRNA3619 binding site (such as the sequence of SEQ ID NO: 45 (RNA) or 46 (DNA), or a sequence differing in 1-3 nucleotides from that sequence, such as the sequence of SEQ ID NO: 69 (RNA, GGGUAGGAUGGAUGAUGCU) or the DNA sequence encoding it), that is mutated (as compared to the miRNA sequence naturally present in said nucleic acid molecule), preferably the mutation is in a translationally neutral manner. Expression of such gene comprising a mutated miRNA binding side in the coding sequence, is higher compared to the native gene in those plants cells/tissues expressing the miRNA3619.

The third aspect of the present invention, thus, relates to a nucleic acid molecule encoding a functional restorer polypeptide for wheat G-type cytoplasmic male sterility, wherein said nucleic acid molecule comprises a miRNA binding site in the coding sequence that is mutated (as compared to a miRNA binding site sequence that is naturally present in said nucleic acid molecule). As used herein, “naturally present”, includes the presence in cultivated plants that may not occur in the wild/nature (such as (hybrid) wheat), but that were not mutated/modified (other than the modifications to breed a commercial crop, including any transgenes or mutants or genome edits that improve the crop), such as not mutated/modified to disrupt/inactivate a miRNA binding site sequence occurring in the coding sequence.

The third aspect of the present invention also relates to a chimeric nucleic acid molecule comprising the following operably linked elements
a. a plant-expressible promoter;
b. the nucleic acid molecule of the present invention, and optionally
c. a transcription termination and polyadenylation region functional in plant cells.

In an embodiment of the third aspect, the nucleic acid molecule encoding a functional restorer polypeptide is a mutated Rf3 gene. Said mutated Rf3 gene comprises at least one mutation in the miRNA binding site having a sequence as shown in SEQ ID NO: 45 or 46 (or 69). Preferably, the nucleic acid molecule encoding a functional restorer polypeptide for wheat G-type cytoplasmic male sterility is a (mutated) Rf3 gene which does not comprise a sequence as shown in SEQ ID NO: 45 (RNA, GGGUAGGUUGGAUGAUGCU) or SEQ ID NO: 69, if it is a mRNA sequence or SEQ ID NO: 46 (DNA, gggtag gttggatgatgct) or the DNA encoding SEQ ID NO: 69, if it is a DNA sequence. Thus, the nucleic acid molecule comprising a miRNA binding site in the coding sequence does not comprise a sequence as shown in SEQ ID NO: 45 or 46 or 69.

Preferably, the Rf3 functional restorer polypeptide as referred to in Section c may comprise
a) an amino acid sequence as shown in SEQ ID NO: 44 or SEQ ID NO: 63, or
b) an amino acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 44 or SEQ ID NO: 63, preferably over the entire length.

Preferably, the Rf3 nucleic acid molecule of the present invention comprises
a) at least one mutation in the nucleic acid sequence as shown in SEQ ID NO: 43 or
b) at least one mutation in a nucleic acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 43, preferably over the entire length,
   wherein one or more nucleotide(s) at a position in the region corresponding to the region from the nucleotide at position 1245 to the nucleotide at position 1263 (or the region corresponding to the region between nucleotide positions 1244 and 1264, not including positions 1244 and 1264) in SEQ ID NO: 43 are mutated.

Alternatively, the Rf3 nucleic acid molecule comprises
a) at least one mutation in the nucleic acid sequence as shown in SEQ ID NO: 62, or
b) at least one mutation in a nucleic acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 62, preferably over the entire length,
   wherein one or more nucleotide(s) at a position in the region corresponding to the region from the nucleotide at position 1239 to the nucleotide at position 1257 in SEQ ID NO: 62 are mutated.

In another embodiment, the nucleic acid molecule encoding a functional restorer polypeptide is a mutated Rf1 gene (herein also referred to as Rf1-09 gene). Said mutated Rf1 gene comprises at least one mutation in the miRNA binding site having a sequence as shown in SEQ ID NO: 67 (gggucgguuggacgaugcu), if it is a mRNA sequence, or SEQ ID NO: 66 (gggtcggttggacgatgct), if it is an DNA sequence. In other words, the nucleic acid molecule comprising a miRNA binding site in the coding sequence that is mutated does not comprise a sequence as shown in SEQ ID NO: 66 or 67.

Preferably, the functional restorer polypeptide encoded by the Rf1 gene as referred to herein may comprise
a) an amino acid sequence as shown in SEQ ID NO: 65, or
b) an amino acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 65, preferably over the entire length.

Also preferably, the Rf1 nucleic acid molecule of the present invention comprises
a) at least one mutation in the nucleic acid sequence as shown in SEQ ID NO: 64 or
b) at least one mutation in a nucleic acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 64, preferably over the entire length,
   wherein one or more nucleotide(s) at a position in the region corresponding to the region from the nucleotide at position 1239 to the nucleotide at position 1257 in SEQ ID NO: 64 are mutated.

In an embodiment of the third aspect of the present invention, the miRNA binding site, e.g., in the Rf1 or Rf3 gene, has been mutated in a translationally neutral manner.

In an embodiment of the third aspect of the present invention, the coding sequence of the Rf gene of the invention, that has been mutated in the miRNA3619 binding site, has not been codon-optimized over the entire coding sequence (changing codons in a translationally-neutral manner to the codon preferences/frequencies (or GC-content) deemed more suitable for (highly-expressing genes in) the respective plant species), such as a coding sequence of the mutated Rf gene that only has changes in 1 to 20, 1 to 19, 1 to 18, 1 to 17, 1 to 16, 1 to 15, 1 to 14, 1 to 13, 1 to 12, 1 to 11, 1 to 10, 1 to 9, 1 to 8, 1 to 7, 1 to 6, 1 to 5, 1 to 4, 1 to 3, or 1 or 2 nucleotides in the coding sequence, compared to the coding sequence that is naturally present, such as the entire Rf coding sequence having less than 20, less than 15, less than 10, less than 9, less than 8, less than 7, less than 6, or less than 5 nucleotides mutated compared to the coding sequence that is naturally present. In one embodiment, a mutated Rf gene according to the third aspect of the current invention has one or more mutations in the miRNA3619 binding site and has less than 20, less than 15, less than 10, less than 9, less than 8, less than 7, less than 6, less than 5, less than 4, or less than 3 nucleotides mutated in the entire coding sequence outside the miRNA binding site (such as in the entire Rf coding sequence outside the miRNA binding site (like the coding sequence of any one of SEQ ID NO 43, 62 or 64), compared to the coding sequence that is naturally present (e.g., to remove long coding regions in other reading frames, to change nucleotides for cloning work).

In an embodiment of the third aspect of the present invention, the mutation of the miRNA binding site mutation results in a lower number of base pairs formed between the binding site and the miRNA as compared to the number of base pairs formed between the unmodified binding site and the miRNA.

In an embodiment of the third aspect of the present invention, the one or more nucleotides have been mutated by substituting, deleting and/or adding one or more nucleotides at a position corresponding to a position in the region from nucleotide position 1245 to nucleotide position 1263 in SEQ ID NO: 43 or in the region from nucleotide position 1239 to nucleotide position 1257 in SEQ ID NO: 64.

In an embodiment of the third aspect of the present invention, the one or more nucleotides have been substituted with one or more different nucleotides.

In an embodiment of the third aspect of the present invention, at least 1, 2, 3, 4, 5, 6, 7, 8, 9, 10, 11, 12, 13, 14, 15, 16, 17, or 18 nucleotides have been substituted in the miRNA binding site with a different nucleotide.

In an embodiment of the third aspect of the present invention, the nucleotide (or nucleotides) corresponding to position 1245, 1246, 1247, 1248, 1249, 1250, 1251, 1253, 1254, 1255, 1256, 1257, 1258, 1259, 1260, 1261, 1262 and/or 1263 in SEQ ID NO: 43 has (have) been substituted with a different nucleotide (or different nucleotides). For example, the nucleotide (or nucleotides) corresponding to position 1245, 1248, 1249, 1250, 1254, 1257, 1260, 1262 and/or 1263 in SEQ ID NO: 43 has (have) been substituted with a different nucleotide (or different nucleotides). Hence, at least one, or several or all of these nucleotide position(s) can be substituted by another nucleotide.

In one embodiment (where the mutated Rf gene encodes the same protein as the gene that is naturally present), the nucleotide (or nucleotides) corresponding to position 1245, 1248, 1250, 1251, 1253, 1254, 1257, 1260, and/or 1263 in SEQ ID NO: 43 has (have) been substituted with a different nucleotide (or different nucleotides).

In an embodiment of the third aspect of the present invention, the nucleotide (or nucleotides) corresponding to position 1239, 1240, 1241, 1242, 1244, 1245, 1247, 1248, 1249, 1250, 1252, 1253, 1254, 1255, 1256 and/or 1257 in SEQ ID NO: 64 has (have) been substituted with a different nucleotide (or different nucleotides). In an embodiment of the third aspect of the present invention, the nucleotide (or nucleotides) corresponding to position 1239, 1242, 1244, 1245, 1247, 1248, 1254, and/or 1257 in SEQ ID NO: 64 has (have) been substituted with a different nucleotide (or different nucleotides).

In an embodiment of the third aspect of the present invention, the miRNA binding site has been mutated by mutagenesis, such as by chemical mutagenesis, such as EMS mutagenesis.

The third aspect of the present invention also relates to a chimeric nucleic acid molecule/gene comprising the following operably linked elements
a) a plant-expressible promoter;
b) the nucleic acid molecule of the third aspect of the present invention; and optionally
c) a transcription termination and polyadenylation region functional in plant cells.

In one embodiment third aspect of the, the plant-expressible promoter and the transcription termination and polyadenylation region in the chimeric Rf gene of the third aspect of the invention are as in the endogenous Rf gene, and only the coding sequence of the nucleic acid molecule of the third aspect of the present invention has been mutated/genome edited. In another embodiment, besides the coding sequence of the Rf nucleic acid molecule of the present invention that has been mutated/genome edited in a miRNA binding site, the plant-expressible promoter and/or the transcription termination and polyadenylation region operably-linked to that coding sequence have also been mutated or genome edited (compared to the promoter and/or transcription termination and polyadenylation region of the endogenous Rf gene) to further improve Rf gene expression.

In one embodiment of the chimeric nucleic acid molecule of the third aspect of the present invention, the promoter is capable of directing expression of the operably linked nucleic acid at least during early pollen development and meiosis.

In one embodiment of the chimeric nucleic acid molecule of the third aspect of the present invention, the promoter is heterologous with respect to the nucleic acid molecule of the third aspect of the present invention. In another embodiment of the chimeric nucleic acid molecule of the third aspect of the present invention, the promoter is the native promoter.

The third aspect of the present invention also relates to a plant cell, such as a cereal plant cell, or plant, such as a cereal plant or seed thereof, such as a wheat plant cell or plant or seed thereof, comprising the nucleic acid molecule of the present invention, or the chimeric gene of the present invention. For example, it may be hexaploid wheat plant or plant cell possessing T. timopheevi cytoplasm.

In an embodiment of the plant cell, the plant or seed of the third aspect of the present invention, the plant cell, plant or seed is a hybrid plant cell, plant or seed.

The third aspect of the present invention also relates to a method for producing a cereal plant cell or plant or seed thereof, such as a wheat plant cell or plant or seed thereof, comprising a functional restorer gene for wheat G-type cytoplasmic male sterility, or for increasing restoration capacity and/or restoration stability for wheat G-type cytoplasmic male sterility (“CMS”) in a cereal plant, such as a wheat plant, comprising the steps of providing said plant cell or plant with the nucleic acid molecule of the third aspect of the present invention or the chimeric gene of the present invention.

The third aspect of the present invention also relates to a method for improving expression of a functional restorer gene for wheat G-type cytoplasmic male sterility, or for increasing restoration capacity and/or restoration stability for wheat G-type cytoplasmic male sterility (“CMS”) in a cereal plant, such as a wheat plant, comprising the step of providing said plant cell or plant with the nucleic acid molecule of the third aspect of the present invention or the chimeric gene of the third aspect of the present invention. The increase of expression of a functional restorer (Rf) gene for wheat G-type cytoplasmic male sterility would also allow for an increase of seed yield and/or improved yield stability as compared to a control plant (see Example 10).

The third aspect of the present invention also relates to a cereal plant cell or cereal plant or seed thereof, such as a wheat plant cell or plant or seed thereof, obtained according to any of the methods of the third aspect of the present invention.

In an embodiment of the plant cell, the plant or seed of the third aspect of the present invention, the plant cell, plant or seed is a hybrid wheat plant cell, plant or seed.

The third aspect of the present invention also pertains to a method for identifying and/or selecting a cereal (e.g., wheat) plant comprising an improved functional fertility restoration (Rf) gene allele for wheat G-type cytoplasmic male sterility (CMS) comprising the steps of:
a) Identifying or detecting in said plant the presence of a nucleic acid of the present invention or the chimeric gene of the present invention, or said modified miRNA binding site, and
b) selecting said plant comprising said nucleic acid molecule or chimeric gene.

The third aspect of the present invention also relates to a method for producing hybrid seed, comprising the steps of:
a) Providing a male cereal parent plant, such as a wheat plant, of the present invention, said plant comprising said improved functional restorer gene allele for wheat G-type cytoplasmic male sterility, wherein said improved functional restorer gene allele is preferably present in homozygous form,
b) Providing a female cereal parent plant that is a G-type cytoplasmic male sterile cereal plant,
c) Crossing said female cereal parent plant with said male cereal parent plant; and optionally
d) Harvesting seeds.

The third aspect of the present invention also relates to the use of the nucleic acid molecule or of the chimeric gene of the present invention for the identification of a plant comprising said functional restorer gene allele for wheat G-type cytoplasmic male sterility.

The third aspect of the present invention also relates to the use of the nucleic acid molecule or of the chimeric gene of the present invention for generating plants comprising said functional restorer gene allele for wheat G-type cytoplasmic male sterility.

The third aspect of the present invention furthermore relates to the use of a plant of the present invention for restoring fertility in a progeny of a G-type cytoplasmic male sterile cereal plant, such as a wheat plant.

The third aspect of the present invention furthermore relates to the use of the plant of the present invention, said plant comprising said improved functional restorer gene for wheat G-type cytoplasmic male sterility, for producing hybrid seed or a population of hybrid cereal plants, such as wheat seed or plants.

The third aspect of the present invention further relates to a polypeptide which is preferably encoded by the nucleic acid of the present invention, wherein said polypeptide comprises at least one substituted amino acid residue in at least one position corresponding to position 415, 416, 417, 418, 419, 420 and/or 421 of SEQ ID NO: 44.

### Detailed Description of the Third Aspect of the Present Invention (Section C, Modified miRNA Binding Site)

The inventors have identified a miRNA binding site for miRNA3619 in the coding sequence of the functional Rf3-58 gene and variants thereof, a gene encoding a functional restorer polypeptide for wheat G-type cytoplasmic male sterility (see Example 7, or FIG. 6A). Advantageously, it was shown that mutations within the identified miRNA binding site lead to increased expression of the Rf3 gene (see Example 8). Wheat plants containing the Rf3 gene with a disrupted miRNA binding site for miRNA3619 had a higher Rf3 gene expression and a higher seed set than control plants with an intact miRNA binding site (see Example 10). Thus, it was shown that the mutation of the miRNA binding site leads to increased expression of the Rf3 gene which allows for an improved fertility restoration in wheat T. timopheevi cytoplasm.

Advantageously, a potential miRNA binding site for miRNA3619 is also present in a functional Rf1 gene (see FIG. 6B for the RNA version, and the underlined sequence in FIG. 14 for the DNA version).

The third aspect of the present invention provides a contribution over the art by disclosing a miRNA binding site in a functional Rf gene coding sequence (such as a Rf1 or Rf3 gene coding sequence), the modification of which increases expression of the Rf gene (e.g., of the Rf1 or Rf3 gene). The finding that a modified miRNA binding site would allow for an increased expression of the functional restorer polypeptide for wheat G-type cytoplasmic male sterility (CMS), and without any obvious phenotypic or developmental side-effects is useful in methods for hybrid seed production, as plants comprising the modified miRNA binding site can be used, e.g., in a method for restoring fertility in progeny of a plant possessing G-type cytoplasmic male sterility, thereby producing fertile progeny plants from a G-type cytoplasmic male sterile parent plant.

Accordingly, the third aspect of the present invention relates to a nucleic acid molecule encoding a functional restorer polypeptide for wheat G-type cytoplasmic male sterility, wherein said nucleic acid molecule comprises a miRNA binding site in the coding sequence that is mutated (i.e., mutated as compared to the naturally occurring miRNA binding site).

In accordance with the third aspect of the present invention, the functional restorer polypeptide for wheat G-type cytoplasmic male sterility has the capacity to restore fertility in the progeny of a cross with a G-type cytoplasmic male sterile cereal plant (when expressed in a (sexually compatible) cereal plant). Thus, it is capable of restoring the fertility in the progeny of a cross with a G-type cytoplasmic male sterility (“CMS”) line, i.e., a line carrying common wheat nuclear genes but cytoplasm from Triticum timopheevii.

Male sterility in connection with the third aspect of the present invention refers to the failure or partial failure of plants to produce functional pollen or male gametes. This can be due to natural or artificially introduced genetic predispositions or to human intervention on the plant in the field.

Male fertility on the other hand relates to plants capable of producing normal functional pollen and male gametes. Male sterility/fertility can be reflected in seed set upon selfing, e.g., by bagging heads to induce self-fertilization. Likewise, fertility restoration can also be described in terms of seed set upon crossing a male sterile plant with a plant carrying a functional restorer gene, when compared to seed set resulting from crossing (or selfing) fully fertile plants. A male parent (or pollen parent), is a parent plant that provides the male gametes (pollen) for fertilization, while a female parent or seed parent is the plant that provides the female gametes for fertilization, said female plant being the one bearing the (hybrid) seeds.

The nucleic acid molecule of the third aspect of the present invention encodes a polypeptide which allows for restoring cytoplasmic male sterility (abbreviated “CMS”). “CMS” refers to cytoplasmic male sterility. CMS is total or partial male sterility in plants (e.g., as the result of specific nuclear and/or mitochondrial interactions) and is maternally inherited via the cytoplasm. Male sterility is the failure of plants to produce functional anthers, pollen, or male gametes although CMS plants still produce viable female gametes. Cytoplasmic male sterility is used in agriculture to facilitate the production of hybrid seed.

“Wheat G-type cytoplasmic male sterility”, as used herein refers to the cytoplasm of Triticum timopheevii that can confer male sterility when introduced into common wheat (i.e., Triticum aestivum), thereby resulting in a plant carrying common wheat nuclear genes but cytoplasm from Triticum timopheevii that renders plants male sterile in absence of fertility restoration (Rf or restorer) genes. The cytoplasm of Triticum timopheevii (G-type) as inducer of male sterility in common wheat has been extensively studied.

Restoration against G-type cytoplasm has, e.g., been described in the art. The restorer genes encoding such polypeptides are also referred to as Rf genes. Most fertility restoration polypeptides come from a clade of genes encoding pentatricopeptide repeat (PPR) proteins (Fuji et al., 2011, PNAS 108(4), 1723-1728—herein incorporated by reference).

In accordance with the third aspect of the present invention, a functional restorer polypeptide for wheat G-type cytoplasmic male sterility is preferably a pentatricopeptide repeat (PPR) protein.

Rf-PPR genes are usually present in clusters of similar Rf-PPR-like genes, which show a number of characteristic features compared with other PPR genes. They are comprised primarily of tandem arrays of 15-20 PPR motifs, each composed of 35 amino acids. PPR proteins are classified based on their domain architecture. P-class PPR proteins possess the canonical 35 amino acid motif and normally lack additional domains. Members of this class have functions in most aspects of organelle gene expression. PLS-class PPR proteins have three different types of PPR motifs, which vary in length; P (35 amino acids), L (long, 35-36 amino acids) and S (short, ˜31 amino acids), and members of this class are thought to mainly function in RNA editing. Subtypes of the PLS class are categorized based on the additional C-terminal domains they possess (reviewed by Manna, 2015, incorporated herein by reference).

In particular, it is envisaged that the functional restorer polypeptide as referred to herein is a Rf3-PPR polypeptide (alternative name: Rf3 polypeptide), or Rf1-PPR polypeptide (alternative name: Rf1 polypeptide). Rf polypeptides are known in the art and are, for example, described in Melonek et al. (2021) and in WO 2018/015403.

In an embodiment of the third aspect of the present invention, the functional restorer polypeptide comprises an amino acid sequence as shown in SEQ ID NO: 44 (herein also referred as Rf3-58) which is an Rf3 polypeptide. In another embodiment, the functional restorer polypeptide comprises an amino acid sequence as shown in SEQ ID NO: 63 (herein also referred as Rf3-29a) which is another Rf3 allele polypeptide. Also included in the third aspect of the current invention are any other functional Rf polypeptides, such as variants of the sequences in SEQ ID NO: 44 or 63, and genes encoding them, particularly Rf genes comprising the sequence of SEQ ID NO: 46 (or a sequence being at least 60%, such as at least 70%, at least 75%, at least 80%, at least 85%, or at least 90% identical to SEQ ID NO: 46, or a sequence having 1, 2, or 3, nucleotides substituted compared to SEQ ID NO 66).

In another embodiment of the third aspect of the current invention, the functional restorer polypeptide comprises an amino acid sequence as shown in SEQ ID NO: 65 (herein also referred as Rf1-09) which is an Rf1 polypeptide. Also included are variants of the sequences in SEQ ID NO: 65, and genes encoding them, particularly Rf1 genes comprising the sequence of SEQ ID NO: 66 (or a sequence being at least 60%, such as at least 70%, at least 75%, at least 80%, at least 85%, or at least 90% identical to SEQ ID NO: 66, or a sequence having 1, 2, or 3, nucleotides substituted compared to SEQ ID NO 66), preferably over the entire sequence, wherein the miRNA3619 binding site naturally present in the coding sequence has been mutated.

Further, it is envisaged that the functional restorer polypeptide is a variant of the above sequences. Preferably, the variant is capable of restoring wheat G-type cytoplasmic male sterility.

Thus, the functional restorer polypeptide may comprise
a) an amino acid sequence as shown in SEQ ID NO: 44, SEQ ID NO: 63 or SEQ ID NO: 65, or
b) an amino acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 44, SEQ ID NO: 63, or SEQ ID NO: 65.

How to calculate the degree of sequence identity between polypeptides is described in Section A. The explanations apply accordingly to Section C.

The functional restorer polypeptide for wheat G-type cytoplasmic male sterility can be a naturally occurring polypeptide, or a polypeptide which does not occur naturally. However, it is envisaged that it is encoded by a non-naturally occurring nucleic acid molecule, regardless whether it encodes a naturally occurring polypeptide or a non-naturally occurring polypeptide. Specifically, the non-naturally occurring nucleic acid molecule comprises a mutated miRNA binding site, in particular a mutated miRNA binding site for miRNA3619.

The miRNA3619 is similar to ata-miR9674a-5p (see e.g., Li et al. (2019)) and tae-miR9674b-5p. The sequence of miR9674a-5p can be, e.g., retrieved in miRbase (www.mirbase.org/; Kozomara, Birgaoanu, and Griffiths-Jones 2019).

Thus, the functional restorer polypeptide is encoded by a nucleic acid molecule having an altered (mutated) miRNA binding site, in particular a mutated miRNA binding site for the miRNA3619.

The nucleic acid molecule may be an RNA molecule, such as an mRNA, or a DNA molecule.

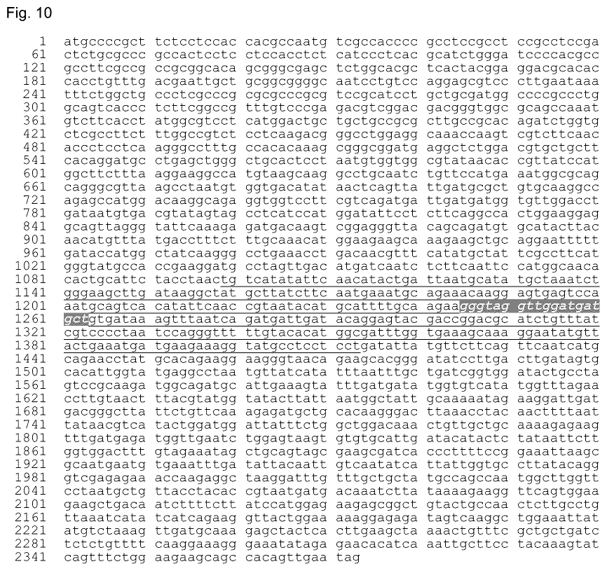

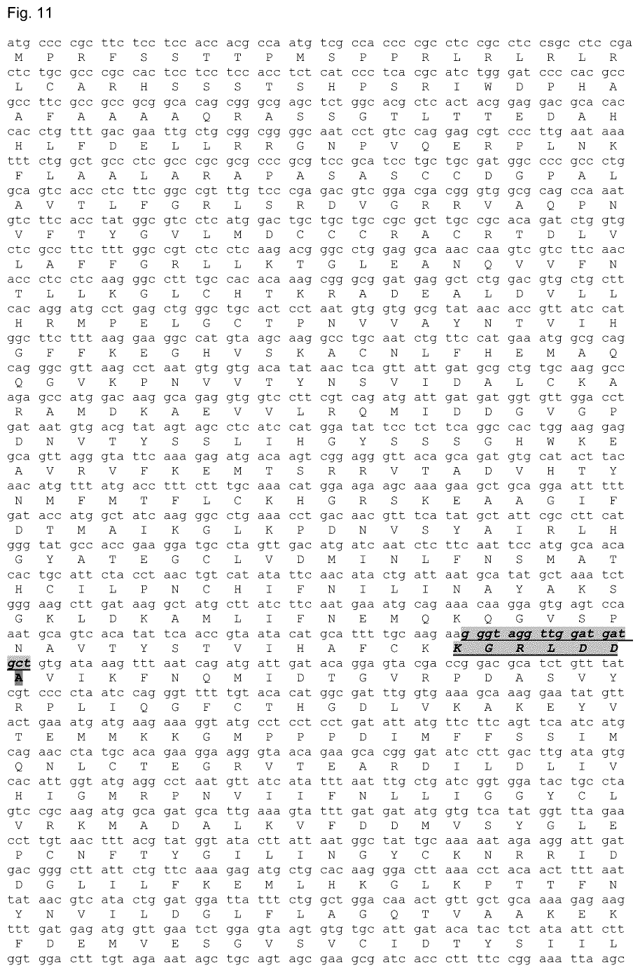

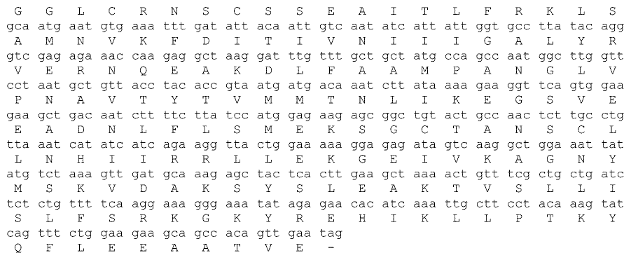

In the studies underlying the third aspect of the present invention, the miRNA binding site for miRNA3619 comprised in the coding sequence of the Rf3 gene was analyzed. The unmodified Rf coding sequence is shown in SEQ ID NO: 43 or 62 (see also FIG. 10). It encodes a polypeptide comprising an amino acid sequence as shown in SEQ ID NO: 44 or 63 (an Rf3 polypeptide, also referred to as Rf3-58 (SEQ ID NO: 44) or Rf3-29a (SEQ ID NO: 63)).

FIG. 6A shows the binding of miRNA3619 to the miRNA binding site in the mRNA of Rf3-58. As can be derived from FIG. 6A, the miRNA binding site for miRNA3619 has a length of 19 nt (shown in capital letters). Within the miRNA binding site, there is only one mismatch to miRNA3619. The mismatch can be found at position 8 of the miRNA binding site (based on the numbering system on top of FIG. 6A). Position 8 of the miRNA binding site corresponds to position 1252 in SEQ ID NO: 43. Thus, the naturally occurring miRNA binding site is complementary to miRNA3619, but comprises one mismatch to said miRNA.

The identified miRNA binding site for miRNA3619 in Rf3-58 mRNA is shown in SEQ ID NO: 45 (GGGUAGGUUGGAUGAUGCU, see also FIG. 6A). The corresponding DNA sequence, i.e., the sequence which encodes the miRNA binding site, is shown in SEQ ID NO: 46 (gggtag gttggatgatgct). The DNA sequence is highlighted in bold in FIG. 10, and can be found at nucleotide position 1245 to nucleotide position 1263 in SEQ ID NO: 43.

The same, identical miRNA binding site can be also found in an allele of the Rf3 gene, such as the RFL29a gene (SEQ ID NO: 62). Specifically, it can be found at nucleotide position 1239 to nucleotide position 1257 in the nucleotide sequence of SEQ ID NO: 62.

The miRNA3619 which binds to the miRNA binding site for miRNA3619 comprises a sequence as shown in SEQ ID NO: 47 (5′-UAGCAUCAUCCAUCCUACCCA-3′, see also FIG. 6A).

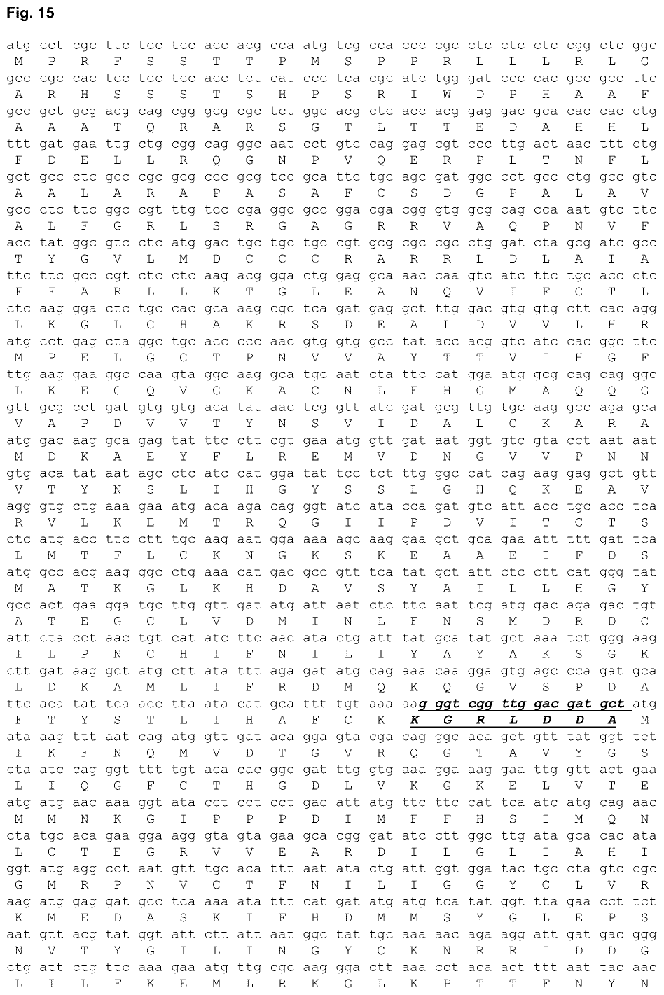

A putative binding site for miRNA3619 can be also found in Rf1-09. The Rf1-09 gene has a coding sequence as shown in SEQ ID NO: 64. It encodes a polypeptide comprising an amino acid sequence as shown in SEQ ID NO: 65. The putative binding site for miRNA3619 is highlighted in the sequence shown in FIG. 15. FIG. 6B shows the binding of miRNA3619 to the miRNA binding site in the mRNA of Rf1-09. As can be derived from FIG. 6B, the miRNA binding site for miRNA3619 has a length of 19 nt (shown in capital letters). Within the miRNA binding site, there are three mismatches to miRNA3619.

As set forth above, the miRNA binding site within the nucleic acid molecule encoding a functional restorer polypeptide for wheat G-type cytoplasmic male sterility has been mutated, i.e., it is modified as compared to the naturally occurring miRNA binding site. However, it is envisaged that the mutated nucleic acid molecule still encodes a functional restorer polypeptide.

In an embodiment, the nucleic acid molecule comprising a miRNA binding site in the coding sequence that is mutated comprises a miRNA binding site which is mutated as compared to the miRNA binding site as shown in SEQ ID NO: 46 (e.g., if the functional restorer gene is an Rf3 gene).

In an embodiment of the third aspect of the present invention, the nucleic acid molecule encoding a functional restorer polypeptide for wheat G-type cytoplasmic male sterility does not comprise a sequence as shown in SEQ ID NO: 46 (gggtag gttggatgatgct). Thus, the nucleic acid molecule comprising a miRNA binding site in the coding sequence that is mutated does not comprise a sequence as shown in SEQ ID NO: 46 (e.g., if the functional restorer gene is an Rf3 gene).

In an embodiment of the third aspect of the present invention, the nucleic acid molecule comprising a miRNA binding site in the coding sequence that is mutated comprises a miRNA binding site which is mutated as compared to the miRNA binding site as shown in SEQ ID NO: 66 (e.g., if the functional restorer gene is an Rf1 gene).

In an embodiment of the third aspect of the present invention, the nucleic acid molecule encoding a functional restorer polypeptide for wheat G-type cytoplasmic male sterility does not comprise a sequence as shown in SEQ ID NO: 66 (gggtcggttggacgatgct). Thus, the nucleic acid molecule comprising a miRNA binding site in the coding sequence that is mutated does not comprise a sequence as shown in SEQ ID NO: 66 (e.g., if the functional restorer gene is an Rf1 gene).

Further, in one embodiment of the third aspect of the present invention it is envisaged that the Rf nucleic acid coding sequence has a mutated miRNA3916 binding site, with 1 to 5, 1 to 4, 1 to 3, or 5, 4, 3, 2 or 1 nucleotide differences compared to the miRNA binding site of SEQ ID NO: (RNA), 46 (DNA) or 66 (RNA) or 67 (DNA).

In one embodiment of the third aspect of the present invention, the mutation in a miRNA binding site in the coding sequence according to this invention, increases expression of a plant gene, particularly of an Rf gene, such as a wheat Rf gene. Once a putative miRNA binding site has been identified in a plant coding sequence, it can easily be tested in a certain plant species if a modification of that binding site increases expression, by testing expression of a reporter/marker gene linked to the part of that gene containing the putative miRNA binding site in a (transient) expression system for such plant species (e.g., expression in protoplasts of said plant species), in comparison to the unmodified miRNA binding site (normalized, to correct for differences in introduction efficiency). A modified version of the miRNA binding site that increases expression then evidences that the native miRNA binding site can reduce expression of that coding sequence in that species, if the miRNA is present/expressed where the coding sequence is expressed (as can be measured by standard tools such as protein or RNA expression, or by measuring the reporter protein activity of a reporter protein fused to the polypeptide encoded by the nucleic acid molecule of the present invention (or portion thereof, see Examples)).

In one embodiment of the third aspect of the present invention, the nucleic acid molecule comprises
a) at least one mutation in the nucleic acid sequence as shown in SEQ ID NO: 43 or
b) at least one mutation in a nucleic acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 43, preferably over the entire length,
   wherein one or more nucleotide(s) at a position in the region corresponding to the region from nucleotide position 1245 to nucleotide position 1263 in SEQ ID NO: 43 are mutated.

For example, the nucleotide (or nucleotides) corresponding to position 1245, 1246, 1247, 1248, 1249, 1250, 1251, 1253, 1254, 1255, 1256, 1257, 1258, 1259, 1260, 1261, 1262 and/or 1263 in SEQ ID NO: 43 has (have) been substituted with a different nucleotide (or different nucleotides), such as the nucleotide (or nucleotides) corresponding to position 1245, 1248, 1249, 1251, 1254, 1257, 1260, 1261 and/or 1263 in SEQ ID NO: 43. Hence, at least one, or several or all of these nucleotide position(s) can be substituted by another nucleotide.

In another embodiment of the third aspect of the present invention, the nucleic acid molecule comprises
a) at least one mutation in the nucleic acid sequence as shown in SEQ ID NO: 62, or
b) at least one mutation in a nucleic acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 62, preferably over the entire length,
   wherein one or more nucleotide(s) at a position in the region corresponding to the region from nucleotide position 1239 to nucleotide position 1257 in SEQ ID NO: 62 are mutated.

In another embodiment of the third aspect of the present invention, the nucleic acid molecule comprises
a) at least one mutation in the nucleic acid sequence as shown in SEQ ID NO: 64, or
b) at least one mutation in a nucleic acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 64,
   wherein one or more nucleotide(s) at a position in the region corresponding to the region from nucleotide position 1239 to nucleotide position 1257 in SEQ ID NO: 64 are mutated.

The term “mutation” as used in the third aspect of the present invention refers to any type of nucleic acid alterations such as the insertion of one or more nucleotides into the miRNA binding site (or to be more precise into the DNA sequence encoding for the binding site in the RNA molecule), the deletion of one or more nucleotides of the miRNA binding site, and a substitution (i.e., change) of one or more nucleotides in the miRNA binding site sequence, or combinations thereof. In one embodiment of the invention, if one or more nucleotides are inserted or deleted, the mutation(s) do not result in a frame shift.

In an embodiment of the third aspect of the present invention, the one or more nucleotides have been mutated by substituting, deleting and/or adding one or more nucleotides at a position corresponding to a position in the region from nucleotide position 1245 to nucleotide position 1263 in SEQ ID NO: 43.

In another embodiment of the third aspect of the present invention, the one or more nucleotides have been mutated by substituting, deleting and/or adding one or more nucleotides at a position corresponding to a position in the region from nucleotide position 1239 to nucleotide position 1257 in SEQ ID NO: 62.

In another embodiment of the third aspect of the present invention, the one or more nucleotides have been mutated by substituting, deleting and/or adding one or more nucleotides at a position corresponding to a position in the region from nucleotide position 1239 to nucleotide position 1257 in SEQ ID NO: 64.

Preferably, the mutation is the substitution of one or more nucleotides in the miRNA binding site. Further, in one embodiment it is envisaged that the miRNA binding site may have been mutated in a translationally neutral manner, and in another embodiment the miRNA binding site has been mutated so that one or more conservative amino acid changes occurred (see, e.g., https://en.wikipedia.org/wiki/Conservative_replacement), such as Lysine being replaced by Histidine or Arginine; Glycine by Alanine, Valine, Leucine or Isoleucine; Arginine by Histidine or Lysine; Leucine by Glycine, Alanine, Valine, or Isoleucine; Aspartate by Glutamate, Asparagine or Glutamine; and Alanine by Glycine, Leucine, Valine, or Isoleucine. Also, the one or more mutations, such as the one or more substitutions may represent conservative nucleotide mutations (i.e., one or more nucleotide substitutions that do not result in any changes of the encoded amino acid residues). Thus, the amino acid sequence of the polypeptide encoded by the nucleic acid molecule of the present invention may be the same as the sequence of the corresponding nucleic acid molecule with an unmodified miRNA binding site. Thus, the nucleic acid molecule of the present invention may code for a polypeptide comprising an amino acid sequence as shown in SEQ ID NO: 44, 63, or 65.

Without being bound to any theory, it is believed that the modification of the miRNA binding site leads to a reduction of binding of the miRNA to the nucleic acid molecule of the present invention (as compared to a nucleic acid molecule comprising an unmodified miRNA binding site), leading to lower levels of miRNA-driven transcript cleavage, and thereby increasing expression of the nucleic acid molecule encoding the functional restorer gene. This improves the restoration capacity.

Thus, the one or more modifications of the miRNA binding site of the third aspect of the invention reduce the binding of the miRNA to the nucleic acid molecule. Thus, the mutation of the miRNA binding site results in a lower number of base pairs formed between the binding site and the miRNA as compared to the number of base pairs formed between the unmodified binding site and the miRNA, resulting in a lower binding efficacy/strength of the miRNA to the miRNA binding site.

This is preferably achieved by substituting one or more nucleotides in the miRNA binding site which do not form Watson-Crick base pairs with the corresponding nucleotide in miRNA3619. The 19 nucleotides of the naturally occurring miRNA binding site in the Rf3 gene form 18 base pairs with miRNA3619 (since there is one mismatch). The 19 nucleotides of the naturally occurring miRNA binding site in the Rf1 gene forms 16 base pairs with miRNA3619 (since there are three mismatches). Preferably, the mutated miRNA binding site forms less than 16 base pairs with miRNA3619, such as less than 15, less than 14, less than 13, less than 12, less than 11, less than 10, less than 9, less than 8, less than 7, less than 6, less than 5, less than 4, less than 3, less than 2, or 0 base pairs.

In an embodiment, the mutated miRNA binding site forms less than 15 base pairs with miRNA3619.

In another embodiment, the mutated miRNA binding site forms less than 13 base pairs with miRNA3619.

In another embodiment, the mutated miRNA binding site forms less than 11 base pairs with miRNA3619.

In an embodiment of the third aspect of the present invention, 1, 2, 3, 4, 5, 6, 7, 8, 9, 10, 11, 12, 13, 14, 15, 16, 17, 18 or 19 nucleotides in the (naturally present) miRNA binding site of the invention are substituted with a different nucleotide.

In one embodiment, the mutated miRNA binding site is no longer a functional miRNA binding site. Thus, the miRNA is not capable of binding to the mutated miRNA binding site (because the complementarity is too low). In other words, the modified miRNA binding site is no longer targeted by miRNA3619.

In the studies underlying the third aspect of the present invention, 1 to 9 nucleotides in the miRNA binding site were substituted in a reporter construct. The enhancing effect on the expression of the reporter was more pronounced when more nucleotides were substituted (see Example 8). Thus, in one embodiment, more than one nucleotide is substituted in the miRNA binding site, such as at least 3 nucleotides, at least 5 nucleotides, or at least 8 nucleotides.

In an embodiment of the third aspect of the present invention, 2 to 19, such as 2 to 18, such as 3 to 15 such as 4 to 12 nucleotides are substituted with different nucleotides. For example, 4 to 12 nucleotides may be substituted, such as 7 to 12 nucleotides.

In an embodiment of the third aspect of the present invention, the nucleotide (or nucleotides) corresponding to position 1245, 1248, 1249, 1251, 1254, 1257, 1260, 1261 and/or 1263 in SEQ ID NO: 43 has (have) been substituted with a different nucleotide (or different nucleotides).

The tested mutated miRNA binding sites are shown in Table 1. In an embodiment of the present invention, the mutated miRNA binding site comprises a nucleic acid sequence selected from the group consisting of SEQ ID NO: 50, 52-61.

In an embodiment of the third aspect of the present invention, the miRNA binding site has been mutated by mutagenesis, such as by EMS mutagenesis or radiation mutagenesis (see also below). Thus, the resulting plant may be a non-transgenic plant.

In an embodiment of the third aspect of the present invention, the miRNA binding site has been mutated by genome editing (see below for more details).

The definitions and explanations provided herein above, preferably, apply mutatis mutandis to the following.

The nucleic acid molecule encoding a functional restorer polypeptide for wheat G-type cytoplasmic male sterility may also be cloned and a chimeric gene may be made, e.g., by operably linking a plant expressible promoter to the nucleic acid molecule and optionally a 3′ end region involved in transcription termination and polyadenylation functional in plants. Such a chimeric gene may be introduced into a plant cell, and the plant cell may be regenerated into a whole plant to produce a transgenic plant.

The the third aspect of present invention thus relates to a chimeric nucleic acid molecule comprising the following operably linked elements
a) a plant-expressible promoter;
b) the nucleic acid molecule of the present invention; and optionally
c) a transcription termination and polyadenylation region functional in plant cells.

As used herein a “chimeric gene” refers to a nucleic acid construct which is not normally found in a plant species. “Chimeric DNA construct” and “chimeric gene” are used interchangeably to denote a gene in which the promoter or one or more other regulatory regions, such as the transcription termination and polyadenylation region of the gene are not associated in nature with part or all of the transcribed DNA region, or a gene which is present in a locus in the plant genome in which it does not occur naturally or present in a plant in which it does not naturally occur. In other words, the gene and the operably-linked regulatory region or the gene and the genomic locus or the gene and the plant are heterologous with respect to each other, i.e. they do not naturally occur together (such as when either the coding sequence or the regulatory elements operably-linked to such coding sequence (such as the promoter) have been modified by nucleotide substitution (e.g., via transformation, genome editing or mutagenesis).

The term “operably linked” as used herein refers to a functional linkage between the promoter sequence and the gene of interest, such that the promoter sequence is able to initiate transcription of the gene of interest.

The term “promoter” as used in the third aspect of the current invention refers to a regulatory nucleic acid sequence capable of effecting expression of the sequences to which they are ligated. The term “promoter” as used in the third aspect of the current invention typically refers to a nucleic acid control sequence located upstream from the transcriptional start of a gene and which is involved in recognizing and binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a—box sequence and/or—10 box transcriptional regulatory sequences. The term “regulatory element” also encompasses a synthetic fusion molecule or derivative that confers, activates or enhances expression of a nucleic acid molecule in a cell, tissue or organ.

In a preferred embodiment of the third aspect, the term “promoter” refers to the promoter as defined in Section A. Accordingly, the promoter is, preferably, the modified promoter comprising at least one heterologous binding site for an EIL3 transcription factor and/or at least one heterologous binding site for a PHD transcription factor as defined in Section A (such as the promoter in any one of the embodiments 1 to 40 in Section A). For example, the promoter is the promoter of an Rf3, such as the Rf3-58, Rf3-29a or the Rf3 Fielder, gene comprising an additional binding site for an EIL3 transcription factor and/or an additional binding site for a PHD transcription factor (preferably both). Alternatively, the promoter is the Rf1-09 promoter comprising at least one heterologous binding site for an EIL3 transcription factor and/or at least one heterologous binding site for a PHD transcription factor as defined in Section A (such as the promoter in any one of the embodiments 1 to 40 in Section A).

In another preferred embodiment of the third aspect, the term “promoter” refers to the promoter as defined in Section B (such as the promoter in any one of the embodiments 1 to 26 in Section B). Accordingly, the promoter is a modified promoter of a functional restorer gene for wheat cytoplasmic male sterility (such as G-type wheat cytoplasmic male sterility) comprising one or more enhancers as defined in (any one of the embodiments of) Section B. For example, the promoter is the promoter of an Rf3, such as the Rf3-58, Rf3-29a or the Rf3 Fielder, gene comprising one or more of said enhancers. Alternatively, the promoter is the Rf1-09 promoter comprising one or more of said enhancers.

In a preferred embodiment of the third aspect, the term “promoter” refers to a promoter of a functional restorer gene for wheat cytoplasmic male sterility comprising the promoter modifications as described in Section A (such as the promoter in any one of the embodiments 1 to 40 in Section A) and in Section B (such as the promoter in any one of the embodiments 1 to 26 in Section B). Thus, the promoter is a modified promoter of a functional restorer gene for wheat cytoplasmic male sterility (such as an Rf3 or Rf1, e.g., Rf3-58, Rf3-29a, Rf3 Fielder or Rf1-09 promoter), said promoter comprising i) at least one heterologous binding site for an EIL3 transcription factor and/or at least one heterologous binding site for a PHD transcription factor as defined in (any one of the embodiments of) Section A, and ii) one or more enhancers as described in (any one of the embodiments of) Section B.

A “plant-expressible promoter” as used in the third aspect of the current invention comprises regulatory elements, which mediate the expression of a coding sequence segment in plant cells. Accordingly, a plant promoter need not be of plant origin, but may originate from viruses or micro-organisms, for example from viruses which attack plant cells. The “plant promoter” can also originate from a plant cell, e.g., from the plant which is transformed with the nucleic acid sequence to be expressed in the inventive process and described herein. This also applies to other “plant” regulatory signals, such as “plant” terminators. The promoters upstream of the nucleotide sequences useful in the methods of the present invention can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described above, be linked operably to or comprise a suitable promoter which expresses the gene at the right point in time and with the required spatial expression pattern.

Preferably, the promoter to be used is a promoter that is capable of directing expression of the operably linked nucleic acid at least during (early) pollen development and meiosis, such as in anther or, more specifically, tapetum, or developing microspores. This can for example be a constitutive promoter, an inducible promoter, but also a pollen-, anther- or, more specifically tapetum- or microspore-specific/preferential promoter.

In an embodiment of the third aspect of the current invention, the promoter is a constitutive promoter. A “constitutive promoter” refers to a promoter that is transcriptionally active during most, but not necessarily all, phases of growth and development and under most environmental conditions, in at least one cell, tissue or organ. Examples of plant expressible constitutive promoters include promoters of bacterial origin, such as the octopine synthase (OCS) and nopaline synthase (NOS) promoters from Agrobacterium, but also promoters of viral origin, such as that of the cauliflower mosaic virus (CaMV) 35S transcript (Hapster et al., 1988, Mol. Gen. Genet. 212: 182-190) or 19S RNAs genes (Odell et al., 1985, Nature. 6; 313(6005):810-2; U.S. Pat. No. 5,352,605; WO 84/02913; Benfey et al., 1989, EMBO J. 8:2195-2202), the enhanced 2×35S promoter (Kay at al., 1987, Science 236:1299-1302; Datla et al. (1993), Plant Sci 94:139-149) promoters of the cassava vein mosaic virus (CsVMV; WO 97/48819, U.S. Pat. No. 7,053,205), 2×CsVMV (WO2004/053135) the circovirus (AU 689 311) promoter, the sugarcane bacilliform badnavirus (ScBV) promoter (Samac et al., 2004, Transgenic Res. 13(4):349-61), the figwort mosaic virus (FMV) promoter (Sanger et al., 1990, Plant Mol Biol. 14(3):433-43), the subterranean clover virus promoter No 4 or No 7 (WO 96/06932) and the enhanced 35S promoter as described in U.S. Pat. Nos. 5,164,316, 5,196,525, 5,322,938, 5,359,142 and 5,424,200. Among the promoters of plant origin, mention will be made of the promoters of the plant ribulosebiscarboxylase/oxygenase (Rubisco) small subunit promoter (U.S. Pat. No. 4,962,028; WO99/25842) from Zea mays and sunflower, the promoter of the Arabidopsis thaliana histone H4 gene (Chaboute et al., 1987), the ubiquitin promoters (Holtorf et al., 1995, Plant Mol. Biol. 29:637-649, U.S. Pat. No. 5,510,474) of Maize, Rice and sugarcane, the Rice actin 1 promoter (Act-1, U.S. Pat. No. 5,641,876), the histone promoters as described in EP 0 507 698 A1, the Maize alcohol dehydrogenase 1 promoter (Adh-1) (from http://www.patentlens.net/daisy/promoters/242.html)). Also the small subunit promoter from Chrysanthemum may be used if that use is combined with the use of the respective terminator (Outchkourov et al., Planta, 216: 1003-1012, 2003).

In another embodiment of the third aspect of the current invention, the promoter is a developmentally-regulated promoter. A developmentally-regulated promoter is active during certain developmental stages, such as during early pollen development, or in parts of the plant that undergo developmental changes.

In another embodiment, the promoter of the third aspect of the current invention is an inducible promoter. An inducible promoter has induced or increased transcription initiation in response to a chemical (for a review see Gatz 1997, Annu. Rev. Plant Physiol. Plant Mol. Biol., 48:89-108), environmental or physical stimulus, or may be “stress-inducible”, i.e., activated when a plant is exposed to various stress conditions, or a “pathogen-inducible”, i.e., activated when a plant is exposed to exposure to various pathogens.

In another embodiment, the promoter of the third aspect of the current invention is an organ-specific or tissue-specific promoter. An organ-specific or tissue-specific promoter is one that is capable of preferentially initiating transcription in certain organs or tissues, such as the leaves, roots, seed tissue etc. For example, a “pollen-specific promoter” is a promoter that is transcriptionally active predominantly in plant pollen. A pollen-specific promoter might still allow for leaky expression in other plant parts.

Pollen/microspore-active promoters include, e.g., a maize pollen specific promoter (see, e.g., Guerrero (1990) Mol. Gen. Genet. 224:161 168), PTA29, PTA26 and PTAI 3 (e.g., see U.S. Pat. No. 5,792,929) and as described in, e.g., Baerson et al. (1994 Plant Mol. Biol. 26: 1947-1959), the NMT19 microspore-specific promoter as, e.g., descibed in WO97/30166. Further anther/pollen-specific or anther/pollen-active promoters are described in, e.g., Khurana et al., 2012 (Critical Reviews in Plant Sciences, 31: 359-390), WO2005100575, WO 2008037436. Other suitable promoters are e.g the barley vrn1 promoter, such as described in Alonso-Peral et al. (2001, PLoS One. 2011; 6(12):e29456).

The transcription termination and polyadenylation region is a terminator. The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene.

The functional restorer gene allele of the third aspect of the current invention can also encode a PPR protein which when expressed is targeted to the mitochondrion. This can, e.g., be accomplished by the presence of a (plant-functional) mitochondrial targeting sequence or mitochondrial signal peptide, or mitochondrial transit peptide. A mitochondrial targeting signal is a 10-70 amino acid long peptide that directs a newly synthesized protein to the mitochondria, typically found at the N-terminus. Mitochondrial transit peptides are rich in positively charged amino acids but usually lack negative charges. They have the potential to form amphipathic a-helices in nonaqueous environments, such as membranes. Mitochondrial targeting signals can contain additional signals that subsequently target the protein to different regions of the mitochondria, such as the mitochondrial matrix. Like signal peptides, mitochondrial targeting signals are cleaved once targeting is complete. Mitochondrial Transit peptides are, e.g., described in Shewry and Gutteridge (1992, Plant Protein Engineering, 143-146, and references therein), Sjoling and Glaser (Trends Plant Sci Volume 3, Issue 4, 1 Apr. 1998, Pages 136-140), Pfanner (2000, Current Biol, Volume 10, Issue 11), Huang et al (2009, Plant Phys 150(3): 1272-1285), Chen et al. (1996, PNAS, Vol. 93, pp. 11763-11768). In one example, such a sequence can be amino acids 1-50 of SEQ ID NO. 62).

The nucleic acid molecule of the third aspect of the present invention or the chimeric gene of the third aspect of the present invention may be introduced into a plant. As used herein, it encompasses any method for introducing a gene into a plant. In an embodiment, the nucleic acid molecule or chimeric gene is introduced into a plant by crossing two plants. For example, the nucleic acid molecule or chimeric gene is introduced into a plant by crossing two plants, whereas one plant comprises the nucleic acid molecule or chimeric gene of the present invention. The second plant may lack said nucleic acid molecule or chimeric gene. In an alternative embodiment, the gene is introduced by genome editing. The term is described elsewhere herein. In a third embodiment, the gene is introduced by transformation. The term “transformation” as referred to herein encompasses the transfer of an exogenous polynucleotide into a host cell. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation, as used herein, means introducing a nucleotide sequence into a plant in a manner to cause stable or transient expression of the sequence. Transformation and regeneration of both monocotyledonous and dicotyledonous plant cells is now routine, and the selection of the most appropriate transformation technique will be determined by the practitioner. The choice of method will vary with the type of plant to be transformed; those skilled in the art will recognize the suitability of particular methods for given plant types. Suitable methods can include, but are not limited to: electroporation of plant protoplasts; liposome-mediated transformation; polyethylene glycol (PEG) mediated transformation; transformation using viruses; micro-injection of plant cells; micro-projectile bombardment of plant cells; vacuum infiltration; and Agrobacterium-mediated transformation. Transgenic plants are preferably produced via Agrobacterium-mediated transformation. The genetically modified plant cells can be regenerated via all methods with which the skilled worker is familiar. After introduction, the plant may be selected for the presence of the nucleic acid molecule or chimeric gene of the present invention.

In one aspect, the chimeric gene is stably integrated into the cereal (e.g., wheat) genome.

The third aspect of the present invention also relates to a plant cell, such as a cereal plant cell, or plant, such as a cereal plant or seed thereof, such as a wheat plant cell or plant or seed thereof, comprising the nucleic acid molecule of the present invention, or the chimeric gene of the present invention.

In an embodiment of the plant cell, the plant or seed of the third aspect of the present invention, the plant cell, plant or seed is a hybrid plant cell, plant or seed.

The term “cereal” relates to members of the monocotyledonous family Poaceae which are cultivated for the edible components of their grain. These grains are composed of endosperm, germ and bran. Maize, wheat and rice together account for more than 80% of the worldwide grain production. Other members of the cereal family comprise rye, oats, barley, triticale, sorghum, wild rice, spelt, einkorn, emmer, durum wheat and kamut.

In one embodiment, a cereal plant according to the invention is a cereal plant that comprises at least a B genome or related genome, such as wheat (Triticum aestivum; ABD), spelt (Triticum spelta; ABD) durum (T. turgidum; AB), barley (Hordeum vulgare; H) and rye (Secale cereale; R). In a specific embodiment, the cereal plant according to the invention is wheat (Triticum aestivum; ABD).

Thus, cereal plants, plant parts, plant cells, or seeds thereof, especially wheat, comprising the nucleic acid molecule or chimeric gene encoding a functional restorer polypeptide as set forth herein are provided, said plant having an improved capacity to restore fertility against wheat G-type cytoplasmic male sterility. In one embodiment, the acid molecule, polypeptide or chimeric gene is heterologous to the plant, such as transgenic, mutated or genome edited cereal plants or transgenic, mutated or genome edited wheat plants. This also includes plant cells or cell cultures comprising such nucleic acid molecule or chimeric gene, independent whether introduced by transgenic methods or by breeding methods. The cells are, e.g., in vitro and are regenerable into plants comprising the nucleic acid molecule or chimeric gene of the invention. Said plants, plant parts, plant cells and seeds may also be hybrid plants, plant parts, plant cells or seeds.

Whenever reference to a “plant” or “plants” according to the invention is made, it is understood that also plant parts (cells, tissues or organs, seed pods, seeds, severed parts such as roots, leaves, flowers, pollen, etc.), progeny of the plants which retain the distinguishing characteristics of the parents (especially the restoring capacity), such as seed obtained by selfing or crossing, e.g. hybrid seed (obtained by crossing two inbred parental lines), hybrid plants and plant parts derived there from are encompassed herein, unless otherwise indicated.

In an embodiment, the plant of the third aspect of the present invention has been generated by chemical mutagenesis, such as by EMS (ethyl methanesulfonate) mutagenesis, NaN₃ (sodium azide) mutagenesis, or ENU (N-ethyl-N-nitrosourea) mutagenesis. Thus, the mutation(s) in the miRNA binding site as referred to herein has (have) been introduced by EMS (Ethyl methanesulfonate) mutagenesis, NaN3 (sodium azide) mutagenesis, or ENU (N-ethyl-N-nitrosourea) mutagenesis. EMS is a mutagenic compound that produces mutations at random positions in genetic material by nucleotide substitution; particularly through G:C to A:T transitions induced by guanine alkylation. Similarly, NaN3 is a mutagenic compound that produces mutations at random positions in genetic material by nucleotide substitution; particularly through A:T to GC transitions and G:C to A:T transitions and G:C to T:A changes and A:T to T:A changes. Similarly, ENU is a mutagenic compound that produces mutations at random positions in genetic material by nucleotide substitution; particularly through A:T to T:A changes and G:C to A:T transitions and A:T to G:C transitions.

In an embodiment, the chemical mutagenesis is EMS (ethyl methanesulfonate) mutagenesis.

In another embodiment, the plant of the third aspect of the present invention has been generated by irradiation induced mutagenesis, in particular gamma irradiation or fast-neutron irradiation, or X-ray irradiation. Thus, the mutation(s) in the miRNA binding site as referred to herein has (have) been introduced by radiation induced mutagenesis.

In yet another embodiment, the plant of the third aspect of the present invention has been generated by genome editing. Thus, the mutation(s) in the miRNA binding site as referred to herein has (have) been introduced by genome editing. Genome editing, as used herein, refers to the targeted modification of genomic DNA using sequence-specific enzymes (such as endonuclease, nickases, base conversion enzymes/base editors) and/or donor nucleic acids (e.g., dsDNA, oligo's) to introduce desired changes in the DNA. Sequence-specific nucleases that can be programmed to recognize specific DNA sequences include meganucleases (MGNs), zinc-finger nucleases (ZFNs), TAL-effector nucleases (TALENs) and RNA-guided or DNA-guided nucleases such as Cas9, Cpf1, CasX, CasY, C2c1, C2c3, certain argonout systems (see e.g. Osakabe and Osakabe, Plant Cell Physiol. 2015 March; 56(3):389-400; Ma et al., Mol Plant. 2016 Jul. 6; 9(7):961-74; Bortesie et al., Plant Biotech J, 2016, 14; Murovec et al., Plant Biotechnol J. 2017 Apr. 1; Nakade et al., Bioengineered 8-3, 2017; Burstein et al., Nature 542, 37-241; Komor et al., Nature 533, 420-424, 2016; all incorporated herein by reference). Donor nucleic acids can be used as a template for repair of the DNA break induced by a sequence specific nuclease, but can also be used as such for gene targeting (without DNA break induction) to introduce a desired change into the genomic DNA. Genome editing also includes technologies like prime editing (can mediate targeted insertions, deletions, and base-to-base conversions without the need for double strand breaks or donor DNA templates), see, e.g., Anzalone et al. 2019)

By using the above technologies, plants comprising a naturally occurring miRNA binding site within a gene for wheat G-type cytoplasmic male sterility can be converted to plants having a mutated miRNA binding site, thereby improving restoration capacity for wheat G-type cytoplasmic male sterility (“CMS”) in a cereal plant.

In accordance with the third aspect of the present invention, plants can be generated by genome editing that are not considered transgenic plants.

The obtained plants according to the third aspect of the invention can be used in a conventional breeding scheme to produce more plants with the same characteristics or to introduce the characteristic of the presence of the restorer gene according to the invention in other varieties of the same or related plant species, or in hybrid plants. The obtained plants can further be used for creating propagating material. Plants according to the invention can further be used to produce gametes, seeds, flour, embryos, either zygotic or somatic, progeny or hybrids of plants obtained by methods of the invention. Seeds obtained from the plants according to the invention are also encompassed by the invention.

In an embodiment, the plant, or plant cell of the third aspect of the present invention has not been obtained exclusively by an essentially biological process for the production of plants.

The third aspect of the present invention also relates to a method for producing a cereal plant cell or plant or seed thereof, such as a wheat plant cell or plant or seed thereof, comprising a functional restorer gene for wheat G-type cytoplasmic male sterility as set forth herein, said method comprising the steps of providing said plant cell or plant with the nucleic acid molecule of the present invention or the chimeric gene of the present invention. The nucleic acid molecule or chimeric gene may be provided as described elsewhere herein, such as by transformation, crossing, backcrossing, genome editing or mutagenesis.

The plant of the third aspect of the present invention or produced by the method of the third aspect of the present invention has at least one, preferably both of the following characteristics:
it has an increased restoration capacity for wheat G-type cytoplasmic male sterility (“CMS”) as compared to a control plant, and/or
it has an increased expression of the functional restorer polypeptide for wheat G-type cytoplasmic male sterility as compared to a control plant.

The choice of suitable control plants is a routine part of an experimental setup and may include a corresponding wild type plant or a corresponding plant comprising the nucleic acid molecule encoding a functional restorer polypeptide for wheat G-type cytoplasmic male sterility with a non-modified miRNA binding site (or chimeric gene comprising said nucleic acid molecule). Thus, the control nucleic acid molecule may comprise, in its coding sequence, the naturally occurring miRNA binding site for miRNA3619. The control plant is typically of the same plant species or even of the same variety as the plant to be assessed. Further, a control plant has been grown under equal growing conditions to the growing conditions of the plants of the invention. Typically, the control plant is grown under equal growing conditions and hence in the vicinity of the plants of the invention and at the same time. A “control plant” as used herein refers not only to whole plants, but also to plant parts, including the anther and pollen.

Whether the expression of the functional restorer polypeptide is increased as compared to the expression in a control plant, or not, can be determined by well-known methods. The terms “increase”, “improve” or “enhance” are interchangeable and mean an increase of expression of at least 15% or 20%, more preferably of at least 30%, at least 40%, at least 60%, at least 80%, or at least 100% in comparison to a control plant as defined herein. Preferably, said increase in expression is at least during (the early phases of) pollen development and meiosis, such as in anther or, more specifically, tapetum, or developing microspores.

Restoration capacity, as used herein, means the capacity of a plant to restore fertility in the progeny of a cross with a G-type cytoplasmic male sterility (“CMS”) line. Whether plant has an increased restoration capacity for wheat G-type cytoplasmic male sterility (“CMS”) compared to a control can be assessed by well-known methods, e.g., by the method described in Example 10. For example, the nucleic acid molecule or chimeric gene of the invention might be introduced into a cereal (wheat) plant that does comprise said molecule or gene in a (wheat) plant having G-type CMS, or in a (wheat) plant lacking G-type CMS which is then crossed with a G-type cytoplasmic male sterile (wheat) plant and evaluating seed set in the progeny. The number of set seed is indicative for the restoration capacity of the plant. A seed set which is at least 10%, at least 20% or at least 30% higher than the seed set in the control plant is considered to be indicative for an increased restoration capacity.

Moreover, pollen accumulation and pollen viability can be quantified in order to assess the restoration capacity. The modification of the miRNA binding site in the Rf3 gene leads to higher numbers of viable pollen (in (wheat) plants with G-type CMS).

The third aspect of the present invention also relates to a method for improving expression of a functional restorer gene for wheat G-type cytoplasmic male sterility, or for increasing restoration capacity for wheat G-type cytoplasmic male sterility (“CMS”) in a cereal plant, such as a wheat plant, comprising the step of providing said plant cell or plant with the nucleic acid molecule of the third aspect of the present invention or the chimeric gene of the third aspect of the present invention. The nucleic acid molecule or chimeric gene may be provided as described elsewhere herein, such as by transformation, crossing, backcrossing, genome editing or mutagenesis (for example by chemical mutagenesis, such as EMS mutagenesis, or mutagenesis arising via somaclonal variation).

The third aspect of the present invention also relates to a cereal plant cell or cereal plant or seed thereof, such as a wheat plant cell or plant or seed thereof, obtained according to the method of any one of the present invention. For example, the plant cell, plant or seed is a hybrid plant cell, plant or seed.

The third aspect of the present invention also relates to a method for identifying and/or selecting a cereal (e.g., wheat) plant comprising an improved functional restorer gene allele for wheat G-type cytoplasmic male sterility comprising the steps of:
a. Identifying or detecting in said plant the presence of the nucleic acid molecule of the present invention or the chimeric gene of the present invention, or a modified miRNA binding site as set forth herein, and
b. selecting said plant comprising said nucleic acid or chimeric gene, or said modified miRNA binding site.

The third aspect of the present invention also relates to a method for producing hybrid seed, comprising the steps of:
a) Providing a male cereal parent plant, such as a wheat plant, of the present invention, said plant comprising a nucleic acid molecule for a functional restorer polypeptide for wheat G-type cytoplasmic male sterility according to the current invention, wherein said nucleic acid molecule is preferably present in homozygous form,
b) Providing a female cereal parent plant, such as a wheat plant, that is a G-type cytoplasmic male sterile cereal plant,
c) Crossing said female cereal parent plant with said male cereal parent plant; and optionally
d) Harvesting seeds.

The third aspect of the present invention also relates to a method for producing hybrid plants, comprising the steps of:
a) Harvesting seeds from a cross of
   a1) a male cereal parent plant, such as a wheat plant, of the present invention, said plant comprising a nucleic acid molecule for a functional restorer polypeptide for wheat G-type cytoplasmic male sterility according to the current invention, wherein said nucleic acid molecule is preferably present in homozygous form, and
   a2) a female cereal parent plant, such as a wheat plant, that is a G-type cytoplasmic male sterile cereal plant, and
b) Growing plants from the seeds harvested in step a).

The method may further comprise the step of harvesting seeds from the plants grown in step b).

As used herein, the term “homozygous” means a genetic condition existing when two identical alleles reside at a specific locus, but are positioned individually on corresponding pairs of homologous chromosomes in the cell. Conversely, the term “heterozygous” means a genetic condition existing when two different alleles reside at a specific locus, but are positioned individually on corresponding pairs of homologous chromosomes in the cell.

In any of the herein described embodiments and aspects the plant may comprise or may be selected to comprise or may be provided with a further functional restorer gene (further to Rf3) for wheat G-type cytoplasmic male sterility (located on or obtainable from the same or another chromosome), such as Rf1 (1A), Rf2 (7D), Rf4 (6B), Rf5 (6D), Rf6 (5D), Rf7 (7B), Rf8, Rf9, 6AS or 6BS.

The third aspect of the present invention also relates to the use of the nucleic acid molecule or of the chimeric gene of the present invention for the identification of a plant comprising said functional restorer gene allele for wheat G-type cytoplasmic male sterility.

The third aspect of the present invention also relates to the use of the nucleic acid molecule or of the chimeric gene of the present invention for generating plants comprising said functional restorer gene allele for wheat G-type cytoplasmic male sterility.

The third aspect of the present invention furthermore relates to the use of a plant of the present invention for restoring fertility in a progeny of a G-type cytoplasmic male sterile cereal plant, such as a wheat plant.

The third aspect of the present invention furthermore relates to the use of the plant of the present invention, said plant comprising said functional restorer gene for wheat G-type cytoplasmic male sterility, for producing hybrid seed or a population of hybrid cereal plants, such as hybrid wheat seed or plants.

### Embodiments of the Third Aspect of the Present Invention (Section C, Modified miRNA Binding Binding Sites)

The nucleic acid molecules plants, constructs, uses etc. as described in section C are further illustrated by the following embodiments and combinations of embodiments as indicated by the respective dependencies and back-references. The definitions and explanations given herein above apply mutatis mutandis to the following embodiments.
1. A nucleic acid molecule encoding a functional restorer polypeptide for wheat G-type cytoplasmic male sterility, wherein said nucleic acid molecule comprises a mutated miRNA binding site in the coding sequence.
2. The nucleic acid molecule of embodiment 1, wherein
   a) the nucleic acid molecule is a mutated Rf3 gene which does not comprise a sequence as shown in SEQ ID NO: 45 (GGGUAGGUUGGAUGAUGCU) or SEQ ID NO: 46 (gggtag gttggatgatgct), or
   b) the nucleic acid molecule is a mutated Rf1 gene which does not comprise a sequence as shown in SEQ ID NO: 67 (gggucgguuggacgaugcu) or SEQ ID NO: 66 (gggtcggttggacgatgct).
3. The nucleic acid molecule of embodiment 1 or 2, wherein the functional restorer polypeptide comprises
   a) an amino acid sequence as shown in SEQ ID NO: 44, 63, or 65 or
   b) an amino acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 44, 63 or 65.
4. The nucleic acid molecule of any one of embodiments 1 to 3, comprising
   a) at least one mutation in the nucleic acid sequence as shown in SEQ ID NO: 43 or
   b) at least one mutation in a nucleic acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 43,
wherein one or more nucleotide(s) at a position in the region corresponding to the region from nucleotide position 1245 to nucleotide position 1263 in SEQ ID NO: 43 are mutated.
5. The nucleic acid molecule of embodiment 1 or 2, wherein said miRNA binding site has been mutated in a translationally neutral or in a conservative manner.
6. The nucleic acid molecule of any one of embodiments 1 to 5, wherein the mutation of the miRNA binding site results in the formation of a lower number of base pairs formed between the binding site and miRNA 3619 as compared to the number of base pairs formed between the unmodified binding site and miRNA3619, for example, wherein less than 13 or less than 11 base pairs are formed.
7. The nucleic acid molecule of any one of embodiments 2 to 6, wherein the one or more nucleotides have been mutated by substituting, deleting and/or adding one or more nucleotides at a position corresponding to a position in the region from nucleotide position 1245 to nucleotide position 1263 in SEQ ID NO: 43.
8. The nucleic acid molecule of embodiment 7, wherein the one or more nucleotides have been substituted with one or more different nucleotides.
9. The nucleic acid molecule of embodiment 8, wherein 1, 2, 3, 4, 5, 6, 7, 8, 9, 10, 11, 12, 13, 14, 15, 16, 17, 18 or 19 nucleotides have been substituted with a different nucleotide.
10. The nucleic acid molecule of any one of embodiments 2 to 9, wherein the nucleotide (or nucleotides) corresponding to position 1245, 1248, 1249, 1250, 1251, 1254, 1257, 1260, 1262 and/or 1263 in SEQ ID NO: 43 has (have) been substituted with a different nucleotide (or different nucleotides).
11. The nucleic acid molecule of any one of embodiments 1 to 10, wherein said miRNA binding site has been mutated by chemical mutagenesis, such as by EMS mutagenesis.
12. A polypeptide encoded by the nucleic acid of any one of embodiments 1 to 9, wherein said polypeptide comprises at least one substituted amino acid residue in at least one position corresponding to position 415, 416, 417, 418, 419, 420 and/or 421 of SEQ ID NO: 44.
13. A chimeric nucleic acid molecule comprising the following operably linked elements
   a. a plant-expressible promoter,
   b. the nucleic acid molecule of any one of embodiments 1-11; and optionally
   c. a transcription termination and polyadenylation region functional in plant cells.
14. The chimeric gene of embodiment 13, wherein said promoter is capable of directing expression of the operably linked nucleic acid at least during early pollen development and meiosis.
15. A cereal plant cell or cereal plant or seed thereof, such as a wheat plant cell or plant or seed thereof, comprising the nucleic acid molecule of any one of embodiments 1-11, or the chimeric gene of embodiment 13 or 14.
16. The cereal plant cell, plant or seed of embodiment 15, which is a hybrid plant cell, plant or seed.
17. A method for producing a cereal plant cell or plant or seed thereof, such as a wheat plant cell or plant or seed thereof, comprising a functional restorer gene for wheat G-type cytoplasmic male sterility, or for increasing restoration capacity for wheat G-type cytoplasmic male sterility (“CMS”) in a cereal plant, such as a wheat plant, comprising the steps of providing said plant cell or plant with the nucleic acid molecule of embodiments 1-11 or the chimeric gene of embodiment 13 or 14.
18. A method for improving expression of a functional restorer gene for wheat G-type cytoplasmic male sterility, or for increasing restoration capacity for wheat G-type cytoplasmic male sterility (“CMS”) in a cereal plant, such as a wheat plant, comprising the step of providing said plant cell or plant with the nucleic acid molecule of embodiments 1-11 or the chimeric gene of embodiment 13 or 14.
19. A cereal plant cell or cereal plant or seed thereof, such as a wheat plant cell or plant or seed thereof, obtained according to the method of any one of embodiments 15 to 16.
20. The plant cell, plant or seed of embodiment 17, which is a hybrid plant cell, plant or seed.
21. A method for identifying and/or selecting a cereal (e.g. wheat) plant comprising an improved functional restorer gene allele for wheat G-type cytoplasmic male sterility comprising the steps of:
   a. Identifying or detecting in said plant the presence of the nucleic acid molecule of any one of embodiments 1-11 or the chimeric gene of embodiment 13 or 14, or said modified miRNA binding site,
   b. and selecting said plant comprising said nucleic acid or chimeric gene.
22. A method for producing hybrid seed, comprising the steps of:
   a. Providing a male cereal parent plant, such as a wheat plant, according to embodiment or 16, and/or comprising the nucleic acid molecule of any one of embodiments 1-11 or the chimeric gene of embodiment 13 or 14, wherein nucleic acid molecule or chimeric gene is preferably present in homozygous form,
   b. Providing a female cereal parent plant that is a G-type cytoplasmic male sterile cereal plant,
   c. Crossing said female cereal parent plant with a said male cereal parent plant; and optionally
   d. Harvesting seeds.
23. Use of the nucleic acid of any one of embodiments 1 to 11 or of the chimeric gene of embodiment 13 or 14 for the identification of a plant comprising said functional restorer gene allele for wheat G-type cytoplasmic male sterility.
24. Use of the nucleic acid of any one of embodiments 1 to 11 or of the chimeric gene of embodiment 13 or 14 for generating plants comprising said functional restorer gene allele for wheat G-type cytoplasmic male sterility.
25. Use of a plant according to embodiment 15 or 16, or a plant obtained or obtainable by the method of claim 16 for restoring fertility in a progeny of a G-type cytoplasmic male sterile cereal plant, such as a wheat plant.
26. Use of a plant according to embodiment 15 or 16, or a plant obtained or obtainable by the method of claim 18 comprising said functional restorer gene for wheat G-type cytoplasmic male sterility, for producing hybrid seed or a population of hybrid cereal plants, such as wheat seed or plants.
27. The chimeric nucleic acid, the plant cell, the plant, the method or the use of any of the preceding embodiments, wherein the promoter is the modified promoter as defined in Section A.
28. The chimeric nucleic acid, the plant cell, the plant, the method or the use of any of the preceding embodiments, wherein the promoter is the modified promoter as defined in Section B.
29. The chimeric nucleic acid, the plant cell, the plant, the method or the use of any of the preceding embodiments, wherein the promoter is a promoter of a functional restorer gene for wheat cytoplasmic male sterility (such as the Rf3-58 or Rf1-09 promoter) comprising the modifications as described in Section A and the modifications as described in Section B.

All patents, patent applications, and publications or public disclosures (including publications on internet) referred to or cited herein are incorporated by reference in their entirety.

The invention will be further described with reference to the examples described herein; however, it is to be understood that the invention is not limited to such examples.

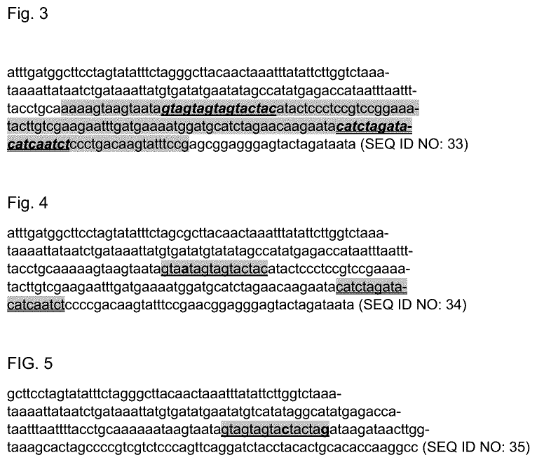

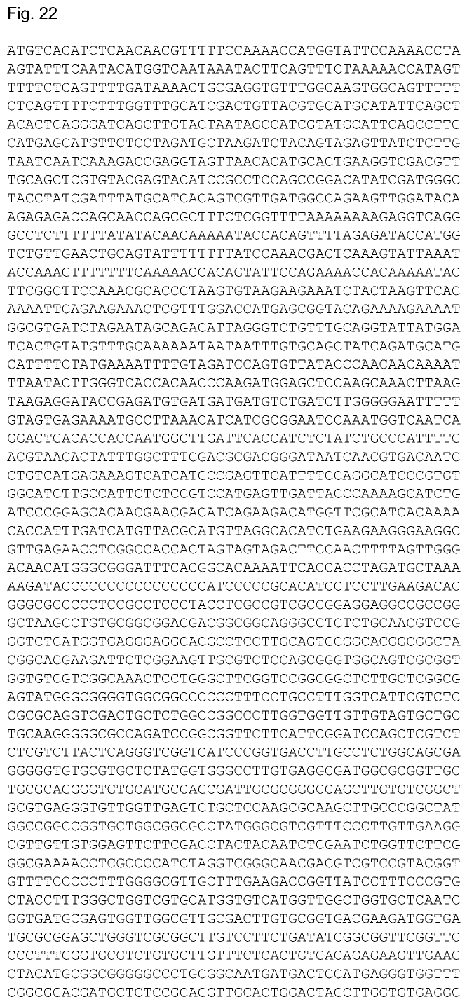

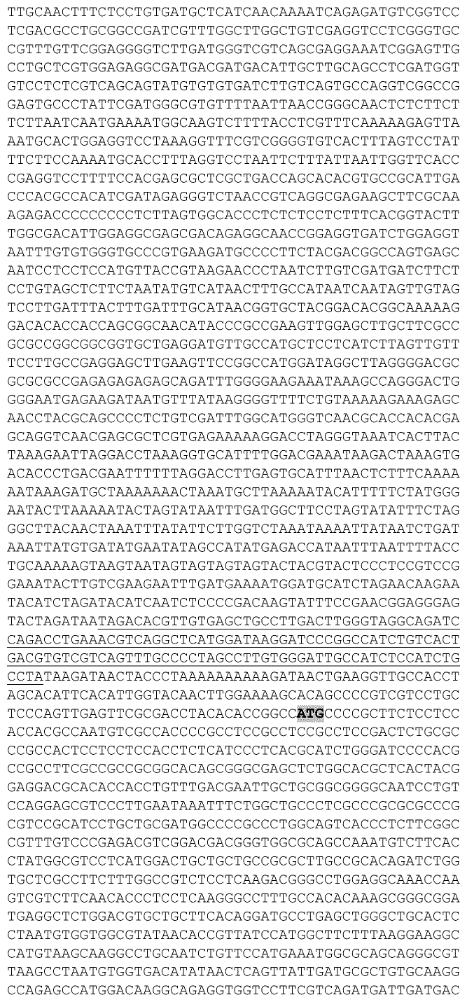

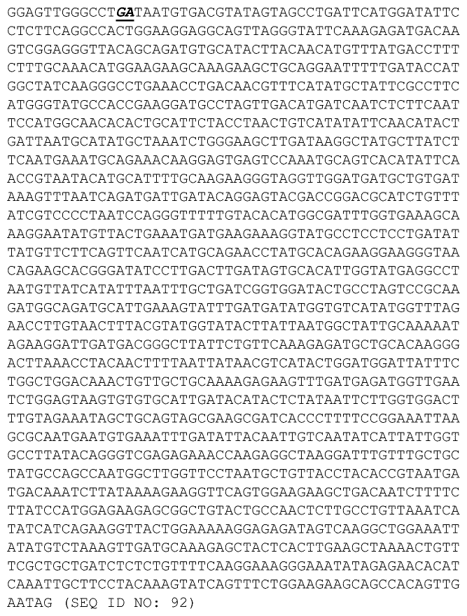

In the description, figures and examples, reference is made to the following sequences:
SEQ ID NO: 1: nucleic acid sequence of the promoter of the Rf3-58 gene from wheat.
SEQ ID NO: 2: nucleic acid sequence of the forward primer T7.
SEQ ID NO: 3: nucleic acid sequence of the reverse primer 3′AD.
SEQ ID NO: 4: amino acid sequence of the PHD transcription factor from the B subgenome of wheat.
SEQ ID NO: 5: nucleic acid sequence of the coding DNA of the PHD transcription factor from the B subgenome of wheat.
SEQ ID NO: 6: amino acid sequence of the PHD transcription factor from the D subgenome of wheat.
SEQ ID NO: 7: nucleic acid sequence of the coding DNA of the PHD transcription factor from the D subgenome of wheat.
SEQ ID NO: 8: amino acid sequence of the PHD transcription factor from the A subgenome of wheat.
SEQ ID NO: 9: nucleic acid sequence of the coding DNA of the PHD transcription factor from the A subgenome of wheat.
SEQ ID NO: 10: nucleotide sequence of a 20 bp fragment bound by the PHD transcription factor (contains PHD transcription factor binding site)
SEQ ID NO: 11: example (partially) palindromic nucleotide sequence as binding site for the PHD transcription factor from the Rf3-58 promoter
SEQ ID NO: 12: example (partially) palindromic nucleotide sequence as binding site for the PHD transcription factor from the Rf1-09 promoter.
SEQ ID NO: 13: amino acid sequence of the EIL3 transcription factor from the B subgenome of wheat.
SEQ ID NO: 14: nucleic acid sequence of the coding DNA of the EIL3 transcription factor from the B subgenome of wheat.
SEQ ID NO: 15: amino acid sequence of the EIL3 transcription factor from the D subgenome of wheat.
SEQ ID NO: 16: nucleic acid sequence of the coding DNA of the EIL3 transcription factor from the D subgenome of wheat.
SEQ ID NO: 17: amino acid sequence of the EIL3 transcription factor from the A subgenome of wheat.
SEQ ID NO: 18: nucleic acid sequence of the coding DNA of the EIL3 transcription factor from the A subgenome of wheat.
SEQ ID NO: 19: nucleotide sequence of the fragment containing the EIL3 transcription factor binding site.
SEQ ID NO: 20: nucleotide sequence of the pRF3-4 GUS expression construct.
SEQ ID NO: 21: nucleotide sequence of the about 2 kb sequence of the Rf3-58 promoter.
SEQ ID NO: 22: nucleotide sequence of the about 1.4 kb sequence of the Rf3-58 promoter.
SEQ ID NO: 23: nucleotide sequence of the about 1.2 kb sequence of the Rf3-58 promoter.
SEQ ID NO: 24: nucleotide sequence of the about 1.2 kb sequence of the Rf3-58 promoter including a duplication of the EIL3 binding site.
SEQ ID NO: 25: nucleotide sequence of the about 1.2 kb sequence of the Rf3-58 promoter including a mutated EIL3 binding site.
SEQ ID NO: 26: nucleotide sequence of the about 1.2 kb sequence of the Rf3-58 promoter including a duplication of the EIL3 and PHD binding sites.
SEQ ID NO: 27: nucleotide sequence of the p35S GFP expression construct.
SEQ ID NO: 28: nucleotide sequence of the pUbi LUC expression construct.
SEQ ID NO: 29: nucleotide sequence of the duplicated fragment comprising the binding sites of PHD and EIL.
SEQ ID NO: 30: short PHD binding site present in the Rf3-58 promoter SEQ ID NO: 31: PHD binding site present in the Rf1-09 promoter, was used as 22 bp bait sequence in Example 3
SEQ ID NO: 32: extended PHD binding site in the Rf3-58 promoter (22 bp), was used in Example 3
SEQ ID NO: 33: Rf3-58 promoter sequence (portion) shown in FIG. 3
SEQ ID NO: 34: RFL29a promoter sequence (portion) shown in FIG. 4
SEQ ID NO: 35: Rf1-09 promoter sequence (portion) shown in FIG. 5
SEQ ID NO: 36: RFL29a promoter sequence
SEQ ID NO: 37: Rf1-09 promoter sequence
SEQ ID NO: 38: short PHD binding site present in the Rf1-09 promoter
SEQ ID NO: 39: short EIL3 binding site present in the RF3-58 promoter and the RFL29a promoter
SEQ ID NO: 40: PHD binding site present in the RFL29a promoter (16 bp)
SEQ ID NO: 41: PHD binding site present in the RFL29a promoter, shorter version of
SEQ ID NO: 40 (15 bp)
SEQ ID NO: 42: PHD binding site present in the Rf3-58 promoter, shorter version of
SEQ ID NO: 11 (15 bp)
SEQ ID NO: 43: Rf3 coding sequence, herein also referred to as Rf3-58
SEQ ID NO: 44: amino acid sequence of the protein encoded by SEQ ID NO: 43
SEQ ID NO: 45: native (naturally occurring) miRNA binding site for miRNA3619 (RNA sequence) in SEQ ID NO: 43 and SEQ ID NO: 62
SEQ ID NO: 46: DNA sequence present at nucleotide position 1245 to nucleotide position 1263 of SEQ ID NO: 43. The sequence encodes the miRNA binding site of SEQ ID NO: 45
SEQ ID NO: 47 sequence of miRNA3619 (lower sequence in the alignment in FIGS. 6A, 6B and 6C)
SEQ ID NO: 48 sequence in FIGS. 6A and 6C, miRNA binding site for miRNA3619 in Rf3 mRNA variants with flanking nucleotides
SEQ ID NO: 49 sequence encoding PPR units 8 to 10 of the wheat Rf3-58 protein, optimized for expression in wheat
SEQ ID NO. 50-61: miRNA binding site sequences tested in the Examples section (see also table 1)
SEQ ID NO: 62: RFL29a Rf3 sequence (Rf3-29a, another Rf3 allele)
SEQ ID NO: 63 amino acid sequence of the protein encoded by SEQ ID NO: 62
SEQ ID NO: 64 Rf1 coding sequence
SEQ ID NO: 65 amino acid sequence of the Rf1 protein encoded by SEQ ID NO: 64
SEQ ID NO: 66 DNA sequence encoding the miRNA binding site for miRNA3619 (see
SEQ ID NO: 67)
SEQ ID NO: 67 native (naturally occurring) miRNA binding site for miRNA3619 (RNA sequence) in Rf1 (SEQ ID NO: 64)
SEQ ID NO: 68 sequence in FIG. 6B, miRNA binding site for miRNA3619 in Rf1 mRNA with flanking nucleotides
SEQ ID NO: 69: miRNA binding site which is 100% complementary to miRNA3619
SEQ ID NO: 70 wheat enhancer sequence EN1390
SEQ ID NO: 71 4138-nt promoter fragment of the wheat Rf3-58 gene
SEQ ID NO: 72 2-kb promoter fragment of the wheat Rf3-58 gene
SEQ ID NO: 73 1423-nt promoter fragment of the wheat Rf3-58 gene
SEQ ID NO: 74 variant of 1423-nt promoter fragment of the wheat Rf3-58 gene lacking the MITE-like insertion
SEQ ID NO: 75 pBay02430: vector containing a wheat-optimized sequence coding for the Streptococcus pyogenes Cas9, with an N- and C-terminal NLS, under control of the PubiZm promoter and 3-prime 35S.
SEQ ID NO: 76 pBay02032: vector containing an eGFP-BAR fusion gene under control of the CaMV 35S promoter and 3-prime35S.
SEQ ID NO: 77 pBas03477: vector containing a Cas9 guide RNA, with protospacer CAGATGATTGATGATGGTGT targeting the Fielder Rf3 gene, under the control of the wheat U6 promoter.
SEQ ID NO: 78 pBas03482: vector containing an 802 bp modified genomic DNA fragment of the Fielder Rf3 gene with a 2 nt insertion to create a functional coding sequence.
SEQ ID NO: 79 pBas03682: vector containing a Cas9 guide RNA, with protospacer AAAAGAAAGAGCAACCTACG targeting the promoter of the Fielder Rf3 gene, under the control of the wheat U6 promoter.
SEQ ID NO: 80 pBas03683: vector containing a Cas9 guide RNA, with protospacer ACGTATAGTAGCCTCATCCA targeting the coding sequence of the Fielder Rf3 gene, under the control of the wheat U6 promoter.
SEQ ID NO: 81 pBas03913: vector containing a 2470 bp modified genomic DNA fragment of the Fielder Rf3 gene to simultaneously introduce the EN1390 enhancer in the promoter and insert 2 nt in the coding sequence to create a functional coding sequence.
SEQ ID NO: 82 sequence of an edited Fielder Rf3 gene with EN1390 enhancer insertion and repaired coding sequence frameshift. nt 1-4532: sequence of the edited promoter. nt 4533-6905: sequence of the edited coding sequence.
SEQ ID NO: 83 repair DNA to simultaneously modify the Fielder Rf3 gene for optimal restoration activity: introduction of the EN1390 enhancer in the promoter, duplication of the region containing the PHD and EIL3 TF-binding sites, insertion of 2 nt in the coding sequence to create a functional coding sequence, and mutation of the miRNA3619 binding site.
SEQ ID NO: 84 sequence of an edited Fielder Rf3 gene with optimal restoration activity (EN1390 enhancer insertion, PHD and EIL3 TF-binding site region duplication, repaired coding sequence frameshift, and miRNA3619 binding site mutation. nt 1-4666: sequence of the edited promoter. nt 4667-7033: sequence of the edited coding sequence
SEQ ID NO: 85 Wheat enhancer sequence EN1390
SEQ ID NO: 86 Wheat enhancer sequence EN5458
SEQ ID NO: 87 Wheat enhancer sequence EN2393
SEQ ID NO: 88 Wheat enhancer sequence EN2968
SEQ ID NO: 89 Wheat enhancer sequence EN1391
SEQ ID NO: 90 Wheat enhancer sequence EN4730
SEQ ID NO: 91 Wheat enhancer sequence EN3681
SEQ ID NO: 92 Sequence from FIG. 22
SEQ ID NO: 93 Sequence from FIG. 29
SEQ ID NO: 94 native Fielder sequence (used for generating the sequence in FIG. 29)

### EXAMPLES

### Example 1—Identification of Transcription Factors Capable of Binding the Promoter Sequence of the RF3 Gene from Wheat

In order to identify transcription factors binding to promoter sequence of the Rf3 gene from wheat (SEQ ID NO: 1), a yeast one-hybrid assay was established as described in Ouwerkerk and Meijer (2011, Methods Mol Biol 678:211-227). Different bait strains each having a different overlapping 250 bp Rf3 promoter fragment (the bait sequence) covering up to 4 Kb of the promoter of the Rf3 gene from wheat (SEQ ID NO: 1) have been cloned in front of a HIS3 reporter gene in a pINT1-HIS3NB vector. Upon binding of a transcription factor protein from a cDNA expression library (the prey) a HIS3 reporter is activated which complements a deficiency in histidine biosynthesis, thereby causing growth of a colony.

The prey library has been derived from developing wheat spikes and was cloned in the Clontech vector pGADT7 AD. The prey library has been introduced in the different bait yeast strains by transformation (Ouwerkerk and Meijer, 2011, Methods and Protocols, Methods in Molecular Biology, vol. 678, Chapter 16, DOI 10.1007/978-1-60761-682-5_16). Growing colonies were recovered from the yeast one-hybrid screens with the bait strain comprising the bait sequence covering the nucleotides from position 3709 to position 3949 of SEQ ID NO: 1 and from the yeast one-hybrid screens with the bait strain comprising the bait sequence covering the nucleotides from position 3519 to position 3754 of SEQ ID NO: 1. The prey sequence in these colonies have been amplified (by PCR) and sequenced using the primer pair of SEQ ID NO: 2 and SEQ ID NO: 3. Two transcription factors have been identified:
a member of the Plant Homeodomain Finger (PHD) family of transcription factors having the amino acid sequence of SEQ ID NO: 4, which binds to the bait sequence covering the nucleotides from position 3519 to position 3754 of SEQ ID NO: 1; and
a member of the Ethylene Insensitive Like (EIL) family of transcription factors having the amino acid sequence of SEQ ID NO: 13, which binds to the bait sequence covering the nucleotides from position 3709 to position 3949 of SEQ ID NO: 1.

### Example 2: Isolation of the Wheat PHD Transcription Factor Sequences and in Silico Expression Analyses

Three homeologs of the PHD transcription factor identified in Example 1 are present in the wheat genome: one on the B subgenome (SEQ ID NOs: 4 and 5, TraesCS6B02G145900), one on the D subgenome (SEQ ID NOs: 6 and 7, TraesCS6D02G107700) and one on the A subgenome (SEQ ID NOs: 8 and 9, TraesCS6A02G117600). The closest ortholog in rice has been identified as Os02g0147800 (also known as LOC_0s02g05450) and in Arabidopsis as At4g29940.

Genevestigator® (genevestigator.com) in silico expression analysis shows that the three homeologs of the PHD transcription factor are low though ubiquitously expressed in wheat. In developing spikes, expression levels are highest in the early stages and decrease during flower development with a minimum expression in mature anthers. Expression in wheat leaves is lower than in developing spikes.

### Example 3: Identification of the Wheat PHD Transcription Factor Binding Site

In order to identify the binding site of the PHD protein, different bait strains each having a different 20 bp fragment (the bait sequence) covering up the 250 bp sequence of the promoter of the RF3 gene from wheat from position 3519 to position 3754 of SEQ ID NO: 1 have been cloned in front of a HIS3 reporter gene in a pINT1-HIS3NB vector as described in Example 1. A yeast one hybrid assay was performed with the prey sequence of the PHD transcription factor. The PHD transcription factor was able to bind to the bait strain comprising the fragment having the nucleotide sequence of SEQ ID NO: 10 similarly as to the 250 bp bait sequence of the promoter of the Rf3 gene from wheat from position 3519 to position 3754 of SEQ ID NO: 1.

Nucleotides being critical for the binding of the PHD transcription factor to the bait sequence of SEQ ID NO: 10 were identified by introducing mutations in the sequence. This mutation analysis resulted in the identification of a (partially) palindromic sequence comprising at least two consecutive GTA sequences being required for the binding of the PHD transcription factor. Examples of such pseudo-palindromic sequences are provided as SEQ ID NOs: 11 and 12.

A set of 20 bait strains (YSA001 to YSA019 and control strain YAW009), were retransformed with either the empty control vector pGADT7 AD (Clontech) or the library clone pGADT7-AD-PHD and screened in Y1H (Yeast One-Hybrid) assays on different concentrations of the His3p competitive inhibitor 3-amino-1,2,4-triazole (hereafter named 3-AT). The bait-sequences in strains YSA001 to YSA012 contain 12 different G to A point mutations based on a 20 bp sequence derived from the Rf3-58 promoter and which was analysed in strain YAW009. This particular bait sequence was found to confer highest activation by pGADT7-AD-PHD from a set of 12 Y1H strains and the activation is equivalent to the entire 254 bp fragment from the Rf3-58 promoter by which pGADT7-AD-PHD was cloned (using strain YEB004).

The results were as follows:

Strain YSA001 with the highlighted G (2^{nd} nt) in: AGTAGTAGTACTAC (SEQ ID NO: 30) mutated to A, conferred the same growth on a 3-AT concentration range (till 20 mM) as control strain YAW009 thus the highlighted G nucleotide has no critical role in PHD binding. Strains YSA002 and YSA003 with G to A changes at the highlighted positions in sequences AGTAGTAGTACTAC (SEQ ID NO: 30, 5^{th} nt changed) and AGTAGTAGTACTAC (SEQ ID NO: 30, 8^{th} nt changed) respectively, showed only little growth at 5 mM 3-AT (and no growth at higher concentrations, up to 10 mM), thus the mutated G-nucleotides are likely to have a critical role in PHD binding.

Strain YSA004 with a single G to A change at the highlighted (11^{th} nt) position in sequence AGTAGTAGTAGTACTACATA (SEQ ID NO: 10), strain YSA005 with a single C to T change at the highlighted (14^{th} nt) position in sequence AGTAGTAGTAGTACTACATA (SEQ ID NO: 10), and strain YSA006 with a single C to T change at the highlighted (17^{th} nt) position in sequence AGTAGTAGTAGTACTACATA (SEQ ID NO: 10), respectively, did not show any growth at medium without histidine or with 1 mM 3-AT or higher, thus the mutated G- or C-nucleotides are likely to have the most critical role in PHD binding. HIS3 reporter activity in these strains was completely silent since no growth was observed on medium without histidine and without 3-AT whereas strains YSA001, YSA002 and YSA003 showed normal growth on medium without histidine and without 3-AT.

Strain YSA007 has quadruple G to A changes at the highlighted positions (2^{nd}, 5^{th}, 8^{th} and 11^{th} nt) in sequence AGTAGTAGTAGTACTACATA (SEQ ID NO: 10), strain YSA008 has double C to T changes at the highlighted positions (14^{th} and 17^{th} nt) in sequence AGTAGTAGTAGTACTACATA (SEQ ID NO: 10), strain YSA009 has the 4 G to A changes as in YSA007 with 2 added C to T changes from YSA008 at the highlighted positions (G to A at 2^{nd}, 5^{th}, 8^{th}, and 11^{th} nt, and C to T at 14th and 17^{th} nt) in sequence AGTAGTAGTAGTACTACATA (SEQ ID NO: 10), strain YSA010 has 2 G to A changes at the highlighted positions (2^{nd} and 5th nt) in sequence AGTAGTAGTAGTACTACATA (SEQ ID NO: 10), strain YSA011 has 2 G to A changes at the highlighted positions (8th and 11th nt) in sequence AGTAGTAGTAGTACTACATA (SEQ ID NO: 10) and strain YSA012 has 2 G to A changes at the highlighted positions (8th and 11th nt) and 2 C to T changes at the highlighted positions (14th and 17^{th} nt) in sequence AGTAGTAGTAGTACTACATA (SEQ ID NO: 10), respectively. Strains YSA007, YSA009 did not show any growth on media without histidine and YSA011 confers growth on medium without histidine but at any concentration of 3-AT (1 mM and higher), growth stops. Strains YSA008, YSA010 and YSA012 show some growth on medium without histidine, but when 3-AT was added at 5 or 10 mM, no growth was observed anymore. Growth for all these strains, except YSA008 (showing little growth on 1 mM 3-AT but none at higher dosages) was inhibited on medium with 3-AT. Y1H bait strains harboring the empty control vector pGADT7 AD never showed any activation at medium without histidine with or without 3-AT. Together, these results confirm the results obtained with the single mutations as present in YSA001 to YSA006 where the importance of certain G- and C-nucleotides in PHD protein binding was shown where the C-nucleotides represent G-nucleotides part of GTA triplets on the bottom strand.

Interestingly, a similar sequence was identified in the promoter from another Rf gene: in the promoter of Rf1-09. In order to check, if this similar sequence from the Rf1 promoter would be bound by the PHD clone too, four HIS3 reporter strains (YSA020 to YSA023) were made which were based on a 22 bp bait sequence AGTAGTAGTACTACTAGATAAG (SEQ ID NO: 31). This sequence was cloned as monomer, dimer, trimer and tetramer respectively in front of the HIS3 reporter in vector pINT1-HIS3NB. As controls, two other sets of multimeric PHD binding sites derived from the Rf3-58 promoter were made. Strains YSA013, YSA014 and YSA015 represent dimer, trimer and tetramers of the sequence AGTAGTAGTAGTACTACATA (SEQ ID NO: 10, binding site in Rf3-58) as used in strains YAW009 and YSA001. In strains YSA016 to YSA019, the 22 bp sequence AGTAGTAGTAGTACTACATACT (SEQ ID NO: 32) from the Rf3-58 promoter was used which is 2 bp longer than the PHD binding site from the Rf3-58 promoter as used in strains YAW009 and YSA001. In this way, the PHD binding sites of Rf3-58 in strains YSA016 to YSA019 are embedded in a 22 bp sequence as in the Rf1-09 sequence as used in YSA020 to YSA023. Strains YSA013 to YSA023 were retransformed with either the empty control vector pGADT7 AD (Clontech) or the library clone pGADT7 AD-PHD, colonies were picked and inoculated again on minimal glucose medium with or without histidine with a concentration range of 3-AT. Growth was assessed by visual inspection. Y1H bait strains harboring the empty control vector pGADT7 AD never showed any activation at medium without histidine with or without 3-AT. Till 40 mM 3-AT, the Rf1-09-based strains YSA022 (trimer) and YSA023 (tetramer) grew to the same extent but the dimeric strain YSA021 grew at 40 mM much slower whereas the monomeric strain YSA020 was already strongly reduced at 25 mM 3-AT. Since YSA020, YSA021 and YSA022 grew well up to 20 mM, 30 mM and 40 mM, respectively, it is clear that adding more repeats of the PHD binding site each time increased activation of the HIS3 reporter and added to the transcription activating properties of the construct. The results are in accordance to results with the Rf3-58 multimeric constructs where YSA014 (trimer) and YSA015 (tetramer) grew well till 40 and 25 mM 3-AT, respectively, whereas the dimeric strain YSA013 started to grow slower after 27.5 mM 3-AT. The Rf3-58 Y1H bait constructs embedded as 22 bp constructs (YSA016 to YSA019) also showed increased activation when the 22 bp PHD binding site was used as dimer (YSA017), trimer (YSA018) or tetramer (YSA019) but showed little activation when present as monomer (YSA016).

### Example 4: Isolation of the Wheat EIL3 Transcription Factor Sequences and in Silico Expression Analyses

Three homeologs of the EIL3 transcription factor identified in Example 1 are present in the wheat genome: one on the B subgenome (SEQ ID NOs: 13 and 14, TraesCS7B01G145400), one of the D subgenome (SEQ ID NOs: 15 and 16, TraesCS7D02G244600) and one on the A subgenome (SEQ ID NOs: 17 and 18, TraesCS7A02G246100).

Genevestigator® (genevestigator.com) in silico expression analysis shows that the three homeologs of the EIL3 transcription factor are low to medium though ubiquitously expressed in wheat. Expression in wheat leaves is lower than in developing spikes.

The Rf3 promoter fragment that binds the EIL3 transcription factor comprises the sequence CATCTAGATACATCAATCT (SEQ ID NO: 19) that matches the Arabidopsis EIL3 recognition motif (2 overlapping AYGWAYCT motifs on different strands) as defined in Yamasaki et al 2005 (J Mol Biol 348, 253-264). This sequence is further referred to as the EIL3 binding site.

### Example 5: Generation of Expression Constructs for in Planta Validation

To validate the role of the binding sites of the transcription factors PHD and EIL3 as expression enhancers, different expression vectors were assembled comprising different fragments length of the promoter sequence of the Rf3 gene from wheat and duplications of a sequence comprising the EIL3 binding site and/or the PHD binding site:

pRf3-4>GUS (SEQ ID NO: 20) contains the about 4 kb sequence of the Rf3 promoter (SEQ ID NO: 1, nucleotides 461 to 4598 of SEQ ID NO: 20), the first intron of the actin 1 gene of Oryza sativa (nucleotides 4601 to 5062 of SEQ ID NO: 20), the coding sequence of the beta-glucuronidase gene of Escherichia coli, including the second intron of the ST-LS1 gene of Solanum tuberosum (nucleotides 5070 to 7070 of SEQ ID NO: 20) and the 3′ untranslated region of the proteinase inhibitor II gene of Solanum tuberosum (nucleotides 7085 to 7316 of SEQ ID NO: 20).

pRf3-2>GUS contains the about 2 kb sequence of the Rf3 promoter (SEQ ID NO: 21) replacing the about 4 kb sequence of the Rf3 promoter in pRf3-4 GUS (this is a 5′ deletion fragments of RF3-4).

pRf3-1.4>GUS contains the about 1.4 kb sequence of the Rf3 promoter (SEQ ID NO: 22) replacing the about 4 kb sequence of the Rf3 promoter in pRf3-4 GUS (this is a 5′ deletion fragments of RF3-4).

pRf3-1.2>GUS contains the about 1.2 kb sequence of the Rf3 promoter (SEQ ID NO: 23) replacing the about 4 kb sequence of the Rf3 promoter in pRf3-4 GUS (this is a variant of RF3-1.4 lacking the MITE insertion that is present in some wheat genotypes and absent in others).

pRf3-1.2-EIL>GUS contains the about 1.2 kb sequence of the Rf3 promoter including a duplication of the EIL3 binding site (SEQ ID NO: 24) replacing the about 4 kb sequence of the Rf3 promoter in pRf3-4 GUS.

pRf3-1.2-EIL*>GUS contains the about 1.2 kb sequence of the Rf3 promoter including a sequence of the EIL3 binding site which has been mutated (SEQ ID NO: 25) replacing the about 4 kb sequence of the Rf3 promoter in pRf3-4 GUS.

pRf3-1.2-PHD-EIL>GUS contains the about 1.2 kb sequence of the Rf3 promoter including a duplication of both the EIL3 and PHD binding sites (SEQ ID NO: 26) replacing the about 4 kb sequence of the Rf3 promoter in pRf3-4 GUS. In addition, different expression vectors were assembled to express either the transcription factors EIL3 or PHD, or the GFP protein:

p35S>GFP (SEQ ID NO: 27) contains the promoter region of the 35S transcript gene of Cauliflower mosaic virus (Odell J T. et al., 1985; nucleotides 461 to 988 of SEQ ID NO: 27), the 5′ untranslated region of the chlorophyl a/b binding protein gene of Petunia x hybrida (Harpster M H. et al., 1988; nucleotides 992 to 1051 of SEQ ID NO: 27), the first intron of the actin 1 gene of Oryza sativa (Mc Elroy et al., 1991; nucleotides 1054 to 1515 of SEQ ID NO: 27), the coding sequence of the enhanced green fluorescent protein gene of Aequorea victoria (GFP; Cormack et al., 1996; nucleotides 1538 to 2254 of SEQ ID NO: 27) and the 3′ untranslated region of the 35S transcript gene of Cauliflower mosaic virus (Sanfaçon H. et al., 1991; nucleotides 2278 to 2502 of SEQ ID NO: 27).

P35S>EIL contains the EIL3 coding sequence (SEQ ID NO: 14) replacing the GFP coding sequence in p35S GFP.

P35S>PHD contains the PHD coding sequence (SEQ ID NO: 5) replacing the GFP coding sequence in p35S GFP.

Furthermore, a control expression vector was assembled to express the firefly luciferase (LUC): pUbi>LUC (pKA63; SEQ ID NO: 28) contains PubiZm, the promoter region of the ubiquitin gene of Zea mays (nucleotides 1 to 1997 of SEQ ID NO: 28), the coding sequence of the luciferase gene from firefly (Photinus pyralis; nucleotides 2024 to 3676 of SEQ ID NO: 28), and 3′35S, the 3′ untranslated region of the 35S transcript gene of Cauliflower mosaic virus (nucleotides 3689 to 3913 of SEQ ID NO: 28).

### Example 6: Protoplast Expression Results

The impact of the above identified transcription factor binding sites on Rf3 promoter activity was tested by transient expression in wheat mesophyll protoplasts. Various promoter>GUS vectors were tested in wheat protoplasts with or without co-transfection of a p35S>GFP, p35S>EIL or p35S>PHD vector. To correct for deficiencies in introduction efficiency, GUS activities of wheat transfected protoplasts were divided by the luciferase activities from the co-introduced control vector pUbi>LUC. Wheat protoplast preparation and PEG transfection of the wheat protoplasts was performed according to Shang et al (2014, Nature protocols 9(10), 2395-2410).

To determine which fragment of the wheat Rf3 promoter would be suitable for testing the impact of the transcription factor binding sites in wheat protoplasts, promoter activity was compared for a 4-kb (pRf3-4), a 2-kb (pRf3-2), and a 1.4-kb (pRf3-1.4) promoter fragment and a variant of the 1.4-kb promoter lacking the MITE insertion that is absent in some wheat genotypes (pRf3-1.2). As shown on FIG. 1A the promoter activity of all fragments tested is comparable. Therefore, the shortest 1.2-kb promoter fragment was chosen to test the impact of the transcription factor binding sites.

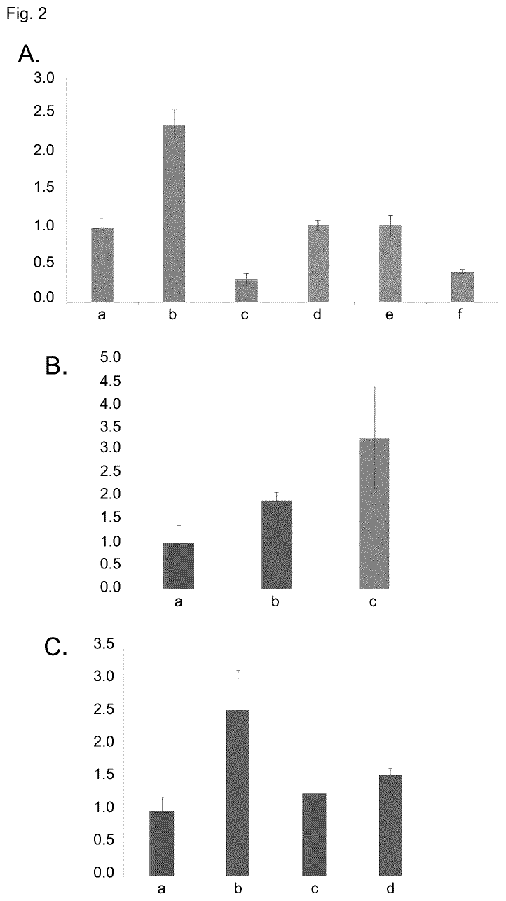

It was furthermore confirmed, as shown in FIG. 1B that the overexpression of the EIL3 or PHD transcription factor does not increase the activity of the co-introduced Rf3 promoter. In a next step, the impact of the EIL3 binding site on Rf3 promoter activity was determined in wheat mesophyll protoplasts. FIG. 2A shows that the promoter in which the EIL3 binding site is mutated had a reduced promoter activity compared to the promoter having the binding site. This further confirmed the identification of SEQ ID NO: 19 as the EIL3 binding site. Duplication of the EIL3 binding site resulted in a higher promoter activity when the EIL3 transcription factor is overexpressed but not when the PHD transcription factor is overexpressed (FIG. 2A). This confirmed that the duplicated sequence is able to increase RF3 promoter activity in an EIL3 transcription factor dependent way. It also shows that such duplication can be used to enhance expression in plant tissues, such as developing spikes, that have higher expression of the EIL3 transcription factor than leaves.

To further test the utility of promoter sequence duplication approaches to increase Rf3 promoter activity, an Rf3 promoter fragment was selected (SEQ ID NO 29) that contains both the EIL3 and the PHD binding sites. The selected fragment is flanked by Cas9 target sites so that it can be duplicated in the wheat genome using a Cas9 nuclease or nickase and sgRNAs targeting these sites. As shown in FIG. 2B, the duplication of this sequence in the promoter increases Rf3 promoter activity in presence of the EIL3 transcription factor even further than duplicating the EIL3 binding site alone.

In addition, FIG. 2C demonstrates that duplication of the sequence containing both the PHD and the EIL3 binding sites also increased Rf3 promoter activity when the PHD transcription factor is overexpressed. This makes this sequence duplication an excellent genome editing strategy to increase wheat Rf3 expression in wheat tissues, such as developing spikes, having higher PHD and EIL3 expression levels than leaves.

### Example 7: Identification of miRNA3619 Binding Site in Rf3 Gene Sequence

To identify possible miRNAs interacting with the Rf3-58 coding sequence, a developmental gene expression atlas of developing spike tissues was generated in progeny from cross of a G-type CMS restorer line with accession number PI 583676 (USDA National Small Grains Collection, also known as Dekalb 582M and registered as US PVP 7400045) and a line containing T. timopheevii cytoplasm. The resultant progeny of this cross contains the CMS cytoplasm and the Rf3-restorer locus with functional Rf3-58 gene. Total RNA, including degraded and IncRNA was extracted from tissue samples and mRNA, sRNA and total mRNAs sequenced and analyzed to determine normalised expression levels across all tissues sampled.

### Tissue Sampling

Five tissue types from each PI 583676 genotype were sampled, and three biological replicates per sample taken. The tissues sampled were:
Unfolded leaf tissue at Zadoks stage Z16
Whole spike (1-2 cm) at Z39-Z41, corresponding to tetrad stage of pollen development
Individual spikelets at Z45-Z48, corresponding to the uninucleate stage of pollen development
Immature anthers at Z50-Z59, corresponding to the binucleate stage of pollen development
Mature anthers at Z60-Z69, corresponding to the trinucleate stage of pollen development

Individual tissue samples were excised, weighed, snap frozen in liquid nitrogen and stored at −80 C until further processing. For total RNA extraction, tissue samples from 10 plants per seed lot were pooled to provide sufficient material for sequencing.

### Total RNA Extraction

Total RNA per biological replicate per tissue was extracted using standard procedures.

### RNA-Sequencing

To quantify gene expression in tissues, 1 μg of total RNA was subjected to a protocol, whereby mRNA transcripts are purified by polyA-tail selection followed by library preparation as according to the Illumina TruSeq stranded mRNA protocol and manufacturers' instructions.

To quantify small RNA molecules including pre-miRNA, immature and mature miRNA, 3 μg of DNA-free, total RNA was loaded on acrylamide gels to purify the small RNA (sRNA) fraction, and this was followed by library construction using the Illumina TruSeq small RNA kit as according to manufacturer's instructions.

For degradome analysis, and quantification of non-coding RNA as well degraded RNA which includes the cleavage products of miRNA activity, up to 10 μg of DNA-free total RNA was used for adapter-based selection of uncapped mRNA fragments followed by library preparation and Illumina—based short-read sequencing.

### Bioinformatics Analysis

miRNA discovery and quantification was carried out using an internal pipeline, using three complementary prediction tools and based on sRNA sequencing data, correlation with mRNA expression levels and mapping of degradome sequencing reads.

In brief, sRNA reads were used to build a catalogue of predicted pre-miRNA and mature miRNA sequences for each tissue and each genotype, complete with tissue-specific expression levels and genome position (based on IWGSC v1 Chinese Spring Reference genome—(Consortium (IWGSC) et al. 2018)).

Also, a list of potential mRNA targets for the predicted miRNA as well as their target cleavage sites was generated based on correlations between the expression patterns of mRNAs and miRNAs. Alignment of degradome reads against the expressed mRNA targets using the PAREsnip2 tool, was used to confirm cleavage of that transcript, at the predicted site, or not (Thody et al. 2018).

From the entire data set of identified miRNA, only one miRNA (mi3619) was predicted to target Rf-PPR genes. This has a category ‘0’ from the PAREsnip2 tool (highest confidence), and mi3619 also had the lowest predicted binding energy to Rf-PPRs.

Aligning degradome reads from (one replicate of one sample) confirmed that cleavage products were present. The miR3619 sequence has matches in miRBase wheat (https://www.mirbase.org) and corresponds most closely to tae-miR9674b. tae-miR9674b has been reported to regulate PPR genes by Li et al. (2019) in a wheat K-type CMS—restoration system based on Ae. kotschyii cytoplasm. tae-miR9674b was reported to target a PPR protein, homologous to the Rf1 protein of rice, but there are no reports that it targets Rf-PPR genes involved in G-type CMS system.

Activity of miR3619 cleaves Rf3-58 CDS at a position corresponding to the beginning of PPR-motif #09 in the translated protein. An identical miRNA site is present in the Rf3-29a allele ‘ high restorer’). miR3619 is predicted to also target nucleic acids encoding other proteins including Ubiquitin-conjugating enzyme. The miR3619 binding site in the Rf3-58 coding sequence is also found at approximately the same position in other G-type CMS Rf coding sequences, such as in the Rf3-29a coding sequence, and in the Rf1-09 coding sequence (SEQ ID NO: 64). Hence, other Rf3 and Rf1 coding sequences also share the same miRNA binding site.

miR3619 Expression Profile in PI 583676

miR3619 is highly expressed in spike tissue of PI 583676 and its progeny, and its expression decreases through the four spike developmental stages measured. miR3619 expression is even higher in young leaves but levels decreased during the 4 different spike-development stages (not shown). This suggests that miR3619 is involved in suppressing expression of Rf-genes most strongly in young leaf tissues where no restoration takes place.

### Example 8: Improving Expression by Modifying miRNA3619 Binding Site in Rf3-58 Sequence

To investigate whether mutating the putative miRNA binding site results in increased Rf3 expression, the Rf3-58 sequence coding for PPR units 8 to 10 was translationally fused to the GUS coding sequence under the control of the 35S promoter (pBas04646). A variant was made in which the Rf3 sequence was replaced by a sequence that is coding for the same amino acid sequence but is codon-optimized for maximum expression in wheat (SEQ ID NO 49), resulting in plasmid pBas04647). In the wheat-codon-optimized coding sequence, the putative miRNA binding site contains 9 mutations which ensure it is no longer a target for miRNA3619. The same mutations were also introduced into the WT coding sequence and the putative miRNA binding site was re-introduced into the wheat-optimized sequence (see Table 1, plasmids pBas04648 and pBas04649, respectively).

TABLE 1
Transient expression plasmids used to evaluate
the impact of miRNA binding site mu-tations on
Rf3 expression. Rf3 CDS = the sequence coding
for PPR units 8 to 10 of the Rf3-58 protein,
either as present in the wheat gene (WT) or
codon optimized for maximum expression in
wheat. Mutated nucleotides are underlined.
All mutations are silent mutations, except
for the mutations in pBas05030 and pBas05032,
which cause conservative amino changes in
the en-coded protein. Numbers indicating
the position of the mutations in the
miRNA3619 binding site sequence are
shown in FIG. 6A.

| | Rf3 CDS outside | | SEQ | Nucleotide |
|---|---|---|---|---|
| Plasmid | of the miRNA | miRNA binding | ID | positions |
| name | binding site | site sequence | NO | mutated |
| pBas04646 | WT | GGGUAGGUUGGAUGAUGCU | 45 | — |
| pBas04648 | WT | AGGACGCCUAGACGACGCG | 50 | 1-4-5-7-8- |
| | | | | 10-13-16-19 |
| pBas04649 | codon optimized | GGGUAGGUUGGAUGAUGCU | 51 | — |
| pBas04647 | codon optimized | AGGACGCCUAGACGACGCG | 52 | 1-4-5-7-8- |
| | | | | 10-13-16-19 |
| pBas04805 | codon optimized | GGGUCGCUUAGACGACGCU | 53 | 5-7-10-13-16 |
| pBas04806 | codon optimized | GGGUCGCUUAGAUGAUGCU | 54 | 5-7-10 |
| pBas04807 | codon optimized | GGGUCGCUUGGAUGAUGCU | 55 | 5-7 |
| pBas04808 | codon optimized | GGGUAGGUUAGACGAUGCU | 56 | 10-13 |
| pBas04809 | codon optimized | GGGUAGGUUGGACGACGCU | 57 | 13-16 |
| pBas04811 | codon optimized | GGGUAGGUUAGAUGAUGCU | 58 | 10 |
| pBas05030 | codon optimized | GGGUAAGUUGGAUGAUGCU | 59 | 6 |
| pBas05031 | codon optimized | GGGUAGAUUGGAUGAUGCU | 60 | 7 |
| pBas05032 | codon optimized | GGGUAGGUUGGAUGAUGUU | 61 | 18 |

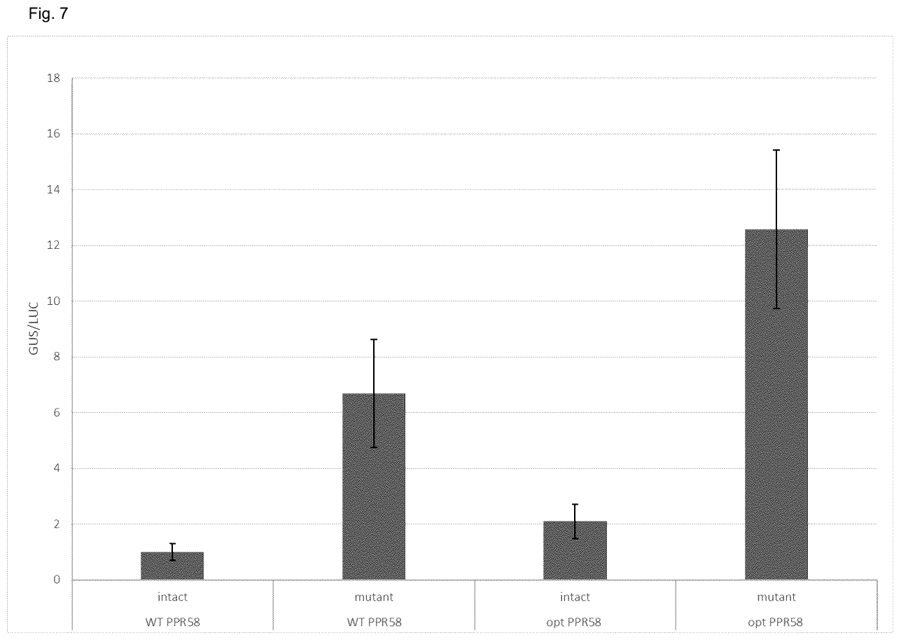

The resulting plasmids were introduced into wheat mesophyll protoplasts and, following an overnight incubation, protein was extracted from the protoplasts, and GUS activities determined. To correct for differences in introduction efficiency, GUS activities of transfected wheat protoplasts were divided by/normalized to the luciferase activities from a co-introduced control vector containing the firefly luciferase gene under control of the maize ubiquitin promoter (pKA63). Wheat protoplast preparation and PEG transfection of wheat protoplasts was performed according to Shang et al. (2014, Nature protocols 9(10), 2395-2410). Strikingly, mutation of the putative miRNA3619 binding site results in a 6-fold higher GUS expression, both for the WT and for the wheat-optimized coding sequence (FIG. 7). This indicates that the Rf3 mRNA is indeed a target for miRNA3619 and that mutation of its binding site can increase Rf3 expression levels in wheat plants driven by endogenous levels of miR3619.

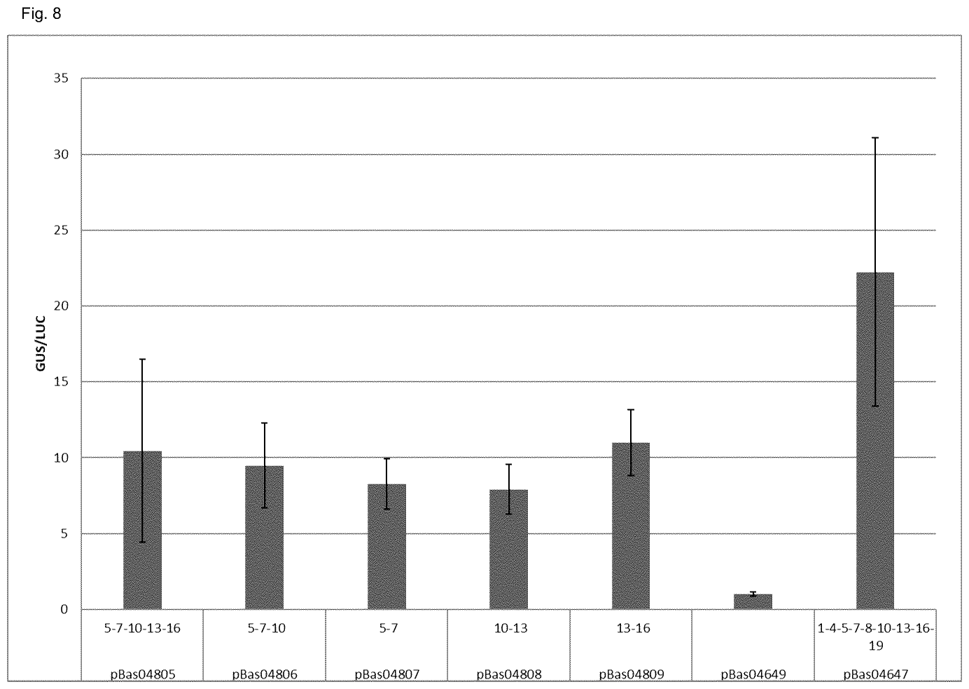

To test whether a similar increase in Rf3 expression could be obtained by fewer mutations in the miRNA3619 target site, mutants that contain 5, 3, or 2 nt mutations in the miRNA3619 binding site were tested in wheat protoplasts. FIG. 8 shows that these mutants also have an increased GUS expression in wheat protoplasts, indicating that a 2 nt mutation is sufficient to increase Rf3 expression.

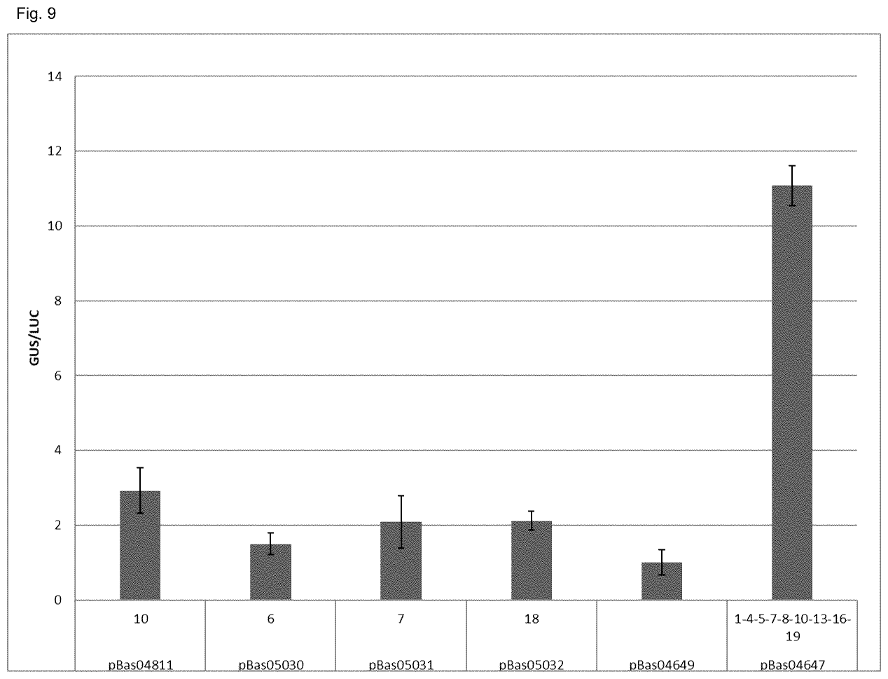

In a next step, the impact of single nt (C-to-T or G-to-A) mutations that can be introduced by EMS was assessed. FIG. 9 shows that these mutations also increased expression up to 3-fold in wheat protoplasts. This indicates that expression of the Rf3 gene in wheat can be increased through the introduction of single nt mutations by genome editing or EMS mutagenesis. Two of these mutations (as present in pBas04811 and pBas05031) are silent mutations and do not change the sequence of the encoded protein.

### Example 9: Genome Editing of miRNA Binding Site to Improve Expression in Wheat

Guide RNAs for CRISPR-mediated genome editing targeting the Rf3 miRNA binding site in the coding sequence are designed by using, e.g., the CAS-finder tool. The guide RNAs are tested for targeting efficiency by PEG-mediated transient co-delivery of the gRNA expression vector with an expression vector for the respective nuclease, e.g. Cas9 or Cpf1, under control of appropriate promoters, to protoplasts of a wheat restorer line containing the candidate PPR-Rf gene of interest, preferably the line designated as T. timopheevii/2*Iowin//2*Quivira, USDA Accession number PI 583676. Genomic DNA is extracted from the protoplasts after delivery of the guide RNA and nuclease vectors. After PCR amplification, integrity of the targeted candidate PPR Rf gene sequence is assessed by sequencing.

The one or two most efficient guide RNAs are used for stable genome editing in the same wheat restorer line also containing the G-type CMS cytoplasm. For this purpose, the selected guide RNA expression vector, together with a nuclease expression module, a repair DNA containing the desired nucleotide mutation(s) and a selectable marker gene, are introduced into embryos isolated from the before mentioned wheat restorer line using, e.g., particle gun bombardment. Transgenic plants showing resistance to the selection agent are regenerated using known methods. Transgenic T0 plants containing changes in the miRNA binding site are identified by PCR amplification and sequencing.

Transgenic T0 plants containing the G-type CMS cytoplasm and likely to contain a mutation in the miRNA binding site of Rf preferably in homozygous state, but alternatively in heterozygous state, are crossed as female parents to a spring wheat line with normal cytoplasm and without PPR-Rf genes. The F1 progeny of the crosses contains the G-type “CMS” cytoplasm and 50% (in case of heterozygous TO) or 100% (in case of homozygous TO) of the F1 progeny will have a modified version of the Rf3 gene. The F1 plants with a modified Rf3 gene are identified using genomic PCR assays, and expression of Rf3 is compared to plants with unmodified Rf3. The F1 plants show increased expression of Rf3 and improved male fertility due to the modification of the miRNA binding site.

The level of male fertility in the F1 progeny with the Rf3 gene having a modification of the miRNA binding site is tested using different assays. In a first assay, pollen accumulation and pollen viability is quantified using the AmphaZ30 device. The modification of the miRNA binding site in the Rf3 gene leads to higher numbers of viable pollen. In another assay, the integrity of anther tissues is inspected microscopically. The knock-out of a functional candidate PPR Rf gene leads to early deterioration of the tapetum layer. In a further assay, seed set per ear following bagging and self-pollination is quantified. The modification of the miRNA binding site in the Rf3 gene leads to a higher number of grains per ear. In all tests the F1 progeny from crosses of non-edited Rf plants to the same spring wheat line serve as a control.

### Example 10: Transgenic Expression of Rf3-58 with Modified miRNA Binding Site to Improve Expression in Wheat

To investigate whether mutating the putative miRNA binding site results in increased Rf3-58 expression in transgenic plants, two constructs were created and transformed into the wheat cultivar Fielder. The first construct, pBAS04254 comprised the native Rf3-58 promoter and the native Rf3-58 coding sequence, including the native miRNA binding site in PPR domain 9, fused to the 3′ Nos terminator sequence. The second construct, pBAS04255 comprised the native Rf3-58 promoter and the native Rf3-58 coding sequence, except for the miRNA binding site in PPR domain 9, which was modified to contain 9 nucleotide changes (AGGACGCCUAGACGACGCG (SEQ ID NO: 50) making it no longer a target for miRNA3619, without affecting the composition of the translated polypeptide, fused to the 3′ Nos terminator sequence. The Rf3-58 transgenes in pBAS04254 and pBAS04255 are collectively referred to as “native” transgenes.

In addition to one of the two native Rf3-58 transgenes, the T-DNA region of the transformation vectors also contained a bar selectable marker gene providing tolerance to the herbicide glufosinate, for selection of transgenic plants, after Agrobacterium-mediated transformation. In total 13 single-copy transgenic events containing pBAS04254 and 16 single-copy transgenic events containing pBAS04255 were selected for further work. Transgenic plants containing a single copy of the transgene cassette were used as pollinators in crosses with male sterile wheat plants containing the G-type CMS cytoplasm.

In each F1 progeny of the 29 single-copy transgenic events, 5 plants hemizygous for the Rf3-58 transgene and 5 plants not containing the Rf3-58 transgene (null) were selected based on copy-number PCR analysis of the bar selectable marker gene. The selected F1 plants were maintained until maturity and were allowed to set seed by self-pollination. Pollen viability was determined in randomly selected plants by iodine staining during flowering of all plant. Spike number and total seed yield were determined for all plants. Expression of the two Rf3-58 transgenes was determined by digital droplet (dd) PCR analysis in young leaves and developing spikes of 3 hemizygous and 2 null plants per event. Two types of control plants were included: (1) 5 homozygous transgenic plants from an event containing a pUbi::Rf358::3′ Nos transgene (pBay01414; containing a codon-optimized coding sequence of Rf3-58 expressed under the Ubiquitin promoter) previously shown to provide a high level of restoration of fertility to plants containing the G-type CMS cytoplasm and (2) 5 plants of the conventional cultivar “Fielder” containing a “normal” wheat cytoplasm and used as transformation donor. The results are summarized in Table 2.

TABLE 2
Summary of the results of pollen viability, spike number,
and seed set of all F1 plants of all single-copy events,
plus control plants, grown to maturity.

| | | | | pollen | average | average |
|---|---|---|---|---|---|---|
| | | | | viability | spike | seed |
| | | | | (% | number | yield |
| | # | copy | # | iodine | per | per |
| Constructs | events | number | plants | positive) | plant | plant |
| pBAS04254 | 13 | 0 | 63 | 25 | 8.6 | 10.3 |
| | | 1 | 56 | 40 | 6.8 | 173.6 |
| pBAS04255 | 16 | 0 | 72 | 29 | 9 | 20 |
| | | 1 | 71 | 49 | 6.9 | 255 |
| pBAY01414 | 1 | 1 | 5 | 54 | 6.8 | 261 |
| Fielder | n.a. | n.a. | 5 | 48 | 7.8 | 379 |

The results presented in Table 2 show that hemizygous plants for both “native” Rf3-58 transgenes have higher pollen viability and higher seed yield per plant, compared to nullsegregants, demonstrating that both “native” transgenes provide effective restoration of male fertility in transgenic plants. The results also suggest that male sterile plants attempt to “compensate” for the reduced fertility by producing more spikes. Further, it is shown that the restoration of fertility by one copy of the “native” transgenes is equal or nearly equal to the level of restoration provided by 2 copies of the codon-optimized transgene driven by the pUbi promoter. Finally, the seed yield data strongly suggest that disruption of the miRNA binding site in pBASO4255 leads to a higher level of restoration compared to the situation with the intact miRNA binding site.

As a next step, we investigated whether the level of restoration correlated with the level of Rf3-58 expression in the transgenic events. For this analysis, we only included plants that have complete data for seed set and spike expression. The results are summarized in Table 3. The expression levels were determined by ddPCR and normalized relative to 2 reference genes in the same experiment.

TABLE 3
Summary of the results of pollen viability, seed set, leaf and spike
expression of RF3-58 for all F1 plants of all single-copy events,
plus control plants, for which expression analysis was performed.

| | | | | pollen | average | | |
|---|---|---|---|---|---|---|---|
| | | | | viability * | seed | | |
| | | copy | | (% iodine | yield per | leaf | spike |
| Constructs | # events | number | # plants | positive) | plant | expression | expression |
| pBAS04254 | 11 | 0 | 21 | 40.0 (5) | 7.2 | 0.10 | 0.01 |
| | | 1 | 31 | 55.6 (18) | 196.5 | 14.17 | 9.80 |
| pBAS04255 | 15 | 0 | 30 | 37.2 (16) | 14.7 | 0.07 | 0.05 |
| | | 1 | 44 | 73.2 (25) | 255.6 | 18.90 | 16.8 |
| | | | | | | | 0 |
| pBAY01414 | 1 | 1 | 3 | 90 (1) | 272.3 | N.A. | N.A. |
| Fielder | n.a. | n.a. | 3 | 80 (1) | 402.0 | N.A. | N.A. |

* pollen viability was determined on the number of plants indicated between brackets

The results of pollen viability and seed set for the subset of plants in table 3 are fully consistent with the results and conclusions for all plants in table 2. The expression results of Table 3 show that the mRNA of the Rf3-58 gene with the disrupted miRNA binding site (pBAS04255) is on average expressed at a higher level (+33% in leaves and +71% in spikes) than the mRNA of the Rf3-58 gene with the intact miRNA binding site (pBAS04254). This is consistent with the intact miRNA binding site promoting mRNA degradation and provides a direct explanation for the increased pollen viability and increased seed set (+30%) in transgenic plants containing the Rf3-58 gene with the disrupted miRNA binding site. Together the data demonstrate that disruption of the native miRNA binding site leads to enhanced expression of the Rf3-58 mRNA, resulting in a higher level of restoration of male fertility and increased seed set.

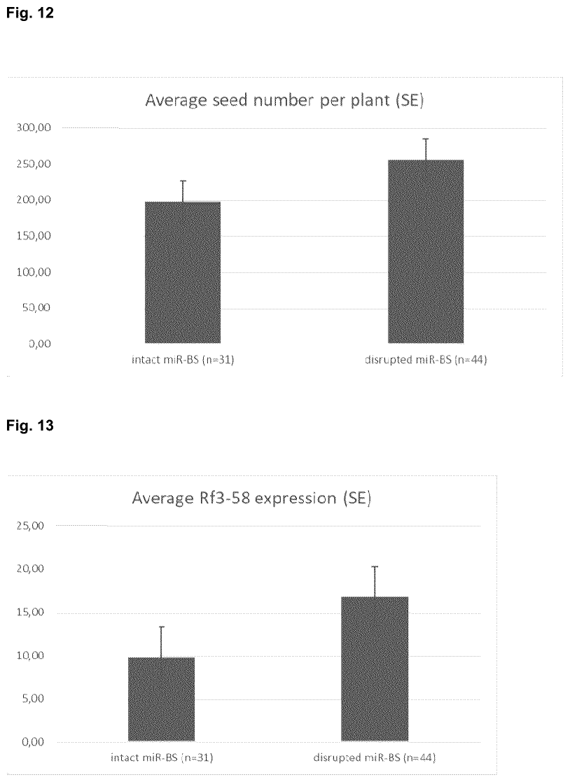

FIG. 12 compares seed production 1-copy plants of pBAS04254 (39) with 1-copy plants of pBAS04255 (47).

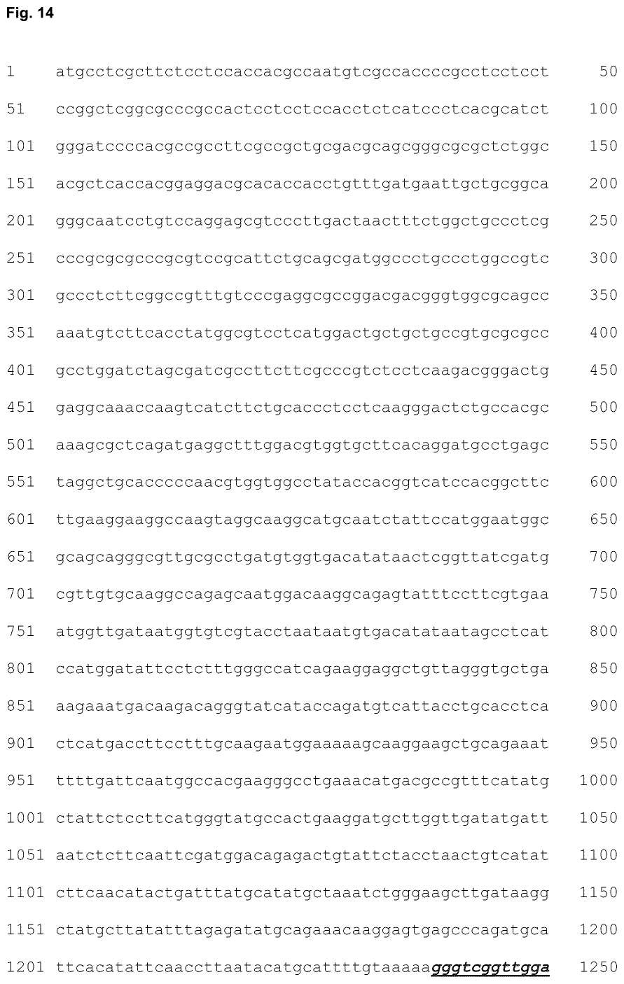

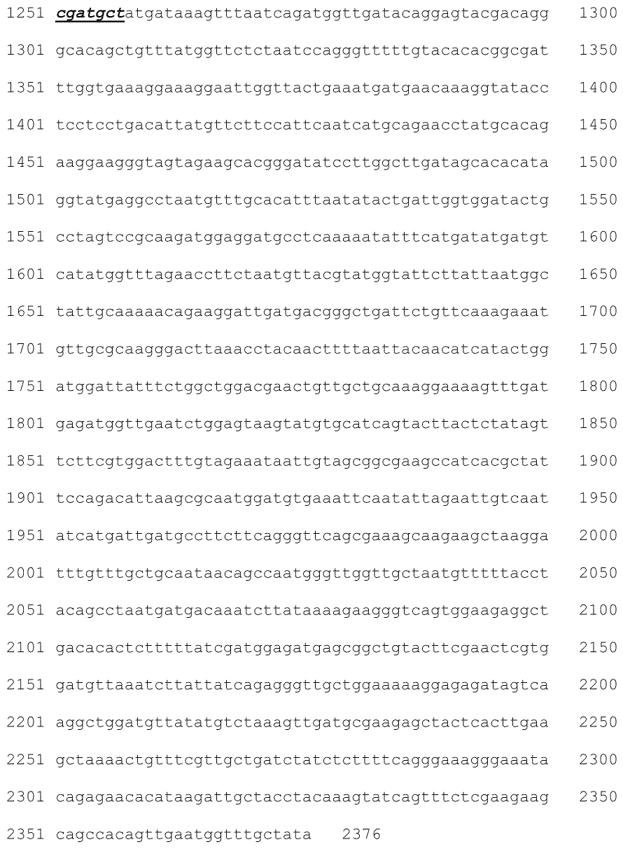

FIG. 13 compares Rf3-58 expression in 1-copy plants of pBAS04254 (39) and 1-copy plants of pBAS04255 (47).

From the bar charts, it is clear that the Rf3-58 transgene with the disrupted miRNA binding site provides a higher level of Rf3-58 expression and a higher level of restoration of seed set compared to the Rf3-58 transgene with the intact miRNA binding site.

### Example 11: Insertion of the Wheat EN1390 Enhancer Increases Wheat Rf3-58 Promoter Activity in Wheat Protoplasts

The wheat Rf3-58 gene encodes a pentatricopeptide (PPR) protein that restores male fertility of wheat G-type cytoplasmic male sterility (“CMS” herein) lines. This PPR gene is primarily expressed in flowering tissues and its promoter shows only low activity in wheat protoplasts (8-10 times below that of p35S, see FIG. 16). A 1423-bp promoter fragment of this Rf3-58 promoter (shown in SEQ ID NO: 73) as well as a 2 kb promoter fragment (shown in SEQ ID NO: 72) has the same level of activity in protoplasts as a 4138-bp promoter fragment (shown in SEQ ID NO: 71). Also, the sequence resembling a Miniature Inverted-repeat Transposable Element (herein “MITE-like insertion”) that is only present in some wheat Rf3 genotypes was found not to affect Rf3 expression in protoplasts. Therefore, it was decided to test the impact of various wheat enhancers by inserting these enhancers in the promoter variant of the 1.4-kb Rf3-58 fragment that lacks the MITE-like insertion (shown as SEQ ID NO: 74). The Rf3 allele in wheat line Fielder is also lacking this MITE-like insertion.

Five enhancers from wheat (EN2393 (SEQ ID NO:87), EN1390 (SEQ ID NO:70), EN5458 (SEQ ID NO:86), EN3681 (SEQ ID NO:91), and nt 1-80 of EN4730 (SEQ ID NO:90); sequences for these are described in WO2021/048316, incorporated herein by reference, the sequence for EN1390 is SEQ ID NO:70 herein) were inserted into the Rf3-58 promoter at the position −127 (relative to the translation start codon) that contains the MITE-like insertion in some wheat Rf3 genotypes. Testing of these promoter variants in wheat protoplasts (see FIG. 17) showed that although each of the enhancers induced some level of expression increase the strongest expression increase (almost 15-fold) was obtained with wheat enhancer EN1390.

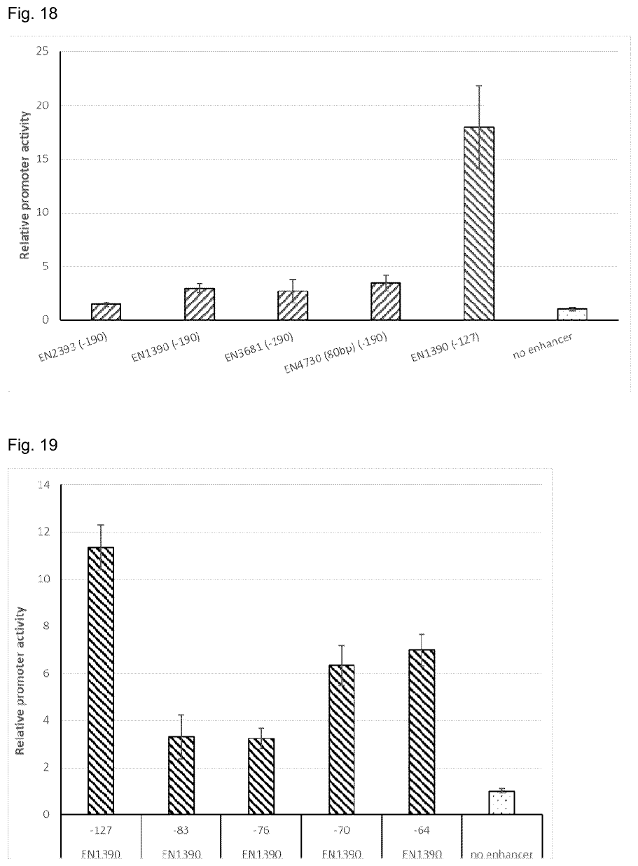

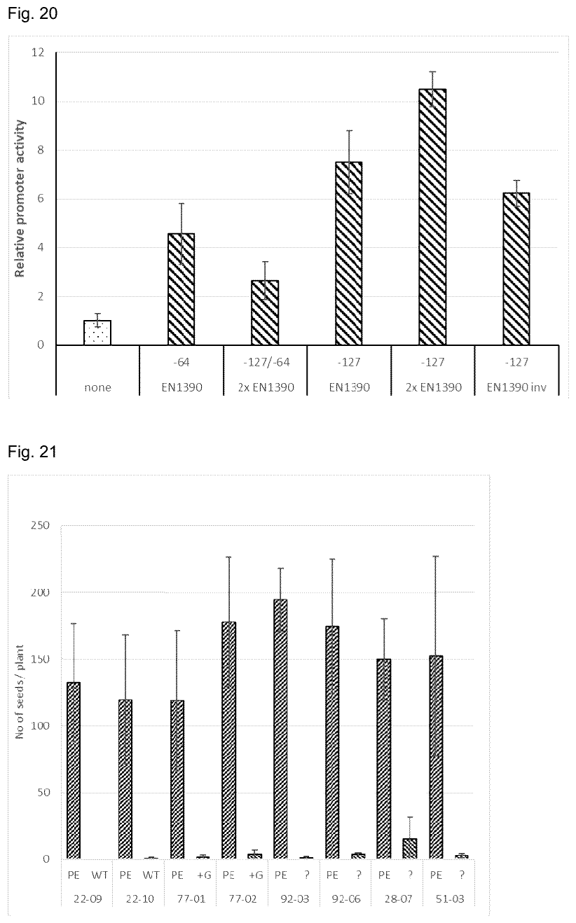

The same enhancers were also tested at position −190 of the Rf3-58 promoter (see FIG. 18). Each of the enhancers increased promoter activity at this position but only up to 3-fold. Activity of EN1390 was much lower at −190 than at −127. Inserting EN1390 closer to the translation start site also resulted in a lower expression increase than inserting at −127 (see FIG. 19). Insertion of EN1390 at nt −127 (relative to the translation start codon) thus provides the strongest increase (more than 10-fold) of Rf3-58 promoter activity in wheat protoplasts. Inserting 2 copies of EN1390 at position −127 increases Rf3-58 promoter activity a bit further and the impact of EN1390 is largely independent of the orientation of the enhancer (see FIG. 20).

### Example 12: Inserting EN1390 in the Rf3-58 Promoter Improves Restoration Capacity of Rf3

To assess the impact of inserting EN1390 on Rf3-58 promoter activity in its genomic context, 2 genome editing experiments were performed. DNA was transferred into immature embryos 2-3 mm in size isolated from sterilized ears of wheat cv. Fielder using standard conditions (e.g., Sparks et al., 2014). A mixture of the Cas9 vector pBay02430 (SEQ ID NO: 75), one or two gRNA expression vectors, a repair DNA, and a plasmid containing an eGFP-BAR fusion gene under control of the 35S promoter (pBay02032, SEQ ID NO: 76) were transferred into the embryos. The further culture of the immature embryos was essentially conducted as previously described (Ishida et al., 2015). After DNA transfer, the immature embryos were transferred to non-selective WLS callus induction medium for about one week, then moved to WLS with 5 mg L-1 phosphinothricin (PPT) for a first selection round of about 3 weeks followed by a second selection round on WLS with 10 mg L-1 PPT for another 3 weeks. PPT resistant calli were selected and transferred to shoot regeneration medium with 5 mg L-1 PPT.

Compared to wheat lines that contain a functional Rf3 restorer gene, Fielder contains a 2-nt (GA) deletion in the Rf3 coding sequence (CDS) causing a frameshift and production of a truncated protein that ends with PPR-unit 4. The encoded protein was expected to have no restoration activity. To check whether repairing the CDS is sufficient to provide restoration activity to Fielder, the missing nucleotides were introduced into the Fielder CDS by genome editing, using pBas03477 (SEQ ID NO: 77) as gRNA expression vector and pBas03482 (SEQ ID NO: 78) as repair DNA. From this genome editing experiment, 7 lines were identified that have G0 plants with 1 Rf3 allele that was precisely edited by the repair DNA (see Table 4). The other Rf3 allele is either WT, has a 1-nt insertion at the target site, or has a modification that prevented amplification of the allele by PCR. These G0 plants were crossed as male to Naxos plants (male sterile plants containing CMS cytoplasm and lacking known functional Rf genes) and G1 seeds were harvested. The resulting G1 plants were grown and G1S1 seeds were produced by selfing. These G1 plants contained one non-functional Naxos Rf58 allele and in about half of the plants the second Rf3 allele is a precisely edited Fielder allele. For each seedlot, the seed set of the plants that do have the precisely edited Fielder allele was compared with that of the plants lacking such edited allele (see FIG. 21). Plant having the precisely edited allele produced on average between 120 and 195 seeds per plant, whereas plants lacking this edited allele typically produced less than 5 seeds. This shows that repairing the frameshift in the Rf3 Fielder gene is sufficient to turn Fielder in to a restoring line.

TABLE 4
Wheat lines with precise edits selected from the genome editing
experiment that only repairs the Rf3 coding sequence. The
genotype was determined by sequencing of the Rf3 gene. The
? allele could not be amplified by PCR, probably due
to a large deletion or re-arrangement at the target site.

| Event name | PPR58 genotype of G0 plant |
|---|---|
| TMTA0423-0022-B01 | 1 precise edit + 1 WT allele |
| TMTA0423-0077-B01 | 1 precise edit + 1 nt (G) insertion |
| TMTA0440-0092-B02 | 1 precise edit + 1 ? allele |
| TMTA0501-0034-B01 | 1 precise edit + 1 ? allele |
| TMTA0502-0028-B02 | 1 precise edit + 1 ? allele |
| TMTA0502-0051-B01 | 1 precise edit + 1 ? allele |
| TMTA0502-0074-B01 | 1 precise edit + 1 nt (T) insertion |

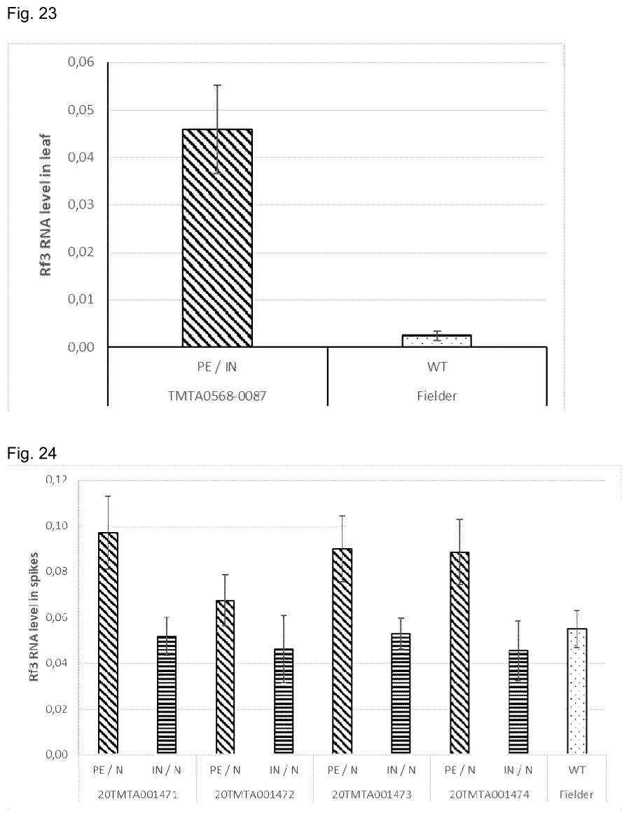

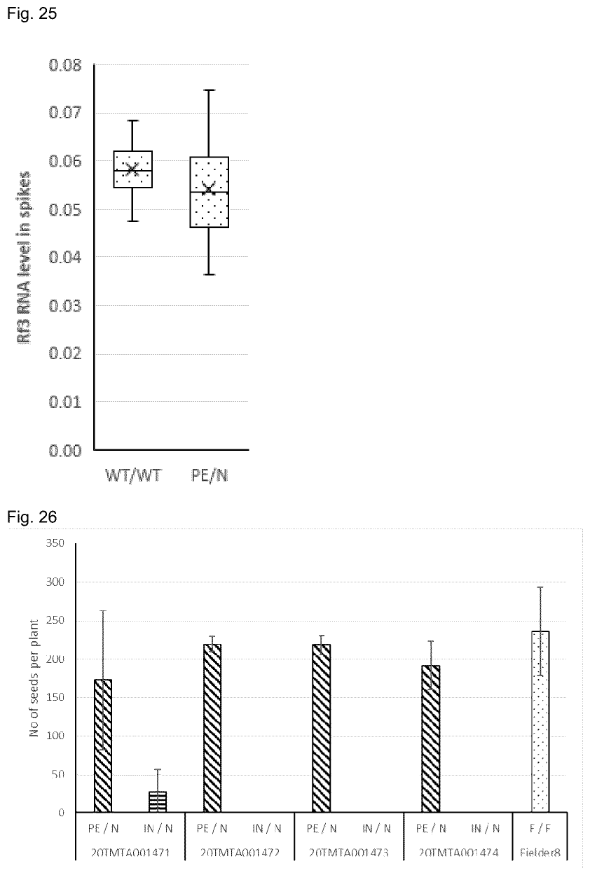

In a second genome editing experiment, the non-functional Fielder Rf3 gene was cut both in the promoter and in the CDS immediately downstream of the frameshift-causing deletion using pBas03682 (SEQ ID NO: 79) and pBas03683 (SEQ ID NO: 80) as gRNA expression vectors. Using pBas03913 (SEQ ID NO: 81) as repair DNA, the frameshift mutation in the Fielder Rf3 CDS was repaired and at the same time the EN1390 enhancer was inserted in the Fielder Rf3 promoter at the location that showed the biggest expression increase in the protoplast experiments. From these experiments, 1 event could be selected that contains 1 precisely edited allele (sequence shown as SEQ ID NO: 82, see also FIG. 7) and 1 indel allele (containing a 1-nt deletion at the cutting site in the CDS and a 2-nt deletion in the promoter, referred herein as “IN”) that is not expected to produce a functional Rf3 protein. 4 G0 plants from this event were analyzed for Rf3 RNA expression in leaves and showed a 20-fold increased expression level (see FIG. 23), showing that EN1390 insertion strongly increased Rf3 expression in leaves. These 4 G0 plants were crossed as male to Naxos plants (male sterile plants containing CMS cytoplasm and lacking known functional Rf genes) and G1 seeds were harvested. The resulting G1 plants were assessed for Rf3 expression levels in developing spike (samples consisting of 4 spikelets from the middle of a spike that is between 2 and 4 cm in length) and fertility restoration. The plants containing a precisely edited allele showed a clearly increased Rf3 expression in the developing spike (see FIG. 24). Taking into account that these plants contain only one precisely edited allele, this corresponds to a 2.2- to 2.5-fold increased expression of the edited allele in the developing spike. Such an expression increase was not observed in edited plants that had only the frameshift in the coding sequence repaired (see FIG. 25). The plants with the EN1390 insertion also showed an excellent seed set, with some of the seedlots having 219 seeds per plant compared to 236 seeds per plant for Fielder without CMS (see FIG. 26). This demonstrates that this edited Rf3 allele has a very high restoration activity and that insertion of EN1390 increases Rf3 promoter activity in the developing spike.

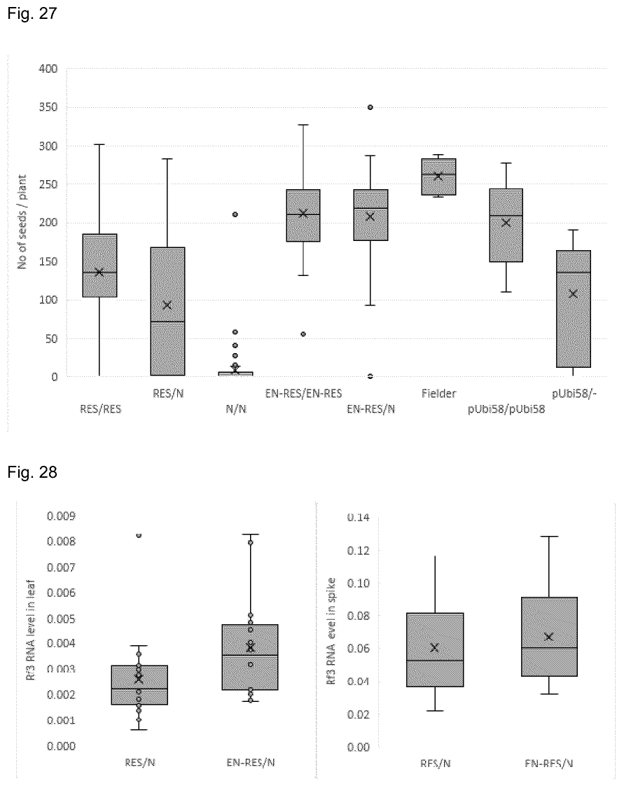

To determine the impact on fertility restoration of the insertion of EN1390 in the Rf3 promoter more accurately, G1S1 plants from edited lines in which the Fielder frameshift was repaired and EN1390 was inserted into the Fielder Rf3 promoter were grown side-by-side with G1S1 plants from edited lines in which only the Fielder frameshift was repaired. All plants contain the CMS cytoplasm and are segregating for the edited Rf3 locus. For both types of edits, 4 segregating seedlots were planted and seed set was determined following selfing for 5 plants per genotype (homozygous (“HH”) edited, hemizygous (“He”) edited, or wild-type (“WT”)) for each seedlot. Most plants that only have the non-functional Rf3 Naxos allele (N/N) show no or a low seedset, whereas plants that have the Fielder frameshift mutation repaired (RES) do have a good seed set (see FIG. 27), with the HH edited plants showing a higher average seedset (135 seeds/plant) than the He edited plants (92 seeds/plant). Most importantly, plants that also have the EN1390 insertion in the Rf3 promoter (“EN-RES”) do show a clearly higher seed set, with the He edited plants having a similar average seed set compared to HH edited plants (208 versus 212 seeds/plant). This level of seed set is comparable to that of homozygous transgenic plants that express an optimized Rf3 coding sequence under the control of the strong maize ubiquitin promoter (200 seeds/plant). In conclusion, these results show that a Rf3 allele that has the EN1390 enhancer inserted into the Rf3 promoter exhibits a clearly improved CMS restoration capacity, especially when the functional restorer is in a hemizygous state.

RNA expression analysis of the plants that have 1 precisely edited allele showed that the EN1390 insertion increased Rf3 expression in leaf by 50%, whereas the impact in developing spike was small (see FIG. 28).

### Example 13: Combination Example

The elements of the above Examples are also combined in a repair DNA (SEQ ID NO: 83) to create a repaired Fielder Rf3 gene with optimal restoration activity, the sequence of which (coding sequence and promoter region) is shown in FIG. 29 and SEQ ID NO: 84. The repaired Fielder gene with a sequence as shown in SEQ ID NO: 84 is modified to create a variant with the above three Rf expression improvement approaches combined (in this Example and in FIG. 29, the above-described duplicated PDH and EIL3 transcription factor sequence from Rf3-58 was used, which differs in some nucleotides from the native Fielder sequence (outside the PDH and EIL3 transcription factor binding sites as shown in FIG. 3), as well a variant thereof without the miRNA inactivation, or a variant thereof without the transcription factor binding region duplication (either missing the duplication of both above transcription factor binding sites, or of one of them), or a variant thereof without the EN1390 enhancer addition. Genome-edited wheat plants containing G-type CMS (so that this improved Rf3 gene replaces the existing native Rf3 gene in Fielder) are generated and tested for Rf gene expression in spike and restoration of G-type CMS.

### REFERENCES

Ahmed T A, Tsujimoto H, Sasakuma T. QTL analysis of fertility-restoration against cytoplasmic male sterility in wheat. Genes Genet Syst. 2001 February; 76(1):33-8. doi: 10.1266/ggs.76.33. PMID: 11376549.
Barkan, Alice, Margarita Rojas, Sota Fujii, Aaron Yap, Yee Seng Chong, Charles S. Bond, and Ian Small. 2012. ‘A Combinatorial Amino Acid Code for RNA Recognition by Pentatricopeptide Repeat Proteins’. Edited by Dan Voytas. PLoS Genetics 8 (8): e1002910. https://doi.org/10.1371/journal.pgen.1002910.
Barkan, Alice, and Ian Small. 2014. ‘Pentatricopeptide Repeat Proteins in Plants’. Annual Review of Plant Biology 65 (1): 415-42. https://doi.org/10.1146/annurev-arplant-050213-040159.
Consortium (IWGSC), The International Wheat Genome Sequencing, IWGSC RefSeq principal Investigators, Rudi Appels, Kellye Eversole, Catherine Feuillet, Beat Keller, Jane Rogers, et al. 2018. ‘Shifting the Limits in Wheat Research and Breeding Using a Fully Annotated Reference Genome’. Science 361 (6403): eaar7191. https://doi.org/10.1126/science.aar7191.
Dahan, Jennifer, and Hakim Mireau. 2013. ‘The Rf and Rf-like PPR in Higher Plants, a Fast-Evolving Subclass of PPR Genes’. RNA Biology 10 (9): 1469-76. https://doi.org/10.4161/rna.25568.
Espley et al., Multiple Repeats of a Promoter Segment Causes Transcription Factor Autoregulation in Red Apples, The Plant Cell, Volume 21, Issue 1, January 2009, Pages 168-183, https://doi.org/10.1105/tpc.108.059329
Fuji et al., 2011, PNAS 108(4), 1723-1728
Gupta P K, Balyan H S, Gahlaut V, Saripalli G, Pal B, Basnet B R, Joshi A K. Hybrid wheat: past, present and future. Theor Appl Genet. 2019 September; 132(9):2463-2483. doi: 10.1007/s00122-019-03397-y. Epub 2019 Jul. 18. PMID: 31321476.
Li H, Guo J, Zhang C, Zheng W, Song Y, Wang Y. Identification of Differentially Expressed miRNAs between a Wheat K-type Cytoplasmic Male Sterility Line and Its Near-Isogenic Restorer Line. Plant Cell Physiol. 2019 Jul. 1; 60(7):1604-1618. doi: 10.1093/pcp/pcz065. PMID: 31076750.
Manna, Sam. 2015. ‘An Overview of Pentatricopeptide Repeat Proteins and Their Applications’. Biochimie 113 (June): 93-99. World wide web at doi.org/10.1016/j.biochi.2015.04.004.
Ishida, Y., Tsunashima, M., Hiei, Y., and Komari, T. (2015). Wheat (Triticum aestivum L.) Transformation Using Immature Embryos. In Agrobacterium Protocols: Volume 1, K. Wang, ed. (New York, N Y: Springer New York), pp. 189-198.
Marand et al 2017; Biochimica and BioBiophysica Acta 1860(131-139)
Mao et al.: Variation in cis-regulation of a NAC transcription factor contributes to drought tolerance in wheat. Mol Plant. 2022 Feb. 7; 15(2):276-292. doi: 10.1016/j.molp.2021.11.007. Epub 2021 Nov. 15. PMID: 34793983.
Melonek et al., Nat Commun 12, 1036 (2021), https://doi.org/10.1038/s41467-021-21225-0 Thody, Joshua, Leighton Folkes, Zahara Medina-Calzada, Ping Xu, Tamas Dalmay, and Vincent Moulton. 2018. ‘PAREsnip2: A Tool for High-Throughput Prediction of Small RNA Targets from Degradome Sequencing Data Using Configurable Targeting Rules’. Nucleic Acids Research, July. https://doi.org/10.1093/nar/gky609.
Anzalone, Andrew V.; Randolph, Peyton B.; Davis, Jessie R.; Sousa, Alexander A.; Koblan, Luke W.; Levy, Jonathan M.; Chen, Peter J.; Wilson, Christopher; Newby, Gregory A.; Raguram, Aditya; (21 Oct. 2019). “Search-and-replace genome editing without double-strand breaks or donor DNA”. Nature. 576 (7785): 149-157
Shahinnia F, Geyer M, Block A, Mohler V, Hartl L. Identification of Rf9, a Gene Contributing to the Genetic Complexity of Fertility Restoration in Hybrid Wheat. Front Plant Sci. 2020 Dec. 10; 11:577475. doi: 10.3389/fpls.2020.577475. PMID: 33362809; PMCID: PMC7758405. Stojalowski, S., Bobrowska, A., Hanek, M. et al. The importance of chromosomes from the sixth homeologic group in the restoration of male fertility in winter triticale with Triticum timopheevii cytoplasm. J Appl Genetics 54, 179-184 (2013). https://doi.org/10.1007/si3353-013-0144-2
Sparks, C. A., Doherty, A., and Jones, H. D. (2014). Genetic Transformation of Wheat via Agrobacterium-Mediated DNA Delivery. In Cereal Genomics: Methods and Protocols, R. J. Henry, and A. Furtado, eds. (Totowa, N J: Humana Press), pp. 235-250.
Zhou, W., Kolb, F. L., Domier, L. L., and Wang, S. (2005). SSR markers associated with fertility restoration genes against Triticum timopheevii cytoplasm in Triticum aestivum. Euphytica 141, 33-40. doi: 10.1007/s10681-005-5067-5

1-95. (canceled)

96. A nucleic acid molecule encoding a functional restorer polypeptide for wheat G-type cytoplasmic male sterility, wherein said nucleic acid molecule comprises a mutated miRNA binding site in the coding sequence.

97. The nucleic acid molecule of claim 96, wherein:
i) the nucleic acid molecule is:
a) a mutated Rf3 gene which does not comprise a sequence as shown in SEQ ID NO: 45 (GGGUAGGUUGGAUGAUGCU) or SEQ ID NO: 46 (gggtaggttggatgatgct), or
b) a mutated Rf1 gene which does not comprise a sequence as shown in SEQ ID NO: 67 (gggucgguuggacgaugcu) or SEQ ID NO: 66 (gggtcggttggacgatgct);
ii) the functional restorer polypeptide comprises:
a) an amino acid sequence as shown in SEQ ID NO: 44, 63, or 65; or
b) an amino acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 44, 63, or 65;
iii) the nucleic acid molecule comprises:
a) at least one mutation in the nucleic acid sequence as shown in SEQ ID NO: 43; or
b) at least one mutation in a nucleic acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 43, wherein one or more nucleotide(s) at a position in the region corresponding to the region from nucleotide position 1245 to nucleotide position 1263 in SEQ ID NO: 43 are mutated;
iv) said miRNA binding site has been mutated in a translationally neutral or in a conservative manner;
v) the mutation of the miRNA binding site results in the formation of a lower number of base pairs formed between the binding site and miRNA 3619 as compared to the number of base pairs formed between the unmodified binding site and miRNA3619, for example, wherein less than 13 or less than 11 base pairs are formed;
vi) one or more nucleotides have been mutated in said miRNA binding site by substituting, deleting and/or adding one or more nucleotides at a position corresponding to a position in the region from nucleotide position 1245 to nucleotide position 1263 in SEQ ID NO: 43, such as wherein the one or more nucleotides have been substituted with one or more different nucleotides;
vii) 1, 2, 3, 4, 5, 6, 7, 8, 9, 10, 11, 12, 13, 14, 15, 16, 17, 18 or 19 nucleotides have been substituted with a different nucleotide;
viii) the nucleotide (or nucleotides) corresponding to position 1245, 1248, 1249, 1250, 1251, 1254, 1257, 1260, 1262 and/or 1263 in SEQ ID NO: 43 has (have) been substituted with a different nucleotide (or different nucleotides) in said mutated miRNA binding site;
ix) said miRNA binding site has been mutated by chemical mutagenesis, such as by EMS mutagenesis;
x) the coding sequence encoding said functional restorer polypeptide has less than 20 nucleotides mutated in the entire coding sequence outside the miRNA binding site; or
xi) the coding sequence encoding said functional restorer polypeptide has been mutated in the miRNA3619 binding site, but has not been codon-optimized over the entire coding sequence.

98. A wheat plant cell or wheat plant or seed thereof comprising the nucleic acid molecule of claim 97.

99. The wheat plant cell, plant or seed of claim 98, which is a hybrid wheat plant cell, plant or seed.

100. A method for producing a wheat plant cell or plant or seed thereof, comprising a functional restorer gene for wheat G-type cytoplasmic male sterility, or for increasing restoration capacity for wheat G-type cytoplasmic male sterility in a wheat plant, or for improving expression of a functional restorer gene for wheat G-type cytoplasmic male sterility in a wheat plant, comprising the steps of providing said plant cell or plant with the nucleic acid molecule of claim 97.

101. A method for producing hybrid cereal seed, comprising
a) providing a male cereal parent plant according to claim 100;
b) providing a female cereal parent plant that is a G-type cytoplasmic male sterile cereal plant;
c) crossing said female cereal parent plant with a said male cereal parent plant; and optionally
d) harvesting seeds.

102. A method for identifying and/or selecting a cereal plant comprising an improved functional restorer gene allele for wheat G-type cytoplasmic male sterility, comprising
a) identifying or detecting in said cereal plant the presence of the nucleic acid molecule of claim 97, or the mutated miRNA binding site of claim 97; and
b) selecting said cereal plant comprising said nucleic acid molecule.

103. The method claim 101, wherein the nucleic acid molecule is in present in homozygous form in said male cereal plant in a).

104. A method for increasing expression conferred by a wheat plant promoter of a functional restorer gene for wheat cytoplasmic male sterility, comprising:
a) introducing at least one nucleic acid expression enhancing nucleic acid (NEENA) molecule into said promoter, wherein said at least one NEENA molecule
i) comprises a nucleic acid sequence as shown in SEQ ID NO: 70, 86, 87, 90 or 91,
ii) comprises a nucleic acid sequence with an identity of at least 90%, at least 91%, at least 92%, at least 93%, at least 94%, at least 95%, at least 96%, at least 97%, at least 98%, or at least 99% to SEQ ID NO: 70, 86, 87, 90 or 91,
iii) comprises a fragment of at least 30, at least 40, in particular at least 50, at least 80, at least 100 or at least 120 consecutive bases of a nucleic acid molecule of i) or ii), or
iv) is the complement or reverse complement of any of the previously mentioned nucleic acid molecules under i) to iii),
wherein the nucleic acid molecule of ii), iii) and iv) is capable of increasing expression conferred by the plant promoter of the functional restorer gene for wheat cytoplasmic male sterility.

105. The method of claim 104, wherein
a) the at least one NEENA molecule
i) comprises a nucleic acid sequence as shown in SEQ ID NO 70,
ii) comprises a nucleic acid sequence with an identity of at least 90%, at least 91%, at least 92%, at least 93%, at least 94%, at least 95%, at least 96%, at least 97%, at least 98%, or at least 99% to SEQ ID NO: 70,
iii) comprises a fragment of at least 30, at least 40, in particular at least 50, at least 80, at least 100 or at least 120 consecutive bases of a nucleic acid molecule of i) or ii), or
iv) is the complement or reverse complement of any of the previously mentioned nucleic acid molecules under i) to iii),
wherein the nucleic acid molecule of ii), iii) and iv) is capable of increasing expression conferred by the plant promoter of the functional restorer gene for wheat cytoplasmic male sterility;
b) the promoter is a promoter of a functional restorer gene for wheat K-type cytoplasmic male sterility or for wheat G-type cytoplasmic male sterility;
c) the promoter is the promoter of an Rf3 or Rf1 gene;
d) the at least one NEENA molecule is introduced into the plant promoter by genome editing;
e) the at least one NEENA molecule is introduced at one or more positions within 1000 bp, such as within 500 bp or within 300 bp or within 200 to 100 bp upstream (5′) to the translation start codon of the gene that is operably linked to said promoter;
f) the at least one NEENA molecule is introduced at a position within 250 to 80 bp, within 200 to 100 bp, 110 to 150, 120 to 140, or within 125 to 135 bp, or within 125 to 130 bp, upstream (5′) to the translation start codon of the gene that is operably linked to said promoter; or
g) the resulting plant promoter has increased activity in developing spikes, such as in Zadok stages Z39-Z41, Z45-Z48, Z50-Z59, and/or Z60-Z69.

106. A promoter obtained or obtainable by the method of claim 104.

107. The promoter of claim 106, wherein the promoter is operably linked to:
a) a nucleic acid of interest encoding a functional restorer polypeptide for wheat cytoplasmic male sterility, and optionally
b) a transcription termination and polyadenylation region functional in plant cells.

108. A cereal plant cell or cereal plant or seed thereof, such as a wheat plant cell or plant or seed thereof, comprising the plant promoter of claim 106.

109. A cereal plant cell or cereal plant or seed thereof, such as a wheat plant cell or plant or seed thereof, comprising the plant promoter of claim 107.

110. A method for increasing expression conferred by a wheat plant promoter of a functional restorer gene for wheat cytoplasmic male sterility, comprising:
a) introducing at least one binding site for an Ethylene insensitive 3-like (“EIL3”) transcription factor and/or at least one binding site for a Plant Homeodomain (“PHD”) transcription factor into said promoter, and/or
b) modifying at least one existing binding site for the EIL3 transcription factor and/or at least one existing binding site for the PHD transcription factor in said promoter such that binding of the EIL3 or PHD transcription factor to said binding site is improved,
wherein the binding site of a) and/or b) increase expression in the presence of an EIL3 transcription factor and/or a PHD transcription factor.

111. The method of claim 110, wherein
a) the binding site for the PHD transcription factor has a sequence as shown in SEQ ID NO: 10, SEQ ID NO 11, SEQ ID NO: 40, SEQ ID NO: 31, SEQ ID NO: 32, SEQ ID NO: 38, SEQ ID NO: 42, SEQ ID NO: 41 or SEQ ID NO: 12, or is a variant thereof;
b) the binding site for the EIL3 transcription factor has a sequence as shown in SEQ ID NO: 19 or SEQ ID NO: 39, or is a variant thereof; or
c) the method of b) or c) above, wherein a fragment having a sequence as shown in SEQ ID NO: 29 is introduced into the promoter, or a sequence differing in 1-5, such as in 1, 2, 3, 4 or 5 nucleotides from the sequence of SEQ ID NO: 29, such as the sequence double underlined in FIG. 29.

112. A promoter obtained or obtainable by the method of claim 110.

113. The promoter of claim 112, wherein that promoter is operably linked to an Rf1 or Rf3 gene promoter expressing the Rf1 or Rf3 fertility restorer protein in wheat, comprising a heterologous or a duplicated EIL3 and/or PHD transcription factor binding site.

114. The method of claim 110, comprising producing a plant promoter having increased activity in the presence of an EIL3 (Ethylene insensitive 3-like) transcription factor and/or a PHD (Plant Homeodomain) transcription factor, and comprising:
a) providing a plant promoter, and
b1) introducing at least one binding site for the EIL3 transcription factor and/or at least one binding site for the PHD transcription factor into the plant promoter, and/or
b2) modifying at least one existing binding site for the EIL3 transcription factor and/or at least one existing binding site for the PHD transcription factor in the promoter such that binding of the EIL3 or PHD transcription factor to said binding site is improved.

115. The method of claim 114, wherein:
i) in step b1), at least one binding site for the EIL3 transcription factor and at least one binding site for said PHD transcription factor are introduced into the plant promoter;
ii) the plant promoter has increased activity in developing spikes, such as in Zadok stages Z39-Z41, Z45-Z48, Z50-Z59, and/or Z60-Z69;
iii) in step b1), the at least one binding site is introduced into the plant promoter by genome editing;
iv) in step b2), the at least one binding site is modified by chemical mutagenesis, by irradiation induced mutagenesis, or by somatic embryogenesis/mutagenesis;
v) the promoter provided in step a) is a promoter of a functional restorer gene for wheat K-type or G-type cytoplasmic male sterility;
vi) the promoter provided in step a) is the promoter of an Rf1 or Rf3 gene;
vii) the promoter provided in step a) comprises a sequence as shown in SEQ ID NO: 23, SEQ ID NO:36 or SEQ ID NO: 37, or the sequence upstream of the ATG translation start codon in SEQ ID NO: 94, or a variant thereof being at least 90% identical thereto;
viii) the binding site for the PHD transcription factor has a sequence as shown in SEQ ID NO: 10, SEQ ID NO 11, SEQ ID NO: 40, SEQ ID NO: 31, SEQ ID NO: 32, SEQ ID NO: 38, SEQ ID NO: 42, SEQ ID NO: 41 or SEQ ID NO: 12, or is a variant thereof with 1, 2 or 3 nucleotide substitutions;
ix) the binding site for the EIL3 transcription factor has a sequence as shown in SEQ ID NO: 19 or SEQ ID NO: 39, or is a variant thereof with 1, 2 or 3 nucleotide substitutions;
x) at least two EIL3 and/or PHD transcription factor binding sites are introduced into the promoter;
xi) a fragment having a sequence as shown in SEQ ID NO: 29 is introduced into the promoter provided in step a);
xii) the EIL3 transcription factor comprises:
a) an amino acid sequence as shown in SEQ ID NO: 13; or
b) an amino acid sequence being at least 50%, 60%, 70%, 75%, 80%, 85%; 86%; 87%; 88%; 89%; 90%; 91%; 92%; 93%; 94%; 95%; 96%; 97%; 98%; or 99% identical to SEQ ID NO: 13;
xiii) the PHD transcription factor comprises:
a) an amino acid sequence as shown in SEQ ID NO: 4; or
b) an amino acid sequence being at least 50%, 60%, 70%, 75%, 80%, 85%; 86%; 87%; 88%; 89%; 90%; 91%; 92%; 93%; 94%; 95%; 96%; 97%; 98%; or 99% identical to SEQ ID NO: 4.

116. A chimeric nucleic acid molecule comprising the promoter of claim 107, wherein the chimeric nucleic acid molecule comprises the following operably linked elements:
a.a nucleic acid molecule encoding a functional restorer polypeptide for wheat G-type cytoplasmic male sterility, wherein said nucleic acid molecule comprises a mutated miRNA binding site in the coding sequence; and optionally
b. a transcription termination and polyadenylation region functional in plant cells, wherein as to said nucleic acid molecule encoding a functional restorer polypeptide for wheat G-type cytoplasmic male sterility:
i) the nucleic acid molecule is:
a) a mutated Rf3 gene which does not comprise a sequence as shown in SEQ ID NO: (GGGUAGGUUGGAUGAUGCU) or SEQ ID NO: 46 (gggtaggttggatgatgct), or
b) a mutated Rf1 gene which does not comprise a sequence as shown in SEQ ID NO: 67 (gggucgguuggacgaugcu) or SEQ ID NO: 66 (gggtcggttggacgatgct);
ii) the functional restorer polypeptide comprises:
a) an amino acid sequence as shown in SEQ ID NO: 44, 63, or 65; or
b) an amino acid sequence being at least 70%, 75%, 80%, 85%; 86%; 87%; 88%; 89%;
90%; 91%; 92%; 93%; 94%; 95%; 96%; 97%; 98%; 99% or 99.5% identical to SEQ ID NO: 44, 63, or 65;
iii) the nucleic acid molecule comprises:
a) at least one mutation in the nucleic acid sequence as shown in SEQ ID NO: 43; or
b) at least one mutation in a nucleic acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 43, wherein one or more nucleotide(s) at a position in the region corresponding to the region from nucleotide position 1245 to nucleotide position 1263 in SEQ ID NO: 43 are mutated;
iv) said miRNA binding site has been mutated in a translationally neutral or in a conservative manner;
v) the mutation of the miRNA binding site results in the formation of a lower number of base pairs formed between the binding site and miRNA 3619 as compared to the number of base pairs formed between the unmodified binding site and miRNA3619, for example, wherein less than 13 or less than 11 base pairs are formed;
vi) one or more nucleotides have been mutated in said miRNA binding site by substituting, deleting and/or adding one or more nucleotides at a position corresponding to a position in the region from nucleotide position 1245 to nucleotide position 1263 in SEQ ID NO: 43, such as wherein the one or more nucleotides have been substituted with one or more different nucleotides;
vii) 1, 2, 3, 4, 5, 6, 7, 8, 9, 10, 11, 12, 13, 14, 15, 16, 17, 18 or 19 nucleotides have been substituted with a different nucleotide;
viii) the nucleotide (or nucleotides) corresponding to position 1245, 1248, 1249, 1250, 1251, 1254, 1257, 1260, 1262 and/or 1263 in SEQ ID NO: 43 has (have) been substituted with a different nucleotide (or different nucleotides) in said mutated miRNA binding site;
ix) said miRNA binding site has been mutated by chemical mutagenesis, such as by EMS mutagenesis;
x) the coding sequence encoding said functional restorer polypeptide has less than 20 nucleotides mutated in the entire coding sequence outside the miRNA binding site;
xi) the coding sequence encoding said functional restorer polypeptide has been mutated in the miRNA3619 binding site, but has not been codon-optimized over the entire coding sequence.

117. A chimeric nucleic acid molecule comprising the promoter of claim 113, wherein the chimeric nucleic acid molecule comprises the following operably linked elements:
a. a nucleic acid molecule encoding a functional restorer polypeptide for wheat G-type cytoplasmic male sterility, wherein said nucleic acid molecule comprises a mutated miRNA binding site in the coding sequence; and optionally
b. a transcription termination and polyadenylation region functional in plant cells, wherein as to said nucleic acid molecule encoding a functional restorer polypeptide for wheat G-type cytoplasmic male sterility:
i) the nucleic acid molecule is:
a)
a mutated Rf3 gene which does not
comprise a sequence as shown in
SEQ ID NO: 45
(GGGUAGGUUGGAUGAUGCU)
or
SEQ ID NO: 46
(gggtaggttggatgatgct),
or
b)
a mutated Rf1 gene which does not
comprise a sequence as shown in
SEQ ID NO: 67
(gggucgguuggacgaugcu)
or
SEQ ID NO: 66
(gggtcggttggacgatgct);
ii) the functional restorer polypeptide comprises:
a) an amino acid sequence as shown in SEQ ID NO: 44, 63, or 65; or
b) an amino acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 44, 63, or 65;
iii) the nucleic acid molecule comprises:
a) at least one mutation in the nucleic acid sequence as shown in SEQ ID NO: 43; or
b) at least one mutation in a nucleic acid sequence being at least 70%, 75%, 80%, 85%; 86%; 87%; 88%; 89%; 90%; 91%; 92%; 93%; 94%; 95%; 96%; 97%; 98%; 99% or 99.5% identical to SEQ ID NO: 43, wherein one or more nucleotide(s) at a position in the region corresponding to the region from nucleotide position 1245 to nucleotide position 1263 in SEQ ID NO: 43 are mutated;
iv) said miRNA binding site has been mutated in a translationally neutral or in a conservative manner;
v) the mutation of the miRNA binding site results in the formation of a lower number of base pairs formed between the binding site and miRNA 3619 as compared to the number of base pairs formed between the unmodified binding site and miRNA3619, for example, wherein less than 13 or less than 11 base pairs are formed;
vi) one or more nucleotides have been mutated in said miRNA binding site by substituting, deleting and/or adding one or more nucleotides at a position corresponding to a position in the region from nucleotide position 1245 to nucleotide position 1263 in SEQ ID NO: 43, such as wherein the one or more nucleotides have been substituted with one or more different nucleotides;
vii) 1, 2, 3, 4, 5, 6, 7, 8, 9, 10, 11, 12, 13, 14, 15, 16, 17, 18 or 19 nucleotides have been substituted with a different nucleotide;
viii) the nucleotide (or nucleotides) corresponding to position 1245, 1248, 1249, 1250, 1251, 1254, 1257, 1260, 1262 and/or 1263 in SEQ ID NO: 43 has (have) been substituted with a different nucleotide (or different nucleotides) in said mutated miRNA binding site;
ix) said miRNA binding site has been mutated by chemical mutagenesis, such as by EMS mutagenesis;
x) the coding sequence encoding said functional restorer polypeptide has less than 20 nucleotides mutated in the entire coding sequence outside the miRNA binding site; or
xi) the coding sequence encoding said functional restorer polypeptide has been mutated in the miRNA3619 binding site, but has not been codon-optimized over the entire coding sequence.
